SEQUENCE LISTING (390 sequences)

SEQ ID NO: 1 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 2 - protein, 5 aa, Homo sapiens
  Site (3): Xaa equals any of the twenty naturally ocurring L-amino acids
WSXWS

SEQ ID NO: 3 - DNA, 86 bp, Homo sapiens
gcgcctcgagatttccccgaaatctagatttccccgaaatgatttccccgaaatgatttccccgaaatatctgccatctcaattag

SEQ ID NO: 4 - DNA, 27 bp, Homo sapiens
gcggcaagctttttgcaaagcctaggc

SEQ ID NO: 5 - DNA, 271 bp, Homo sapiens
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 6 - DNA, 32 bp, Homo sapiens
gcgctcgagggatgacagcgatagaaccccgg

SEQ ID NO: 7 - DNA, 31 bp, Homo sapiens
gcgaagcttcgcgactccccggatccgcctc

SEQ ID NO: 8 - DNA, 12 bp, Homo sapiens
ggggactttccc

SEQ ID NO: 9 - DNA, 73 bp, Homo sapiens
gcggcctcgaggggactttcccggggactttccggggactttccgggactttccatcctgccatctcaattag

SEQ ID NO: 10 - DNA, 256 bp, Homo sapiens
ctcgaggggactttcccggggactttccggggactttccgggactttccatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 11 - DNA, 552 bp, Homo sapiens
  SITE (186): n equals a,t,g, or c
ggcacgagcttgttcttatgggctttatatgtcatctatatgttgatgaaaataaatttctatcccttgcccaagcctaaacttcatactagcatatccaactgcctactggacatctccatttataagcctagtagcctaataagcataacctcagacttaccaggcctcacactgaagtcatgnaacttcagcccaacccccatgccagggcaaaaccttgttgttacctcttattcctctcttgcctcatcccatccatgttcagtctgtcagtggatcctgtgagtccagtcttgaggatagttccaggatctgatcacttctcactgcctcttttgctgccaccacctctggcctggataattgcagcagcctcccagttagccttgctgtgtccatccttgttttccccttctgtctgctctcaacagaggagctagtgattctcttaggacagaataaatcatttaggttttcttcacatggtcctgaagaagcttcctacctcactcagtgtaaaaaccaaaaaaaaaaaaaaaaaaaactcga

SEQ ID NO: 12 - DNA, 1434 bp, Homo sapiens
cattaaactctttttatcgggaatagtatgatattttcaatgtcactccattcatgttgatttggagctgacagttattttgtgtaagcagagatttaattttatattgaaagtcagtgcaaaattatgaataggatatactaataaatacaaagtaataacaaaagtcaaagcagtgttctaaataaaaattctgggttccttaaaaattattttaaatttatcttgaaatagttttcttagattaatctcaggatatgagaaagtcaattaagtgtgagtaaagttagtatcattaaacaaattgtctattaaatgcamgagtggtaatatacagaatttatcaggcattaccaagtctaggcacatataggaaatgcagcactcagaatggtttcaatgtagtagttgatgcttgtaaggtaggggagcttattcagacatagtagatagtttctctaatgctgtstcaattgctggcctttggctacctgtacttccscattatggcagcccattcagtcttgagttttcttctctggacaccttatgctctgaaatcatgagcgaggctgattcaattggtgatttgggtagaaagcagtatgttttgctgacattaagatgtaggttatagataggtttagcctttaagtgtatgtttttatactttaaaataagaaatataaccttttaagctattccacctcctcccccagcctatctcaaactggtggaatatatggagagatcttgaaagaagtaaaataaaccttcactgctccactccaggtgaatccgcccactcccactgacctagtagaatttgtaatttaatacttaccttctatttctgaaatcagttgtgaactgttgccttatgttcagargtttaagaacctcmgtgaattcattttttaaaatctgctattctgagaagcattgaatgaattcttaacaagaagactcatctgtagctgtttgctgactcctatgagccccataagggttctgtgcttagcattaacaaaataaggtttataggtaaagccaatgtattaatttttttttgcatggagggctttaaaatttgtgctctttttcatattttattcatattcaatttatggtttgtaactgctttttagggagataattatatgttataaattagttttggggggaataattgtgcaaagaggataatttaatttacgtgcttctgttattcagaataaagagagaagactacgctgcatattcaagagttgtaccttaacattggtgaaacattttttctaagattttcaaaaggaatatgtgtaaattgagaaatcataaccactgtcctaacttggtaaacaaactgttcttaaataaagtatttaatgattttaaaaaaaaaaaaaaaaaaaaaaaaaaaaaaaaaaaaaa

SEQ ID NO: 13 - DNA, 1881 bp, Homo sapiens
  SITE (70): n equals a,t,g, or c
atttttccttttccttaacaatacctttggccatttttttccagttcactatgtttgtatactaactttncttcagccttttaatgcgaagcaactagtagagcatgctttcaggatctgacagcnctgctagtagagcgaagtatttattaatacagaattaaccttmgcccctttaaagtcaagtctgtctaatctaactagcgcctcgctttgccttctcacaatgctcactagccatcatgctcacccttctcttccagatccacttcctcatgatactgtcttctaactgggcttacttaaaggatgcgagcaaaatgcaggcttaccaggatatcaaagcaaaggaagaacaggaactgcaagatatccagtctcggtcaaaagaacaactcaattcttacacataaatgtttgccagagtgtttcggccgacgtatttacagctctgacaaatcatcagacagctgctctgcagtacagatgtgtatcccaccaaactaatgtagatgtacaaacacttcactgtctgtctcaagctgctgggatgtatctctaggaaaaccttccagtgggtaaatctttttctttagaacaaatattggaggtttcatgttagccattttaaaaggcaacactttgacaaaatgatcgttcatactttgggaatttgtggcatgttcacatttattgctagggcaattctaccaagacactcaatggaatatgtcacactccttaatagggacctgtgactccttaataaggacctgtgacatgcccagcatcaagggataagaccgtaaattcacatatatgccatctgtcctcaagtgttatctacataggaaataaaatggaattgatgtaaagttccatttctgacagctgacatttattaaactttggatcaaagataatgtgattcttatgattgatttctcaaactagcttttccctcccaagtccaggacccattaatttcctgagccaatcagaaatatatttttcaataatgctaaaattagctacaattctgctgaccctactattaaagaatctggatgctggactcactgacaagctttccagaagcaattttataacagatttcattttaacaaaatactgatccaattttcattattcttgagaaatgtcagctttgccttaatgagtatttgctttaaatttctaagaatttatatcataactagagacccaaatatctttcacagaattttgttccataaatgtttttcttaattattaagaagtgttaccttattaaaatgaccaccattctaaaccatttttcagtggtctggatacgaagtttacagtttcataccaactatctaaaacctaattgcaaattgaccacagacctctaacctcctacttttatagacttgaatacttaagtaatttaaattagggttggtatttcatttttttcttatctaaatcttagtttcctggaataataaagtttgatgttcagcaagagaactgcttgagtttaagccattttcaaaagaaacttgccttttacattattgtgttccagaacattaagtgactgtaggtactgggtattagtgatggtaaactttgtgttgctctttatgaaatgatccatataactgttgggtgcatcagtgcttttcaaaggggctgcttactatagggttaactatgtatattcattgttaagagttaacttgtggtttggctgttycctggattttataacatacatgtgcagaaatgtattcaaatgaaaggaagcatacctttatcaagatgctattaaaattgaacatcaagtataaaaaaaaaaaaaaaaaaaaattnctgcggccgacaagggaattc

SEQ ID NO: 14 - DNA, 1060 bp, Homo sapiens
gaattcggcacgagggtggaggacaaccgtttacctccrccccgctggaaatcctgttctttctgaacgggtggtataatgctacctatttcctgctggaacttttcatatttctgtataaaggtgtcctgctaccatatccaacagctaacctagtactggatgtggtgatgctcctcctttatcttggaattgaagtaattcgcctgttttttggtacaaagggaaacctctgccagcgaaagatgccactcagtattagcgtggccttgaccttcccatctgccatgatggcctcctattacctgctgctgcagacctacgtactccgcctggaagccatcatgaatggcatcttgctcttcttctgtggctcagagcttttacttgaggtgctcaccttggctgctttctccagtatggacasgatttgaagtacagaatttcagccagcagcccatcaggctgacaccacacatattgcttctggtactttagccacaccagtgagaattggtggggcaagttgtcctgagaaaggctgtgtggcttttcttcagcacagacatttgggcaagcaactcagcataaggccagtgggtaccatcttctaaaccaggaccatcagcccaagagactcttctacactccagtatagggaggggcaaggttattcccatcctgccccttctcagaaccagtcccctgctgacctcaagttctcctccttgatcaccgtggccagagcatctcgtgtggaccatctaggctccttgggcttcaagcaggacctgagccacatgctccctgtacgagctgtgctatacctgtcccacatgagcacggagagcctcatgttggtgggtttccagagtgatgtgaaagcctctcaccccaatcctcggagactgagttccacaacttttttagtagctcatagtgttatttttctactctcttcatgaaactaactttattttataataaatatrtattttctgttgtgggggaaaaaaaaaaaaaaaaacttcgagggggggcaccggtacccaatcgaccc

SEQ ID NO: 15 - DNA, 1255 bp, Homo sapiens
ttcccaactttctgccacacttaaattacgttcctccatttcagttttgtcttttctgtctaaagttcagtcaaagagtatcaaaaaattatgtttcagctagactggtgtaatgtataagtttttgtatcttgtattagaggatttcgtagcttttattagaggctcatttccacctcagcatacaagatcgttagtcttttggcatgtgtgccaattagaatactaaagcaagtccaagcacatttttctcttctcacgtttctaataagtgttagggactttgcctcttttacttaccacgtccccaaaagtgtcaggtagacatgtcacaaatggctctgtagagagccatgggaagagagaggaggtggatgtggaacataaagggttcagaaactccagaagaggagtgggttttggatagaagcatttgaggacagctgctccaaagccttatgtgtatgatgaaacttaaccacggggaagagactcttcagtagcctgttctgtctggtgatttttattttaagtgaacctttggatctatctttaactctctttattgtgagtctaaattccaattctgcagcagatcagtaaactcacagtatttttcctgtggaaatctattcaataaggaaaccaagacaggataataaaatttaaaaaaaaaacaactttgaattcccctgcctaggtcttccagttgttttccagcacatacctcaggtatgactttgctagcyggggacaaaattagcaccttccgawtctctagtccaaatgaactttgtgctaaataaaaaattattatactacataataaagttacagayagcaggaaatgcaagagctaggagattcctagattatatctgccaagcaaataccttaaacatccacctgaaatcctactaccccctcttctgagataatttgcccagcccttctcttcccacacactcactcaatgtcacccccttctaatccccaaaactgtttttgtggtctttgtagcctatagtagttttctcacatctttccccctagacttttctgtttttcagtttcagacaaaaaaactcttcagctttttccagtgtgtctccttaacagtaactttaccacttgaaatcttatttcatagaaaaactaaattggtgtggaaaggctgcacacaataaagttatattattatccatgaaaaaaaaaaaaaaaaaaaaaaaaaaaaaaaaaaaaaaaaaaaactcga

SEQ ID NO: 16 - DNA, 1036 bp, Homo sapiens
gcgcgtaatacgactcactsatagggcgaattggagctccaccgcggtggcggccgctctagaactagtggatcccccgggctgcaggaattcggcacgagtgaagtactgcgtggtgtatgataacaacagcagcaccctggagatactcttaaaagatgatgatgatgattcagactctgatggtgatggcaaagatcttgtgcctcaagcagccattgagtatggcaggatcctgacccgcctcacccaccaccccgtctacatcytgaaagggggctatgagcgcttctcaggcacgtaccactttctccggacccagaagatcatctggatgcctcaggaactggatgcatttcagccataccccattgaaattgtgccagggaaggtcttcgttggcaatttcagtcaagcctgtgaccccaagattcagaaggacttgaaaatcaaagcccatgtcaatgtctccatggatacagggcccttttttgcaggcgatgctgacaagcttctgcacatccggatagaagattccccggaagcccagattcttcccttcttacgccacatgtgtcacttcattgaaattcaccatcaccttggctctgtcattctgatcttttccacccagggtatcagccgcagttgtgccgccatcatagcctacctcatgcatagtaacgagcagaccttgcagaggtcctgggcctatgtcaagaagtgcaaaaacaacatgtgtccaaatcggggattggtgagccagctgctggaatgggagaagactatccttggagattccatcacaaacatcatggatccgctctactgatcttctccgaggcccaccgaagggtactgaagagcctcacctgggggcattttgtgggtggagggccagagtgtgtatacccaggcttgtctggaaggagaaggcctttgctgcctgaaagtcwmaaaaaaaaaaaaaaaaackcgagggggggcccggtacccagcttttgttccctttagtgagggttaatttgcgcgtccg

SEQ ID NO: 17 - DNA, 1014 bp, Homo sapiens
gaattcggcacgagtttacatcagaaaagagctggaagtcttcctgcaatgaaggagaatcctcttctacttcttatatgcatcaraggtcacctggtggtcccaccaaactgattgagatcatctcagactgcaactgggaggaagatcggaacaagattttragcatcttatcccagcacatcaatagcaacatgccacaatcacttaaggtgggcagcttcatcattgagttggcttctcagcgaaagagccggggtgagaagaaccctcctgtttattcttctcgtgtgamaatctctatgccatcatgtcaagaccaagatgatatggctgagaaatctggatcagagactcctgatggtccattgtcccctgggaaaatggaggatatctctcctgtgcagacagatgccctggattcagtgagggagagattacatggaggcaaaggtctgcctttttatgcagggctttctcctgcagggaagcttgtggcctataaacgtaaacccagttcaagtacatctgggcttatccaggtgagaattatctttaatctgggtatagcacctttgtatacacctaggtagtatcatgatttttcagagccctttatggtcctgatatcctttatcttgacatttcctgggaactgggtgacaaaattattatctctttttgtaataggcctagtttagatgcatacctagagtgaatttttgtcacatttatgaacagaaacgtagagccttgtattagttttaattttctttctaatcttcccagaaagttgctcttcataaactttattgcctgcaggctctagtgatactttgacaataaagcaagggtaatcagggattcagtctagctcttggaatttattattagcagataggtttcaaaacaaaaccatggttagaacggtaggtgtaaggggaagatgaaattgacttaaagataggcaatatatgtttagaaacttggggaaaaaaaaaaaaaaaaactcga

SEQ ID NO: 18 - DNA, 1287 bp, Homo sapiens
  SITE (1282): n equals a,t,g, or c
gaattcggcacgagatttactaaaatgatgtaataaataacatgttaatagactcaagctttaccttatgaaattgatgtatttttaccagttatttctaatgtaacattgaatatataagatctgacaaatgtatgtttaaacatgaattagaagagttgagaactaccattatgtatagggattctcatagtgtcttggcccttaattggaaagttgtggcaactttaaagtactttttactgtatgttataattctttataacttagagagagacaatggtcactcaaactatgagaactatgaattaggagataaaagtttaaatttgttgttgttttataacagtatgtacaagttagttttcccttatatatttacgttttcaagttttttaatctcatcatatacatccatactctataaaatgttttatattcaaagaactgtaaaatcctaaacattagttttcactattgaaattgttttttaaagataggcataaatagttgtccttagacttattcatacaaatatagtcatttacttctatgtagtttgagattctgagagttattccaactttatgaagattgatttcaatgtgcctgctaagtcctaaaagattcagaaagaaaatttatatattattgatttaaatatcatcctttaaatatgttgtataacattcaatatagtttatgtatcagtgattgtattttattctgaatgcatgatctcaagccttaactactataatctttttctgcccctcagaaattgaataacctaaccaagatgcctttaggggatgccctaagtaaatgtaatttcagatttcagggttttttttttttcctctctaagtgttccttccctttcttctcctgctctccatcatgttatggagaccagtgaggaaccagtgttaacttggtgacaatgtgacagctggtgctttatctaagctccgttttctatttcttgggaatgctttattgtggaaactgcttcagatacttaaattgaatcataacttgcttctgtaaattgcgtaaagacaacaaactgattttagtttgaaaagtttatcttttacttgtaaaccttgtttgccagttaccttccgaaagctgtgtaaagagttatttttaacaaagtcttaacaatatatgttactttttagatactatagaaaataataaatataacctgtaaaccacaaaaaaaaaaaaaaaaaaactcgagggggggcccggtacccaatcgcsgwgtgatggngctat

SEQ ID NO: 19 - DNA, 1105 bp, Homo sapiens
gaattcggcacgagtggcaacacaagcacctagctcagagatcttgaagaatgaaatgagattatgtaaataacaacttaccacagtgcttggcacatagtaagtgctcaatgtcagctatgattattattattcccttcttaacacacaaagaaggaggggatccaaaaataacagtgtgccacagtttgaaaggcatttatttgatcttgtctctaaatttccattttacatgtagcacttacccggtggaagtgaaaatacagtgaacgctaaaaagccctgtgtctctcggtggtgtctggacaaccctggcaactcggaacatgaaggagagaacaagaattccctgtgcttttccttttcttcttttccaaacacgtgtgcagacttcccctgcatttcagccccaccctctttattttactgcctaatctataaaggaggattaacagcagcacgctgctttggcatagagcagattctgggtgaggacctgtaggtagagtttaatgaatacaattttctaggactgtgagtgcatatttttagctccatgctgggcttcagcgttggctcttgagacagatgaacagactctttgatcagacttgggtgttgctccaagaagaacttttctcagaaagtcgttaggaaaaaaaattgtcttctgttgcccttattcctaatgtgcactctatagattcagattccagataacttgtcctgatctcagtaaattaattgcattgcaacattgagttacaccactgtggaaagaaaaagtacttctgggcaggaacagatccactttctcacaaaagagaatggctggtgttcaagtgtgtggttgccatcctttcccttttgagagtagggtagaggtagttaaccttcctgggggaggtttggcctagacaacatcatagacactatatcccccctggagttaccaaacaataaaactgcttcctttgccaaacacaaagaatggtctggagttggatattagcaaacagcaaaccacataaagaagacaaaaaaaaaaaaaaaaaaaaaactcgagggggggcccgtacccaatcgcctgtgatgtatcgtatac

SEQ ID NO: 20 - DNA, 1089 bp, Homo sapiens
gaattcggcacgaggagaagatcgctcacaagagtttgaacataagctggaccacaaaggatagagtaaatgtggaaagatggaaaagaaaaaaagaaacctacaaacaccagatatgtagcccaaaagcccagcttctataacttgttcatggctaccgtacatagaagcacccaggactgcaatcccttttgtatacaagtttcttttctttctgagccaagtcaagaaacctgaaaactataaggcaggaaaaaagaagaagattaagrttatccatgatttcatcactcgggatgaccagtgttattgtactatttatcttaaaagtgtttttcaaatatttttctacaacatcatttttaaatgcttgcatacattttatacataaatgtaaactagttaactaattcctctattgctggaattttaagatgtctctaaatgatataaacaatatttcaaattttgtgattgggaatgtggattctagaatatgagtgtcaaggtccaagatttgtctccactgtttgttaggtgaattgcataaactctataaactcagtttcctactttaaaaaacagaagtgtgtcagtgacagtggtgtatgcctgtagtcctagctattctagaggcagaggggagaggatcacttgagtccaggagtttaaagctgtagtgtgccatgatctcacctgtgaatagccactgcactccagcctagacaacacagtgagacctcatctctaaaaaagaaaatagggggctaggcgtggtgttacgcctgtaatcccagcactttgggaggctgaggcaggtggatcacgtggtcaggagttcgagaccagcctggccaacatggtgaaaccccgtctctaccaaaaatacaaaaattagctgggtgtggaggtgcatgcctataatcccagctactcaggaggctgaggcaggagaatcgcttgaacccgggaggcggtggttgcagtgagcgaagatagtgccattgcactccagcctgggtgacagggtgagactctgtctcaaaaaaaaaaaaaaaaaaaaaaaaaaaaaaaaaaaaaaaactcgta

SEQ ID NO: 21 - DNA, 2831 bp, Homo sapiens
  SITE (182): n equals a,t,g, or c
gggtttcctcagacagtgttggaggattcagatacagtgaaagatatgatcctgagcccaaatcaaaatgggatgaggagtgggataaaaacaagagtgcttttccattcagtgataaattaggtgagctgagtgataaaattggaagcacaattgatgacaccatcagcaagttccggagnaagatagagaagactctccagaaagatgcagcgacanaatkgaggaaaagaaagcgagaagaggcagatctcccaaaggtgaattcaaagatgaagaggagactgtgacgacaaagcatattcatatcacacaggccacagagaccaccacaaccagacacaagcgcacagcaaatccttccaaaaccattgatcttggagcagcagcacattacacaggggacaaagcaagtccagatcagaatgcttcaacccacacacctcagtcttcagttaagacttcagtgcctagcagcaagtcatctggtgaccttgttgatctgtttgatggcaccagccagtgcaacaggaggwtcagctgatttattcggaggatttgctgactttggctcagctgctgcatcaggcagtttcccttcccaagtaacagcaacaagtgggaatggagactttggtgactggagtgccttcaaccaagccccatcaggccctgttgcttccagtggcgagttctttggcagtgcctcacagccagcggtagaacttgttagtggctcacaatcagctctaggcccacctcctgctgcctcaaattcttcagacctgtttgatcttatgggctcgtcccaggcaaccatgacatcttcccagagtatgaatttctctatgatgagcactaacactgtgggacttggtttgcctatgtcaagatcacagcctttgcaaaatgttagcacagtgctgcagaagcctaatcctctctataatcagaatacagatatggtccagaaatcagtcagcaaaaccttgccctctacttggtctgaccccagtgtaaacatcagcctagacaacttactacctggtatgcagccttccaaaccccagcagccatcactgaatacaatgattcagcaacagaatatgcagcagcctatgaatgtgatgactcaaagttttggagctgtgaacctcagttctccatcgaacatgcttcctgtccggccccaaactaatgctttgatagggggacccatgcctatgagcatgcccaatgtgatgactggcaccatgggaatggcccctcttggaaatactccgatgatgaaccagagcatgatgggcatgaacatgaacatagggatgtccgctgctgggatgggcttgacaggcacaatgggaatgggcatgcccaacatagccatgacttctggaactgtgcaacccaagcaagatgcctttgcaaatttcgccaattttagcaaataagagattgtaaaagaagcagattgaatgaagaatttttagctgtgcagataggtgatgttgggatggaaaatgctaatcaactaccctttcttttatcaagtaattaaaataaatctacataaagaaccaaaaaggctgttttataaaagtgaaatatccagtatttcagagggccaggcaagagcacttcagatgaggcagtcaaaatcatttttttccrgtgaggatagaccacaagtgggtggtgagaccattgaaagcctttatcaactgaagagtccatttaacagcataatttgtgggaagactggaatagggctgaataaatgtgtttgaatctctaattttatactttcttttcctgaggaacttgatttttctgtccctggatcgccttgtcataattgggtctgttccttttactaccactcttgagtccatatatgaaatcattaaagttggatgatcagttttttataaaaatatatatttttgtccaagaaaaaaaaaagcatacatatgtgattatggctaaatcaaaggtaactggaatgtatatacttttgctaatgttccagcaacactgctattatactatccaaatttttattgtaacaaaacctctttaagcaattggtgattgccatgggacttttcccatgtcttctgctgtaattatcctgtgcagaactaggaagaaatttttttcaggactgctctatggtttcctttaaaagaaaaaaacttctgtttgtttttagcagtcattatttacaatttgcagtgattaacttggcaaggcttccttccgtgtttatccctgtagccatcatttaagtcaggaacagtcagaaaaatatttattttattttttttttgggtgtctgcaaaggtaaaaatccattaaaaccttaagttaaatataaatgttacaactcaatgtttgcttttagattttatacagtatttgttttgttttggttttgagtgtatataatgcagcattagcaatatggttccaatagaggagttaaatatatattgttaaaggagacctgtagcagtcaaagattttattgatttaatgacaaaggaaattaatgaaaatgtttttgtttttctgctgtaattctgcattaagctcacatgaaaatcaygattctagagtttggaatgcaaaattaattgttttaccctcaagctgggaatatttttcaaaataaatactataatatagatatcaaattattacctccccatgttatgttgaaaatttttttattaaattgataaaactttatttccattatattcataatgttctgttatacataacattaaaatgttcattaaaaaaaaaaaaaaaaaactcgagactag

SEQ ID NO: 22 - DNA, 1448 bp, Homo sapiens
  SITE (1422): n equals a,t,g, or c
gaattcggcacgagcaactgccctgatcaccccccgtcccagcccttgagtgaacgtccttctgagcggcttcctggggtcctccccacgtcccaaaggccggcaagatggtgtcctggatgatctgtcgcctggtggtgctggtgtttgggatgctgtgtccagcttatgcttcctataaggctgtgaagaccaagaacattcgtgaatatgtgcggtggatgatgtactggattgtttttgcactcttcatggcagcagagatcgttacagacatttttatctcctggttccctttctactatgagatcaagatggccttcgtgctgtggctgctctcaccctacaccaagggcgccagctgctttaccgcaagtttgtccacccgtccctgtcccgccatgagaaggagatcgacgcgtacatcgtgcaggccaaggagcgcagctacgagaccgtgctcagcttcgggaagcggggcctcaacattgccgcctccgctgctgtgcaggctgccaccaakagtcagggggcgctggccggcaggctgcggagcttctccatgcaggacctgcgctccatctctgacgcacctgcccctgcctaccatgaccccctctacctggaggaccaggtgtcccaccggaggccacccattgggtaccgggccgggggcctgcaggacagcgacaccgaggatgagtgttggtcagatactgaggcagtcccccgggcgccagcccggccccgagagaarcccctaatccgcagccagagcctgcgtgtggtcaagargaagccaccggtgcgggarggcacctcgcgctccctgaaggttcggacgargaaaaagactgtgccctcagacgtggacagctagggtctgctgcatctgcccccttcttacctcgtgccctgcakggctccagggctatttggagggaccttgggctgcacatctggcctgcctgcaccagctgcctgggcyccaccctcctgactcctgctgatggttaagggccgggagcagatgctgccaaggccacatgcagggatgcacccacaatgtaccaaagcaggctgggcccagggttctatttattgccttgctctgccctctcccttccccggttgtgggacaagagccctccctgaacccctgcaaccctccctgaacccctgcaaatgaaaccaaacgtccacctgggtgtgttcattccttcctgtccttcaaagtacttgatagcctttcataaggcctggcacatgtgtcctggttgtgtgtgtgtgtgttggtgagtgaggtcaggtttgcgagtgttttgataaataaatacataaaggggcaaaaaaaaaaaaaaaaaaaaaaaaacaaaaaaaaaaaaaaaaaaaaaaaaaaaaaaaaaaaaaaaaaaaaaaaanaaaaaaaaaaanaaaaaaaaaaaggg

SEQ ID NO: 23 - DNA, 1211 bp, Homo sapiens
  SITE (131): n equals a,t,g, or c
agagaaagtggagacggacctgagcccgagggagaggcaggcagaggctgaggctgattccaccccagcctgcctgggacaaccctccttagccgcagccccttccagttccctgaggggttctgcccctnccccctctctgggggcaccaaccccccagggtcctgcatcccaccatgtcgatggctgtggaaacctttggcttcttcatggcaactktggggctgctgatgctgggggtgactctgccaaacagctactggcgagtgtccactgtgcacgggaacgtcatcaccaccaacaccatcttcgagaacctctggtttagctgtgccaccgactccctgggcgtctacaactgctgggagttcccgtccatgctggccctctctgggtatattcaggcctgccgggcactcatgatcaccgccatcctcctgggcttcctcggcctcttgctargcatakcgggcctgcgctgcaccaacattgggggcctggagctctccaggaaagccaagctggcggccamcgcaggggccctccacattctggccggtatctgcgggatggtggcmatctcctggtacgcttcaacatcacccgggacttcttcgaccccttgtaccccggaaccaagtacgagytgggccccgscctctacctggggtggagcgcctcactgwtctccatcctgggtggcctctgcctctgctccgcctgctgctgcggctctgacgargaccagccgccagcgcccggcggscctaccargctcccgtgtccgtgatgcccgtcgccacctcggaccaagaaggcgacagcagctttggcaaatacggcagaaacgcctacgtgtagcarctctggcccgtgggscccgctgtcttcccactgccccaaggarargggacntggccggggcccattcccctatagtaacctcaggggccggccacgccccgctcccgtagccccgccccggccacggccccgtgtcttgcactctcatggcccctccaggccaagaamtgctcttgggaagtcgcatatctcccctctgaggctggatccctcatcttctgaccctgggttctgggctgtgmaggggacggtgtccccgcacgtttgtattgtgtataaatacattcattaataaatgcatattgtgaccgttaaaaaaaaaaaaaaaaaaaaaaaaaaaaactcga

SEQ ID NO: 24 - DNA, 1060 bp, Homo sapiens
  SITE (453): n equals a,t,g, or c
gccacttcttccaaatacagtagatgtgtctgctgtgtatttatacaacatcctgaactacttaacatgctgtttatttacttgtttgtattccccattagaataggctctgagaaagcaaagactgtatctgtcttgcttatcattgtatccctgacagctcgcccactggctggcttttaataagcacaccataaatatttacttgaaatactcatttttaaaatgaacagatgaatgaatgatagatggatggtggatggcattatgtagctaaaaattgtgtcctgtctctacctatttttgaagaccatcctttagtttgcgtttcctgccatgtttgaggggcctttttttggtccataactcttgtcttttattcaaattaaaacaccgaacaaaagcacattcgattattgrccatgrggttttttattcygctgtcagtgtcanccycmtgtctaaatccccyggggtcaaacttacatatatctggatagcccttttkgatgacgatggtagtctaatttgtgtgttatgtgctcttgaaatgttttgctgtaaagacactagaactgaattttgctttattgccaatgatgatgaatgttaaaaaaaacaactcagtaacattcaaaccaatttccaagtttgttcttcagccagaggaacttgcacactgactttttgtaaaggtagcagatttattgtgttgtaattcatacaccataaaattcaccattttaaagtttccaatttagtggtttttagtatgtttacagagtcatgcaaccatcaccacagtatcattgcaggatgtttttatcatccctcaaagaaatccagacccacaggaggctgaggcaggagaatcgcttgaacccggaaggcggaggtttcagtgagtccgagatagcgccactgcactccagcctagtgacagagcaagactctgtctcaaaaaaaaaaaaaaaaaaaaaaaaaaaaaaaaaaaaaaaaaaaaaaaaaactcgaagggggggcccggtacccaatcgtccctatnatgagtcgtattaca

SEQ ID NO: 25 - DNA, 1057 bp, Homo sapiens
  SITE (348): n equals a,t,g, or c
gaattcggcacgagcggcacgagattttaggtaaatgacgaagggaatgtggtgaatgtcactgtccagagccataaatcagacaaaaccatacatagcatgctgaaaaacttttgtaatggaacacccaacaaatgacacctaacctgtctgtgatccaacaagtccgataacatgctgctgtatttgtattctctgggaatctcagtattaataatttcatttcccacaaattctagcattcatgtaaggaaaaacatggctaatcaatatcttaaaggggcaatctttcagagcagtggttttcaaagtgtggccggacagcattggcagcatcttaatctcctngggactttgttaaaaatgcaaattctcagccccaccctagtcctactgaattgggaaactggcgtgggacccagcagtctttgttttaacatgttctccaagtgattctgatgcctgttcaaacttgggaaacacttttagagcacttgaggaacctaaaagatgactggttcagcattttgtgtggtagataagaaagaaattatcacaaaaaatcagaaatgaacagtgagagaaaaataggaccccagacagtttataccttccatttgctgttttaaaagtgtgagcctgccaagtcaacaagtatgcctttagcgcacatgtaaatagcctgcacttcctaaatctcgtgtggcctcccatggttacattcttcaaaggtwaactgagttgagaggaagattcagcatttaaaagagaagggttgaaaaagattgtgtgtgtgtgtgtgtgtgtgtttaattggcccagggttacttaaataaatcataaccattttgccacattctgtaactgtttagctaaggtcaaattaagtttaccctatggattttgtttcatcttttgtttcgtgtatatactgtttgcctttttcataaaaatcttggatttgttatatattgttcctgttatttttgacatctttgctattgtaaataaattactattttgttttaagttaaaaaaaaaaaaaaaaaaacwcgta

SEQ ID NO: 26 - DNA, 980 bp, Homo sapiens
tcgacccacgcgtccgcggcgcgctcacaatggagctctcggagtctgtgcagaaaggcttccagatgctggcggatccccgctccttcgactccaacgccttcacgcttctcctccgggcggcattccagagtctgctggacgcccaggcggacgaggccgtgttagatcatccagacttgaaacatatcgacccagtggttttaaaacattgtcatgcagcagctgcaacttacatactagaggcaggaaagcaccgagctgacaagtcaactctaagcacttatctagaagactgtaaatttgacagagagcgaatagaactgttttgcacggaatatcagaataataagaattccctagaaatcctactgggaagtataggcagatctctccctcatataacggatgtttcttggcgcttggaatatcagataaagaccaatcaacttcataggatgtacagacctgcatatttggtgaccttaagtgtacagaacactgattccccatcctatccagagattagttttagttgcagcatggaacaattacaggacttggtggggaaacttaaagatgcttcgaaaagcctggaaagagcaactcagttgtaacttggggaagttaacgatccgcccgagtgcagaggaaaaccagaaacgccttgccttcagctgaaccaccgtttgtgcgagctggatgtccttttcagtagaaaagaattttccttttgaatttataccattcatcaattttgacactttaaaaacgtgtgaaagggttaagagggaaagatactgcccaagtatttgaatcgtttagtagtaactgtccatttatcctattttgatctttttcaagtcttctgaaaggaagtagacagtattacaccctgaataaataaggtgttgttttccacaaaaaaaaaaaaaaaaaaaaaaaaaaaaaaaaaaaaaaaaaaaaaaaaaagggcggcc

SEQ ID NO: 27 - DNA, 755 bp, Homo sapiens
  SITE (748): n equals a,t,g, or c
gaattcggcacgagattgtgcacatgtaccctaaaacttaagatgtaataataataaaataaaataaaataaattaaaaaataaaaataaaaacaratttaatgatagggtacttaatgaaagtwttggtggtccttgaatgacgtattttacactacatatgtacctacttttctattctcctcctcagatgggaaaggtctagataaactggcctctatcccgcagctcttctccacaatggttaagaacagttcaacacggaggaccagcagtaaatgacctttaaaaagtgtaataataactattgcccaaaataatcttattaatcatagaaaatggcttctattcttctgctccttgttctgtcacacagctgttgctgtaaaaacacttgtttacaggttctatgtaattttgactcagtccataatctctccaccctaattttaaaaattatcatcagggtggatgtgctagtatactaagaaacatctgttaatattatttattttctttatttaatctttttcatagattcacttgttttaaaatatcttaggtttataatctctttgcaaagctcaataaatcattttaacagctaaaaataaaaacttaaaaatgaactccagataaatatgaagattcaaaactatgtggaatctctgcccccctcttaatactcaccaataaattctacttcctgtcmaaaaaaaaaaaaaaaaaaaaaaaaaaaaaaaaaaanaaaaaaa

SEQ ID NO: 28 - DNA, 946 bp, Homo sapiens
  SITE (5): n equals a,t,g, or c
tcgcnactatagggaactggtcnctgcaggtccggtcggaattccgggtcgacccacgcgtccggtaaatgttttatgtgttcgcctactgatcccattcgttgcttctattgtaaatatttgtcatttgtatttattatctctgtgttttccccctaaggcataaaatggtttactgtgttcatttgaacccatttactgatctctgttgtatatttttcatgccactgctttgttttctcctcagaagtcgggtagatagcatttctatcccatccctcacgttattggaagcatgcaacagtatttattgctcagggtcttctgcttaaaactgaggaaggtccacattcctgcaagcattgattgagacatttgcacaatctaaaatgtaagcaaagtagtcattaaaaatacaccctctacttgggctttatactgcatacaaatttactcatgagccttcctttgaggaaggatgtggatctccaaataaagatttagtgtttattttgagctctgcatcttaacaagatgatctgaacacctctcctttgtatcaataaatagccctgttattctgaagtgagaggaccaagtatagtaaaatgctgacatctaaaactaaataaatagaaaacaccaggccagaactatagtcatactcacacaaagggagaaatttaaactcgaaccaagcaaaaggcttcacggaaatagcatggaaaaacaatgcttccagtggscacttcctaaggaggaacaaccccgtctgatctcagaattggcaccacgtgagcttgctaagtgataatatctgtttctactacggatttaggcaacaggacctgtacattgtcacattgcattatttttcttcaagcgttaataaaagttttaaataaaaaaaaaaaaaaaaaaaaaaaaaaaaaaaaaaagggcggcc

SEQ ID NO: 29 - DNA, 971 bp, Homo sapiens
gcttctatccatttattcaagcacatattggtcacctactgtgtgcctggcactcatgtcacaaagataagttcctgattcggtacacttactgagcacctgctgtgtgcagggagctgagctatgggatgggaatgggagtaaacaaggtacttttyacttttttctttttttcctcactgctagacggtgtgggaacttctcactcattggcttctttcccacacacctgaagagcactgactgtgtgccgggcactagtgatacaaaagagtgtgacagttgttcagtctgcattttcgatcatgggctacatgccgagtgctggggcacagagatgaacaagatcggttccttcacttcttcatgccacaagtgtttattgagcacctgtgtgccaggcctcacagactcccagttgggttgaagaatggttgactgagtttgattcttcctgtaccctcggtcgtctgagctgtgtgcagacaacatccccccaccacccaagagggagggtagctcttccgccaccaggggcaagcacaggtcctggtggccccacgccacatgttagcccccctggagggggcgccagttggagacgggggctgggtgtccctggcccactcccggtcccctgtgctttacctccttgcccttgtgtctcaggtgtggtccctgcctgcttgatgaagttgctctgttcaagcctttggtgggatcatgtgtttgggggcttttaggggacccagctgcactggggcactgcccgtggcctgggtaggacatttcccagcaagggctggaggagttgccgtgccttcagcctgaatcgaatgtcagaaccagccagcggtgcttcaccctcttggggataacttgcttagttttttaataaatgttcctggttggttttcacagcaaaaaaaaaaaaaaaaaaaaaaaaaaaaaaaaaaaaaaaaaaaaaaaag

SEQ ID NO: 30 - DNA, 1008 bp, Homo sapiens
  SITE (421): n equals a,t,g, or c
gcggcacgagctggaggtcactttccaaccagagctgtgctggagtccagtaggtgggaggctgtgctttgagggactaaaggaagcctgtatcttgtggtgagggttccacctcacaagttacagatctcagttccatttggctctagcagcaatgtggccacttctgttgcggttactctttcttcacctttttcttgccaaaaataaacttatctttaaatgaaaactaaattatttcttatattttggtcctttgttatagctgagattgggaatttttctttctttcttgaatccttacttccctaccctgcctccccaccaatggaaatctgtgcttcataagcattttagattccagaaagctctttaggttaaactacaaccctctcacctcaaagaatttgtgggccagggnaagtcagtgacttatgtgaagtcttgcggctaattaatggtagagctggagttaggacacatgtctcacagttcctagttcgttttgctttgatgtgcttgaaattcagttttgacattaatttttctggatactactcccataaaatgttctttgaaaaatacttgcttctttctagtttttctcgcctggtttaaatattgtcctgagtgtgggaaccccataactgtcttgtgggttagaatttagatggaaggatttggggccctgtctctagtatcataagacatttaaccttgctgcttttttcttctaggttcactctttgaatttcctggataagagttctggagatggcagcttattggacacatggattttcttcagatttgcacttactgctagctctgctttttatgcaggagaaaagcccagagttcactgtgtgtcagaacaactttctaacaaacatttattaatccagcctctgcctttcattaaatgtaaccttttgccttccaaattaaagaactccatgccactcctcaaaaaaaaaaaaaaaaaaaaaaaaaaaaaaaaaaaaaaaa

SEQ ID NO: 31 - DNA, 990 bp, Homo sapiens
aattcggcacgagtggacaaccatcagggagccaggacacagaggggcagagcaagtcagcattggcgccccttcctcagatccctatcatcttgggaaacagtagcccagaggttcaggaagatgttaacttaaatgttcggggtgccccagtctgttcagcatggctgaaatccacactccgtattcttccttgaagaaactgttatctttactcaatggcttcgtggctgtgtctggcatcatcctagttggcctgggcattggtggtaaatgtggaggggcctctctgacgaatgtcctcgggctgtcctccgcatacctccttcacgttggcaacctgtgcctggtgatgggatgcatcasggtactgcttggctgtgccgggtggtatggagcgactaaagagagcagaggcacgytcttgtttgttggagatgtggccttggaacacamcttcgtgaccctgaggaagaattacagaggttacaacgagccagacgactattctacacagtggaacttggtcatggagaagctaaagtgctgtggggtgaataactacacagatttttctggctcttccttcgaaatgacaacgggccacacytaccccaggagttgctgtaaatccatcggaagtgtgtcctgtgacggacgcgatgtgtctccaaacgtcatccaccagaagggctgtttccataaactcctaaaaatcaccaagactcagagcttcaccctgagtgggagctctctgggagctgcagtgatacagttgccaggaattcttgccactttgctgctgtttatcaagctgggctgacacccaggcctggagaagatgagacacctgggcccatctggctgctggagattcagtctcagttttatttctctgtggcactcactgcttctggaggggagactgttaataaaagatttgggaaaaaaaaaaaaaaaaaaaaaaaaaaaaaaaaaaaaaaaactcga

SEQ ID NO: 32 - DNA, 1131 bp, Homo sapiens
gaattcggcacgaggcctatgtcatcctggctgtgtgcttggggggaatgatcgggatctctgccagcttctcagccctcctggagcagatcctctgtgcaagcggccactccagtgggttttccggcctctgtggcgctctcttcatcacgtttgggatcctgggggcactggctctcggcccctatgtggaccggaccaagcacttcactgaggccaccaagattggcctgtgcctgttctctctggcctgcgtgccctttgccctggtgtcccagctgcagggacagacccttgccctggctgccacctgctcgctgctcgggctgtttggcttctcggtgggccccgtggccatggagttggcggtcgagtgttccttccccgtgggggagggggctgccacaggcatgatctttgtgctggggcaggccgagggaatactcatcatgctggcaatgacggcactgactgtgcgacgytcggagccgtccttgtccacctgccagcagggggaggatccacttgactggacagtgtctctgctgctgatggccggcctgtgcaccttcttcagctgcatcctggcggtcttcttccacaccccataccggcgcctgcaggccgagtctggggagcccccctccacccgtaacgccgtgggcggcgcagactcagggccgggtgtggaccgagggggagcaggaagggctggggtcctggggcccagcacggcgactccggagtgcacggcgaggggggcctcgctagaggaccccagagggcccgggagcccccacccagcctgccaccgagcgactccccgtgcgcaaggcccagcagccaccgacgcgccctcccgccccggcagactcgcaggcagggtccaagcgtccaggtttattgacccggctgggtctcactcctccttctcctccccgtgggtgatcacgtagctgagcgccttgtagtccaggttgcccgccacatcgatggaggcgaactggaacatctggtccacctgcgggcgggggcgaaagggctccttgcgggctccgggagcgaattacaagcgcgcacctgcagcggccccgggtgtggtttctgcgccgcgggagggggagctgtgcc

SEQ ID NO: 33 - DNA, 1293 bp, Homo sapiens
  SITE (1): n equals a,t,g, or c
naaggannccaaaccgcagaaagtnacccgtcacgtaaagggaacaaaagcctggaggtagcgcgcctgcaggtcgacactagtggatccaaagaattcggcacgagaccaaccccaagtgctcctatatccctccctgtaagagagaaaatcagaagaatttggaaagtgtcatgaattggcaacagtactggaaagatgagattggttcccagccatttacttgctattttaatcaacatcaaagaccagatgatgtgcttctgcatcgcactcatgatgagattgtcctcctgcattgcttcctctggcccctggtgacatttgtggtgggcgttctcattgtggtcctgaccatctgtgccaagagcttggcggtcaaggcggaagccatgnaagaagcgcaagttctcttaaaggggaaggaggcttgtagaaagcaaagtacagaagctgtactcatcggcacgcgtccacctgcggaacctgtgtttcctggcgcaggagatggacagggccacgacagggctctgagaggctcatccctcagtggcaacagaaacaggcacaactggaagacttggaacctcaaagcttgtattccatctgctgtagcaatggctaaagggtcaagatcttagctgtatggagtaactatttcagaaaaccctataagaagttcattttctttcaaaagtaacagtatattatttgtacagtgtagtatacaaaccattatgatttatgctacttaaaaatattaaaatagagtggtctgtgttattttctatttccttttttatgcttagaacaccagggtttaaaaaaaaaaaaaargtgaggacatctgggtctcatttgcttctgctaggttaaacttttacttgacaacaaggattcctgctgaagtctgaaccttactgtgtaaccctcagtttccactattaaagagtatcttttgacgtctgcttggaaaatgaatagtatactggtaactcagtctccagtcacctctgtgtctcttaagcaagagattctaaaagattgggaaaacatatcctccaamacctgcctttgcctaaccattatttttcaccagattacttcttaagagagggaggtgattctgaagaaggcttctatctcaaaaagcactgggcttccttattcatctgttcttgttgtttttgacggagttaaaaaagtttgtgtgcaatacaatataaatgatgtgaaggacactcttaaaaaaaaaaaaaaaaaaaaaaatngctgcggccgacaagggaattc

SEQ ID NO: 34 - DNA, 1014 bp, Homo sapiens
ggcacgaggtcagccagaacatgtctttcaacctgcaatcatcaaagaaactgttcattttcttaggaaaatcactgtttagtcttctggaggctatgatttttgccttactcccaaagccacggaagaacgttgctggtgaaatagtcctcatcacaggtgctggaagtggactcggaaggctcttagccttgcagtttgcccggctgggatctgttcttgttctctgggatatcaataaggaggggaatgaggaaacatgtaagatggctcgggaagctggagccacaagagtgcacgcctatacctgcgattgcagccaaaaggaaggagtgtatagagtagccgaccaggttaaaaaagaagtcggcgatgtttccatcctaatcaacaatgccggaatcgtaacaggcaaaaagttccttgactgtccagatgagcttatggaaaagtcatttgatgtgaatttcaaagcacatttatggacttataaagcctttctacctgctatgattgctaatgaccatggacatttggtttgcatttcaagttcagctggattaagtggagtaaatgggctggcagattactgtgcaagtaaatttgcagcctttgggtttgctgaatctgtatttgtagaaacatttgtccaaaaacaaaaggggatcaaaaccacgattgtgtgccccttttttataaaaactggaatgtttgaaggttgtactacaggctgtccttctctgttgccaattctggaaccaaaatatgcagttgaaaaaatagtagaagctattctacaagaaaaaatgtacttgtatatgccaaaagttgttatacttcatgatgtttcttaaaaaggtaattacatcagcttctattacttccctaacatgccagtctacagttttactcccaaatcccacccaggaaaaagccactttwaaaaatacctgataaattaaaattcattaatttaattctaaaaaaaaaaaaaaaaaaaaaaaaaaaaaaaaa

SEQ ID NO: 35 - DNA, 1222 bp, Homo sapiens
  SITE (4): n equals a,t,g, or c
actnatcttgaggtgacactatgagaaggtacgcctgcaggtaccgatccgnaattcccgggtcgacccacgcgtccngaaatttacaatttctgaccatccacaacctattgatccactgttaaagaactgcataggtgatttcctaaaaactttggaagacccagatttgaatgtgagaagagtagccttggtcacatttaattcagcagcacataacaagccatcattaataagggatctattggatactgttcttccacatctttacaatgaaacaaaagttagaaaggagcttataagagaggtagaaatgggtccatttaaacatacggttgatgatggtctggatattagaaaggcagcatttgagtgtatgtacacacttctagacagttgtcttgatagacttgatatctttgaatttctaaatcatgttgaagatggtttgaaggaccattatgatattaagatgctgacatttttaatgttggtgagactgtctaccctttgtccaagtgcagtactgcagaggttggaccgacttgttgagccattacgtgcaacatgtacaactaaggtaaaggcaaactcagtaaagcaggagtttgaaaaacaagatgaattaaagcgatctgccatgagagcagtagcagcactactaaccattccagaagcagagaagagtccactgatgagtgaattccagtcacagatcagttctaaccctgagctggcggctatctttgaaagtatccagaaagattcatcatctactaacttggaatcaatggacactagttagatgtttgttcaccatggggaccattacatatgaccatacaatgcactgaattgacaggttaatcataagacatggaaagagaagtgtctaaaagcttcaaaatgttccacttttttttccttcatggagactgtttgtttggctttcttccattgttgtttttgtagcatttatttcagaaatgtgtatttccataatccagaggttgtaaaaccactagtgttttagtggttacagcaacatttgaaatggaaactaaaagttaggattttatggagtatggagatagggtccagtatctatttaccctgtaatgtttaggattaaaatgttaaaattttgtgaccatgaatttctttcttttataaattttctcatttaaaaatcaaaaaaaaaaaaaaaaaaaaaaaaactcga

SEQ ID NO: 36 - DNA, 901 bp, Homo sapiens
  SITE (895): n equals a,t,g, or c
gaattcggcacgagcacttgagaggtgtacaggagagagttaatctttctgcacctctgctacctaaagaagacccaatcttcacatatttatctaaacggttaggaaggagtatagatgacataggtcacctcattcatgaaggcctacagaagaacacttcctcgtgggtactgtataacatggcttcattttactggagaattaagaatgagccatatcaggtagtagaatgtgccatgcgagcacttcacttctcttccaggcacaataaagacattgccctggtcaacctggcaaacgttctacacagagcacacttctctgctgatgctgctgtcgtggtccatgcagctctggatgacagtgacttcttcaccagctattacactttggggaatatatatgcaatgcttggggaatataaccactcagtgctctgttatgaccacgctttgcaggccagacctgggtttgagcaagctataaagaggaagcatgctgtcctatgtcagcaaaaactggagcagaaattggaggctcagcatagatctctccagcgaacactgaatgagttaaaagagtatcaaaagcagcatgaccactacctgagaccaggaaatcctagaaaaacataaactgattcaggaggagcaaatcttaagaaatatcattcatgagactcagatggcaaaagargcacaattaggaaatcatcagatatgccgactggtcaaccagcagcatagtttacattgccagtgggamcagcctgtwcgctatcatcgtggagatatctttgaaaatgtggactatgttcargtctttttcttggtccartctaattcttataaacgtttgctttataaagattttttaaaactttaaaaaaacngcacga

SEQ ID NO: 37 - DNA, 954 bp, Homo sapiens
gaattcggcacgagcccacaccaaacctgtggacgccgacccgggaccgccgctggctggctgctggctcactcgaccgtcatggagaccctgggggcccttctggtgctggagtttctgctcctctccccggtggaggcccagcaggccacggagcatcgcctgaagccgtggctggtgggcctggctgcggtagtcggcttcctgttcatcgtctatttggtcttgctggccaaccgcctctggtgttccaaggccagggctgaggacgaggaggagaccacgttcagaatggagtccaacctataccaggaccagagtgaagacaagagagagaagaaagaggccaaggagaaagaagagaagaggaagaaggagaaaaagacagcaaaggaaggagagagcaacttgggactggatctggaggaaaaagagcccggagaccatgagagagcaaagagcacagtcatgtgaagattcctggctgcctcttccaggcagtcccccagagatgcctcttctgccccctaaaagcagtgccctggacttgaagcccgtgaaatgactccatctgggattcagaatacagtgttctcaagtgaagaaggcttggaacccaccccacctccctcattgggggctctctgggcaaacatggttttcatgcacccctcttcctgagcttggtccctgcctggtgattcttcttatactcggagagcatccctggttgaggagacacccgcaatcctccacgatctcatggctccacctgcttctccccactgcctgatttcttttctctctgcctgatgtctactgaacagaacttcccctctcccatgcacccactgccagctgagagctgcttcccaatggcctgcattaaagcattcgtaacagccaaaaaaaaaaaaaaaaaaaaaaaaaaaaaaaaaaaaaaaaaaaactcga

SEQ ID NO: 38 - DNA, 890 bp, Homo sapiens
aattcggcacgagattcactaaacactgcaatacaagcttggcaacagaacaaatgccctgaggtagaggagttggtcttcagccattttgtgatctgtaatgacacacaggagacactgcggtttggccaggtggatactgatgaaaatattctgctggcgagtctccacagtcaccagtacagctggcgctctcacaaatccccacagctgttacacatctgtattgaaggttggggcaactggcgttggtcagagcctttcagtgtggaccatgccgggacttttattagaacaattcagtacaggggtcgaactgcttctctcatcatcaaggttcagcaactcaatggagtacaaaaacagattatcatctgtggaagacagatcatctgtagttacttgtctcaaagcatagaactaaaagtcgttcagcattacattggtcaagatggacaagctgtagttcgggaacattttgactgcctcacagccaaacagaaattgccttcgtacatactagaaaacaatgaactgacggagctgtgtgtgaaggccaaaggagatgaagactggtcaagagatgtgtgcctggaatccaaagcccctgagtacagcattgtcattcaggtgccatcttcaaacagttccattatttatgtctggtgcacagttttgactttagaacccaactctcaagtgcaacaacgaatgattgtgttcagccctctttttatcatgaggagtcatcttccagaccccattatcatacatttggagaaaaggagtctgggattgagtgaaacacaaattattccaggaaaagggcaggaaaaaccactgcaaaacatagaacctgaccttgtacatcacctgacattccaagcaag

SEQ ID NO: 39 - DNA, 1070 bp, Homo sapiens
  SITE (1016): n equals a,t,g, or c
acagcctttgttaccttccgagccacccgaaaacctctagtacagacaaccccaaggttggtttataagtggttcctgctaatctataaaatcagctatgccactggcattgttggctacatggctgtcatgtttaccctctttggtcttaacttattattcaagatcaaaccagaagatgccatggactttggcatctcccttctcttctatggcctctactatggagttctggaacgggactttgcagaaatgtgtgcagactacatggcatctaccatarggttctasagcgagtcgggcatgcctaccaaacatctttcagacagtktgtgtgctktktgtgggcagcagatctttgtggacgtcatgaagaggggatcattgagaacacgtataggctgtcctgcaatcatgtcttccacgagttctgcatccgtggctggtgcatcgtgggaaagaagcaaacgtgtccctactgcaaagagaaggtagacctcaagaggatgttcagcaatccctgggagaggcctcacgtcatgtatgggcaactgctggactggcttcgatacttggtagcctggcagcctgtcatcattggtgtagtccaaggcatcaactacatcctgggcctggaatagtgatgaagagcatcagtggaaaacccaccccacacgccatggacctcagggcactctcctccctgcccacaaagacctcctgggtgggaaagactcaaaggggcgcttgggccactcaggacccctccggctgtgtcggactggggagggatatgatggagagccagccagtggggctgkcagcagtggggggctttttaaaagaaaactattttgatgaatatatttaaaaaacctttttttattgtggagcataggaattgcccccctccaggcttcaccctccctgcctaagcaggttgggggcagagccatgacatttttggtttaaaggagccttctcatctctggccgagaacactgctgggctcccangtagctgaangcctcagcccayccatncccttcttccctgtgtggggctcaagc

SEQ ID NO: 40 - DNA, 772 bp, Homo sapiens
gcaaccagtatgaaaaggctttctcatccaagtatctgcagaactggtctcccactaagccaacaaaagagagcatctcttctcatgaaggctacactcaaattattgccaacgatcgtggtcatctactgccttctgtgccccgttccaaggcaaatccttggggttccttcatgggcacctggcaaatgcctctgaagataccccctgctcgggtgaccctgacctcccgtacaactgctggtgctgcctccctcaccaaatggatacagaaaaatcctgatttactccaaggcctccaatgggctgtgtcctgaaatcttaggcaagccccatgatccagacagtcagaagaaactcagaaagaagtctatcacaaagactgtacaacaagcacgaagtccaaccatattccaagctccccagctgccaacctcaattccccagatgaactccaaagctcacamccctctgcaggtcatactccaggtccccaaagaccagccaaatyctaagagcccacctggragtccacgtatgctagaactctgggcagggcctaatctagctgaggtccagaaatacaaacctggaacttcatatggaccaagtggccacacactgaaaaacccgtatagcgactcagtgaaataaacaagagcccccagtcagaactgtgaaacagggaaattttggggtggsagtaaaagsaaatttggaaaataaacttttttttgttgaatcttttaaaaaaaaaaaaaaaaaactcgta

SEQ ID NO: 41 - DNA, 787 bp, Homo sapiens
  SITE (444): n equals a,t,g, or c
ggtggtgtgcgccacccagaggctctctgtgggtccctagtggggaaaatgactcctccccacctacagtcttggtcagcagccccactgagctgtgttcatgttgacttccagctccaaccttatctcctgggtcctgccagagttgtcctctctgttgtgggttttcttgttctggaaaaggcagtgtggtgactgggcgggccggaagaccaggtccagggtctcaggagttgtcactaatttcccactccattccccttcactccgttacagctcctttttggaatgaggggacgatgctcaggaagagaggaggtattggaaaggaaagagaccccttcatcttcctttttagccctgctcaacctggctggctatttctgggagggccctttagagttgctgtgggcctctgcctatgtctgtgcagggcataggcantgcacasacagttgccacacccagggtggamaaatccccatggtggccttgtctgctgtcanttgcataggaaatctgataacctaagatttttttttattttttattttgagacagagtcttgctctgtcccccaagttggagtgcaatggcatgatcttggctcactgctacctccaatcctggatttgagctactcaggaggctgaggtcaggggaatcgctggaacgcgggaggcggagcttgcagtgagccgagatcatgtcactgccctccagcctgggcgacacagtgagactccatctcaaaaaaaaaaacaaaaaaaaaaaaaaaaaactcgta

SEQ ID NO: 42 - DNA, 652 bp, Homo sapiens
  SITE (392): n equals a,t,g, or c
aattcggcacaggggggccaccacacccggcctgtacatgctgttttgcatcttgctttatacgttggggagtgccagatgtcaccatctttcgttcttcctctggggctggtcaaatccccctgagaaaactcctctggcctcctggcggggggtgaaggccaggctgccagggccaggctgccagcttctgggagctgcaggggcagaggcagggagctgtcaggcattcagccagcaagacgcactcagtacccacttggggttcagaatccccctccctcatcttcagatgggccagatgtccccaaagccagcggcccctttctgtttcaccctgtctacagaataaacccccagtcactgggggtgggggaagagtaaggggagangggaaacgagatttggaggtctagctgctgctgaaacagccctcagttcgtctttattttgccttctgcaaaactggcctggtgttgccagctccttttgaggactttgctamcggttctcagcatccctcaattgctggcttaggattcatgggtttttaggggtggggtgggattagcatgtccagctgctttccagtttccaaagttctgtccctatcatattgcctctgatttaaaaaaaaaaaaaaaaaactcga

SEQ ID NO: 43 - DNA, 1520 bp, Homo sapiens
  SITE (799): n equals a,t,g, or c
gaattcggcacgagtcacccttttcagtgagttagtcgtgacatttcttacactgtgagggggagtggtaattactttacagggaggtatggggccatggtgtttgactcttctttcaaccacttctgggttttttagtgaaaacctctatctaacactgatactttcatttctgttgtctattgagtcagttaacactgatccatttatttttcagttcccaaaatcttgctttgccattgcttctattttattgtctgggggtgtttaacacctgtttgcattttttacagtcatttagtttccagattttagtaagggacagagggaatagatggactcattcatgatgtagaaacaaatactccctgtcttgtcttacakgaaaaattattcttaaactagcctgtcttkgagaacctgatcaaagtataaaaaatactttttggcttatttcttagtgagtcamtattccatattttgaaggtgttaagaggtatggtaaaggtggtacttgaacatttccaagcaaacgtgtgatgaaatcttycatcaatgtcttagcaatggtatatgatttttttagtcttagcaattttagataagttttttttttgtcttgtttttttgagacggagtcttgctctgtcgcctaggctacagtgtagtggcgtgatctcggctcactgcagcctctgcctccgagcggggtccagcgattctcctgcatcagcctcctgggtagttgggattacaggtgcatgccaccacacccaactgatttttgtatttttagtanagacagggtttcaccatcttggcctgactggtcccgaactgatctcaggtgatctgcccacctcgggctcccaaagtgctgggattacaagcgtgagccactgcgtggcctgagcactwagggcgcaangaraagccngtactggnawtwttacactactcrgcacargacmggntttaatctttttcttgggggacaagattggaaaattgaggtctgnagcagacctgaagagaggcatccagcaactctgagattaattcatcatgatcattcgttattgtttggaattgacgtttagctgtgttcctcactcagatacgtgcatgatagctgcttgctaatttggtcttagctcacatttcacctagaatgtatggtctccctctcccctgcaaaatatcccactgttgctaatctgtctgcctcataatttccatgagattgagcatcttgtttgttttgtcaccactatataacagcatgttggaaacaaagcagtaataaagctagaaaaaccaagcgaatacactggattaaaaaaaatactgtttcctagaattaaagaaataaatgaggccgggcgcagtggtgcctgtaatcccagcagtttgggaggctgaggctagtggatcatgtggccgagatcgcgtcactgcactccagtctagcaacagagcgataccttgtttcttacttaaaaaaaaaaaaaaaaactcga

SEQ ID NO: 44 - DNA, 796 bp, Homo sapiens
ggcacgaggtgacgtgtttctgcatctgttgccatgacaagctccctgcttcacccattgctgtatccccagcacctctctcactgcctggcaagggaaagcactcagaagacgctgaatgaccargtagagtgatgggttgtacagcactgttactccttttccatctctgtgtcccatgtgaaccttatggcacccatgagaaggagcttgtaccaggtttatactttctagtttacagatgagaaaacaggatcagagtggtacagatattggtctaagtcacagagaaagtgaattgtaaaagcagaaacagagcacaggctgcctgacttctagtccagtgctttttgctcaaattgcctcttatttctcaggttattcttgaaatggcagatggggattctgtttaatgaaacaaaagtgacaattctttctttcttggagagaaggtggagacagggtctcactctatcacacaggctggagtgcagtggctcaatcatggctcactgcagcctcaatctcctgggctcaagtgattcttccaccttagcctccttgactcactgggactacaggtgcacaccaccatacctggctaatttttaaagttttttgtagagacagggtctcactatattgtgcattctggtcttgaactcctggtcccaagtgatcttcctgcctcggctttccaaagtgctggaattacaggcatcacccccatgcctagcctgaaaattctttctatgtccttaacatcttctttcccagtatttctccatccactcga

SEQ ID NO: 45 - DNA, 1378 bp, Homo sapiens
gatctctgtgtttacctgtataaatattttccctgttctttttatgacttgtatatttctggtataggtttgttgcaaatggttatttaatcttgactaggtgagaagtcatagaaattctcctaatttcaacatctatttattcatggatctatattatttttgtgtgggagaaaaacttttctatttaaagataatttacaaacgatcataatctcttttaggtatgtctatttttacttgtcaaaaacacataacatttacaataggatattttgaaatgtttattttagtcctattatattgacattgttatgcaacatattcckaaaakgttttkgtcttgcaargctaaatatcaatacccattaaaaaactatggaattttacccatttcctgggcacttttcaaacaccactctgttttctctaagagtgtactggcttcatatatctcatacaatctctgtctttttgtgactggctcattttattttgcacaatatcatcaagctttatagttgttagaatattttctgctttttaaatactgggtgatatttaagtattttgtattttagattatatctactgagtaatttggkgacaaatttgcackgcttttacctattggctttcagtaacaatgctgcaataatkacmggtatgcaaatgacctatatgatcatatatgtgtaagtttatatatgtgccgcattctgttctactagtgtacgtttttacctttgtactcataccaaattgttacaattctgtagctctgtaatgtgtttcaaaatcagaaactgtaatgccttcaaaattgtttattttattgcagatttttgggtactttattatctcttaagactttatatactttgggggttgctgtttctatttcttcaaaaatgcatgagaaattkgamcaacattgcattaaatctgtaaattacattgagcaggatggacatcttcacaagattaattattttaacatttcaacaagcatgctcaagagtgtattgttttaatttctatgtatttgtgaatttttcagttttttcttcttactgttctatactcatttcattttggtcatataaagtaatccataaaaatttagttttaaataatttgttaagacttcttttttggtttaccaggttttctatcaaggagaatttcgtatgaggtatttagaaggctgtttatcattatgttgttgagtgttctttatgcctctgttattaataattgttttatactcccttcaagtccggtttctttaccaatattttgtctttttaaaatctttattacagaaagtgaagcattaaaatattctactataaaaaaaaaaaaaaaaaaaactcga

SEQ ID NO: 46 - DNA, 597 bp, Homo sapiens
tggcggccgctctagaactagtggatcccccgggctgcaggaattcggcacgagcccggccgccatcttgggtcatcgatgagcctcgccctgtgcctggtcccgcttgtgagggaaggacattagaaaatgaattgatgtgttccttaaaggatgggcaggaaaacagatcctgttgtggatatttatttgaacgggwttacagatttgaaatgaagtcacaaagtgagcattaccaatgagaggaaaacagacgagaaaatcttgatggcttcacaagacatgcaacaaacaaaatggaatactgtgatgacatgaggcagccaagctggggaggagataaccacggggcagagggtcaggattctggccctgctgcctaaactgtgcgttcataaccaaatcatttcatatttctaaccctcaaaacaaagctgttgtaatatctgatctctacggttccttctgggcccaacattctccatatatccagccacactcatttttaatatttagttcccagatctgtactgtgacctttctacactgtagaataacattactcattttgttcaaaaaaaaaaaaaaaaaactcga

SEQ ID NO: 47 - DNA, 600 bp, Homo sapiens
agaactagtgatcccccgggctgcaggaattcggcacgaggacctctgaccatcaggcttctgggaaccataggctatacccacaccacagagcatcgataaactattttgatgtttctcttgctttcagaaagacagcttccaagattcaagcccaggtggtgccggtctttttttggaggtgctaattaataatttaacttcatctaatgataattttatcttgttgcagtttgtggatttatgattatctcatccatccggtgcctagtgttgggcatagagtgtgtctctgctgtctgccagaatctgctactgggagaatttccccactgggagagggacccaggaaatggcatggtcttagaaggtctcctgaacacatttccttgggagggctcctgttatcttcaaggttgatggctttctgcaatctctcaagggctgttttgcctggaaacaggacgatggagacagagacctatcagctgtgggcatctcaatatcagcggaaatgggtatcaagaagtctcagccaggtgcagtgcttgcgcctgtaatcccaacactttgggaggctgaggtaggtagatcactcga

SEQ ID NO: 48 - DNA, 911 bp, Homo sapiens
  SITE (6): n equals a,t,g, or c
cccgcnggtaaagggaacaaaatcgtggagcgccaccggsggtggcggccrcgtctagaactagtggatcccccgggctgcaggaattcggcacgagcacctatccaccttggatcgtagcgtgatatggtctaaatctatactgaatgcgcgttgcaagatatgtcgaaagaaaggcgatgctgaaaacatggttctttgtgatggctgtgataggggtcatcatacctactgtgttcgaccaaagctcaagactgtgcctgaaggagactggttttgtccagaatgtcgaccaaagcaacgttctagaagactctcctctagacagagaccatccttggaaagtgatgaagatgtggaagacagtatgggaggtgaggatgatgaagttgatggcgatgaagaagaaggtcaaagtgaggaggaagagtatgaggtagaacaagrtgaagatgactctcmagaagaggamgaagtcaggtmagtcctaamatgcaataaaatgagtcagtaagtcttagttagacaatttctccactattcaaatacaaatggaatagttagggtctgtaacttagtttaaaactaatatataggctggacacggtagcttatgcctataatcccagcactttgggaggctgaggcaggcagatcacctgaggtcaggagttcgagatcagcctggccaacatggtgaaaccccgtctctactaaaaattgaaaaattagccaaggtgttggtggacatctgtaatcccagctactcgggaggctgaggtaggagagctgcttgaacccgggagcggaggttgcagtgaggtaacggatcacgcmattgcactycagtctgggtgacaagagcgagactccatctcaaaaaaaaaaaaaaaaaaaaaaaaaaaactcga

SEQ ID NO: 49 - DNA, 1863 bp, Homo sapiens
  SITE (172): n equals a,t,g, or c
gaattcggcacgaggatgatatggacatatgtagcccagtggcattgtactttctgctgacagctgcacacattacagctgtctccaaacccacagtgatgcttagggaaagaccctgctcaggacccagcaggtcagcaccccagagcagactgataggtccgtgggaccnatgttagagcagaaaatttgggctcagcacattttactgttagtagagagccaggaaacgttttctgggttggggattttgtgggattttttaatttttttagtaggttttgtttaacctctgtgcagtttgtatgaatgaattgctatacatttataaggagccagggtctggagggttgctatcactttgtccagcccaaataccttcctgggcaactcctaccatttgtttgcagttgcctctactagctgatggcagtatgctggaaagaggttgtactataaagagagttctttccttctactccagagttgttgtgtagctttgccattgaaccgatcaatttttaaactctttaaagaagcagcctggccaacatagtgaagccccgtctctactaaaaatacaaaaaattagctgggcatggtggtgggcgcctgtagtcccggctgcttgagaggctcaggcaggagaatcgcttgaacctgggagtggaggttgcggtgagccgagattgcaccattgtattccaccccgggtgacagtgcaagactccatctcaaaaaaaaaaaaaaaatttggcatcatttacaatttcatagaattactgtgaaggcctttctagttgagatgttggggtatttgggattctaattgttaaccccagaagaaggtaatttagcttgtatttatttaaaacccatttagccttttacttatatctggtagaattccagtgatcatcctaataaggtatatttcagaataatttttttttccttcagaataacttagaatcagatgctataagggctcctaggagcagtgtgaaatttccgtaaagataaatttgaatgttgtaaccaagtttatattaaaccaagaggccatttccaatatgattttttgtttctttttaacttgttaagtccctaagagattacatgctagggcttgagtcatttctattgtagataatgatggcccacacagtcaccttcaactatccacataagctaggctttccgcttttgccacggacagtgtgaccaagatatttccagagtaaataacccaccacaaccttggtaattcctcttttcttcttaagctccaggaagcgaaagcagaaggactcttttcagactgccctctgtagcctacattgcagctttccaaaacaggcagctagcactgggaaagcccatgtggtgaccccatatttttctgaggttcttcttttccatggtgttactttattatcagaaagtaaattcagaaaacaggtcttgcccttagcagacaagaaccacaccagtttcttgtaaaggtaacggatacattgggattcaggagtgacacagaggtccagccccagaacttgtaaggattttgtttgaacactgagcagatgcctcctccctgccacccatcacactagttagggctggccatgaattctatgccagagtcactcctgcagtctgctagggatgggccttcttatcccactctcgcacacatcccagtctagtctttgccttcacagagtcctccttgacacccctgacttaatgatagttgctgttttggagtagrattgatcaggtttaagtcatcctgctcaggttggggcatagtgggntcatgnctgttantttcaggcatttggggaagccaaagtggaa

SEQ ID NO: 50 - DNA, 810 bp, Homo sapiens
  SITE (688): n equals a,t,g, or c
gatcctccacatccttccatggctctgaagaataaattcagttgtttatggatcttgggtctgtgtttggtagccactacatcttccaaaatcccatccatcactgacccacactttatagacaactgcatagaagcccacaacgaatggcgtggcaaagtcaaccctcccgcggccgacatgaaatacatgatttgggataaaggtttagcaaagatggctaaagcatggggcaaaccagtgcaaatttgaacataatgactgtttggataaatcatataaatgctatgcagctttkgaawawgttggagaaaatatctggttaggtggaataaagtcattcacaccaagacatgccattacggcttggtataatgaaacccaattttatgattttgatagtctatcatgctccagagtctgtggccattatacacagttagtttgggccaattcattttatgtcggtkgtgcarttgcaatgtgtcctaaccttgggggagcttcaactgcaatatttgtatgcaactacggacctgcaggaaattttgcaaatatgcctccttacgtaagaggagaatcttgctctctctgctcaaaagaagagaaatgtgtaaagaacctctgcaaaaatccatttctgaagccaacggggagagcacctcagcagacagcctttaatccattncagcttaggttttcttcttctgagaatcttttaatgtcatttatatacaaaagaaattctcaaatgttaaaataaaggaatagtttattgcttaaaaaaaaaaaaaaaaaaaaaaaactcga

SEQ ID NO: 51 - DNA, 956 bp, Homo sapiens
aattcggcacgagctaaagcatggtttccaagatgctacaggcagcgagcctctctctagtgacctgggtagtttgcacggtttggctggaaaccacagtccccccatctctgccagaaccccccatgtggccactgtcctcagacagctcctggagcttgtggataagcactggaatggctccggctccctcctcctcaacaagaagtttctcggtcctgcccgagatttgcttctgtctttggtagtcccggstccttctcagccgaggtgttgctcacatcctgaagacacgatgaaagcattctgcaggagggagcttgaactgaaggaggctgcgcactggtccctaatgacatggaaagtttgaagcaaaaactggtcagagtgctggaggaaaacctcattttgtcagaaaaaattcaacagttggaggaaggtgctgccatctcaattgtgagtgggcaacagtcacatacttatgatgatcttctgcacaaaaaccaacagctgaccatgcaggtggcttgcctgaaccaggagcttgcccagctgaaaaagctggagaagacagttgccattctccatgaaagtcagagatccctggtggtaactaatgagtatctgctgcagcagctgaataaggagccaaaaggttattccgggaaagcgctcctgcctcctgagaagggtcatcatctggggagatcatcgccctttgggaaaagcacgttgtcttcctcctcaccagtggcacatgagactggtcagtatctaatacagagcgtcttggatgctgccccagagcctggcttatagagctagcatggaactcacaccacagcttccctggtccacagaggstctcaccgccattgcaccagtatggtggtatgtactcacaaagattaagaaagaaatgtattctgaytaaaaaaaaaaaaaaaaaaactcga

SEQ ID NO: 52 - DNA, 300 bp, Homo sapiens
gaccatatgttgcaggaagtcaaactggactttttgtggctactaaatttgcctttaatcttattgttctcaattttggaatcaagtatgaaaatctgcacaaatgcaatgtttacaagaactggttgattctgggaggcatctgctacagtctctttttatatggatatgtacatgtcctattctacaaaaatgattaaagataaaaacatacttgtatcccactgctactttagctgtcaaatttggtgtttcatcacattaaaagcaataaatcagtagttggtaatgtaaaaaaaa

SEQ ID NO: 53 - DNA, 841 bp, Homo sapiens
  SITE (836): n equals a,t,g, or c
gaagggtcggggagatatttccgttagacatcgctgaaacacagactgggatcaaactgtgctcatagtcctaaggatctccagcaccctgccggtggcactactgagagacgaggtgccagggtggttcctgaaartgcctgagccccaacttatcagcaaggagctcatcatgctgacagaagtcatggaggtctggcatggcttagtgatcgcggtggtgtccctcttcctgcaggcctgcttcctcaccgccatcaactacctgctcagcaggcacatgggtaactggctcagcatcctcttccctcctagtcactctcagagaccattctcgagcctccagcaggacagaccctttggagttcccaaacgtcactcaaaaactaccagaggacccaccggccaaattccttcccaccgctccccctccccccaataactgtatctgggtaatccccactctgacctcaccttttaaccaactatttctggctggaagtggccatccacatccgtctactacccagaccttctgcctagacacagcttttgcaatgtcctacgaggaagtgctcgtgtaacctggtctaattaattttcttcatccctgttaaaggactgaatatgaagaaatgtccttgaattacaacagaaggaaatatggttggacttagagattagtttaaattcttgaactgataaacaatagaaggtagtgaagctcggtcctggaaaggcatttcaattagggaaaataaaacaatgctgctttggttgtgctaagaaaaaaaaaaaaaaaaaaaaactcgtagggggggtcttggtacccaatngtcct

SEQ ID NO: 54 - DNA, 634 bp, Homo sapiens
gattaatcccctcaaccttctttctgagttcccatttcacagatgggtaaaactgaggtttactcctcgtctagcttcactgaatggcagagcccatagcttgtctttgcctaatctgctgcataatcatttcagcaacaactcaaatgccttttgagggttcttgcttctgtttggtgccttgtaattttcaaccatattttagacactttaggcctaatgatctaaggcatatggtttttacccatggtctgtgggcccttgagaagctgagtcctctgaaagaaaatcagaatgttgcatgcatctgtattttttgtcttagatttcacttgattctcaaatggatccttgactcccccaaagtttaatttattcaacaaatcttttttttcctccatactttttattctgaaacatattcccccaatttttaacttctgaaaaatttcagacaagttattggaatagggtagtgagtatctatgaacctttcatataggtttactttaaaaaaaatacaagagacagggtcttgctctgtggcccaggctagagtgctatgattgtgccactgcagcctgggtgacagaacaagaccctgtctttaaaaaaaaaaaaaaaaaaactcgta

SEQ ID NO: 55 - DNA, 863 bp, Homo sapiens
  SITE (7): n equals a,t,g, or c
gggcagnagttccatttctgccgtggtcccagcagcgtcgctgtgggtctggcctgggttgcgtgtgtttcgtatgtgggccgtgctccctgcttggttcccttttcctggaacgtgtcactgcctccctgtctcgctccgtggacatttctgggaggtcaggccgtggccacctggccccctgttcaggtctgaggctcccacctgcttaggttcgggaagctcaggagtgaggccatgccctcctcaggacatcccatccaagccagccatgtccggtgatgggccgctgcccggnaaagtccttttccttcttgtaactgagaagaacttgccttgagccacgtcaagtcccgtccgtcgcagccactgcccacaagcgtgagtctgctgtgagccagcggctccatggcagggcatcccagcgccattcctgccttcacacacacttgctgccgtttccctgtgctgggggctgtgcargtctgcctcggtgtggacttttctcttaggaaagagccccaggtcggccgagcacggtggctcatgcctgtaatcccagcactttgggaggctgaggcgggcagatcacgaggccaagagatcaagacaatcctggccaacatggtgaaatcccgtctctactttttaagtattttatacttaaaatttttgtattttatacaaaaattagcgggcttggtggcagatgcctgtagtcccagctactcgggaggctgaggcaggaaaatcacttgaacctgagaggcggagattgcagtgagccaagatggcgtccactgcattccagcctgggcgacagagcaagactctatctcaaaaaaaaaaaaaaaaaactcgta

SEQ ID NO: 56 - DNA, 712 bp, Homo sapiens
  SITE (20): n equals a,t,g, or c
tgttgtttggaattgtggancggattaacaatttcaccacgggnaaccggctttgncccatggattccgccaaggcccgaatttaccccttcactaaaggggaaccaaaagctggagctccaccgcgntggcggccgctctagaactagtggatcccccgggctgcaggattcggcacgaggtttcctgtcagtgctattgagattttattttattaatgtctgcacttagttttacttcctactttctacttttattgagagttaaacctgttgaagtctcaggttcaattcctcaccctgagcaacctaatgttttatgtcttgttcttcctacatttggttattgaaactgaagttttaggttaccagatttgatagaagcacataagactacttactgctttagtctcaattattaattgagaaattatcaattaacaataaggatttctcttatttttccccaagataagttatatatttaaagtgtgttttatagtagaaaggttttagaatatttgggttgctacattaattgaaatggcagctgaagatgtgatttccagccagggatttattaaaaaaaaaaaaaaaaaactcgagggggggccgtacccaatcgncctatagtgagtcgtatacaatcacgggcgtcgttacacgtcggactggaaacctgcgtaccactancgctgcncacaccccttcgc

SEQ ID NO: 57 - DNA, 925 bp, Homo sapiens
gatttaaatgtgttgtttctttttaaaaacattgaatctgtggttgggttatttctgtcaatttatttgccttccttgccaagtcacactttgcctaattgatgtcctgtgtgttttccattccgttcatgctgaattatcttaggtcaaagaggaaatcatctttctgcctccaaccttcttacttgcctctaatcccctttcttgactcttccaagtcaggattctcaccaaggaagctatctgccttctttgggaatgttgggcttatgaagacttggagataatggggttcatgtattcagactctttrgcatwtacagtagagtttctaatgttgtcagcattccctagtgggcagttacaagttaggttgggattctaatcatatttatgatastcacagattaaattgcactttgtctctgccccagtctttgattcccttttggccagcagtttttaggtctgtcagtactgcactgcargaatggcagattttgggatctctgctggccagtttgtggcagtggtctgggataagtcatccccagtggaggctctgaaaggtctggtggataagcttcaagcgttaaccggcaatgagggccgcgtgtctgtggaaaacatcaagcagctgttgcaatgtaagtacccacccacgttgtctttatgaggctggaggggtttccatgggagtgttgcatttctgtggttccttgatatctgagttttcatttagggtggcatgtgatagtggtggctggtcaccctgttgtttttcagttgagatatatcggaggaaccacccccaataattcaacgtaggttcttttctattttccctaagtgtcggctggtctgagaaataaagggaaaggatacaaaaaagaaaaaaataaaaaaaaaaaaaaaaaaaactcga

SEQ ID NO: 58 - DNA, 601 bp, Homo sapiens
gctgccaggaattccggcacggggaacagtgtaatattgaagcaaatgctgtataacaaccacctggaagcccctcatgtatctctttttgaaaacactcctctctttctccactctaatgatgaccaccgccttgtcttttatggtaatcactgttctttgggttttattactgcatttattggctaatatatgcatccctagaaaatgtagttttgcctgcttttatataaatggaatattactgcatgcagtcttttgatttgtgattgttttgctctaaggcttgtaagggtcatccatgttttgcatatagtttgtttattgtcattgccatagagtaaatcattgtatgaatatactgcagtttatttactgttgacatatgtttcagttgtttttaactactaggaaatgctactctgtacattcttgtatatgtaccttggtgcacatatgtatgtttttctagagtatatacagtggcatgggattgctgaattaaaaggtttgtatatcttatactagaagataataaaaacttttcctgatggattctgccaattcaaaaaaaaaaaaaaaaaaaaaaaaaaaaactcga

SEQ ID NO: 59 - DNA, 730 bp, Homo sapiens
gggagaacttctttattcacatattgcattgttttacaaatggaacctgcgagtctatggatgccatctttttaacatggtctggaactgaacctacaatatttctgagaaaattgactttgcttctttgagaacagcatggtgagtctactatccttgacttttcatcaatttgtttcatcactaaagtatttcaagttgctgtctacgtcaaggcaagaaattctgtagggtttcagctgaaaaatcagaagccacacaggcttgctggaacacacagctgcatttccagctctgattttaaatgtgcwctatctggatccatattctggcacaatctgcctcttgtgatgaagatgaaaatggttaccttaaagttctcttcggtcaggccttcttcagttttagcatctctaatcattgcagcaacgtatcgcttcaccaggttcctcataacttcctgaggcattttagaacaagagtattgatactcaatgagtaaataaatttcctcctgagtcagttctgaaggggggactgcattttattttagtgaaaatttcaagacatagtacaaggacaacttacttggtattggtgatgtcttctcaagttatcagcagctcgcctctgaaaaggaaaaggacattcctttctggttatactgttatattactattctaaaaaataatttatttttttaatcgaaaaaaaaaaaaaaaaaactcga

SEQ ID NO: 60 - DNA, 845 bp, Homo sapiens
ctcgagttttttttttttttttttgatacattgccctttttctcctctctatccaatattcagttgcttttttttttttggaaacatagtcttgctcttgtcacccaggctggagtgtaatggrcgcgatctcggctcactgcaacctctaccttccaggttcaagtgattctcctgcctcagcctctggagtagctgtgattacaggcgtatgccaccacgcccaagtaatttttgtgtttttagtagagacagcgtttcaccatgttgtccaggctggtctcaaactcctgacctcaggccatccacccaccttgggctcccacagtgctgggattacaggtgtgagccactgtacctggcctcctttcaactttatattcacctatttttatcttttttaaaagcaccacttgcctttgttttaattctctgtcaagcaattaattgagattttcatcctgctggcaaccactctagttctgcagcatcctcatgagaagcaaggtccacttctcatttgtcttcctttcaactgtttctatttccacactttcactagcaatattaatttcaaccaatttctaagacaagaagtatgccactgaatgtataactgatcttaacaacagattaacaaattgttgattccctgccattttcaaaatatcaaatatacaagacagtaatttttttaaaatattttttcatctctgagggaaaggatgatttgaagactcctttcaaattcccaagaaagcctctcaatattatctgtgtaaactactgattcacaggaataaatatttgttatttaatataaactaaaatgaaaaaaaaactcc

SEQ ID NO: 61 - DNA, 958 bp, Homo sapiens
ggcacgagccctgcggctccttagtcacctctgatagcagattgagggaggaaaacaggtaaggcatgaggaaatggccaggttgggttaacccactggtttcaaccagttcaggaatgaggttatttggccatgactggctgatcttgagctcaaggatctgcttcaaatgcacacaggcctagttgaagtttaaaccccagcaaaacattcctccctgtaaatggaaaatcctacttctacccccaccctgccctgttttttgttttttttttccccaagatcattagatgtcctcacccctcctcactgcctcctctctgggacaggctgggaccttgaggaagataaagccttccttgactacccatcatattcagtgtccctgttcctcactcagagaggaaggcagaaccagtcaggcttatttcagtaagttccacagttctacaagactgcaggaattctccttaagggaggagagcaagcaggtgtggccccagcttctggaaatggcagaagagagggttttctcattgaatgggggtgggggctcgtgtgtcctgggaaaccccatcagtcccttcatttcttgagactcaactcctgggaggagagggtctcaagagttgtccctggaaggagggcgggggcagtctgcatctatttcaggttgtggctcttggttctaggactcttacttctctggctaagggctcagcttcttgggacttcaaccatcttctttctgaaagaccaaatctaatgtaaccagtaacgtgaggactgccaagtatggctttgtccctatgactcagaggagggtttgtcgggcaaattcaggtggatgaagtatgtgtgtgcgtgtgcatgggagtgtgcgtggactgggatatcatctctacagcctgcaaataaaccagacaaacttaaaaaaaaaaaaaaaaaaaaaaaaaaa

SEQ ID NO: 62 - DNA, 582 bp, Homo sapiens
  SITE (20): n equals a,t,g, or c
ccgtttgccggcccgcctcntgggacntggtggtccccccccgggcctncagggattcggcmcgrgtgcatacatgcctacctatgtatatataaacaaacatttttgtaaacagctcagtgaggactttggactggcataaatcataggaatatgattatgaggatacatccaattttcagattgggcaatgtatacagtttattatcatttctgattttgggtagagttagtactaagaacagcattgaagaaaagcagtataacattaaaattaagaagatttaaaatacaagaggattcataacagtcacttttaaaatattgttttggctttctactttggagctgtaattttaaaaaaagaatgaacaggtttttgtatgaatatgttagaatgactaattatagagcatctttcaactggaatacatgtagatactaacacctggttgtatttgatgtaatttcagtgcatacagtgtgtgtaatctgtattaagtgaaatacttatgaataaagttgtttctgcattgcaaaaaaaaaaaaaaaaaaaaaaaaaaaaaaaaaaaaaaaactcga

SEQ ID NO: 63 - DNA, 752 bp, Homo sapiens
ggcacgaggggagaggcaggcatttgcattcagtcttgaaggctgaatagggcagggtaggcacagtgattccagagagaagtctttgctcctccatctatggaaaaacttctcacattgtatttattactatatgtttcttactggagtgtctctcctactggacagggagcaggtttatttattgctcagtcctcagcccctggacttaggcagactcatagtagacatttgggaaatgcttgggaaagaaaggaggggaggagagaggaaggactccatggccatgtctaaatgcccagcaatgtcatagaggttatgggggtgcaggagaagacacagccctccctctggcagctaggatagagcctagctgctgttaaagacaggcagctcattcctcacctgggccaagctgcagctggtcatctctgcccctttctccttccatcttatgggagcttttatggagtcagaagtgagtgaggcagacctgggagagccctacactcaggaagaatgtaggctgcagaaaggaacaggtgtcctggagttagctcaggaaggtcttgaaggaaggggttaacyagcagatggcaacccagtgacttttgttgctctctgaagccacagaggaaaacagtagcaacrrratraaataaaataaaataaaaaataataaaaaagcaaagttcccaaggaaataagatgggggaattcgatatcaagcttatcgataccgtcgacctcga

SEQ ID NO: 64 - DNA, 706 bp, Homo sapiens
ggaaagaaatccctactgtgtggcaccaggacctgtgtgacctgcaaggcgcctgttttcctcaacaaagcctcttttataccacttgctccccacaccaccctggccccttccacttgctcaaaaacactgagctccttttcactgtggggccattgaatatgctgttttccctccctagaaccttttcctctcattcttcacctgcccaactcatatttatccatgcagcctcagttttaatggcatttcctcccaggccttccaagaccactctccctcaggcagctttcctgacatctttagcctgcccgctcatgctctctaccttttttctgtatcaaaatgcctttgtttgcaagtaacagaaggcctgacttaacctgcctttaaacagtaaggacacaagtatgcctatgttattagaggtctgcaggtaaggcacgtaaagggtcatctttttccagtgtcttcaactccatttctctgaggttccatcagctacattctgtgccatgactttatcctcagtgcatttttcagatggtaatcaaatggctgtaacatgttcacctctagctcagcatgatactcagaggaagaatagagttgcttctaggagttttgtgatgagaatgagggaatttctttccctggagcctccagcaagcttgtcattaagtacctcctcaggtttctggctcga

SEQ ID NO: 65 - DNA, 400 bp, Homo sapiens
tcgacccacgcgtccgccctgcatggcgagatgtcctcctttcccgggccacagtgtgtgcaactaataaacctcctccatctcatctgcccagtgtcgggtcttgtgtgttcagccatcaccatagccctcaggcagaagtccatccctcaccaacagggaagagaggcagtgatcaaaacacctcctccaggaagtcttccctgaagttcgtagtctggcttcagtgccacttcttccctgccctcatattcgctaaccgccacttactgcctggttttcagcctcactaggatgtgggccactaagggccaacatggtcctacttgcagctgcattatcagggcctaccataacaccttccaaatgcttaaaaaaaaaaaaaaaaaaaagggcggcc

SEQ ID NO: 66 - DNA, 773 bp, Homo sapiens
gcacaggtatgttttctgatggcacaggcgaggtcacagaaaagtggatggcaggcgttgctgtctgtcagaataacacgaaagtgagagaaggccgctctttcagaataacaccacaagtgggagaaggccgctccctcagggctggccatgaataaatggggatttctgcctgttytctccctcccgcctcactcccttttcctgcagaggcagctcctgagccattgccgagcaggatgctagttttagcatggattacatttccaccgtgtaaagcctgctgcatgatgtgcatcttctccagccgcctccttcagcaggagarggtttgcacarttgtccagggaarggaacctaggggcatggcccaacgggacagaggatttgartccctctgattatgagcaggttaatttaaaagtgaaaaccatggttacccattgccctttaaaaamcacccaggggccgggcacagtggctcatgcctgtaattcccagcacttttggaggccgaggcaggcagatcacaaggtcaggagatggagaccatcctggctaacatggtgaaaccccgtctctactaaaaaagtacaaaaaattagccaggcgtggtggcgggccgagtagtcccagctgctcgagaggctgaggcaggagaatggcgtgaacctgggaggcggagcttgcagtgagcctagatcgcgccactgcactccagcctgggtgacagagcaagactccatctcaaaaaaaaaaaaaaaaaactcgta

SEQ ID NO: 67 - DNA, 647 bp, Homo sapiens
ggcacgaggtttgatatatttttttctcatctttttgctgttacttatatgtaactatctttaacaagtttgagatcttgttatatattttcatttgttgctttataaccatttctctatattactaagtttaattaaggtctggaatttttttagatggtgtatcatgggtataatatttatttagttgttttcctcttgttatatttagattgaggcagtgctacaggctttaactagagaggtggttggctgttcaggactgggaggtggaggactagcaggaacagaggtatagcaggagagcatgcctactatgggtataggggcagtaaggagagcagctgaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagattaaaacactgaactgacaattaacagcccaatatctacaatcaaccaacaagtcattattaccctcactgtcaacccaacacaggcatgctcataaggaaaggttaaaaaaaaaaaaaaaaaactcga

SEQ ID NO: 68 - DNA, 675 bp, Homo sapiens
ggactactccattcctctggatgtaaaatctacattctcttgcctgaggtggatacgtttgcttgggttctgtttaaggagatggggccagcagtgtgtttcagggcctgtgaaatgtgttctctatccgggcttttgcttaatctctgttttcagtcttgcctatcagtcccactgtcgggggtacctcgtgtctgagtctagaacctttccaggttgctgtgggacagattagcctccttgttctcagtatcccctgacctccacctttattgctttgctccatgaattaaccatttccatgtactgtcatgtctaatgaagatgaattctcttctgttggtaaccccattccttttttgtaattgtgtgcttatacaatgtttattcttcactgtatttctattggagcctcaggacaaagagcagatggtgagaatctgtgttcagtgttaagttttccttctgtaagacatgtgcaacttgtgtttttcactgaatagatcatggacttaatgcatatagagctactttgtttttcatgattgtgccttcaattatatgtagaaatataatttgtgaattgcctgatgaaattttcctaattttgaattatctttgcattcctataataaacactgttagaatggcaaaaaaaaaaaaaaaaaaactcga

SEQ ID NO: 69 - DNA, 889 bp, Homo sapiens
gtacaggtgcatgccactgcacccagctcattgccttttgttttgtatgttaaagcagatttagcccatgaacttggagacagttttgctgagcagaacttcatctcttggctttgctgtttgtttgccttgttttttttgttggttttacttagttttgtttttggagctaacatccataacttttgctatgtatgatataatcccctgtatgaccctgggcaagtaacttaacccattcagggtccaggttcctcttatgggaaagggatgcttgataagacactgttcatggttccttgcagtttactattatgatagatattcgatgacctaaaaattaaaccagtttcctttttcaaatttaatttttycgggaggtggaggaagattttcattccttatggtttgagaaacatcgctttcatacatgtctagggtaaccaagttctctaatgaatggcaatagtgatgtattttyctwaaatccttttctaamcagcattatgggtttgtgctgtaccggacaacacttcctcaagattgcagcaacccagcacctctctcttcacccctcaatggagtccacgatcgagcatatgttgctgtggatggggtaagaatcgtctctgaactgtgcctggcttttctccactatcttgaaatcagatgggaggaggcttttttctgggtgggactgaggaggcacactgaagtcccccaggtcatcggggctgggccattgcctttttccccaccctgggtagtcgtggacagaagcttgggatgggatggagaggagagatcgtgctgtgtgtcatgtctgttgttcaagtaaataaaagttgccctgacttcaaaaaaaaaaaaaaaaaaaaaaaaaactcga

SEQ ID NO: 70 - DNA, 888 bp, Homo sapiens
  SITE (347): n equals a,t,g, or c
ggcacgagaactgccgtccaatctatgagctgggcccttccttccctcttctttcttcttttctctcccttccttcttccttcaggtttaactgtgattaggagatataccaataacagtaataattatttaaaaaaccacacacaccagaaaaacaaaagacagcagaaaataaccaggtattcttagagctatagatttttggtcacttgcttttatagactattttaatactcagcactagagggagggagggggagggaggagggagcaggcaggtcccaaatgcaaaagccagagaaaggcagatggggtctccggggctgggcaggggtgggagtggccantgttggcggttcttagagcagatgtgtcattgtgttcatttagagaagtgggtgaaggttcctgggatcttaggtaaagactagacgccgcctagtactggtctctactgtgctggctcaggagttctgagaactggaaggacttagcctcaacctgagttctgcacacaccccttccccttaaggaaggcagctctgagaggcagcaggacttgatccaaacccacagtcttgtcctggaggcagcaggggtgaaggtggagggtccagggccatgaggagcccccttgccatcagagcctggcctaaccaccctcttctctacttacacacacatgcattttataatagctctgacccaacctggccactctgcagagactgggacagacaggtgcaggcaatgggccctcccacacccagtcacctacaaggaattttcaaatccacttttaaaacagaaaccggtaaatgcgccgtattgtatattttatttaaataaaaaaaattccagcaaaaaaaaaaaaaaaaaaaactcgta

SEQ ID NO: 71 - DNA, 796 bp, Homo sapiens
gaaaaaaaagaaaaagccaaaaaaaaaagaagaagaagtaccactgctaggatttgaacccagatctagctgactcaagaaccatgccctatctctgtgtccatgttgtcaccacttaatcacttgtattttcccttcaggtttctctgtatgctgtgttctctcccaagagtggtcttccaactcacccctattaaggaagctttcccaagccaggagcttacctttccgtgcacacattgaatgatgatcatttgtcattctgtcttgccttacaaaagaggaccagctccttgaggataggaaccttgtccttatctccctgttcccctgtatgggggccagctcctggcaggtgcatagtaaataatgagtgataaacttgttggaaagaccatgcaggaaccaagcaactcttttcctctgcctcaatgcagttagttcaagaacttactaagaaaagagttgttggccaggcacagtggcacaggcctgtaatcccagcactgtgggagaccaaggcaggcaaattgcttgagctcaggagtttgagaccagcctggacaatatggcgaaaccccatctctatgaaaaattggaaaagtagccaggcatggtggcatgcacctgtggtcccagctactttggaggctgaggtgggcgaatcactttagyccggggaggtcgaggatgcagtgagctgagattgcgccactgaactccagcttgggcgacaaaatgagaccctgtctcaaaaaaaaaaaaaaaagaaaaaaaaaaaaaaaaaaaactcgta

SEQ ID NO: 72 - DNA, 532 bp, Homo sapiens
  SITE (434): n equals a,t,g, or c
ggcacgagtaaaaggtgccatctatgaatcagaaagtacgcccttaccagacaccgaatctaccagctcctggacagaacagactaagatacattccaagaagcagtttctttggagacagaggcgtaactgtgcatatggacaaggtttatatttctgttcaaagtggccatccatatgcttctaggcttcctttgtctctggtatcaagtgtatgtatgtatgtatgtatgtacttatttatttatttatttattattttctcttttttctctgccccatatgatctgcaagaaaagtgtcaagtttataatgagctccccaaagccaccatctgggtagcctcacatctttttcatcccctgtgcctcttccctgcttttgtcctactctagccagactcgtgccgaagggggggccggtamccaattcgncctatagtgagtcgtattacaattcactggccgtcgtttamaaagtcgtgactggggaaaacctggsggtacccaacttwaatcgccttgaagnaaat

SEQ ID NO: 73 - DNA, 546 bp, Homo sapiens
ggcacgagctctccagcacctccttggaacagatgccctgctactttacaaggcttgtggaaaagagaaagagaacagtagcaaaagcctgtgtagttcatgaatagaagttagcatcgtagtgagtaagcagtactgatgatctgtgaaatgattctctgtggacttgagcatgctaaaaagatcttgaaaaaggaaaacataaatctttccaaaacctcacatgacccctgtatgctttcgccttcttgaagctttggaggagagcataggtgtggatgaaatggagtcttttaaaagttgttttggtttttgtttttgtgtgtgggtttttaaagagagcatatcctgccacgtagaagaaaatccagggggtggctgtcctcctacaggaaggaggtaaacaagcatttttccttaagggctctattccctcagcctcgctccctcgaaggccacacttggaggccaggaagttaatccattaaaaaaaaaaaaaaaaaaaaaaaaaaaaaaaaaaaaaaaaaaaaaaaaaaaaaactcgta

SEQ ID NO: 74 - DNA, 715 bp, Homo sapiens
ggcacgagctttccctcagtccaatcttgcaattgctatgtcagtttcagttcacaataataccagtgcagacatggctccttaagattttctccttttccctcacgcgggtcccaattctaaattcccaagggctgacatgattgacatttgccatagcctgaggagggagcatttccttttgtggtctttccttggtttgttttattgggcagtgaatggcaagtctgtctgtgtttctttgcttcaccccaaacaccttggcaaaaatgaaagccttctaatttagctgtgtcctcctttacttatgtcaggaagcctgagccataacctttgattaaaaaaatttttttttgttttttgtttttgagacagggtcttgctctgtcacccaggctgaaatgcagtggcacgactgcagctcattgcagccttgacctcactggagtgtagtggcatgactgcagctcactgcagtcccaagtagctggcacttacaggcaggtgccaccatgcctggctaatttttaaattttttgtagaaacagggtcttgctggctgggcacggtggctcacacctgtaatcccagcactttgggaggccaaagcgggcggatcacgaggtcaggagtttgagaccagcctggccaacatggtgaaatcctgtttccactaaaaataccaaaaaaaaaaaaaaaaaaaaaaaaactcgta

SEQ ID NO: 75 - DNA, 406 bp, Homo sapiens
aggttttccagaaagttatcagatcttgctttcctgattagcagcagttagcggggtggataaaagcaccccttcagagcaatctcatttccatttctttcaggccacttattttttccaactttttttccgtatcttcataaatgtttcactcttctttgttagtatttcttagtctcttgagtcaagaaatatttactgagtatgattgcatgcataagtagtgtgcgttagagatacgatacctgtaagacaccacagtgctgggtagatccgggtgccattgtctgttgccagggccgaagttggcattttgtaagtgttcgaataagcaccatgccgtgggataagaaataaaagtgtgtgcctcatctgtaaaaaaaaaaaaaaaaaactcgaggggggg

SEQ ID NO: 76 - DNA, 542 bp, Homo sapiens
  SITE (429): n equals a,t,g, or c
gatcttaagcatttttaagcacccctggatagctctcaatgacaccctgcgctggctgtcctggagtcacctgggggagggagggaatgggttgctagatggtgcatgtcagtaatttgccttggtgtttgatgacattaagtatattcgcattgttgtgcaaccatcactgccatccatccacagaacgcctttcctcttgcaaaactgaaactccgtagtcagtaagcaacaactccccagtccctcatcctccacctcagcctctggaaaccactagtctactttctatctctgtgagtttgacactctcagtaccttgtacaggtggaaccatacagtatttgtctttttgtgactggcttatgtcacctagaatagtatcctcgaagggggggccggtacccaattcgccctatagtgagtcgnattacaatcaatgggccgtcgttttacaacgtcgtgactggggnaaaacctggcggtacccaacttaatggcttgcagganatcccccnttcggcagtggggtaataacgaag

SEQ ID NO: 77 - DNA, 420 bp, Homo sapiens
ggcacgagggacaagaaggcctttctctcgagtcggcatggttccacttctctgactgcatcgggaattacctctcctttgggccaaagacaaaaaagaatgcagacttgtttccaggatgattaaattacattcagcatattcttcccgagtgcgtcccgtcttagtggggtttagagctgcgttcaggccagctgggctccggttacctctaatgaggatgatgatctggaggcttagcgataattctgcactgattctcttgtgcctgcagaacctgtgttggccaacttggatggcaggggaagatcaacagaaggtgccctccacccacgtcctcccagcgctcaccttggtcagcctgggggccaactcgtgccgaattcgatatcaagcttatcgataccgtcgacctcgtag

SEQ ID NO: 78 - DNA, 465 bp, Homo sapiens
  SITE (446): n equals a,t,g, or c
gattttttcccatcgtggaacagagtcttgccaacttatacctctctctgagccttagtctcctcgtttgtaaaatgagagttaaaatctacctcatggaatcattgctaagattaagcaagatatataagtagagcttgtgcacatggtaggtacttggagaatgttatttctccttccctcttactcatctggacaagtttaactagaattctaaacagttaaatatgtatcaatcctttgtattaaatatcttggtggtaaaatgttaaaatattgatgtgaataacagctggtattgaatattcaaattaggggaactctttcattgttttaagataacatctgtacatttaatctgtgccatgcaataaaacagcttttcctgaaaaaaaaaaaaaaaaaaaaaaaaaaaaaaactcgagggggggsccggtacccaattngccctatagtgagtcgtat

SEQ ID NO: 79 - DNA, 889 bp, Homo sapiens
ggcacgaggttacttattgctcctacttcatatcatatgtggttctacaacctacattatcttgtctatgtcttttaactagctgtgtgttcttacataagatctgcagaccttggttctcaactgcaaaagcatattgattaaatgattactgtttttacctgcaatactttaatttttggatttgggattaataatgtaaaaaagactaacatatatgtgggattacaaaactgttttgttagccttcaaacaactgatgaactgcatcaggagctgtcttatacttattgttctgctattaatacttaatgcactgccttgtaaagagctgattgctacttaaaaactctgcttaaatgaaaaaccaaaacataaaagaattaaaccaaacatacttactctcccatagcccatggtggacagcaacataaaggagggaaatgtttctgttgatctttggcttcaaggattaataccagatttggataccggttagttagataattggtaaggaatcccataaagttgtaaattacataagcttcatagcattctctgcaggtatccacatatattgcaattccgggatatttcaaagctatccactatgaaaaagcacagatgttaaagatagttgcagctaagataaaatgaatcaccactccattcatggtactcacaataagctaatttttatgcttgagatgtcttgtcatatacttacatgggactctctaaaatttatcattatgagggctatcaatctgtgaaatgaatgcttaaaagcaataaacatcttagatattggtaaacaaaaacaagtgtttgaggggtaaataatgaataaagagagaagctaaagtaaaaaaaaaaaaaaaaaaaaaaa

SEQ ID NO: 80 - DNA, 470 bp, Homo sapiens
ggcacgagggaaatcttgcacataggcaggtaaataattataaatggtgaagtggattattctgagctgcttaattttaaagggaaagagaactttaaactcttcaaccttttatgctgctaataagagttccacaatcaatagaaatctatcttggcaggcacttccttttacccactagaattttttcccttgggagttcacgatccccagaaactgtgatatgagccattcaatattgatgtactaaaacagtgctctgcttaaatacagtttttcaacatacagtcttggaagaaacaaaatccaaaataaattccaatagtccagtaacaggaataaagacaactattgcaaattaaatcttacagacttatatgaaagctgttgttaacagctgggtactagttatttgaaaagtttctcgtgccgaattcgatatcaagcttatcgataccgtcgacctcgta

SEQ ID NO: 81 - DNA, 1090 bp, Homo sapiens
  SITE (8): n equals a,t,g, or c
cattgacntcaatgggagtttgttttgncacccaaaatccaangggactttccnaaattgtcgtaaccaactcccccccattgaccccaaatggncggtaggcgttgtacgggtgggaggtctatataagcagagctcgtttagtgaaccgtcaagatccgcctggagacgccatccacgctgttttgaccctccatagaagacaccgggaccgatccagcctccggactctagcctaggcttttgcaaaaagctatttaggtgacactatagaaggtacgmctgcaggtaccggtccggaattcccgggtcgacccacgcgtccgccagcctggaggcccagacgtggcgcagcgactcggaggttcgcctccagcttgcgcatcatctgcggccgggtcccgatgagcctcctgttgcctccgctggcgctgctgctgcttctcgcggcgcttgtggscccagccamrgccgccactgcctaccggccggactggaaccgtctgagcggcctaacccgcgcccgggtagagacctgcgggggnatgacagctgaaccgcctaaaggagagkgaaggctttcgtcacgcaggacattccattctatcacaamctggtgatgaaacacctccctggggccgaccctgagctcgtgctgctgggccgccgctacgaggaactagagcgcatcccactcagtgaaatgacccgcgaagagatcaatgcgctagtgcaggagctcggcttctaccgcaaggcggcgcccgacgcgcaggtgccccccgagtacgtgtgggcgcccgcgaagcccccagaggaaacttcggaccacgctgacctgtaggtccgggggcgcggcgganctgggacctacctgcctgagtcctggagacagaatgaagcgctcagcatcccgggaatacttctcttgctgagagccgatgcccgtccccgggccagcagggatggggttggggaggttctcccaaccccactttcttccttccccagctccactaaattccctcctgccttaaaaaaaaaaaaaaaaaaaaaaaaaaaaaaaaaaaaaaaaaaaaaaaaaaaaaaa

SEQ ID NO: 82 - DNA, 698 bp, Homo sapiens
gtctagtttatgtttttccactggacagggagctccttgaggaccttgtcttgctcgctgcccccaccctaaaacttgctgtaaagcagttcctggaacagagcaggtgctcagtagtactggttgcatgaatgaatgaatgaatgaataggttttcctcttttagacacattgggagatgggcctatggtttcctatgctcattttgacccagagatttgtgtcctgtgactcacatccagacccaaaacacacacatacacacgcacacataaatacacacacacacagacacgtgcacacacagacacacatgcacacacacatacacacaccttggtttgaagagaagagggatgggaacagacattctacgcatgcctacagtgcaccactgtgcataggtaactgatgctgtataagcactcaaggattatctccatttttagccagagaaactgaggcttgctttctgctgtgtctccagtgcctagcactgtgcctggcataaacatctgctgaactgaattgcactagattcaagaggctcagaaaacagttcaaggtcacccaactagcaagttgtggagccagaatctgtgctcagggctgttcagtccccagccagtgccgggtagcagccataggcacctgcacaaactccagcgacctcgttaacttccaaacacggtctcgta

SEQ ID NO: 83 - DNA, 868 bp, Homo sapiens
cacgcgtccgcggacgcgtgggcggacgcgtgggcaaaaatcttaaaagcactttatcatttcatttccctgcactgtaatttttttaaatgatcaaaaacggtatcataccaaggcttacttatattggaatactattttagaaagttgtgggctgggttgtatttataaatcttgttggtcagatgtctgcaatgagtaaatttagcaccattatcaggaagctttctcaccaatgacaacttcattggaagattttaatgaaagtgtagcatactctaggaaaaaaatatgaatattttagcatctatgtattgaaaattatgttgaataaatgtcagactattttttacataacgttgcttctgtttaattttgtcacgttcagaggtggggggtaggagatgtaagcccttgacagcaaaataattccttttgcttgatttcagacagttgcatcagctcctttgttctgtgttcatgttacacttatttaggtggctgaatccacagaggagcctgctggttctaatcggggacagtatcctgaggattcctcaagtgatggtttaaggcaaagggaagttcttcggaacctttcttcccctggatgggaaaacatctcaaggtgagtgttataataaagatcttggcttatgcaacatgaatgttcctcgtttgcatcaatttaagaataaggtatgtttacacgtatataatcagaacttttaaacatacagaattttgctttataaatagcttcgctttaaagatctcttatatatttaacttttcttaatacacagccttttagtacacacaaatttaaaaagtaggtaatgcatatattgaaaaaaaaaaaaaaaaa

SEQ ID NO: 84 - DNA, 629 bp, Homo sapiens
ggcacgagaacctttggggctgacacaagatcctttagtgtttgggatgacctctttcctgcagacttcttcccctatccctaactcatgcatggaaaacgtttgtcaggctggtttcccgagcctcctgcacctcaacatcacgctcacccttttgggtttagcccagtgttatttagcaaatttctccagctgcagggaaggatcagagcactatctttttttttttttttttctcctggagccaggactgcacaaggcaatggccaaatttagttgaattcagcctaccatcctttgctgatgactcagctctatgccaagtactggagccacagagatgggtcagtcccagcccctgtcctcaggaagcccatggtcagggaaacgttgtagggataagtaatagagggcagttgccttcagggctcctggtggctgctggtccctatggtgccttgatgtgaattagaagacggtgccctttccaggtggattcagacctacactagaacgcacagctttgggagtgacacacaggttggattttagcaccccttgccccttggccagaggtgccctgctgcacggccatacgctgcagcctcgagggacacacaggccaaagt

SEQ ID NO: 85 - DNA, 837 bp, Homo sapiens
  SITE (474): n equals a,t,g, or c
gcttccaggctccagcctctgcccgcactgcttgcagtaccctactcatgtgtcctcttcatgtgcccctccccggtcatatgggtcccttctggcccctgcccagcttatactctgtccgatcctcacagtcaccctgtcccctttgcttttctttgctgccactgcaggcccacctcagcctcctgcacactctcttcagatcagcctcccaatctccagcgtctggagtgttctggggctgcctgagagagagacatgaatacatgtcaccctgccttcctcacatgtaccagaagtttgatttttttttttttttttgactgagtcttgctctgtcaccaagctggagtgcagtggcacgctcggctcactgcaacctccacctcccgggttgcagcgattctcctgcctcagcctcccgagtagctgggattacaggcatgcaccagcatgcccagctaatttttgtanttttagtagagacagggtttcaccatgttggccaggatggktttgatctcttaacctcgtgatccgcccgccttggcctctcaaagtgctggaattacaggcgtgagccaccacgcccggccctgattattattattattattttaaacaataatctgggccaggcacagtggctcacacctgtaatcccaacactttttgggaggctgaggcaggaggattattgagcccaggaatttgagactagcctgagcaacatagtgagaccctgtctctacaaaaagtaaaaaattagtccaggcatggtggcacatgcctgtagtcccagctactcaggaggctgagataggaggatcactcgta

SEQ ID NO: 86 - DNA, 903 bp, Homo sapiens
ggcacagccttccccctgcccttcctgcctggctcactcctggccacccttcagactcctctctctgcctcctccagctggcgcctcacttggtgatggccgtgtctgttccatggcccctcccagaggkacttggtttctcctgctgtcattgcgtctcccttacggggccgcatgctgggttttcttaccatttcctgcatcctgcagagccgagggcgtggcagcaccaatcaagtgtagtaggaatgagtaggaaacaagcatccttctccatggcacagaarggagtctgtcaccttggaaagtcaytcaagagaggatccaagaaagcgtcttgccctamctacccctcctttagcaagtgaggatcttcgagggraggggagtttccaagtcaactggtgacaaagccaggatgagaagacactcccagaccactgtggctaatgacacacactgcccggccatgccatctgccagcgctggaggtggccgctcaacacaggaaggtcaaggtcatgttagcagctcccccacccagcaggggaaagggaaagacttgcactggggagcagttttatttatttttatttatttattattaattatttttagatggagtcttgctctgtcacccaggctgatgcagtggtgagattttagttcactgcaacctctacctcctgggttcgagcgattctcctgccttagcctcctgagtagctgggactataggtgtggtggtgcatgccggtaatcccagctactcgggaggctgaggcaggagaatcacttgaacctggaaggcggaggttgtggtgagccgagatcacaccattgcactccagcctggacaacaagagtgaaatccgtctcaaaaaaaaaaaaaaaaaactcgta

SEQ ID NO: 87 - DNA, 725 bp, Homo sapiens
aggttctaagcattttgcttgacctgactcatttaatcctcacaaaactctacaagataagtatattctcactactttacaggctaaaaatctgaggcacagaaaagttactgaagctccaaggtcacactgtgtaccataagtggaagagctaggatgcaaacccaggcagccgggttccagagcagtgttctaactactaccctctgttgcctctcattcatcccatgaccttcttttgtcttacctacactgggatgtgtttgggacatgcattttgcttgttgctatctcattcttgcagaatgcattgtacttgctatttgtgtctattcacagttcaggttttgccaggcaagtacaatgaaggaggagaggggcaaaggaattgagggtgcctacaagggagtagttagagagatggatgtgaaatctaagctgggcaaattgagaagtaaggacatgatataggtgatgggcagtaaaaatatgtaatgtcagcagtttaaaggactggatggggcagatattaattggagttgcaggactaaaggagttcaaaatataggaaatgaataccagagacagagagagggctgaagtcaaaatgttggaggtggtacttattattaacaacaaggtctagaggatgaccgcagaattggggtccaaggtgacacatggctgacagctgtcattgaccacactgtaatgcagaactcgta

SEQ ID NO: 88 - DNA, 606 bp, Homo sapiens
tggtcccccgggctgcagattcggccgagaattacacgaattaawttattcatgaggctacatttcatttcatatgcatgtttccaggttgtattctcttgtgcaatctgtgtatgttctttgtcttatctttttctatgggaatatttgctttttattcacttataagagcaatgcatgtatcaaggttagattttaattttgcaacatattttgtggcataatcaggtttaaaatgcttgaagttaccatatatgtaaattttttcttcatgttctttgcatttaagtgactggaagagttcattccttccactgaaatcactgaataactaccttggctacttggtgccaatgatgaaggcatcatatttatacccctcaaaggattcacagtccaggaagaagcagacaaacgaagactttcataagtgctatggagagccaaggaaccatctcgatctgctgggaattcctggggcaggaaactgaggatgggactgtggtccaaggaggcagactctgaccaggctgggacagggaaggggagcgttcaggtcaaggtggtcggccttctgtcagagcatactgcattacagtactcgta

SEQ ID NO: 89 - DNA, 1142 bp, Homo sapiens
  SITE (39): n equals a,t,g, or c
tgaacagtgcaggtagatactggactgggggcagatctnagggagaggggtttaagtagtgggaggacactggggataggggcttggggctatttacctgccattttaagtagtttgctattttagcagccaacaataactattggtgctgaataccagccctgcagtgtagcatgagacaggtccatgcacacatgcattaggaaaacaccttcatgaagcaggattctgcctgggctgatgcacacaacctctatggagggtgaaacagtgtttctgaagaccgtagtttgggaacccctgacatatgagcaatgcccccttagataagctcaagttacaggaatgtytgagggtggaaggtgtggatatgtgcttttgcctgtytccctcttacagtgtctggccatggggcataaacactacccagcagtaggtaggytggccaagagaagccagcttgcatcaccagcatcatctagggaatggaatcatggcagtaatacgttgcttaggaaacaaaagctctatggacacatcttccaccttctcagtcccagaaaccrtatgtactgtgaccccgctcaytaggcccagccctcgggaagagtgtgggcccttgaaaagggaagactgagtgagcaaaatgatgagaaaactacaaaatgggcagaggtcagtctgacacattcattctctgtcaagctcaggaagtactggtccctgatcttggagatgctgtgtgagtggcagggggactcctgctgggtaaatattctatatgtggatgcctggacaggcccctatcccaggccctgcttgtcagaagctccccttgggccgagcgcggtggctcacacttgtaatcttggcactttgggaggccgaggcaggtggattgcctgagttcaggagttcaaaaccaggctgggcaacatggtgaaaccctgtctctactaaaaaaaaactaaccaggcgtggtggtgcatgcctgtaattccagctactagggaggctgaggcaggccaatcacttgaacccaggaggtggaggttgcagtgagctgagatcacgccactgcactctagcctgggcaacagagcgagactctgtctcaaaaaaaaaaaaaaaaaaaaactcga

SEQ ID NO: 90 - DNA, 596 bp, Homo sapiens
  SITE (4): n equals a,t,g, or c
gganaccngctttgccccttggtttccncaaagctcgaatttaccctcactaagggnaccnaaagctggagctcccaccgcgttggcggcccgctctagaactagtggaccccccgggctgcaggaattcggcacgagtcctgacctcaggtgatccacccacctcggcttcccaaagtgctaggattataggcttgagctactgtgcccggcccatggtgtttttctttagggctcttcctacagccttgagaagtagataggcatcagagtatggtactataggaatcagaaaaattcaaaacaaatgtggattaagtgtttaggctctatgtggctcacgcagccagaatccttaagtctgtgtgtttctgtgtctcaagactgggctcacattctggctttgtccataacaatgctctgggatttcagggagttccctcatttgtaaaatgagggggtcagagcaggtgatatccatgtttcttccctttctgatattgttgtctgtggcatattctttgtatggcgaatttaataaattatattaatgtgtctctttgaaaaaaaaaaaaaaaaaaaaaaaaaaactcgta

SEQ ID NO: 91 - DNA, 633 bp, Homo sapiens
ggcagagtgtctctcaatggcttctttcttgaagggcatcacagccactgtacttatcaatgcctgtgtagccaacacagtagctcctctacattacaaggatatgattattcctaaacttgtcgatgatctaggaaaagtaaaaatcactaagtcaggatttctcacttttatggacacttggagcaatccactggaggaacacaatcaccaaagtcttgttccattggaaaaggcgcaggtgcccttcttgtttattgttggcatggatgatcaaagctggaagagtgaattctatgctcagatagcctctgaaaggctacaagctcatgggaaagaaagaccccagataatctgttacccagaaactggtcactgtattgacccaccttattttcctccttctagagcttctgtgcacgctgttttgggtgaggcaatattctatggaggtgagccaaaggctcactcaaaggcacaggtagatgcctggcagcaaattcaaactttcttccataaacatctcaatggtaaaaaatctgtcaagcacagcaaaatataacattgtagccacagaccagataccattaataaaaatcctattcataaaaaaaaaaaaaaaaaaaactcgta

SEQ ID NO: 92 - DNA, 725 bp, Homo sapiens
ggcagagcttccctagcaataattactttgcttaatttacttttttcattcttgtgcgttcctttatatttcatatattaaatatccatcaacattatataggggtctttaaacattatgtaacaagatacatattgaatgtattacactgcagcttgccttttcatttcagtgttgtttttaggtttatctgtgttgataagcgttgctgtagttcattcattttttaaacattgtatagtatttcatgatgattaaaccacaatttatttattctcctgttgatagacaattaggatgttttcagttttttgctgtgacaaatactcccgttatgggcattattttgtctcctttttacatagatacaaaagtttccctacggtatataccaagaaatggaatttctgagtttttagggtatggacattctcagctttactagattttgcctagttcatctccaaaactgtggtactaatatactttcccaccagcagtatataagagggcctgtttctccacatctttgttaaaactatatattgtcaaatttttaaattttgccaatctgggccagacactggggctcacatctgtaatcctgtaatcctagcattttggaaagcagaggcaagaggatcgcttgaggccaagagtttgggaccagcctgggcaacagagcaagaccccgactctacaaaaaaaaaaaaaaaaaactcgta

SEQ ID NO: 93 - DNA, 601 bp, Homo sapiens
tcccccgggctgcaggaattcggcacgaggtcggcacgacactgccccaaaatcaaaatggctcaagtccactttcaaaaatgtcagtgctcaccaacagtgggtgaaaaggctgcctgacccagcttctcagagagccagtgcctcaaatccaatgcatggcaattgctctggggcccctggttttaagctggctttgttatttgtggctgacactggaaagcctctgcacaaacaagatggcaagtgatgagccggtcagtcatcactgccttcccagactctctgaaccacccttgacattctgcctggaagcagggggcttggtggaggtgggtgacctcttgaagtcccgggccaggcctgtgattctgtaatctttgctttaccataattagggagggaggcagaagagcaggaggagaaaccatttattacttctctgggattttgacagcttggaaaaagagagagacagagaaacagtccagagaaggagccagccacagtgagtttaacctctcagtaaaataaaaatgggctggacgcacctcatcagctgccctctgtcaatacccgggcccatctggcaggactcgta

SEQ ID NO: 94 - DNA, 692 bp, Homo sapiens
ggcacgagctaaaagagctagtttgagtaagctgtgtaagacagctgctgctaaatagaaccaaattcacctgcctatggccggccacccagtgttctttctgctcatccacctactgcccttagacttcagcatgggctggacccagaccccaggatctaacaactggcgacgaggatggaaggaggtgagtgggtcttcagcccctgagggctcccgggacggctacgtggccgcagcatgagctgtggtacccggtcgcagtggtgctgcttggatgagccccagtggaaacatgggaggcagtgtacagatcccctatgagtgtggagaaggcgctgaatcacctggaaatgcacagcattgaaaggaacatacctttgccagcagagtcagatgggcatttgcgactatgctgagggaaatgaatgcccaatccctgcaggatgcagcgcagggaggaggaacctccgttgcaggcttgcccggtagtccgtcagaaaatagagcatgaacagctgttgggccccaagaggaggcccagagaccccccatcgtggtggaacacatttcctatggtgcctgtgtccccgctgaattgagggagttaagcaactaatgtcgccagttgtgtacagacttagtgcaagtcattcgggagaaggacatttgcgcaacctagtcctactcga

SEQ ID NO: 95 - DNA, 1005 bp, Homo sapiens
  SITE (506): n equals a,t,g, or c
ggcacgagctcgtgccgttggttttccctctgtctgttcagtgggttctgaacattctttgatggctggatcctactcctctgacatcttagtgttggcaagatcttggaccctcctccttctttctgttttgaggttgcagaccgttggctcatcagtcacactggactcacaggtgggtattatttggcctgcagttttcaaaataggaaatcgtgttaaaaaacaaaatcaaataaaagaaaaacgacaacaacaaaaccaaaactgaacttccaatttatcttggagaattagcagacctagtaaaatgagttctgtattctcatatggcaataattttctggagctgagtacctgcttcttgggtcattcttaatcaactcattctttccaaacatcttatacccagcctgtgtcattcatttaggtgagctgacaaaggctagtaggaatataaatttatgacccttagtttatactctccccagtggatcttatttaantacccattwaaataccatatgctttaaaaagtcttctttcataacattgagtgcacacaatatgccctgaactatgtaccagacactggggatacgcggtgaatkacgcaagtcactctacttccaaagaacttaccttctatagaggggagacacacacaacagtgataacataaagccaaataatatttgggctgggcgcagtsgctcatgcctgtaatcccagcacttcgagaggctgaggcgagcggatcacgaggtcaagagattgagaccaacctggccaacatggtgaaatcctgtctctactaaaaatacaaaaattagctgggtgtggtggcaggtgcctgtaatcccagctacttgggaggctgaggcaggagaattgcttgaacctgggaggcgaaggttgcattgagccgagattgtgccactgcactccagcctggtgacagagcgagactccatctcaaaaaaaaaaaaaaaaaaaaaactcgta

SEQ ID NO: 96 - DNA, 612 bp, Homo sapiens
gggatctgtgtaagacaaaattaatagctgtatctggagtactctaaatgtggatttatacactaacctatatattgatcaattcctctatgcttgctttggttttgagcaaattatatttaaataagtttgttgctaggaatgtcttaaaaagctactcaccctttttgttagaagtaagtaaatgattatgtcaggacctgccattaacttggtatagtacgaatatatcctcagaatactgataaaatggtatgtcttgaaacaaatcacaaactgtcaatatgttggtgatgaatttcttctgttttcatttggatcagtagtggggcagttcaccaagtgtgagatcgacatttaatgttttcatgaaatgcaaacccatcagtggctaatttgttaaaaaatagatgttgggcttttcttaaggctaaattgttcccatttgttttagagaacaactcacttagcctatgagtttatgcaatttggcagaaagtgaaaacatatttggaagtattgaaagtcactcattgttgatcttttatattggaatgyccaaggttgcatcatcagagtgtcgttatgaaaaaaaaaaaaaaaaaactcgta

SEQ ID NO: 97 - DNA, 670 bp, Homo sapiens
gctcgtgccgaactcgtgccgacgaaaagctgccaagttgaaaatggacgagtaatcgcctgctttgattcattgaaaaactaaatctccatacccacttcatccgtgtttttggcttatgtatgggatgctagaatggcctatctccatgtattttgttgcatttctccattgcttcttgtgttctggcgggaatcttggtgattcttttcaagcactacctgagctctgtgccaattgttcctcttctcccagggtgttgtgctgcgtggtcatgtctccacttccttagccctgtccattgacagaaccttgggttctgtgatggctgcctctaaacccttgtgaaagcggggaatattcctccccctgctgctacagttgagcaccgtgctgggtaccatgttgccctctacacttgctttcagttgttaaggcttcccaagctttggctgtggctcagtgatcctgctgtcaaaaccctgaaactttcctagcctggacactcagtggtagcagcaggtgttgggatttctccaagcccctaagactctgggaggaagagaatggctgtttgacatagacctcaggagttttcaaagcaccaagaaacctctccagaagatatgtaaagattttaaagggaaaaaaaaaaaaaaaaaaaactcga

SEQ ID NO: 98 - DNA, 619 bp, Homo sapiens
gcggcacgagtgatatttcacgtcacatggctagtgagtgggtaggcctctcttcacttattacacttctgcttctaagctgtgttctttcctgtattacactggaggaaggagaaaaagaacttgtatttggtccttgactgggtggaatatcctttaatgtggctgtaaggacatgggtagaatactctggtcaattcatttcttatttaaatagtgacaaaggtatgtccatgttaaccatttctcacttatgctttatacataaggatggcttatagggaatgttgctttattatatcacttaaaatgtttggtcaggcaatagtgactcatgcctttaatcccagtacttttgaaggacaagtcaggaggatcgcttgagaccaggaactcaggaccagcctggacgacaaaacaggatctcgtctctacaaaaaataaaatagtcgagtgtggtgatgcagtattgtagtcccagctatttgggaggctgaggtgggagtatcgcttkagaccaggagttcaaggatatagtgaatgatgatcgctccactgcattccagcctggacaacaaagcaaaaccctatttctaaaaaaaaaaaaaaaaaactcgta

SEQ ID NO: 99 - DNA, 703 bp, Homo sapiens
gcttggttacgtttatagcttcaacacgcctctcattktaggtttatacatgtgtttgcttgctcatttattttgtcatcatttgctcattttattaccagttattgagwgcctactgtgtaccaggcactgggcaaggggcattctgtgagagagggtatggtacctgcgggcttaagtagtccgtgggcttgtgaggaaaacgctagattagatcttgattactgtaaatgtcaartatggccaagtgtgggatttcgtggcaggagtgagctttcctggaatttgtctttcttgcctcaatttgcctgatagtcatttcatgctagggatgttttaaagtctctggggaggccctgcagtgtagaggaaaatgctgatccacaccagaaatgcgaacctggctctctgcccttgggcaagtcacttaaccctcctgagcctcagtttccatctgtcacttagagctgattatacctacttaacacccaggctttttgtgaggggcattatctcattagagataatgtttttaaaagctctttgtaaattgtgtagcattcaaatggaagttattgttatttttattattgagtgccttctaattcaacactgggatagtaacaaaagaagagaggggttattatcacccctcttccctgtcacgtttagattggggcaaggaaaggttctcaccctgcga

SEQ ID NO: 100 - DNA, 762 bp, Homo sapiens
gtttttctccttcttagtatcttttgcatatagaaaataattactatgaaattatagatttgacgtgcaaaggctatttcttgaattttattaaaatgcaaaaagatgcatccatgtcttctctaaaaggactgcgtattcctccacacttggggaaatgcagcttgtgctatttcacaggctcatcatgccccttttttttgccaggacgctggttgattaatgccatgcttggggagtgctccagccagaaatgagggctatcgcctgtggccaataacagagcagattctcaataaacatccccttggtgttacacttaatggggcttgcttttccaaactgctccctttcctgggctctgagcagctgagccgagagctcgtaagctctgctgccccagaacattgtgcattcyttgattttgaaaartctttcctgaagsctcctcttgggtcattggatcagcccaagagcaaaggatttaaaagggccaatttgatagggacagctcatagccctgtgtaagaccactgggcatttttcctgtttggggaaatggttactggattagcattttgctgtacagggcggtctgcaagaatgtgtgctcttgcctgtcctcaaagcaggcttgtgaggagctttctgttcccagccctgccatttcctcccaattggctgggccagatgctccagacacagttaatgagatgctgagtgaaacagagccgctggctcacatggcctcagcctcctcga

SEQ ID NO: 101 - DNA, 650 bp, Homo sapiens
  SITE (497): n equals a,t,g, or c
ggcacgaggtgtcctgcccaccccagtgcgggtcagtagaaggccagaagcaggggatgggagaaggcaggtgggagggcgtgacagcggcgaggatgaggaaggcagccaggcctgcaggcagccctgagagcatgaagcagaggggtgagcaggttcccctcctcctgccacccttgctcctctctaccaggctctggccttgctggggtgtacccacagaatctgtaggctctggcctagccagaaagagtgtgggtgcttctcagggtcataattaccccatgccccacagggtgtgagtcactggtagcagagtcctccccaatcccccccagaagagtgtggtgaaaggcccgggccactggggtgtcgagagtgccaggcctgacctactgggggtggtgtcagtaggggccatataccctgttctcamgacaaccccaggccaactcagatttgtggagcggccatcccacctccttccggctcttcancctcacaggagcctggtgggtcgggaaaactgaggcctagagaggcaaaatgatgatacaatgaagagtgagtacatgtggaacaccctctgtgcctcacactccactaagctcctcacaccattcacttactcaggcctcaccggccctcga

SEQ ID NO: 102 - DNA, 360 bp, Homo sapiens
ggcacagctgatgtttaaaatacacgaaaaatcttgtaaccctattttggcatatctttttcttcttctttttggtttttgtttaatatggaagtggacagtgcctctcttgacctctggaaggccctatgaaaacctgaaaccgaggcaaggtgacaaagtctggtcattcagcactaagggccgcctcagattacttctttacttagaaaaacaaaatgttgttgcaaaagattcagagtcacaaatattcttcccgggcctgtcagtttctgaattcttagatttttcatttaatttagccatcagggaatttctgagactagaaatacctaggcagaacccaaacaaaatctcgta

SEQ ID NO: 103 - DNA, 817 bp, Homo sapiens
ggcacgagctcaggttgcggccggagagaaaggcctggggaccacctgactctgggccacccgggcctcctcaggtcttcggccagcgctgtcctgcccacggtagttggggttccaatggctgcggcttcttcctgtctgtggcttggacatgccattggccgcgtctctatttcctcatctgcgactcgggtgaccacagttctcagttcaccgtgttcggtagaggtgacatgaagtgcctggcacccatgtgggtttccctgtgggattctgacccgcttcggagctgcctcctgctcctcatcccacacttctctgtgtttctcatcctggcggctgtgtcctgtctgcccctctcaactgcaacacgctggagaggtcgggaccctgtcttgctcattatctgtctactaaagaacctgcaaaatggaaaaataacaatatgtgctgaattaattattagcttaaaatttaaaacttaagtagcatgatttgagtgcagccagcatcacctgccgtgagatcggtgctgtctacaggaggatggagcttttggtgaaccactgagctgggagtagctacgggcacctttacccagtcccaaaatgtggaacatttgagtttaaaaagcagaaaactctacagttaaaagccaatattaaggttgagtccattaatctaaattaatctgattttttatttctttaaataaaaaagtaatcctatgcaatcaaagttaaagttcgtatatggctccctatgaggtactacattccctgaagtgtcacaaaaaaaaaaaaaaaaaaaaaaaaaa

SEQ ID NO: 104 - DNA, 881 bp, Homo sapiens
ggcacgagtatgactaataaggtaatctgtccttgttaacaagcctgtatttgttatacctgtacttaaagtaaaattcaaactccttaccctgtcctacaaggctctacctgatctgggccctacctcatctctaacatcatcttatgctattttctttcttgttcaccagagccacaccagctacctttctgtccctccttgttagacttatttctgctttagagcacccttgctgctgccaccacctgaaatgcttctcttctggtattttattttggtgagaacacctggcatgagatctaccctctaacagatttttaagtgtataatacagtattgctgtctgtaggcacaatgctgcacagcagatctctagaacttaccttgtataactgaaattttatactcattgattagcaacagccccaaattattgaaacctccttgaagcctaaatttcagaaatgttcaaatgttttgaaaatggatattctgaattatcttattagcatctacctataattagcactgaaaatagtaatttttttaataaagaatcagttaagggccgggtgtggtcctcacgcctgtaatcccagcactttgggaggctgaggcgggaggatcacaaggtcgggagatcgagaccatcctggctaacaccgtgaaaccctgtctctactaaaaaaatacaaaaaaaatcagctgggcgtggtggcaggtgccaatagtcccagctacttgggaggctgaggtcaggagaatggcgtgaacccaggagggttgcagtgagccaagttctcgccactgcactccagcctgggcgacagagcgagactctgtctcaaaaaaaaaaaaaaaaaaaaaaaaactcgtag

SEQ ID NO: 105 - DNA, 655 bp, Homo sapiens
ggcagagctggtctcgaactcctgacctcaggtgatctgcccaccttggcctcccaaagtgctgggattacaggcataagccattgcgctcggctgagattagcaataattaatgtgatatgaaaatattttctttttcttcatgacaaattcatggctaatactgccaggatttttttgttgttgcccatattcataatagaaggaaatgctaatatgaaaataaagatgtcactttttccccaatccatgcaatttccccctaaattgtatccatgacctacctgagggggatccatggactctcaggttaagacccctctactgaagggtagcagagtacagtttcaaaattactgattaagagcgtgggctcaccaggagttcaagcccagccggggcaacaggatgagacctcatctttacaaaaaatgaacaaaattaggcatggtggtgcttgtctgcagtcccagctacttgggagactgagttgagaggatcacttgaggctgagaggttgagggtgcagttgagctgagattgcaccactgcactccagcctgagtgacagagtgagatcctgactcaaaaaaaaaaaaaaaaaaaaagaaaaaaaaaaaaaaaaaaaagaaaaaaaaaaaaaaaaaaactcgta

SEQ ID NO: 106 - DNA, 606 bp, Homo sapiens
  SITE (9): n equals a,t,g, or c
ccccccggnctgccaggantttcggcacgagtctctctgtcaactctatttgtatttctataatggaaactcaaatttgcctaactcagattgtagcacttttcttcctcaggctagtcctaggaaaactcacttgttttttgtatggaaaactagtgttagtagaagcctttattcttgcatagcccccaaatcagctttttcagctataatttagtaagtctaatgtgttcgactgaagtactttttttttgtaataacaagtgaaaaataatgaagagtgtgtcctggcgcatggctcacgcctgtaatcccagcacttcgggaggccggagcygaggcagcggatcacttgagggtcaggagttcaagaccagcttgaccaacatggtgaagtcctgtctctattaaaaatacaaaaattagccaggtgtggtagtgcatgtctgtaatcccagctacttgggaggctgagacaggagaattgcttggacctgggaggcggaggttgcagtgaggtgagattgcggcattgcactccagcctggacaacaagagtgaaactttgtctcaaaaaaaagaaagaaaaaaaaaaaaaaaactcga

SEQ ID NO: 107 - DNA, 657 bp, Homo sapiens
  SITE (634): n equals a,t,g, or c
gagtttgtraacctatattcacagcattaactaatcatgattcgccccatatttcactggttatgctttggttatcttagaaaagaacccagggcatttatgaggtaaaacttgcagggcagattacaggcatgagccaccgcgcctagacttattagtcttttttaatgggatgacagcagctgggrtgtatatattcctgcaggaaagaaaaggaaatggcttcacattgctggatgggagcagtatgtgtgttgtttctgggtataatcttcctagctgcacttttcccatacatttctttctactaaaaatcatgaaagtttgaattatagttcctctcacaggattgaaagcaagtatcagaggagtcatccattcaaaacacagttcttccactgcagtatccgatatgttttgtatgtgcgctaggctgtcttttcattcagtctacaatacagttcaccagtgtggagaccttttgccctgcctgatttgttttgttttgttttactcactcttttcaatgacttttggttttggccagtatgaagagtaatggatgttggaataccttctgccagttaaaaaaaaaaaaaaaaaaaaaagggcggccgctctagaaggatccaanttaagtaagcgtgtcnctccnct

SEQ ID NO: 108 - DNA, 605 bp, Homo sapiens
acgagctggaaatcaatgatcagtcataaaatcagactgggaaactraggcacagagaggggcatggatttgggcattggtccaggttatgaagcacatccaccagggtggcctggtggagttaaaggccatccctactgggcaggatgtgctggtgccagttgggtgagttcagaggtggttgggagagagaaatgctcagagctctctgtctgtctacctgtccctgactctcagtgccagcacccacccaccccatggtccccactcatccgggagcttacagcagcccctccacctctatccagccattttctctagccataacattggtgactggcaaagtgtcccagcacaaggcctggcacacagttggtgcttagtgtttgctaaatgaatgaatggattaataagaacgaatattgtgcagaaaaagtaaattcttctggacacttccagcctatatgtggaggggacaaagttttttgttgttgttgttgttgttgttgttgtgttttttgagacagtgtcgttctgttgcccaggctggagtgcagtggtgcgatcacagctcactgcagccttgatctcctcagcctctcgta

SEQ ID NO: 109 - DNA, 504 bp, Homo sapiens
ggcacgagccaacagccgttttgaaggtagaggagagagatgttgtggtatttkttccccaccaccccactccctgcccaggtgcagttttggtggtgcctgtgttgctgctacatccatggctcctggtggggacccctctcccaaagctccagctcctgcaatgcttcagtaactgcactcagctcaggctgttgtagacctagggccagcagtcccacagtgcctcaccatcgcttgttccctatgcctgcccacacatctgtaaatagtcccttcatttcacatccttcagttagaccctttgagtatgccatctgcttccggtcaggacaatgattgattctatctgaatcaaacctgtcctttatttgaacaggacatcaagtctagaaaaacaagttaacaccttgagataacaaacaaatccagaatttgggaccatttactagtctggttctttcaaaggtcaatgttataaaaaaaaaaaaaaaaaactcgtag

SEQ ID NO: 110 - DNA, 770 bp, Homo sapiens
gctaaaattcaacaaggtgagtggccggcagtggaaggctgttgctcattctgatttctgttggctctatttcatgctaamccagttttttttgtttgtttgtttccactttataacatatggatttctatgccacactacccgtaactttgaaaaataactttaggctgcagttttcagcaaacaggacagtccttagctgccacatagctcaacataaagtgcacaaaaaacttcacggtgggacagtgaatcataaattcccaaactgacgtgtgtctacagaacagatgagaactgttactcagtgtgtatcttaggagcttttctgcagtttcctcacactccgtcacatttaaaatgtggacacttgtttatttcattagggaggaggcgagggactaatgtccaccctgcccagagtatttcgaatatccttagtgaagaggaggaaagcaagaattctgttctaaaggccaccaggctaagcactagaatcgcattctcttcctgtttgtatgtttatgtcagcagttgccacagatgtgttaatattgttttcctggtagagaattaaggtgttcgttcatctcaaaacaaatcccgtaacctgcacacaaaactccagcttcctaatgcaaagagaagagaatattgattataagctgcttgatattctttttattcccagcccctcaaaataccagcctggaagtctggacattactaaaatttaccagtctcaaaaaaaaaaaaaaaaaaaactcgag

SEQ ID NO: 111 - DNA, 751 bp, Homo sapiens
ccacgcgtccgcggacgcgtgggagtcatctgtcttaagttggaaaaaagtttcatatgattctttcccatttcccctgtattccttttcattataccctcattccttgaacagaattgttattgttttgtttttccatccacacccctataatgcaaccttcctgtgtaaattttaggcttaagcttttctattcacatacttttatgctgaggcttggatttttatttgggctgttagatgcccattttgacattgacattaggggtttcaaaccatccttaaaaggttagatgtgacttgcaatgttattgaacaatttgatgatccgggatattatggctctatgaaatctccatggttcttggagctagcttgtttttattctgggaagaattttctagctccccagcttacggcctgaatggttagagtccagccagtgctgtttgactttatagttcaaagggggtcatttctgtggtcactatcctatttaacagtcatgtcatggtatgtcaaggtaggtcatcatacaaataatctgcattctgttttgactgttttatttttaaaaataatatctcctccttttaaactttaaaaaatttagtaaagtttagtaaactttcaaaaatttagtaaaaaatgtagtaaaaattcacttccttcattatgctttttgaaatctggctttttttctcattcttcccctattaatggttcttaaaaaaaaaaaaaaagggcggcc

SEQ ID NO: 112 - DNA, 543 bp, Homo sapiens
  SITE (22): n equals a,t,g, or c
cgtcgcccgcttggagggtcgncactagtggatccaaaggantcggcacgngctaccccttgccmaagcctaaacttcatactagatatccaactgcctactggacatctccatttataagcctagtagcctaataagcataacctcagacttaccaggcctcacactgaagtcatgaacttcagcccaacccccatgccagggcaaaaccttgttgttacctcttattcctctcttgcctcatcccatccatgttcagtctgtcagtggatcctgtgagtccagtcttgaggatagttccaggatctgatcacttctcactgcctcttttgctgccaccacctctggcctggataattgcagcagcctcccagttagccttgctgtgtccatccttgttttccccttctgtctgctctcaacagaggagctagtgattctcttaggacagaataaatcatttaggttttcttcacatggtcctgaagaagcttcctacctcactcagtgtaaaaaccaaaaaaaaaaaaaaaaaaaactcga

SEQ ID NO: 113 - DNA, 846 bp, Homo sapiens
ggagtttttttttcattttagtttatattaaataacaaatatttattcctgtgaatcagtagtttacacagataatattgagaggctttcttgggaatttgaaaggagtcttcaaatcatcctttccctcagagatgaaaaaatattttaaaaaaattactgtcttgtatatttgatattttgaaaatggcagggaatcaacaatttgttaatctgttgttaagatcagttatacattcagtggcatacttcttgtcttagaaattggttgaaattaatattgctagtgaaagtgtggaaatagraacagttgaaaggaagacaaatgagaagtggaccttgcttctcatgaggatgctgcagaactagagtggttgcccagcaggatgaaaatctcaattaattgcttgacagagaattaaaacaaaggcaagtggtgcttttaaaaaagataaaaataggtgaatataaagttgaaaggaggccaggtacagtggctcacacctgtaatcccagcactgtgggagcccaaggtgggtggatggcctgaggtcaggagtttgagaccagcctggacaacatggtgaaacgctgtctctactaaaaacacaaaaattacttgggcgtggtggcatacgcctgtaatcacagctactccagaggctgaggcaggagaatcacttgaacctggaaggtagaggttgcagtgagccgagatcgcgyccattacactccagcctgggtgacaagagcaagactatgtttccaaaaaaaaaaaagcaactgaatattggatagagaggagaaaaagggcaatgtatcaaaaaaaaaaaaaaaaaactcgag

SEQ ID NO: 114 - DNA, 890 bp, Homo sapiens
aggttacttattgctcctacttcatatcatatgtggttctacaacctacattatcttgtctatgtcttttaactagctgtgtgttcttacataagatctgcagaccttggttctcaactgcaaaagcatattgattaaatgattactgtttttacctgcaatactttaatttttggatttgggattaataatgtaaaaaagactaacatatatgtgggattacaaaactgttttgttagccttcaaacaactatgaactgcatcaggagctgtcttatacttattgttctgctattaatacttaatgcactgcctgtaaagagctgattgctacttaaaaactctgcttaaatgaaaaaccaaaacataaaagattaaaccaaacatacttactctcccatagccctggtggacagcaacataaggagggaaatgtttctgttgatctttggcttcaaggattaataccagatttggataccggttagttagataattggtaaggaatcccataaagttgtaaattacataagcttcatagcattctctgcaggtatccacatatattgcaattccgggatatttcaaagctatccactatgaaaaagcacagatgttaaagatagttgcagctaagataaaatgaatcaccactccattcatggtactcacaataagctaatttttatgcttgagatgtcttgtcatatacttacatgggactctctaaaatttatcattatgagggctatcaatctgtgaaatgaatgcttaaaagcaataaacatcttagatattggtaaacaaaaacaagtgtttgaggggtaaataatgaataaagagagaagctaaagtaaaaaaaaaaaaaaaaaaaaaaactcgtagggggg

SEQ ID NO: 115 - protein, 86 aa, Homo sapiens
  SITE (2): Xaa equals any of the naturally occurring L-amino acids
MXLQPNPHARAKPCCYLLFLSCLIPSMFSLSVDPVSPVLRIVPGSDHFSLPLLLPPPLAWIIAAASQLALLCPSLFSPSVCSQQRS

SEQ ID NO: 116 - protein, 20 aa, Homo sapiens
  SITE (20): Xaa equals stop translation
MAAHSVLSFLLWTPYALKSX

SEQ ID NO: 117 - protein, 39 aa, Homo sapiens
  SITE (39): Xaa equals stop translation
MLKLATILLTLLLKNLDAGLTDKLSRSNFITDFILTKYX

SEQ ID NO: 118 - protein, 88 aa, Homo sapiens
  SITE (86): Xaa equals any of the naturally occurring L-amino acids
MLLLYLGIEVIRLFFGTKGNLCQRKMPLSISVALTFPSAMMASYYLLLQTYVLRLEAIMNGILLFFCGSELLLEVLTLAAFSSMDXIX

SEQ ID NO: 119 - protein, 39 aa, Homo sapiens
  SITE (39): Xaa equals stop translation
MYKFLYLVLEDFVAFIRGSFPPQHTRSLVFWHVCQLEYX

SEQ ID NO: 120 - protein, 27 aa, Homo sapiens
  SITE (27): Xaa equals stop translation
MMMMIQTLMVMAKILCLKQPLSMAGSX

SEQ ID NO: 121 - protein, 22 aa, Homo sapiens
  SITE (13): Xaa equals any of the naturally occurring L-amino acids
MKENPLLLLICIXGHLVVPPNX

SEQ ID NO: 122 - protein, 96 aa, Homo sapiens
  SITE (96): Xaa equals stop translation
MYRDSHSVLALNWKVVATLKYFLLYVIILYNLERDNGHSNYENYELGDKSLNLLLFYNSMYKLVFPYIFTFSSFLISSYTSILYKMFYIQRTVKSX

SEQ ID NO: 123 - protein, 36 aa, Homo sapiens
  SITE (36): Xaa equals stop translation
MKERTRIPCAFPFLLFQTRVQTSPAFQPHPLYFTAX

SEQ ID NO: 124 - protein, 38 aa, Homo sapiens
  SITE (38): Xaa equals stop translation
MTSVIVLFILKVFFKYFSTTSFLNACIHFIHKCKLVNX

SEQ ID NO: 125 - protein, 342 aa, Homo sapiens
  SITE (342): Xaa equals stop translation
MLQPTHLSLQLRLQCLAASHLVTLLICLMAPASATGGSADLFGGFADFGSAAASGSFPSQVTATSGNGDFGDWSAFNQAPSGPVASSGEFFGSASQPAVELVSGSQSALGPPPAASNSSDLFDLMGSSQATMTSSQSMNFSMMSTNTVGLGLPMSRSQPLQNVSTVLQKPNPLYNQNTDMVQKSVSKTLPSTWSDPSVNISLDNLLPGMQPSKPQQPSLNTMIQQQNMQQPMNVMTQSFGAVNLSSPSNMLPVRPQTNALIGGPMPMSMPNVMTGTMGMAPLGNTPMMNQSMMGMNMNIGMSAAGMGLTGTMGMGMPNIAMTSGTVQPKQDAFANFANFSKX

SEQ ID NO: 126 - protein, 219 aa, Homo sapiens
  SITE (139): Xaa equals any of the naturally occurring L-amino acids
MVSWMICRLVVLVFGMLCPAYASYKAVKTKNIREYVRWMMYWIVFALFMAAEIVTDIFISWFPFYYEIKMAFVLWLLSPYTKGASCFTASLSTRPCPAMRRRSTRTSCRPRSAATRPCSASGSGASTLPPPLLCRLPPXVRGRWPAGCGASPCRTCAPSLTHLPLPTMTPSTWRTRCPTGGHPLGTGPGACRTATPRMSVGQILRQSPGRQPGPERXPX

SEQ ID NO: 127 - protein, 266 aa, Homo sapiens
  SITE (15): Xaa equals any of the naturally occurring L-amino acids
MSMAVETFGFFMATXGLLMLGVTLPNSYWRVSTVHGNVITTNTIFENLWFSCATDSLGVYNCWEFPSMLALSGYIQACRALMITAILLGFLGLLLXIXGLRCTNIGGLELSRKAKLAAXAGALHILAGICGMVAISWYASTSPGTSSTPCTPEPSTSWAPXSTWGGAPHXSPSWVASASAPPAAAALTXTSRQRPAXLPXSRVRDARRHLGPRRRQQLWQIRQKRLRVAXLARGXRCLPTAPRXXDXAGAHSPIVTSGAGHAPLPX

SEQ ID NO: 128 - protein, 39 aa, Homo sapiens
  SITE (39): Xaa equals stop translation
MLFIYLFVFPIRIGSEKAKTVSVLLIIVSLTARPLAGFX

SEQ ID NO: 129 - protein, 93 aa, Homo sapiens
  SITE (93): Xaa equals stop translation
MLLYLYSLGISVLIISFPTNSSIHVRKNMANQYLKGAIFQSSGFQSVAGQHWQHLNLLGTLLKMQILSPTLVLLNWETGVGPSSLCFNMFSKX

SEQ ID NO: 130 - protein, 196 aa, Homo sapiens
  SITE (196): Xaa equals stop translation
MELSESVQKGFQMLADPRSFDSNAFTLLLRAAFQSLLDAQADEAVLDHPDLKHIDPVVLKHCHAAAATYILEAGKHRADKSTLSTYLEDCKFDRERIELFCTEYQNNKNSLEILLGSIGRSLPHITDVSWRLEYQIKTNQLHRMYRPAYLVTLSVQNTDSPSYPEISFSCSMEQLQDLVGKLKDASKSLERATQLX

SEQ ID NO: 131 - protein, 49 aa, Homo sapiens
MASILLLLVLSHSCCCKNTCLQVLCNFDSVHNLSTLILKIIIRVDVLVY

SEQ ID NO: 132 - protein, 55 aa, Homo sapiens
  SITE (55): Xaa equals stop translation
MVYCVHLNPFTDLCCIFFMPLLCFLLRSRVDSISIPSLTLLEACNSIYCSGSSAX

SEQ ID NO: 133 - protein, 33 aa, Homo sapiens
  SITE (33): Xaa equals stop translation
MGVNKVLFTFFFFSSLLDGVGTSHSLASFPHTX

SEQ ID NO: 134 - protein, 24 aa, Homo sapiens
  SITE (24): Xaa equals stop translation
MWPLLLRLLFLHLFLAKNKLIFKX

SEQ ID NO: 135 - protein, 220 aa, Homo sapiens
  SITE (68): Xaa equals any of the naturally occurring L-amino acids
MAEIHTPYSSLKKLLSLLNGFVAVSGIILVGLGIGGKCGGASLTNVLGLSSAYLLHVGNLCLVMGCIXVLLGCAGWYGATKESRGTXLFVGDVALEHXFVTLRKNYRGYNEPDDYSTQWNLVMEKLKCCGVNNYTDFSGSSFEMTTGHTYPRSCCKSIGSVSCDGRDVSPNVIHQKGCFHKLLKITKTQSFTLSGSSLGAAVIQLPGILATLLLFIKLGX

SEQ ID NO: 136 - protein, 303 aa, Homo sapiens
  SITE (303): Xaa equals stop translation
MIGISASFSALLEQILCASGHSSGFSGLCGALFITFGILGALALGPYVDRTKHFTEATKIGLCLFSLACVPFALVSQLQGQTLALAATCSLLGLFGFSVGPVAMELAVECSFPVGEGAATGMIFVLGQAEGILIMLAMTALTVRRSEPSLSTCQQGEDPLDWTVSLLLMAGLCTFFSCILAVFFHTPYRRLQAESGEPPSTRNAVGGADSGPGVDRGGAGRAGVLGPSTATPECTARGASLEDPRGPGSPHPACHRATPRAQGPAATDAPSRPGRLAGRVQASRFIDPAGSHSSFSSPWVITX

SEQ ID NO: 137 - protein, 41 aa, Homo sapiens
  SITE (41): Xaa equals stop translation
MRLVPSHLLAILINIKDQMMCFCIALMMRLSSCIASSGPWX

SEQ ID NO: 138 - protein, 278 aa, Homo sapiens
  SITE (278): Xaa equals stop translation
MSFNLQSSKKLFIFLGKSLFSLLEAMIFALLPKPRKNVAGEIVLITGAGSGLGRLLALQFARLGSVLVLWDINKEGNEETCKMAREAGATRVHAYTCDCSQKEGVYRVADQVKKEVGDVSILINNAGIVTGKKFLDCPDELMEKSFDVNFKAHLWTYKAFLPAMIANDHGHLVCISSSAGLSGVNGLADYCASKFAAFGFAESVFVETFVQKQKGIKTTIVCPFFIKTGMFEGCTTGCPSLLPILEPKYAVEKIVEAILQEKMYLYMPKVVILHDVSX

SEQ ID NO: 139 - protein, 111 aa, Homo sapiens
  SITE (111): Xaa equals stop translation
MLTFLMLVRLSTLCPSAVLQRLDRLVEPLRATCTTKVKANSVKQEFEKQDELKRSAMRAVAALLTIPEAEKSPLMSEFQSQISSNPELAAIFESIQKDSSSTNLESMDTSX

SEQ ID NO: 140 - protein, 133 aa, Homo sapiens
  SITE (133): Xaa equals stop translation
MRALHFSSRHNKDIALVNLANVLHRAHFSADAAVVVHAALDDSDFFTSYYTLGNIYAMLGEYNHSVLCYDHALQARPGFEQAIKRKHAVLCQQKLEQKLEAQHRSLQRTLNELKEYQKQHDHYLRPGNPRKTX

SEQ ID NO: 141 - protein, 131 aa, Homo sapiens
  SITE (131): Xaa equals stop translation
METLGALLVLEFLLLSPVEAQQATEHRLKPWLVGLAAVVGFLFIVYLVLLANRLWCSKARAEDEEETTFRMESNLYQDQSEDKREKKEAKEKEEKRKKEKKTAKEGESNLGLDLEEKEPGDHERAKSTVMX

SEQ ID NO: 142 - protein, 106 aa, Homo sapiens
  SITE (106): Xaa equals stop translation
MTHRRHCGLARWILMKIFCWRVSTVTSTAGALTNPHSCYTSVLKVGATGVGQSLSVWTMPGLLLEQFSTGVELLLSSSRFSNSMEYKNRLSSVEDRSSVVTCLKAX

SEQ ID NO: 143 - protein, 62 aa, Homo sapiens
  SITE (62): Xaa equals stop translation
MPLALLATWLSCLPSLVLTYYSRSNQKMPWTLASPFSSMASTMEFWNGTLQKCVQTTWHLPX

SEQ ID NO: 144 - protein, 38 aa, Homo sapiens
  SITE (38): Xaa equals stop translation
MKATLKLLPTIVVIYCLLCPVPRQILGVPSWAPGKCLX

SEQ ID NO: 145 - protein, 64 aa, Homo sapiens
  SITE (64): Xaa equals stop translation
MLTSSSNLISWVLPELSSLLWVFLFWKRQCGDWAGRKTRSRVSGVVTNFPLHSPSLRYSSFLEX

SEQ ID NO: 146 - protein, 105 aa, Homo sapiens
  SITE (105): Xaa equals stop translation
MLFCILLYTLGSARCHHLSFFLWGWSNPPEKTPLASWRGVKARLPGPGCQLLGAAGAEAGSCQAFSQQDALSTHLGFRIPLPHLQMGQMSPKPAAPFCFTLSTEX

SEQ ID NO: 147 - protein, 61 aa, Homo sapiens
  SITE (61): Xaa equals stop translation
MGPWCLTLLSTTSGFFSENLYLTLILSFLLSIESVNTDPFIFQFPKSCFAIASILLSGGVX

SEQ ID NO: 148 - protein, 37 aa, Homo sapiens
  SITE (37): Xaa equals stop translation
MGCTALLLLFHLCVPCEPYGTHEKELVPGLYFLVYRX

SEQ ID NO: 149 - protein, 32 aa, Homo sapiens
  SITE (32): Xaa equals stop translation
MCKFIYVPHSVLLVYVFTFVLIPNCYNSVALX

SEQ ID NO: 150 - protein, 16 aa, Homo sapiens
  SITE (16): Xaa equals stop translation
MSLALCLVPLVREGHX

SEQ ID NO: 151 - protein, 59 aa, Homo sapiens
  SITE (59): Xaa equals stop translation
MIISSIRCLVLGIECVSAVCQNLLLGEFPHWERDPGNGMVLEGLLNTFPWEGSCYLQGX

SEQ ID NO: 152 - protein, 87 aa, Homo sapiens
  SITE (87): Xaa equals stop translation
MLKTWFFVMAVIGVIIPTVFDQSSRLCLKETGFVQNVDQSNVLEDSPLDRDHPWKVMKMWKTVWEVRMMKLMAMKKKVKVRRKSMRX

SEQ ID NO: 153 - protein, 53 aa, Homo sapiens
  SITE (51): Xaa equals any of the naturally occurring L-amino acids
MDICSPVALYFLLTAAHITAVSKPTVMLRERPCSGPSRSAPQSRLIGPWDXCX

SEQ ID NO: 154 - protein, 78 aa, Homo sapiens
  SITE (78): Xaa equals stop translation
MALKNKFSCLWILGLCLVATTSSKIPSITDPHFIDNCIEAHNEWRGKVNPPAADMKYMIWDKGLAKMAKAWGKPVQIX

SEQ ID NO: 155 - protein, 72 aa, Homo sapiens
  SITE (72): Xaa equals stop translation
MLQAASLSLVTWVVCTVWLETTVPPSLPEPPMWPLSSDSSWSLWISTGMAPAPSSSTRSFSVLPEICFCLWX

SEQ ID NO: 156 - protein, 41 aa, Homo sapiens
  SITE (41): Xaa equals stop translation
MLQEVKLDFLWLLNLPLILLFSILESSMKICTNAMFTRTGX

SEQ ID NO: 157 - protein, 85 aa, Homo sapiens
  SITE (85): Xaa equals stop translation
MEVWHGLVIAVVSLFLQACFLTAINYLLSRHMGNWLSILFPPSHSQRPFSSLQQDRPFGVPKRHSKTTRGPTGQIPSHRSPSPQX

SEQ ID NO: 158 - protein, 96 aa, Homo sapiens
  SITE (96): Xaa equals stop translation
MAEPIACLCLICCIIISATTQMPFEGSCFCLVPCNFQPYFRHFRPNDLRHMVFTHGLWALEKLSPLKENQNVACICIFCLRFHLILKWILDSPKVX

SEQ ID NO: 159 - protein, 89 aa, Homo sapiens
  SITE (89): Xaa equals stop translation
MWAVLPAWFPFPGTCHCLPVSLRGHFWEVRPWPPGPLFRSEAPTCLGSGSSGVRPCPPQDIPSKPAMSGDGPLPGKVLFLLVTEKNLPX

SEQ ID NO: 160 - protein, 44 aa, Homo sapiens
  SITE (44): Xaa equals stop translation
MSALSFTSYFLLLLRVKPVEVSGSIPHPEQPNVLCLVLPTFGYX

SEQ ID NO: 161 - protein, 46 aa, Homo sapiens
  SITE (46): Xaa equals stop translation
MCCFFLKTLNLWLGYFCQFICLPCQVTLCLIDVLCVFHSVHAELSX

SEQ ID NO: 162 - protein, 62 aa, Homo sapiens
  SITE (62): Xaa equals stop translation
MYLFLKTLLSFSTLMMTTALSFMVITVLWVLLLHLLANICIPRKCSFACFYINGILLHAVFX

SEQ ID NO: 163 - protein, 31 aa, Homo sapiens
  SITE (31): Xaa equals stop translation
MVSLLSLTFHQFVSSLKYFKLLSTSRQEILX

SEQ ID NO: 164 - protein, 94 aa, Homo sapiens
  SITE (2): Xaa equals any of the naturally occurring L-amino acids
MXAISAHCNLYLPGSSDSPASASGVAVITGVCHHAQVIFVFLVETAFHHVVQAGLKLLTSGHPPTLGSHSAGITGVSHCTWPPFNFIFTYFYLF

SEQ ID NO: 165 - protein, 71 aa, Homo sapiens
  SITE (71): Xaa equals stop translation
MENPTSTPTLPCFLFFFSPRSLDVLTPPHCLLSGTGWDLEEDKAFLDYPSYSVSLFLTQRGRQNQSGLFQX

SEQ ID NO: 166 - protein, 43 aa, Homo sapiens
  SITE (43): Xaa equals stop translation
MRIHPIFRLGNVYSLLSFLILGRVSTKNSIEEKQYNIKIKKIX

SEQ ID NO: 167 - protein, 65 aa, Homo sapiens
  SITE (65): Xaa equals stop translation
MEKLLTLYLLLYVSYWSVSPTGQGAGLFIAQSSAPGLRQTHSRHLGNAWERKEGRREEGLHGHVX

SEQ ID NO: 168 - protein, 68 aa, Homo sapiens
  SITE (68): Xaa equals stop translation
MLFSLPRTFSSHSSPAQLIFIHAASVLMAFPPRPSKTTLPQAAFLTSLACPLMLSTFFLYQNAFVCKX

SEQ ID NO: 169 - protein, 59 aa, Homo sapiens
  SITE (59): Xaa equals stop translation
MSSFPGPQCVQLINLLHLICPVSGLVCSAITIALRQKSIPHQQGREAVIKTPPPGSLPX

SEQ ID NO: 170 - protein, 54 aa, Homo sapiens
  SITE (30): Xaa equals any of the naturally occurring L-amino acids
MLVLAWITFPPCKACCMMCIFSSRLLQQEXVCTXVQGXEPRGMAQRDRGFESLX

SEQ ID NO: 171 - protein, 20 aa, Homo sapiens
  SITE (20): Xaa equals stop translation
MVYHGYNIYLVVFLLLYLDX

SEQ ID NO: 172 - protein, 39 aa, Homo sapiens
  SITE (39): Xaa equals stop translation
MGPAVCFRACEMCSLSGLLLNLCFQSCLSVPLSGVPRVX

SEQ ID NO: 173 - protein, 54 aa, Homo sapiens
  SITE (54): Xaa equals stop translation
MNLETVLLSRTSSLGFAVCLPCFFCWFYLVLFLELTSITFAMYDIIPCMTLGKX

SEQ ID NO: 174 - protein, 55 aa, Homo sapiens
  SITE (55): Xaa equals stop translation
MSWALPSLFFLLFSPFLLPSGLTVIRRYTNNSNNYLKNHTHQKNKRQQKITRYSX

SEQ ID NO: 175 - protein, 47 aa, Homo sapiens
  SITE (47): Xaa equals stop translation
MLSPLNHLYFPFRFLCMLCSLPRVVFQLTPIKEAFPSQELTFPCTHX

SEQ ID NO: 176 - protein, 55 aa, Homo sapiens
  SITE (55): Xaa equals stop translation
MLLGFLCLWYQVYVCMYVCTYLFIYLLFSLFSLPHMICKKSVKFIMSSPKPPSGX

SEQ ID NO: 177 - protein, 27 aa, Homo sapiens
  SITE (27): Xaa equals stop translation
MKWSLLKVVLVFVFVCGFLKRAYPATX

SEQ ID NO: 178 - protein, 50 aa, Homo sapiens
  SITE (50): Xaa equals stop translation
MIDICHSLRREHFLLWSFLGLFYWAVNGKSVCVSLLHPKHLGKNESLLIX

SEQ ID NO: 179 - protein, 27 aa, Homo sapiens
  SITE (27): Xaa equals stop translation
MFHSSLLVFLSLLSQEIFTEYDCMHKX

SEQ ID NO: 180 - protein, 41 aa, Homo sapiens
  SITE (41): Xaa equals stop translation
MVHVSNLPWCLMTLSIFALLCNHHCHPSTERLSSCKTETPX

SEQ ID NO: 181 - protein, 65 aa, Homo sapiens [sequence omitted]

SEQ ID NO: 182 - protein, 20 aa, Homo sapiens
  SITE (20): Xaa equals stop translation
MVGTWRMLFLLPSYSSGQVX

SEQ ID NO: 183 - protein, 42 aa, Homo sapiens
MWDYKTVLLAFKQLMNCIRSCLILIVLLLILNALPCKELIAT

SEQ ID NO: 184 - protein, 46 aa, Homo sapiens
  SITE (46): Xaa equals stop translation
MVKWIILSCLILKGKRTLNSSTFYAANKSSTINRNLSWQALPFTHX

SEQ ID NO: 185 - protein, 72 aa, Homo sapiens
  SITE (19): Xaa equals any of the naturally occurring L-amino acids
MSLLLPPLALLLLLAALVXPAXAATAYRPDWNRLSGLTRARVETCGGMTAEPPKGEXRLSSRRTFHSITXWX

SEQ ID NO: 186 - protein, 78 aa, Homo sapiens
  SITE (78): Xaa equals stop translation
MGLWFPMLILTQRFVSCDSHPDPKHTHTHAHINTHTHRHVHTQTHMHTHIHTPWFEEKRDGNRHSTHAYSAPLCIGNX

SEQ ID NO: 187 - protein, 26 aa, Homo sapiens
  SITE (26): Xaa equals stop translation
MLNKCQTIFYITLLLFNFVTFRGGGX

SEQ ID NO: 188 - protein, 63 aa, Homo sapiens
  SITE (63): Xaa equals stop translation
MENVCQAGFPSLLHLNITLTLLGLAQCYLANFSSCREGSEHYLFFFFFLLEPGLHKAMAKFSX

SEQ ID NO: 189 - protein, 92 aa, Homo sapiens
  SITE (92): Xaa equals stop translation
MCPLHVPLPGHMGPFWPLPSLYSVRSSQSPCPLCFSLLPLQAHLSLLHTLFRSASQSPASGVFWGCLRERHEYMSPCLPHMYQKFDFFFFFX

SEQ ID NO: 190 - protein, 48 aa, Homo sapiens
  SITE (48): Xaa equals stop translation
MAPPRGTWFLLLSLRLPYGAACWVFLPFPASCRAEGVAAPIKCSRNEX

SEQ ID NO: 191 - protein, 45 aa, Homo sapiens
  SITE (45): Xaa equals stop translation
MCLGHAFCLLLSHSCRMHCTCYLCLFTVQVLPGKYNEGGEGQRNX

SEQ ID NO: 192 - protein, 48 aa, Homo sapiens
MFPGCILLCNLCMFFVLSFSMGIFAFYSLIRAMHVSRLDFNFATYFVA

SEQ ID NO: 193 - protein, 82 aa, Homo sapiens
  SITE (2): Xaa equals any of the naturally occurring L-amino acids
MXEGGRCGYVLLPVSLLQCLAMGHKHYPAVGRLAKRSQLASPASSREWNHGSNTLLRKQKLYGHIFHLLSPRNXMYCDPAHX

SEQ ID NO: 194 - protein, 40 aa, Homo sapiens
  SITE (40): Xaa equals stop translation
MWLTQPESLSLCVSVSQDWAHILALSITMLWDFREFPHLX

SEQ ID NO: 195 - protein, 182 aa, Homo sapiens
  SITE (182): Xaa equals stop translation
MASFLKGITATVLINACVANTVAPLHYKDMIIPKLVDDLGKVKITKSGFLTFMDTWSNPLEEHNHQSLVPLEKAQVPFLFIVGMDDQSWKSEFYAQIASERLQAHGKERPQIICYPETGHCIDPPYFPPSRASVHAVLGEAIFYGGEPKAHSKAQVDAWQQIQTFFHKHLNGKKSVKHSKIX

SEQ ID NO: 196 - protein, 40 aa, Homo sapiens
  SITE (40): Xaa equals stop translation
MYYTAACLFISVLFLGLSVLISVAVVHSFFKHCIVFHDDX

SEQ ID NO: 197 - protein, 73 aa, Homo sapiens
  SITE (73): Xaa equals stop translation
MAIALGPLVLSWLCYLWLTLESLCTNKMASDEPVSHHCLPRLSEPPLTFCLEAGGLVEVGDLLKSRARPVILX

SEQ ID NO: 198 - protein, 56 aa, Homo sapiens
  SITE (56): Xaa equals stop translation
MAGHPVFFLLIHLLPLDFSMGWTQTPGSNNWRRGWKEVSGSSAPEGSRDGYVAAAX

SEQ ID NO: 199 - protein, 70 aa, Homo sapiens
  SITE (70): Xaa equals stop translation
MAGSYSSDILVLARSWTLLLLSVLRLQTVGSSVTLDSQVGIIWPAVFKIGNRVKKQNQIKEKRQQQNQNX

SEQ ID NO: 200 - protein, 47 aa, Homo sapiens
  SITE (47): Xaa equals stop translation
MWIYTLTYILINSSMLALVLSKLYLNKFVARNVLKSYSPFLLEVSKX

SEQ ID NO: 201 - protein, 55 aa, Homo sapiens
  SITE (55): Xaa equals stop translation
MLEWPISMYFVAFLHCFLCSGGNLGDSFQALPELCANCSSSPRVLCCVVMSPLPX

SEQ ID NO: 202 - protein, 38 aa, Homo sapiens
  SITE (38): Xaa equals stop translation
MASEWVGLSSLITLLLLSCVLSCITLEEGEKELVFGPX

SEQ ID NO: 203 - protein, 34 aa, Homo sapiens
  SITE (21): Xaa equals any of the naturally occurring L-amino acids
MCLLAHLFCHHLLILLPVIEXLLCTRHWARGILX

SEQ ID NO: 204 - protein, 22 aa, Homo sapiens
  SITE (22): Xaa equals stop translation
MQLVLFHRLIMPLFFARTLVDX

SEQ ID NO: 205 - protein, 56 aa, Homo sapiens
  SITE (56): Xaa equals stop translation
MKQRGEQVPLLLPPLLLSTRLWPCWGVPTESVGSGLARKSVGASQGHNYPMPHRVX

SEQ ID NO: 206 - protein, 116 aa, Homo sapiens [sequence omitted]

SEQ ID NO: 207 - protein, 84 aa, Homo sapiens
  SITE (84): Xaa equals stop translation
MKCLAPMWVSLWDSDPLRSCLLLLIPHFSVFLILAAVSCLPLSTATRWRGRDPVLLIICLLKNLQNGKITICAELIISLKFKTX

SEQ ID NO: 208 - protein, 46 aa, Homo sapiens
  SITE (46): Xaa equals stop translation
MLFSFLFTRATPATFLSLLVRLISALEHPCCCHHLKCFSSGILFWX

SEQ ID NO: 209 - protein, 42 aa, Homo sapiens
  SITE (42): Xaa equals stop translation
MANTARIFLLLPIFIIEGNANMKIKMSLFPQSMQFPPKLYPX

SEQ ID NO: 210 - protein, 41 aa, Homo sapiens
  SITE (41): Xaa equals stop translation
METQICLTQIVALFFLRLVLGKLTCFLYGKLVLVEAFILAX

SEQ ID NO: 211 - protein, 31 aa, Homo sapiens
  SITE (31): Xaa equals stop translation
MASHCWMGAVCVLFLGIIFLAALFPYISFYX

SEQ ID NO: 212 - protein, 40 aa, Homo sapiens
MWRGQSFLLLLLLLLLCFLRQCRSVAQAGVQWCDHSSLQP

SEQ ID NO: 213 - protein, 100 aa, Homo sapiens
  SITE (5): Xaa equals any of the naturally occurring L-amino acids
MLWYXFPTTPLPAQVQFWWCLCCCYIHGSWWGPLSQSSSSCNASVTALSSGCCRPRASSPTVPHHRLFPMPAHTSVNSPFISHPSVRPFEYAICFRSGQX

SEQ ID NO: 214 - protein, 29 aa, Homo sapiens
  SITE (3): Xaa equals any of the naturally occurring L-amino acids
MLXQFFLFVCFHFITYGFLCHTTRNFEKX

SEQ ID NO: 215 - protein, 47 aa, Homo sapiens
  SITE (47): Xaa equals stop translation
MQPSCVNFRLKLFYSHTFMLRLGFLFGLLDAHFDIDIRGFKPSLKGX

SEQ ID NO: 216 - protein, 86 aa, Homo sapiens
  SITE (86): Xaa equals stop translation
ELQPNPHARAKPCCYLLFLSCLIPSMFSLSVDPVSPVLRIVPGSDHFSLPLLLPPPLAWIIAAASQLALLCPSLFSPSVCSQQRSX

SEQ ID NO: 217 - protein, 25 aa, Homo sapiens
  SITE (25): Xaa equals stop translation
MAGNQQFVNLLLRSVIHSVAYFLSX

SEQ ID NO: 218 - protein, 15 aa, Homo sapiens
  SITE (15): Xaa equals stop translation
MWDYKTVLLAFKQLX

SEQ ID NO: 219 - protein, 82 aa, Homo sapiens
  SITE (49): Xaa equals any of the naturally occurring L-amino acids
MLMKINFYPLPKPKLHTSISNCLLDISIYKPSSLISITSDLPGLTLKSXNFSPTPMPGQNLVVTSYSSLASSHPCSVCQWIL

SEQ ID NO: 220 - protein, 15 aa, Homo sapiens
PNKHNLRLTRPHTEV

SEQ ID NO: 221 - protein, 95 aa, Homo sapiens
  SITE (62): Xaa equals any of the naturally occurring L-amino acids
GTSLFLWALYVIYMLMKINFYPLPKPKLHTSISNCLLDISIYKPSSLISITSDLPGLTLKSXNFSPTPMPGQNLVVTSYSSLASSHPCSVCQWIL

SEQ ID NO: 222 - protein, 29 aa, Homo sapiens
MLMKINFYPLPKPKLHTSISNCLLDISIY

SEQ ID NO: 223 - protein, 28 aa, Homo sapiens
  SITE (20): Xaa equals any of the naturally occurring L-amino acids
KPSSLISITSDLPGLTLKSXNFSPTPMP

SEQ ID NO: 224 - protein, 25 aa, Homo sapiens
GQNLVVTSYSSLASSHPCSVCQWIL

SEQ ID NO: 225 - protein, 28 aa, Homo sapiens
GTSLFLWALYVIYMLMKINFYPLPKPKL

SEQ ID NO: 226 - protein, 70 aa, Homo sapiens
LAPRFAFSQCSLAIMLTLLFQIHFLMILSSNWAYLKDASKMQAYQDIKAKEEQELQDIQSRSKEQLNSYT

SEQ ID NO: 227 - protein, 37 aa, Homo sapiens
LAPRFAFSQCSLAIMLTLLFQIHFLMILSSNWAYLKD

SEQ ID NO: 228 - protein, 33 aa, Homo sapiens
ASKMQAYQDIKAKEEQELQDIQSRSKEQLNSYT

SEQ ID NO: 229 - protein, 27 aa, Homo sapiens
LISQTSFSLPSPGPINFLSQSEIYFSI

SEQ ID NO: 230 - protein, 56 aa, Homo sapiens
  SITE (13): Xaa equals any of the naturally occurring L-amino acids
IRHEGGGQPFTSXPLEILFFLNGWYNATYFLLELFIFLYKGVLLPYPTANLVLDVV

SEQ ID NO: 231 - protein, 89 aa, Homo sapiens [sequence omitted]

SEQ ID NO: 232 - protein, 116 aa, Homo sapiens
TRTISPRDSSTLQYREGQGYSHPAPSQNQSPADLKFSSLITVARASRVDHLGSLGFKQDLSHMLPVRAVLYLSHMSTESLMLVGFQSDVKASHPNPRRLSSTTFLVAHSVIFLLSS

SEQ ID NO: 233 - protein, 276 aa, Homo sapiens
  SITE (7): Xaa equals any of the naturally occurring L-amino acids
RVIRLTXRANWSSTAVAAALELVDPPGCRNSARVKYCVVYDNNSSTLEILLKDDDDDSDSDGDGKDLVPQAAIEYGRILTRLTHHPVYILKGGYERFSGTYHFLRTQKIIWMPQELDAFQPYPIEIVPGKVFVGNFSQACDPKIQKDLKIKAHVNVSMDTGPFFAGDADKLLHIRIEDSPEAQILPFLRHMCHFIEIHHHLGSVILIFSTQGISRSCAAIIAYLMHSNEQTLQRSWAYVKKCKNNMCPNRGLVSQLLEWEKTILGDSITNIMDPLY

SEQ ID NO: 234 - protein, 37 aa, Homo sapiens
  SITE (7): Xaa equals any of the naturally occurring L-amino acids
RVIRLTXRANWSSTAVAAALELVDPPGCRNSARVKYC

SEQ ID NO: 235 - protein, 34 aa, Homo sapiens
VVYDNNSSTLEILLKDDDDDSDSDGDGKDLVPQA

SEQ ID NO: 236 - protein, 36 aa, Homo sapiens
AIEYGRILTRLTHHPVYILKGGYERFSGTYHFLRTQ

SEQ ID NO: 237 - protein, 35 aa, Homo sapiens
KIIWMPQELDAFQPYPIEIVPGKVFVGNFSQACDP

SEQ ID NO: 238 - protein, 36 aa, Homo sapiens
KIQKDLKIKAHVNVSMDTGPFFAGDADKLLHIRIED

SEQ ID NO: 239 - protein, 35 aa, Homo sapiens
SPEAQILPFLRHMCHFIEIHHHLGSVILIFSTQGI

SEQ ID NO: 240 - protein, 36 aa, Homo sapiens
SRSCAAIIAYLMHSNEQTLQRSWAYVKKCKNNMCPN

SEQ ID NO: 241 - protein, 27 aa, Homo sapiens
RGLVSQLLEWEKTILGDSITNIMDPLY

SEQ ID NO: 242 - protein, 196 aa, Homo sapiens
  SITE (98): Xaa equals any of the naturally occurring L-amino acids
IRHEFTSEKSWKSSCNEGESSSTSYMHQRSPGGPTKLIEIISDCNWEEDRNKILSILSQHINSNMPQSLKVGSFIIELASQRKSRGEKNPPVYSSRVXISMPSCQDQDDMAEKSGSETPDGPLSPGKMEDISPVQTDALDSVRERLHGGKGLPFYAGLSPAGKLVAYKRKPSSSTSGLIQVRIIFNLGIAPLYTPR

SEQ ID NO: 243 - protein, 16 aa, Homo sapiens
EFGTSLHQKRAGSLPA

SEQ ID NO: 244 - protein, 37 aa, Homo sapiens
IRHEFTSEKSWKSSCNEGESSSTSYMHQRSPGGPTKL

SEQ ID NO: 245 - protein, 33 aa, Homo sapiens
IEIISDCNWEEDRNKILSILSQHINSNMPQSLK

SEQ ID NO: 246 - protein, 36 aa, Homo sapiens
  SITE (28): Xaa equals any of the naturally occurring L-amino acids
VGSFIIELASQRKSRGEKNPPVYSSRVXISMPSCQD

SEQ ID NO: 247 - protein, 34 aa, Homo sapiens
QDDMAEKSGSETPDGPLSPGKMEDISPVQTDALD

SEQ ID NO: 248 - protein, 24 aa, Homo sapiens
CNIEYIRSDKCMFKHELEELRTTI

SEQ ID NO: 249 - protein, 127 aa, Homo sapiens
  SITE (8): Xaa equals any of the naturally occurring L-amino acids
HHQQVPEXDREDSPERCSDXXEEKKARRGRSPKGEFKDEEETVTTKHIHITQATETTTTRHKRTANPSKTIDLGAAAHYTGDKASPDQNASTHTPQSSVKTSVPSSKSSGDLVDLFDGTSQCNRRXS

SEQ ID NO: 250 - protein, 95 aa, Homo sapiens
  SITE (60): Xaa equals any of the naturally occurring L-amino acids
VSSDSVGGFRYSERYDPEPKSKWDEEWDKNKSAFPFSDKLGELSDKIGSTIDDTISKFRXKIEKTLQKDAATXXRKRKREEADLPKVNSKMKRRL

SEQ ID NO: 251 - protein, 45 aa, Homo sapiens
RQSIFISHRPQRPPQPDTSAQQILPKPLILEQQHITQGTKQVQIR

SEQ ID NO: 252 - protein, 190 aa, Homo sapiens
  SITE (72): Xaa equals any of the naturally occurring L-amino acids
DQDGLRAVAALTLHQGRQLLYRKFVHPSLSRHEKEIDAYIVQAKERSYETVLSFGKRGLNIAASAAVQAATXSQGALAGRLRSFSMQDLRSISDAPAPAYHDPLYLEDQVSHRRPPIGYRAGGLQDSDTEDECWSDTEAVPRAPARPREKPLIRSQSLRVVKXKPPVREGTSRSLKVRTXKKTVPSDVDS

SEQ ID NO: 253 - protein, 14 aa, Homo sapiens
AASWGPPHVPKAGK

SEQ ID NO: 254 - protein, 38 aa, Homo sapiens
DQDGLRAVAALTLHQGRQLLYRKFVHPSLSRHEKEIDA

SEQ ID NO: 255 - protein, 36 aa, Homo sapiens
  SITE (34): Xaa equals any of the naturally occurring L-amino acids
YIVQAKERSYETVLSFGKRGLNIAASAAVQAATXSQ

SEQ ID NO: 256 - protein, 34 aa, Homo sapiens
GALAGRLRSFSMQDLRSISDAPAPAYHDPLYLED

SEQ ID NO: 257 - protein, 35 aa, Homo sapiens
QVSHRRPPIGYRAGGLQDSDTEDECWSDTEAVPRA

SEQ ID NO: 258 - protein, 35 aa, Homo sapiens
  SITE (20): Xaa equals any of the naturally occurring L-amino acids
PARPREKPLIRSQSLRVVKXKPPVREGTSRSLKVR

SEQ ID NO: 259 - protein, 25 aa, Homo sapiens
  SITE (15): Xaa equals any of the naturally occurring L-amino acids
PVREGTSRSLKVRTXKKTVPSDVDS

SEQ ID NO: 260 - protein, 153 aa, Homo sapiens
  SITE (45): Xaa equals any of the naturally occurring L-amino acids
LCHRLPGRLQLLGVPVHAGPLWVYSGLPGTHDHRHPPGLPRPLAXHXGPALHQHWGPGALQESQAGGXRRGPPHSGRYLRDGGXLLVRFNITRDFFDPLYPGTKYELGPXLYLGWSASLXSILGGLCLCSACCCGSDEDQPPAPGGPTXLPCP

SEQ ID NO: 261 - protein, 20 aa, Homo sapiens
GVLPLPPLWGHQPPRVLHPT

SEQ ID NO: 262 - protein, 34 aa, Homo sapiens
LCHRLPGRLQLLGVPVHAGPLWVYSGLPGTHDHR

SEQ ID NO: 263 - protein, 37 aa, Homo sapiens
  SITE (11): Xaa equals any of the naturally occurring L-amino acids
HPPGLPRPLAXHXGPALHQHWGPGALQESQAGGXRRG

SEQ ID NO: 264 - protein, 35 aa, Homo sapiens
  SITE (13): Xaa equals any of the naturally occurring L-amino acids
PPHSGRYLRDGGXLLVRFNITRDFFDPLYPGTKYE

SEQ ID NO: 265 - protein, 36 aa, Homo sapiens
  SITE (4): Xaa equals any of the naturally occurring L-amino acids
LGPXLYLGWSASLXSILGGLCLCSACCCGSDEDQPP

SEQ ID NO: 266 - protein, 23 aa, Homo sapiens
  SITE (20): Xaa equals any of the naturally occurring L-amino acids
SACCCGSDEDQPPAPGGPTXLPC

SEQ ID NO: 267 - protein, 33 aa, Homo sapiens
VDQMFQFASIDVAGNLDYKALSYVITHGEEKEE

SEQ ID NO: 268 - protein, 15 aa, Homo sapiens
IRHEAYVILAVCLGG

SEQ ID NO: 269 - protein, 48 aa, Homo sapiens
FAPGARKEPFRPRPQVDQMFQFASIDVAGNLDYKALSYVITHGEEKEE

SEQ ID NO: 270 - protein, 185 aa, Homo sapiens
  SITE (105): Xaa equals any of the naturally occurring L-amino acids
WIQRIRHETNPKCSYIPPCKRENQKNLESVMNWQQYWKDEIGSQPFTCYFNQHQRPDDVLLHRTHDEIVLLHCFLWPLVTFVVGVLIVVLTICAKSLAVKAEAMXEAQVLLKGKEACRKQSTEAVLIGTRPPAEPVFPGAGDGQGHDRALRGSSLSGNRNRHNWKTWNLKACIPSAVAMAKGSRS

SEQ ID NO: 271 - protein, 36 aa, Homo sapiens
WIQRIRHETNPKCSYIPPCKRENQKNLESVMNWQQY

SEQ ID NO: 272 - protein, 35 aa, Homo sapiens
WKDEIGSQPFTCYFNQHQRPDDVLLHRTHDEIVLL

SEQ ID NO: 273 - protein, 35 aa, Homo sapiens
  SITE (34): Xaa equals any of the naturally occurring L-amino acids
HCFLWPLVTFVVGVLIVVLTICAKSLAVKAEAMXE

SEQ ID NO: 274 - protein, 36 aa, Homo sapiens
AQVLLKGKEACRKQSTEAVLIGTRPPAEPVFPGAGD

SEQ ID NO: 275 - protein, 43 aa, Homo sapiens
GQGHDRALRGSSLSGNRNRHNWKTWNLKACIPSAVAMAKGSRS

SEQ ID NO: 276 - protein, 55 aa, Homo sapiens
  SITE (52): Xaa equals any of the naturally occurring L-amino acids
KLFYKKKCTCICQKLLYFMMFLKKVITSASITSLTCQSTVLLPNPTQEKATXKNT

SEQ ID NO: 277 - protein, 152 aa, Homo sapiens
  SITE (21): Xaa equals any of the naturally occurring L-amino acids
HYEKVRLQVPIRNSRVDPRVXKFTISDHPQPIDPLLKNCIGDFLKTLEDPDLNVRRVALVTFNSAAHNKPSLIRDLLDTVLPHLYNETKVRKELIREVEMGPFKHTVDDGLDIRKAAFECMYTLLDSCLDRLDIFEFLNHVEDGLKDHYDIK

SEQ ID NO: 278 - protein, 37 aa, Homo sapiens
  SITE (21): Xaa equals any of the naturally occurring L-amino acids
HYEKVRLQVPIRNSRVDPRVXKFTISDHPQPIDPLLK

SEQ ID NO: 279 - protein, 34 aa, Homo sapiens
NCIGDFLKTLEDPDLNVRRVALVTFNSAAHNKPS

SEQ ID NO: 280 - protein, 37 aa, Homo sapiens
LIRDLLDTVLPHLYNETKVRKELIREVEMGPFKHTVD

SEQ ID NO: 281 - protein, 44 aa, Homo sapiens
DGLDIRKAAFECMYTLLDSCLDRLDIFEFLNHVEDGLKDHYDIK

SEQ ID NO: 282 - protein, 79 aa, Homo sapiens
IRHEHLRGVQERVNLSAPLLPKEDPIFTYLSKRLGRSIDDIGHLIHEGLQKNTSSWVLYNMASFYWRIKNEPYQVVECA

SEQ ID NO: 283 - protein, 42 aa, Homo sapiens
IRHEHLRGVQERVNLSAPLLPKEDPIFTYLSKRLGRSIDDIG

SEQ ID NO: 284 - protein, 37 aa, Homo sapiens
HLIHEGLQKNTSSWVLYNMASFYWRIKNEPYQVVECA

SEQ ID NO: 285 - protein, 27 aa, Homo sapiens
EFGTSPHQTCGRRPGTAAGWLLAHSTV

SEQ ID NO: 286 - protein, 296 aa, Homo sapiens
NSARDSLNTAIQAWQQNKCPEVEELVFSHFVICNDTQETLRFGQVDTDENILLASLHSHQYSWRSHKSPQLLHICIEGWGNWRWSEPFSVDHAGTFIRTIQYRGRTASLIIKVQQLNGVQKQIIICGRQIICSYLSQSIELKVVQHYIGQDGQAVVREHFDCLTAKQKLPSYILENNELTELCVKAKGDEDWSRDVCLESKAPEYSIVIQVPSSNSSIIYVWCTVLTLEPNSQVQQRMIVFSPLFIMRSHLPDPIIIHLEKRSLGLSETQIIPGKGQEKPLQNIEPDLVHHLTFQA

SEQ ID NO: 287 - protein, 26 aa, Homo sapiens
NKCPEVEELVFSHFVICNDTQETLRF

SEQ ID NO: 288 - protein, 25 aa, Homo sapiens
HICIEGWGNWRWSEPFSVDHAGTFI

SEQ ID NO: 289 - protein, 27 aa, Homo sapiens
VVREHFDCLTAKQKLPSYILENNELTE

SEQ ID NO: 290 - protein, 27 aa, Homo sapiens
EDWSRDVCLESKAPEYSIVIQVPSSNS

SEQ ID NO: 291 - protein, 27 aa, Homo sapiens
IIHLEKRSLGLSETQIIPGKGQEKPLQ

SEQ ID NO: 292 - protein, 27 aa, Homo sapiens
NSARDSLNTAIQAWQQNKCPEVEELVF

SEQ ID NO: 293 - protein, 34 aa, Homo sapiens
QETLRFGQVDTDENILLASLHSHQYSWRSHKSPQ

SEQ ID NO: 294 - protein, 40 aa, Homo sapiens
QYRGRTASLIIKVQQLNGVQKQIIICGRQIICSYLSQSIE

SEQ ID NO: 295 - protein, 41 aa, Homo sapiens
NSSIIYVWCTVLTLEPNSQVQQRMIVFSPLFIMRSHLPDPI

SEQ ID NO: 296 - protein, 162 aa, Homo sapiens
  SITE (44): Xaa equals any of the naturally occurring L-amino acids
LIIQDQTRRCHGLWHLPSLLWPLLWSSGTGLCRNVCRLHGIYHXVLXRVGHAYQTSFRQXVCXXWAADLCGRHEEGIIENTYRLSCNHVFHEFCIRGWCIVGKKQTCPYCKEKVDLKRMFSNPWERPHVMYGQLLDWLRYLVAWQPVIIGVVQGINYILGLE

SEQ ID NO: 297 - protein, 164 aa, Homo sapiens
  SITE (95): Xaa equals any of the naturally occurring L-amino acids
TAFVTFRATRKPLVQTTPRLVYKWFLLIYKISYATGIVGYMAVMFTLFGLNLLFKIKPEDAMDFGISLLFYGLYYGVLERDFAEMCADYMASTIXFXSESGMPTKHLSDSXCAXCGQQIFVDVMKRGSLRTRIGCPAIMSSTSSASVAGASWERSKRVPTAKRR

SEQ ID NO: 298 - protein, 25 aa, Homo sapiens
LIIQDQTRRCHGLWHLPSLLWPLLW

SEQ ID NO: 299 - protein, 26 aa, Homo sapiens
  SITE (19): Xaa equals any of the naturally occurring L-amino acids
SSGTGLCRNVCRLHGIYHXVLXRVGH

SEQ ID NO: 300 - protein, 24 aa, Homo sapiens
  SITE (9): Xaa equals any of the naturally occurring L-amino acids
AYQTSFRQXVCXXWAADLCGRHEE

SEQ ID NO: 301 - protein, 30 aa, Homo sapiens
GIIENTYRLSCNHVFHEFCIRGWCIVGKKQ

SEQ ID NO: 302 - protein, 31 aa, Homo sapiens
TCPYCKEKVDLKRMFSNPWERPHVMYGQLLD

SEQ ID NO: 303 - protein, 26 aa, Homo sapiens
WLRYLVAWQPVIIGVVQGINYILGLE

SEQ ID NO: 304 - protein, 28 aa, Homo sapiens
TAFVTFRATRKPLVQTTPRLVYKWFLLI

SEQ ID NO: 305 - protein, 29 aa, Homo sapiens
YKISYATGIVGYMAVMFTLFGLNLLFKIK

SEQ ID NO: 306 - protein, 22 aa, Homo sapiens
PEDAMDFGISLLFYGLYYGVLE

SEQ ID NO: 307 - protein, 28 aa, Homo sapiens
  SITE (16): Xaa equals any of the naturally occurring L-amino acids
RDFAEMCADYMASTIXFXSESGMPTKHL

SEQ ID NO: 308 - protein, 32 aa, Homo sapiens
  SITE (4): Xaa equals any of the naturally occurring L-amino acids
SDSXCAXCGQQIFVDVMKRGSLRTRIGCPAIM

SEQ ID NO: 309 - protein, 25 aa, Homo sapiens
SSTSSASVAGASWERSKRVPTAKRR

SEQ ID NO: 310 - protein, 20 aa, Homo sapiens
HEFCIRGWCIVGKKQTCPYC

SEQ ID NO: 311 - protein, 28 aa, Homo sapiens [sequence omitted]

SEQ ID NO: 312 - protein, 19 aa, Homo sapiens
WIPRAAGIRHEPGRHLGSS

SEQ ID NO: 313 - protein, 116 aa, Homo sapiens [sequence omitted]

SEQ ID NO: 314 - protein, 21 aa, Homo sapiens
CCSLWIYDYLIHPVPSVGHRV

SEQ ID NO: 315 - protein, 26 aa, Homo sapiens
ISPLGEGPRKWHGLRRSPEHISLGGL

SEQ ID NO: 316 - protein, 29 aa, Homo sapiens
RAVLPGNRTMETETYQLWASQYQRKWVSR

SEQ ID NO: 317 - protein, 149 aa, Homo sapiens
  SITE (128): Xaa equals any of the naturally occurring L-amino acids
WIPRAAGIRHEHLSTLDRSVIWSKSILNARCKICRKKGDAENMVLCDGCDRGHHTYCVRPKLKTVPEGDWFCPECRPKQRSRRLSSRQRPSLESDEDVEDSMGGEDDEVDGDEEEGQSEEEEYEVEQXEDDSXEEXEVRXVLXCNKMSQ

SEQ ID NO: 318 - protein, 11 aa, Homo sapiens
MRVARYVERKA

SEQ ID NO: 319 - protein, 22 aa, Homo sapiens
EHLSTLDRSVIWSKSILNARCK

SEQ ID NO: 320 - protein, 32 aa, Homo sapiens
TVPEGDWFCPECRPKQRSRRLSSRQRPSLESD

SEQ ID NO: 321 - protein, 6 aa, Homo sapiens
IRHEDD

SEQ ID NO: 322 - protein, 183 aa, Homo sapiens
  SITE (29): Xaa equals any of the naturally occurring L-amino acids
QRWLKHGANQCKFEHNDCLDKSYKCYAAXEXVGENIWLGGIKSFTPRHAITAWYNETQFYDFDSLSCSRVCGHYTQLVWANSFYVGXAXAMCPNLGGASTAIFVCNYGPAGNFANMPPYVRGESCSLCSKEEKCVKNLCKNPFLKPTGRAPQQTAFNPXQLRFSSSENLLMSFIYKRNSQMLK

SEQ ID NO: 323 - protein, 6 aa, Homo sapiens
DPPHPS

SEQ ID NO: 324 - protein, 29 aa, Homo sapiens
  SITE (12): Xaa equals any of the naturally occurring L-amino acids
CLDKSYKCYAAXEXVGENIWLGGIKSFTP

SEQ ID NO: 325 - protein, 40 aa, Homo sapiens
  SITE (32): Xaa equals any of the naturally occurring L-amino acids
ETQFYDFDSLSCSRVCGHYTQLVWANSFYVGXAXAMCPNL

SEQ ID NO: 326 - protein, 28 aa, Homo sapiens
STAIFVCNYGPAGNFANMPPYVRGESCS

SEQ ID NO: 327 - protein, 26 aa, Homo sapiens
  SITE (9): Xaa equals any of the naturally occurring L-amino acids
PQQTAFNPXQLRFSSSENLLMSFIYK

SEQ ID NO: 328 - protein, 164 aa, Homo sapiens
TEGGCALVPNDMESLKQKLVRVLEENLILSEKIQQLEEGAAISIVSGQQSHTYDDLLHKNQQLTMQVACLNQELAQLKKLEKTVAILHESQRSLVVTNEYLLQQLNKEPKGYSGKALLPPEKGHHLGRSSPFGKSTLSSSSPVAHETGQYLIQSVLDAAPEPGL

SEQ ID NO: 329 - protein, 5 aa, Homo sapiens
SMVSK

SEQ ID NO: 330 - protein, 38 aa, Homo sapiens
MESLKQKLVRVLEENLILSEKIQQLEEGAAISIVSGQQ

SEQ ID NO: 331 - protein, 31 aa, Homo sapiens
DLLHKNQQLTMQVACLNQELAQLKKLEKTVA

SEQ ID NO: 332 - protein, 27 aa, Homo sapiens
SSPFGKSTLSSSSPVAHETGQYLIQSV

SEQ ID NO: 333 - protein, 50 aa, Homo sapiens
  SITE (34): Xaa equals any of the naturally occurring L-amino acids
NTDWDQTVLIVLRISSTLPVALLRDEVPGWFLKXPEPQLISKELIMLTEV

SEQ ID NO: 334 - protein, 31 aa, Homo sapiens
  SITE (27): Xaa equals any of the naturally occurring L-amino acids
VLIVLRISSTLPVALLRDEVPGWFLKXPEPQ

SEQ ID NO: 335 - protein, 24 aa, Homo sapiens
  SITE (2): Xaa equals any of the naturally occurring L-amino acids
GXSSISAVVPAASLWVWPGLRVFR

SEQ ID NO: 336 - protein, 55 aa, Homo sapiens
  SITE (8): Xaa equals any of the naturally occurring L-amino acids
VCQYCTAXMADFGISAGQFVAVVWDKSSPVEALKGLVDKLQALTGNEGRVSVENI

SEQ ID NO: 337 - protein, 35 aa, Homo sapiens
MADFGISAGQFVAVVWDKSSPVEALKGLVDKLQAL

SEQ ID NO: 338 - protein, 12 aa, Homo sapiens
SKCCITTTWKPL

SEQ ID NO: 339 - protein, 44 aa, Homo sapiens
MSSPLLTASSLGQAGTLRKIKPSLTTHHIQCPCSSLREEGRTSQ

SEQ ID NO: 340 - protein, 12 aa, Homo sapiens
GLWTGINHRNMI

SEQ ID NO: 341 - protein, 10 aa, Homo sapiens
FQREVFAPPS

SEQ ID NO: 342 - protein, 88 aa, Homo sapiens
IGQGRHSDSREKSLLLHLWKNFSHCIYYYMFLTGVSLLLDREQVYLLLSPQPLDLGRLIVDIWEMLGKERRGGERKDSMAMSKCPAMS

SEQ ID NO: 343 - protein, 28 aa, Homo sapiens
KNFSHCIYYYMFLTGVSLLLDREQVYLL

SEQ ID NO: 344 - protein, 25 aa, Homo sapiens
VDIWEMLGKERRGGERKDSMAMSKC

SEQ ID NO: 345 - protein, 110 aa, Homo sapiens
KEIPTVWHQDLCDLQGACFPQQSLFYTTCSPHHPGPFHLLKNTELLFTVGPLNAYFSKFHSSTRLQEFSLREESKQVWPQLLEMAEERVFSLNGGGGSCVLGNPISPFIS

SEQ ID NO: 346 - protein, 32 aa, Homo sapiens
CDLQGACFPQQSLFYTTCSPHHPGPFHLLKNT

SEQ ID NO: 347 - protein, 26 aa, Homo sapiens
FTVGPLNAYFSKFHSSTRLQEFSLRE

SEQ ID NO: 348 - protein, 27 aa, Homo sapiens
VWPQLLEMAEERVFSLNGGGGSCVLGN

SEQ ID NO: 349 - protein, 10 aa, Homo sapiens
STHASALHGE

SEQ ID NO: 350 - protein, 27 aa, Homo sapiens
  SITE (7): Xaa equals any of the naturally occurring L-amino acids
MGISACXLPPASLPFPAEAAPEPLPSR

SEQ ID NO: 351 - protein, 27 aa, Homo sapiens
GLLHSSGCKIYILLPEVDTFAWVLFKE

SEQ ID NO: 352 - protein, 77 aa, Homo sapiens
DYSIPLDVKSTFSCLRWIRLLGFCLRRWGQQCVSGPVKCVLYPGFCLISVFSLAYQSHCRGYLVSESRTFPGCCGTD

SEQ ID NO: 353 - protein, 26 aa, Homo sapiens
KSTFSCLRWIRLLGFCLRRWGQQCVS

SEQ ID NO: 354 - protein, 28 aa, Homo sapiens
LYPGFCLISVFSLAYQSHCRGYLVSESR

SEQ ID NO: 355 - protein, 8 aa, Homo sapiens
GTRTAVQS

SEQ ID NO: 356 - protein, 11 aa, Homo sapiens
LTQEPCPISVS

SEQ ID NO: 357 - protein, 16 aa, Homo sapiens
LCIWTRFIFLFKVAIH

SEQ ID NO: 358 - protein, 65 aa, Homo sapiens
IFPKPHMTPVCFRLLEALEESIGVDEMESFKSCFGFCFCVWVFKESISCHVEENPGGGCPPTGRR

SEQ ID NO: 359 - protein, 28 aa, Homo sapiens
ESIGVDEMESFKSCFGFCFCVWVFKESI

SEQ ID NO: 360 - protein, 18 aa, Homo sapiens
DFLLFPHAGPNSKFPRAD

SEQ ID NO: 361 - protein, 56 aa, Homo sapiens
LHRELPLLWAKDKKECRLVSRMIKLHSAYSSRVRPVLVGFRAAFRPAGLRLPLMRM

SEQ ID NO: 362 - protein, 55 aa, Homo sapiens
  SITE (15): Xaa equals any of the naturally occurring L-amino acids
AFAKSYLGDTIEGTXAGTGPEFPGRPTRPPAWRPRRGAATRRFASSLRIICGRVP

SEQ ID NO: 363 - protein, 92 aa, Homo sapiens
  SITE (3): Xaa equals any of the naturally occurring L-amino acids
RRXKAFVTQDIPFYHXLVMKHLPGADPELVLLGRRYEELERIPLSEMTREEINALVQELGFYRKAAPDAQVPPEYVWAPAKPPEETSDHADL

SEQ ID NO: 364 - protein, 44 aa, Homo sapiens
VAESTEEPAGSNRGQYPEDSSSDGLRQREVLRNLSSPGWENISR

SEQ ID NO: 365 - protein, 30 aa, Homo sapiens
AREPLGLTQDPLVFGMTSFLQTSSPIPNSC

SEQ ID NO: 366 - protein, 15 aa, Homo sapiens
FQAPASARTACSTLL

SEQ ID NO: 367 - protein, 33 aa, Homo sapiens
VLLCHQAGVQWHARLTATSTSRVAAILLPQPPE

SEQ ID NO: 368 - protein, 37 aa, Homo sapiens [sequence omitted]

SEQ ID NO: 369 - protein, 121 aa, Homo sapiens
  SITE (43): Xaa equals any of the naturally occurring L-amino acids
HSLPPALPAWLTPGHPSDSSLCLLQLAPHLVMAVSVPWPLPEXLGFSCCHCVSLTGPHAGFSYHFLHPAEPRAWQHQSSVVGMSRKQASFSMAQKGVCHLGKSXKRGSKKASCPXYPSFSK

SEQ ID NO: 370 - protein, 34 aa, Homo sapiens
DANPGSRVPEQCSNYYPLLPLIHPMTFFCLTYTG

SEQ ID NO: 371 - protein, 79 aa, Homo sapiens
PSFVLPTLGCVWDMHFACCYLILAECIVLAICVYSQFRFCQASTMKEERGKGIEGAYKGVVREMDVKSKLGKLRSKDMI

SEQ ID NO: 372 - protein, 24 aa, Homo sapiens
IGIRVWYYRNQKNSKQMWIKCLGS

SEQ ID NO: 373 - protein, 45 aa, Homo sapiens
QCSGISGSSLICKMRGSEQVISMFLPFLILLSVAYSLYGEFNKLY

SEQ ID NO: 374 - protein, 67 aa, Homo sapiens
YFMMIKPQFIYSPVDRQLGCFQFFAVTNTPVMGIILSPFYIDTKVSLRYIPRNGISEFLGYGHSQLY

SEQ ID NO: 375 - protein, 17 aa, Homo sapiens
KGCLTQLLREPVPQIQC

SEQ ID NO: 376 - protein, 54 aa, Homo sapiens
FCNLCFTIIREGGRRAGGETIYYFSGILTAWKKRETEKQSREGASHSEFNLSVK

SEQ ID NO: 377 - protein, 20 aa, Homo sapiens
ARARAVGFPSVCSVGSEHSL

SEQ ID NO: 378 - protein, 17 aa, Homo sapiens
KTKSPYPLHPCFWLMYG

SEQ ID NO: 379 - protein, 66 aa, Homo sapiens
  SITE (43): Xaa equals any of the naturally occurring L-amino acids
PTVYQALGKGHSVREGMVPAGLSSPWACEENARLDLDYCKCQXWPSVGFRGRSELSWNLSFLPQFA

SEQ ID NO: 380 - protein, 102 aa, Homo sapiens
  SITE (67): Xaa equals any of the naturally occurring L-amino acids
LMPCLGSAPARNEGYRLWPITEQILNKHPLGVTLNGACFSKLLPFLGSEQLSRELVSSAAPEHCAFXDFEKSFLKXPLGSLDQPKSKGFKRANLIGTAHSPV

SEQ ID NO: 381 - protein, 67 aa, Homo sapiens
  SITE (67): Xaa equals any of the naturally occurring L-amino acids
LMPCLGSAPARNEGYRLWPITEQILNKHPLGVTLNGACFSKLLPFLGSEQLSRELVSSAAPEHCAFX

SEQ ID NO: 382 - protein, 35 aa, Homo sapiens
  SITE (9): Xaa equals any of the naturally occurring L-amino acids
DFEKSFLKXPLGSLDQPKSKGFKRANLIGTAHSPV

SEQ ID NO: 383 - protein, 44 aa, Homo sapiens
HEVSCPPQCGSVEGQKQGMGEGRWEGVTAARMRKAARPAGSPES

SEQ ID NO: 384 - protein, 75 aa, Homo sapiens
  SITE (45): Xaa equals any of the naturally occurring L-amino acids
VTGSRVLPNPPQKSVVKGPGHWGVESARPDLLGVVSVGAIYPVLXTTPGQLRFVERPSHLLPALXPHRSLVGREN

SEQ ID NO: 385 - protein, 77 aa, Homo sapiens
HELRLRPERKAWGPPDSGPPGPPQVFGQRCPAHGSWGSNGCGFFLSVAWTCHWPRLYFLICDSGDHSSQFTVFGRGD

SEQ ID NO: 386 - protein, 33 aa, Homo sapiens
KPLFLHSPQISFFSYNLVSLMCSTEVLFFCNNK

SEQ ID NO: 387 - protein, 33 aa, Homo sapiens; identical to SEQ ID NO: 386

SEQ ID NO: 388 - protein, 50 aa, Homo sapiens
LHFSHTFLSTKNHESLNYSSSHRIESKYQRSHPFKTQFFHCSIRYVLYVR

SEQ ID NO: 389 - protein, 17 aa, Homo sapiens
ERILCRKSKFFWTLPAY

SEQ ID NO: 390 - protein, 52 aa, Homo sapiens
GFQTILKRLDVTCNVIEQFDDPGYYGSMKSPWFLELACFYSGKNFLAPQLTA

(19) United States
(12) Patent Application Publication
(10) Pub. No.: US 2003/0144492 A1
(43) Pub. Date: Jul. 31, 2003

(54) 101 human secreted proteins

(75) Inventors: Roxanne Duan, Bethesda, MD (US); Steven M. Ruben, Brookeville, MD (US); Kimberly A. Florence, Rockville, MD (US); John M. Greene, Gaithersburg, MD (US); Craig A. Rosen, Laytonsville, MD (US); Paul Young, Gaithersbury, MD (US); Ann M. Ferrie, Painted Post, NY (US); Guo-Liang Yu, Berkeley, CA (US); Fouad Janat, Westerly, RI (US); Jian Ni, Germantown, MD (US); Kenneth C. Carter, North Potomac, MD (US); Gregory A. Endress, Florence, MA (US); Ping Feng, Germantown, MD (US); David W. Lafleur, Washington, DC (US); Yanggu Shi, Gaithersburg, MD (US)

Correspondence Address: HUMAN GENOME SCIENCES INC, 9410 KEY WEST AVENUE, ROCKVILLE, MD, 20850

(73) Assignee: Human Genome Sciences, Inc., Rockville, MD

(21) Appl. No.: 10/195,730

(22) Filed: Jul. 16, 2002

Related U.S. Application Data
(63) Continuation of application No. 09/281,976, filed on Mar. 31, 1999.
Application No. 09/281,976 is a continuation-in-part of application No. PCT/US98/20775, filed on Oct. 1, 1998.
(60) Provisional application No. 60/060,837, filed on Oct. 2, 1997.
(60) Provisional application No. 60/060,862, filed on Oct. 2, 1997.
(60) Provisional application No. 60/060,839, filed on Oct. 2, 1997.
(60) Provisional application No. 60/060,866, filed on Oct. 2, 1997.
(60) Provisional application No. 60/060,843, filed on Oct. 2, 1997.
(60) Provisional application No. 60/060,836, filed on Oct. 2, 1997.
(60) Provisional application No. 60/060,838, filed on Oct. 2, 1997.
(60) Provisional application No. 60/060,874, filed on Oct. 2, 1997.
(60) Provisional application No. 60/060,833, filed on Oct. 2, 1997.
(60) Provisional application No. 60/060,884, filed on Oct. 2, 1997.
(60) Provisional application No. 60/060,880, filed on Oct. 2, 1997.

(51) Int. Cl. C12Q 1/68; C07H 21/04; C12N 9/00; C12P 21/02; C12N 5/06

(52) U.S. Cl. 536/23.2; 435/6; 435/183; 435/69.1; 435/320.1; 435/325; 530/350

(57) ABSTRACT

The present invention relates to novel human secreted proteins and isolated nucleic acids containing the coding regions of the genes encoding such proteins. Also provided are vectors, host cells, antibodies, and recombinant methods for producing human secreted proteins. The invention further relates to diagnostic and therapeutic methods useful for diagnosing and treating disorders related to these novel human secreted proteins.

### FIELD OF THE INVENTION

This invention relates to newly identified polynucleotides and the polypeptides encoded by these polynucleotides, uses of such polynucleotides and polypeptides, and their production.

### BACKGROUND OF THE INVENTION

Unlike bacterium, which exist as a single compartment surrounded by a membrane, human cells and other eucaryotes are subdivided by membranes into many functionally distinct compartments. Each membrane-bounded compartment, or organelle, contains different proteins essential for the function of the organelle. The cell uses “sorting signals,” which are amino acid motifs located within the protein, to target proteins to particular cellular organelles.

One type of sorting signal, called a signal sequence, a signal peptide, or a leader sequence, directs a class of proteins to an organelle called the endoplasmic reticulum (ER). The ER separates the membrane-bounded proteins from all other types of proteins. Once localized to the ER, both groups of proteins can be further directed to another organelle called the Golgi apparatus. Here, the Golgi distributes the proteins to vesicles, including secretory vesicles, the cell membrane, lysosomes, and the other organelles.

Proteins targeted to the ER by a signal sequence can be released into the extracellular space as a secreted protein. For example, vesicles containing secreted proteins can fuse with the cell membrane and release their contents into the extracellular space—a process called exocytosis. Exocytosis can occur constitutively or after receipt of a triggering signal. In the latter case, the proteins are stored in secretory vesicles (or secretory granules) until exocytosis is triggered. Similarly, proteins residing on the cell membrane can also be secreted into the extracellular space by proteolytic cleavage of a “linker” holding the protein to the membrane.

Despite the great progress made in recent years, only a small number of genes encoding human secreted proteins have been identified. These secreted proteins include the commercially valuable human insulin, interferon, Factor VIII, human growth hormone, tissue plasminogen activator, and erythropoeitin. Thus, in light of the pervasive role of secreted proteins in human physiology, a need exists for identifying and characterizing novel human secreted proteins and the genes that encode them. This knowledge will allow one to detect, to treat, and to prevent medical disorders by using secreted proteins or the genes that encode them.

### SUMMARY OF THE INVENTION

The present invention relates to novel polynucleotides and the encoded polypeptides. Moreover, the present invention relates to vectors, host cells, antibodies, and recombinant methods for producing the polypeptides and polynucleotides. Also provided are diagnostic methods for detecting disorders related to the polypeptides, and therapeutic methods for treating such disorders. The invention further relates to screening methods for identifying binding partners of the polypeptides.

### DETAILED DESCRIPTION

Definitions

The following definitions are provided to facilitate understanding of certain terms used throughout this specification.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide.

In the present invention, a “secreted” protein refers to those proteins capable of being directed to the ER, secretory vesicles, or the extracellular space as a result of a signal sequence, as well as those proteins released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of the coding sequence, but do not comprise all or a portion of any intron. In another embodiment, the nucleic acid comprising the coding sequence does not contain coding sequences of a genomic flanking gene (i.e., 5′ or 3′ to the gene in the genome).

As used herein, a “polynucleotide” refers to a molecule having a nucleic acid sequence contained in SEQ ID NO:X or the cDNA contained within the clone deposited with the ATCC. For example, the polynucleotide can contain the nucleotide sequence of the full length cDNA sequence, including the 5′ and 3′ untranslated sequences, the coding region, with or without the signal sequence, the secreted protein coding region, as well as fragments, epitopes, domains, and variants of the nucleic acid sequence. Moreover, as used herein, a “polypeptide” refers to a molecule having the translated amino acid sequence generated from the polynucleotide as broadly defined.

In the present invention, the full length sequence identified as SEQ ID NO:X was often generated by overlapping sequences contained in multiple clones (contig analysis). A representative clone containing all or most of the sequence for SEQ ID NO:X was deposited with the American Type Culture Collection (“ATCC”). As shown in Table 1, each clone is identified by a cDNA Clone ID (Identifier) and the ATCC Deposit Number. The ATCC is located at 10801 University Boulevard, Manassas, Va. 20110-2209, USA. The ATCC deposit was made pursuant to the terms of the Budapest Treaty on the international recognition of the deposit of microorganisms for purposes of patent procedure.

A “polynucleotide” of the present invention also includes those polynucleotides capable of hybridizing, under stringent hybridization conditions, to sequences contained in SEQ ID NO:X, the complement thereof, or the cDNA within the clone deposited with the ATCC. “Stringent hybridization conditions” refers to an overnight incubation at 42° C. in a solution comprising 50% formamide, 5× SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

Also contemplated are nucleic acid molecules that hybridize to the polynucleotides of the present invention at lower stringency hybridization conditions. Changes in the stringency of hybridization and signal detection are primarily accomplished through the manipulation of formamide concentration (lower percentages of formamide result in lowered stringency); salt conditions, or temperature. For example, lower stringency conditions include an overnight incubation at 37° C. in a solution comprising 6× SSPE (20× SSPE=3M NaCl; 0.2M NaH₂PO₄; 0.02M EDTA, pH 7.4), 0.5% SDS, 30% formamide, 100 ug/mil salmon sperm blocking DNA; followed by washes at 50° C. with 1× SPE, 0.1% SDS. In addition, to achieve even lower stringency, washes performed following stringent hybridization can be done at higher salt concentrations (e.g. 5× SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The polynucleotide of the present invention can be composed of any polyribonucleotide or polydeoxribonucleotide, which may be unmodified RNA or DNA or modified RNA or DNA. For example, polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the polynucleotide can be composed of triple-stranded regions comprising RNA or DNA or both RNA and DNA. A polynucleotide may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Acadermic Press, New York, pgs. 1-12 (1983); Seifter et al., Meth Enzymol 182:626-646 (1990); Rattan et al., Ann NY Acad Sci 663:48-62 (1992).)

“SEQ ID NO:X” refers to a polynucleotide sequence while “SEQ ID NO:Y” refers to a polypeptide sequence, both sequences identified by an integer specified in Table 1.

“A polypeptide having biological activity” refers to polypeptides exhibiting activity similar, but not necessarily identical to, an activity of a polypeptide of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention.)

Polynucleotides and Polypeptides of the Invention

Features of Protein Encoded by Gene No:1

In specific embodiments, polypeptides of the invention comprise the following armino acid sequence:PNKHNLRLTRPHTEV (SEQ ID NO:220); MLMKINFYPL PKPKLHTSISNCLLDISIYKPSSLISITSDLPGLTLKSXNFSPTPMPGQNLVVTSS SLASSHPCSVCQWIL (SEQ ID NO:219); MLMKINFYPLPKPKLHTSISNCLLDIS IY (SEQ ID NO:222): KPSSLISITSDLPGLTLKSXNFSPTPMP (SEQ ID NO:223); GQNLVVTSYSSLASSHPCSVCQWIL (SEQ ID NO:224); GTSLFLWALYVIYML MKINFYPLPKPKLHTSISNCLLDISIYKPSSLISITSDLPGLTLKSXNFSPTPMPG QNLVVTSYSSLASSHPCSVCQWIL (SEQ ID NO:221); and/or GTSLFLWALYVI YMLMKINFYPLPKPKL (SEQ ID NO:225). Polynucleotides encoding these polypeptides are also encompassed by the invention.

This gene is expressed primarily in CD34 positive blood cells.

Therefore, polynucleotides and polypeptides of the invention are useful as reagents for differential identification of the tissue(s) or cell type(s) present in a biological sample and for the diagnosis of diseases and conditions, which include, but are not limited to, abnormalities of the immune system, in addition to reproductive disorders. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., immune, hematopoietic, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

The tissue distribution in CD34 positive blood cells suggests that the protein product of this clone is useful for the diagnosis and treatment of diseases and disorders of the immune system. Similarly, the expression of this gene product in immune cells suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:11 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 538 of SEQ ID NO:11, b is an integer of 15 to 552, where both a and b correspond to the positions of nucleotide residues shown in SEQ ID NO:11, and where b is greater than or equal to a +14.

Features of Protein Encoded by Gene No: 2

This gene is expressed primarily in healing wound tissue, Hodgkin's lymphoma, and to a lesser extent, in other tissues.

Therefore, polynucleotides and polypeptides of the invention are useful as reagents for differential identification of the tissue(s) or cell type(s) present in a biological sample and for the diagnosis of diseases and conditions, which include, but are not limited to, proliferative, immune, or hematopoietic diseases and/or disorders, particularly Hodgkin's lymphoma. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in Hodgkin's lymphoma tissue suggests that the protein product of this clone is useful for the diagnosis and/or treatment of Hodgkin's lymphoma and treatment of wounds. Expression within wounded tissue and other cellular sources marked by proliferating cells suggests that this protein may play a role in the regulation of cellular division, and may show utility in the diagnosis and treatment of cancer and other proliferative disorders. Similarly, embryonic development also involves decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:12 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1420 of SEQ ID NO:12, b is an integer of 15 to 1434, where both a and b correspond to the positions of nucleotide residues shown in SEQ ID NO:12, and where b is greater than or equal to a +14.

Features of Protein Encoded by Gene No: 3

The translation product of this clone was shown to have homology to the human M6 membrane glycoprotein which is thought to be important in myelination of central nervous system neurons during development (See Genbank Accession No.bbs|137975).

In specific embodiments, polypeptides of the invention comprise the following amino acid sequence: LAPRFAFSQCSLAIMLTLLFQIHFLMILSSNWAYLKDASK MQAYQDIKAKEEQELQDIQSRSKEQLNSYT (SEQ ID NO:226); LAPRFAFSQC SLAIMLTLLFQIHFLMILSSNWAYLKD (SEQ ID NO:227); ASKMQAYQDIKAK

EEQELQDIQSRSKEQLNSYT (SEQ ID NO:228); and/or LISQTSFSLPSPGPINFL SQSEIYFSI (SEQ ID NO:229). Polynucleotides encoding these polypeptides are also encompassed by the invention. This gene is expressed primarily in fetal brain, and to a lesser extent, in schizophrenic hypothalamus.

Therefore, polynucleotides and polypeptides of the invention are useful as reagents for differential identification of the tissue(s) or cell type(s) present in a biological sample and for the diagnosis of diseases and conditions, which include, but are not limited to, developmental or neural disorders, particularly neurological and psychogenic disorders. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., developmental, neural, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder. The tissue distribution in neural tissues suggests that the protein product of this clone is useful for the diagnosis and/or treatment of certain neurological psychogenic disorders, including schizophrenia. Moreover, the protein product of this clone is useful for the detection/treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein as well as antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:13 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1867 of SEQ ID NO:13, b is an integer of 15 to 1881, where both a and b correspond to the positions of nucleotide residues shown in SEQ ID NO:13, and where b is greater than or equal to a +14.

Features of Protein Encoded by Gene No: 4

In specific embodiments, polypeptides of the invention comprise the following amino acid sequence: IRHEGGGQPFTSXPLEILFFLNGWYNATYFLLELFIFLYKG VLLPYPTANLVLDVV (SEQ ID NO:230); MVHTRCSGHGDQGGELEVSRGLVLR RGRMGITLPLPILECRRVSWADGPGLEDGTHWPYAELLAQMSVLKKSHTAFL RTTCPTNSHWCG (SEQ ID NO:231); and/or TRTISPRDSSTLQYREGQGYSHPAPS QNQSPADLKFSSLITVARASRVDHLGSLGFKQDLSHMLPVRAVLYLSHMSTE SLMLVGFQSDVKASHPNPRRLSSTTFLVAHSVIFLLSS (SEQ ID NO:232). Polynucleotides encoding these polypeptides are also encompassed by the invention.

The gene encoding the disclosed cDNA is believed to reside on chromosome 11. Accordingly, polynucleotides related to this invention are useful as a marker in linkage analysis for chromosome 11.

This gene is expressed primarily in adult brain.

Therefore, polynucleotides and polypeptides of the invention are useful as reagents for differential identification of the tissue(s) or cell type(s) present in a biological sample and for the diagnosis of diseases and conditions, which include, but are not limited to, neural disorders, particularly neurodegenerative diseases. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., neural, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 118 as residues: Thr-17 to Lys-25.

The tissue distribution in adult brain suggests that the protein product of this clone is useful for the diagnosis and treatment of neurodegenerative diseases. Moreover, the protein product of this clone is useful for the detection/treatment of behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:14 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1046 of SEQ ID NO:14, b is an integer of 15 to 1060, where both a and b correspond to the positions of nucleotide residues shown in SEQ ID NO:14, and where b is greater than or equal to a +14.

Features of Protein Encoded by Gene No: 5

This gene is expressed primarily in 12 week old early stage human and infant brain.

Therefore, polynucleotides and polypeptides of the invention are useful as reagents for differential identification of the tissue(s) or cell type(s) present in a biological sample and for the diagnosis of diseases and conditions, which include, but are not limited to, neural or developmental disorders, particularly neurodegenerative conditions. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., developmental, neural, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 119 as residues: Phe-20 to Arg-26.

The tissue distribution in neural and developmental tissues suggests that the protein product of this clone is useful for the diagnosis and/or treatment of neurodevelopmental diseases. The protein product of this clone would also be useful for the detection/treatment of neurodegenerative disease states, behavioural disorders, or inflamatory conditions such as Alzheimers Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:15 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1241 of SEQ ID NO:15, b is an integer of 15 to 1255, where both a and b correspond to the positions of nucleotide residues shown in SEQ ID NO:15, and where b is greater than or equal to a +14.

Features of Protein Encoded by Gene No: 6

The translation product of this clone was shown to have homology to the conserved MAP kinase phosphatase which is known to be important as an antagonist in MAP kinase activation (See Genbank Accession No.gi|1050849). As such, a role in development or in cellular metabolism may be anticipated.

In specific embodiments, polypeptides of the invention comprise the following amino acid sequence: RVIRLTXRANWSSTAVAAALELVDPPGCRNSARVKYCV VYDNNSSTLEILLKDDDDDSDSDGDGKDLVPQAAIEYGRILTRLTHHPVYILK GGYERFS GTYHFLRTQKIIWMPQELDAFQPYPIEIVPGKVFVGNFS QACDPKIQ KDLKIKAHVNVSMDTGPFFAGDADKLLHIRIEDSPEAQILPFLRHMCHFIEIHH HLGSVILIFSTQGISRSCAAIIAYLMHSNEQTLQRSWAYVKKCKNNMCPNRGL VSQLLEWEKTILGDSITNIMDPLY (SEQ ID NO:233); RVIRLTXRANWSSTAVA AALELVDPPGCRNSARVKYC (SEQ ID NO:234); VVYDNNSSTLEILLKDD DDDSDSDGDGKDLVPQA (SEQ ID NO:235); AIEYGRILTRLTHHPVYILKGG YERFSGTYHFLRTQ (SEQ ID NO:236); KIIWMPQELDAFQPYPIEIVPGKVF VGNFSQACDP (SEQ ID NO:237); KIQKDLKIKAHVNVSMDTGPFFAGDADKL LHIRIED (SEQ ID NO:238); SPEAQILPFLRHMCHFIEIHHHLGSVILIFSTQGI (SEQ ID NO:239); SRSCAAIIAYLMHSNEQTLQRSWAYVKKCKNNMCPN (SEQ ID NO:240); and/or RGLVSQLLEWEKTILGDSITNIMDPLY (SEQ ID NO:241). Polynucleotides encoding these polypeptides are also encompassed by the invention.

The gene encoding the disclosed cDNA is believed to reside on chromosome 7. Accordingly, polynucleotides related to this invention are useful as a marker in linkage analysis for chromosome 7.

This gene is expressed primarily in fetal kidney, liver, and spleen.

Therefore, polynucleotides and polypeptides of the invention are useful as reagents for differential identification of the tissue(s) or cell type(s) present in a biological sample and for the diagnosis of diseases and conditions, which include, but are not limited to, developmental, immune, or haemopoietic disorders. Similarly, polypeptides and antibodies directed to those polypeptides are useful to provide immunological probes for differential identification of the tissue(s) or cell type(s). For a number of disorders of the above tissues or cells, particularly of the haemopoietic system or developing immune system, expression of this gene at significantly higher or lower levels may be detected in certain tissues or cell types (e.g., developmental, immune, hematopoietic, hepatic, and cancerous and wounded tissues) or bodily fluids (e.g., lymph, bile, amniotic fluid, serum, plasma, urine, synovial fluid or spinal fluid) taken from an individual having such a disorder, relative to the standard gene expression level, i.e., the expression level in healthy tissue from an individual not having the disorder.

The tissue distribution in fetal liver, combined with the homology to a signal transduction regulatory protein suggests that the protein product of this clone is useful for the diagnosis and treatment of hematopoietic disorders involving blood stem cell formation, such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Protein as well as antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:16 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1022 of SEQ ID NO:16, b is an integer of 15 to 1036, where both a and b correspond to the positions of nucleotide residues shown in SEQ ID NO:16, and where b is greater than or equal to a +14.

Features of Protein Encoded by Gene No: 7

In specific embodiments, polypeptides of the invention comprise the following amino acid sequence: IRHEFTSEKSWKSSCNEGESSSTSYMHQRSPGGPTKLIEII SDCNWEEDRNKILSILSQHINSNMPQSLKVGSFIIELASQRKSRGEKNPPVYSS RVXISMPSCQDQDDMAEKSGSETPDGPLSPGKMEDISPVQTDALDSVRERLH GGKGLPFYAGLSPAGKLVAYKRKPSSSTSGLIQVRIIFNLGIAPLYTPR (SEQ ID NO:242); EFGTSLHQKRAGSLPA (SEQ ID NO:243); IRHEFTSEKSWKSSC NEGESSSTSYMHQRSPGGPTKL (SEQ ID NO:244); IEIISDCNWEEDRNKILSI LSQHINSNMPQSLK (SEQ ID NO:245); VGSFIIELASQRKSRGEKNPPVYSSRV XISMPSCQD (SEQ ID NO:246); and/or QDDMAEKSGSETPDGPLSPGKMEDIS PVQTDALD (SEQ ID NO:247). Polynucleotides encoding these polypeptides are also encompassed by the invention.

This gene is expressed primarily in human fetal heart.

Therefore, polynucleotides and polypeptides of the invention are useful as reagents for differential identification of the tissue(s) or cell type(s) present in a biological sample and for the diagnosis of diseases and conditions, which include, but are not limited to, developmental or cardiovascular disorders, particularly fetal cardiac defects. Similarly, polypeptides and antibodies directed to those polypeptides are useful to provide immunological probes for differential identification of the tissue(s) or cell type(s). For a number of disorders of the above tissues or cells, particularly of the cardiac system, expression of this gene at significantly higher or lower levels may be detected in certain tissues or cell types (e.g., developmental, cardiac, musculoskeletal, and cancerous and wounded tissues) or bodily fluids (e.g., lymph, amntiotic fluid, serum, plasma, urine, synovial fluid or spinal fluid) taken from an individual having such a disorder, relative to the standard gene expression level, i.e., the expression level in healthy tissue from an individual not having the disorder.

The distribution in fetal heart tissue suggests that the protein product of this clone is useful for the diagnosis and treatment of fetal cardiac defects. Similarly, expression within fetal tissue suggests that this protein may play a role in the regulation of cellular division, and may show utility in the diagnosis and treatment of cancer and other proliferative disorders. Similarly, embryonic development also involves decisions involving cell differentiation and/or apoptosis in pattern formation. Thus, this protein may also be involved in apoptosis or tissue differentiation and could be useful in cancer therapy. Protein as well as antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:17 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1000 of SEQ ID NO:17, b is an integer of 15 to 1014, where both a and b correspond to the positions of nucleotide residues shown in SEQ ID NO:17, and where b is greater than or equal to a +14.

Features of Protein Encoded by Gene No: 8

In specific embodiments, polypeptides of the invention comprise the following amino acid sequence: CNIEYIRSDKCMFKHELEELRTTI (SEQ ID NO:248). Polynucleotides encoding these polypeptides are also encompassed by the invention.

The gene encoding the disclosed cDNA is believed to reside on chromosome 2. Accordingly, polynucleotides related to this invention are useful as a marker in linkage analysis for chromosome 2.

This gene is expressed primarily in fetal cochlea, other fetal tissues, and to a lesser extent in placenta.

Therefore, polynucleotides and polypeptides of the invention are useful as reagents for differential identification of the tissue(s) or cell type(s) present in a biological sample and for the diagnosis of diseases and conditions, which include, but are not limited to, developmental disorders, particularly of auditory tissues. Similarly, polypeptides and antibodies directed to those polypeptides are useful to provide immunological probes for differential identification of the tissue(s) or cell type(s). For a number of disorders of the above tissues or cells, particularly of the fetal developmental systems, expression of this gene at significantly higher or lower levels may be detected in certain tissues or cell types (e.g., developmental, auditory, and cancerous and wounded tissues) or bodily fluids (e.g., lymph, amniotic fluid, cochlear fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 122 as residues: Met-1 to His-6, Glu-33 to Asn-43.

The tissue distribution suggests that the protein product of this clone is useful for the diagnosis and treatment of fetal developmental disorders, particularly of auditory tissues. Similarly, expression within fetal tissues and other cellular sources marked by proliferating cells suggests that this protein may play a role in the regulation of cellular division, and may show utility in the diagnosis and treatment of cancer and other proliferative disorders. Similarly, embryonic development also involves decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:18 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1273 of SEQ ID NO:18, b is an integer of 15 to 1287, where both a and b correspond to the positions of nucleotide residues shown in SEQ ID NO:18, and where b is greater than or equal to a +14.

Features of Protein Encoded by Gene No: 9

This gene is expressed primarily in nine week old early stage human.

Therefore, polynucleotides and polypeptides of the invention are useful as reagents for differential identification of the tissue(s) or cell type(s) present in a biological sample and for the diagnosis of diseases and conditions, which include, but are not limited to, fetal developmental disorders. Similarly, polypeptides and antibodies directed to those polypeptides are useful to provide immunological probes for differential identification of the tissue(s) or cell type(s). For a number of disorders of the above tissues or cells, particularly of the fetal developmental systems, expression of this gene at significantly higher or lower levels may be detected in certain tissues or cell types (e.g., developmental, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 123 as residues: Met-1 to Arg-6.

The tissue distribution suggests that the protein product of this clone is useful for the diagnosis and/or treatment of some types of fetal developmental disorders. Moreover, the expression within embryonic tissue suggests that this protein may play a role in the regulation of cellular division, and may show utility in the diagnosis and treatment of cancer and other proliferative disorders. Similarly, embryonic development also involves decisions involving cell differentiation and/or apoptosis, in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:19 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1091 of SEQ ID NO:19, b is an integer of 15 to 1105, where both a and b correspond to the positions of nucleotide residues shown in SEQ ID NO:19, and where b is greater than or equal to a +14.

Features of Protein Encoded by Gene No:10

This gene is expressed primarily in epididymus.

Therefore, polynucleotides and polypeptides of the invention are useful as reagents for differential identification of the tissue(s) or cell type(s) present in a biological sample and for the diagnosis of diseases and conditions, which include, but are not limited to, reproductive disorders, particularly male sterility. Similarly, polypeptides and antibodies directed to those polypeptides are useful to provide immunological probes for differential identification of the tissue(s) or cell type(s). For a number of disorders of the above tissues or cells, particularly of the male reproductive system, expression of this gene at significantly higher or lower levels may be detected in certain tissues or cell types (e.g., reproductive, cancerous and wounded tissues) or bodily fluids (e.g., lymph, seminal fluid, serum, plasma, urine, synovial fluid or spinal fluid) taken from an individual having such a disorder, relative to the standard gene expression level, i.e., the expression level in healthy tissue from an individual not having the disorder.

The tissue distribution in epididymus suggests that the protein product of this clone is useful for the diagnosis and treatment of male sterility, and/or could be used as a male contraceptive. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:20 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1075 of SEQ ID NO:20, b is an integer of 15 to 1089, where both a and b correspond to the positions of nucleotide residues shown in SEQ ID NO:20, and where b is greater than or equal to a +14.

Features of Protein Encoded by Gene No:11

The translation product of this gene shares sequence homology with a mitotic phosphoprotein which is thought to be important in initiating and coordinating cell division processes.

In specific embodiments, polypeptides of the invention comprise the following amino acid sequence: HHQQVPEXDREDSPERCSDXXEEKKARRGRSPKGEFKD EEETVTTKHIHITQATETTTTRHKRTANPSKTIDLGAAAHYTGDKASPDQNAS THTPQSSVKTSVPSSKSSGDLVDLFDGTSQCNRRXS (SEQ ID NO:249); VSSDSVGGFRYSERYDPEPKSKWDEEWDKNKSAFPFSDKLGELSDKIGSTIDD TISKFRXKIEKTLQKDA ATXXRKRKREEADLPKVNSKMKRRL (SEQ ID NO:250); and/or RQSIFISHRPQRPPQPDTSAQQILPKPLILEQQHITQGTKQVQIR (SEQ ID NO:251). Polynucleotides encoding these polypeptides are also encompassed by the invention.

The gene encoding the disclosed cDNA is believed to reside on chromosome 5. Accordingly, polynucleotides related to this invention are useful as a marker in linkage analysis for chromosome 5.

This gene is expressed primarily in placenta, and to a lesser extent in t-cells.

Therefore, polynucleotides and polypeptides of the invention are useful as reagents for differential identification of the tissue(s) or cell type(s) present in a biological sample and for the diagnosis of diseases and conditions, which include, but are not limited to, spontaneous abortion and in utero developmental problems, in addition to immune disorders, such as autoimmune conditions. Similarly, polypeptides and antibodies directed to those polypeptides are useful to provide immunological probes for differential identification of the tissue(s) or cell type(s). For a number of disorders of the above tissues or cells, particularly of the immune and reproductive systems, expression of this gene at significantly higher or lower levels may be detected in certain tissues or cell types (e.g., developmental, immune, hematopoietic, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 125 as residues: Ser-65 to Gly-71, Ser-155 to Leu-160, Gln-168 to Asp-179, Leu-189 to Pro-196, Gln-210 to Ser-218, Gln-224 to Pro-231, Val-326 to Asp-331.

The tissue distribution in placental tissue combined with the homology to mitotic phosphoprotein suggests that the protein product of this clone is useful for the treatment and diagnosis of diseases that arise in utero due to cell division abnormalities during fetal development. Alternatively, expression within T-cells suggests that the secreted protein may also be used to determine biological activity, to raise antibodies, as tissue markers, to isolate cognate ligands or receptors, to identify agents that modulate their interactions and as nutritional supplements. It may also have a very wide range of biological acitivities. Typical of these are cytokine, cell proliferation/differentiation modulating activity or induction of other cytokines; immunostimulating/immunosuppressant activities (e.g. for treating human immunodeficiency virus infection, cancer, autoimmune diseases and allergy); regulation of hematopoiesis (e.g. for treating anaemia or as adjunct to chemotherapy); stimulation or growth of bone, cartilage, tendons, ligaments and/or nerves (e.g. for treating wounds, stimulation of follicle stimulating hormone (for control of fertility); chemotactic and chemokinetic activities (e.g. for treating infections, tumors); hemostatic or thrombolytic activity (e.g. for treating haemophilia, cardiac infarction etc.); anti-inflammatory activity (e.g. for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:21 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 2817 of SEQ ID NO:21, b is an integer of 15 to 2831, where both a and b correspond to the positions of nucleotide residues shown in SEQ ID NO:21, and where b is greater than or equal to a +14.

Features of Protein Encoded by Gene No:12

The translation product of this gene shares sequence homology with the human polyposis locus 1 gene (TB2/DP1, Genbank Acc. No. gi|190162) and the murine counterpart of the human TB2/DP1 (Genbank Acc. No. gi|1142716). TB2/DP1 is thought to be important in the development of colorectal cancer, particularity those associated with familial adenomatous polyposis (FAP) disease. Triggering of murine mast cells by IgE plus antigen results in a decrease of TB2/DP1 mRNA up to 60% after 2 h implying a possible role of this gene in regulation of the allergic effector cell. Reverse transcription-polymerase chain reaction (RT-PCR) analysis shows an ubiquitous expression pattern in a number of mouse cell lines and tissues.

In specific embodiments, polypeptides of the invention comprise the following amino acid sequence: AASWGPPHVPKAGK (SEQ ID NO:253); DQDGLRAVAA LTLHQGRQLLYRKFVHPSLSRHEKEIDAYIVQAKERSYETVLSFGKRGLNIAA SAAVQAATXSQGALAGRLRSFSMQDLRSISDAPAPAYHDPLYLEDQVSHRRP PIGYRAGGLQDSDTEDECWSDTEAVPRAPARPREKPLIRSQSLRVVKXKPPVR EGTSRSLKVR TXKKTVPSDVDS (SEQ ID NO:252); DQDGLRAVAALTLHQGR QLLYRKFVHPSLSRHEKEIDA (SEQ ID NO:254); YIVQAKERSYETVLSFGKRG LNIAASAAVQAATXSQ (SEQ ID NO:255); GALAGRLRSFSMQDLRSISDAPA PAYHDPLYLED (SEQ ID NO:256); QVSHRRPPIGYRAGGLQDSDTEDECWSD TEAVPRA (SEQ ID NO:257); PARPREKPLIRSQSLRVVKXKPPVREGTSRSLK

VR (SEQ ID NO:258); and/or PVREGTSRSLKVRTXKKTVPSDVDS (SEQ ID NO:259). Polynucleotides encoding these polypeptides are also encompassed by the invention.

This gene is expressed primarily in T cells, and to a lesser extent, in fetal skin.

Therefore, polynucleotides and polypeptides of the invention are useful as reagents for differential identification of the tissue(s) or cell type(s) present in a biological sample and for the diagnosis of diseases and conditions, which include, but are not limited to, cancer, colorectal cancer, particularly, familial adenomatous polyposis (FAP); or other proliferating disorders. Similarly, polypeptides and antibodies directed to those polypeptides are useful to provide immunological probes for differential identification of the tissue(s) or cell type(s). For a number of disorders of the above tissues or cells, particularly of the colon, expression of this gene at significantly higher or lower levels may be detected in certain tissues or cell types (e.g., colon, immune, developmental tissues, integumentary, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 126 as residues: Met-99 to Ala-114.

The tissue distribution in T-cells and fetal skin, combined with the homology to the DP 1 gene of the FAP locus, suggests that the protein product of this clone is useful for treatment and/or diagnosis of colo-rectal cancer particularly, familial adenomatous polyposis, as well as other cancers. It may also be useful in treating allergic disorders. Expression within fetal tissue and other cellular sources marked by proliferating cells suggests that this protein may play a role in the regulation of cellular division, and may show utility in the diagnosis and treatment of cancer and other proliferative disorders. Similarly, embryonic development also involves decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:22 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1434 of SEQ ID NO:22, b is an integer of 15 to 1448, where both a and b correspond to the positions of nucleotide residues shown in SEQ ID NO:22, and where b is greater than or equal to a +14.

Features of Protein Encoded by Gene No:13

The translation product of this gene shares sequence homology with the Claudin multigene family (e.g. Genbank Acc. Nos. gnl|PID|e1363658, gi|3335182, and gi|3236224); members of which are involved in the formation of tight junction strands in various tissues. Claudin multigene family members are thought to encode four-transmembrane domain proteins. The translation product of this gene also shares sequence homology with a transmembrane protein (Genbank Acc. No. gi|2150013) that is deleted in Velo-cardio-facial syndrome (VCFS) and DiGeorge syndrome (DGS). VCFS and DGS are characterized by a wide spectrum of phenotypes including cleft palate, conotruncal heart defects, and facial dysmorphology. The translation product of this gene also shares sequence homology with a murine oligodendrocyte-specific protein related to peripheral myelin protein-22 (PMP-22, Genbank Acc. No. gi|633767). PMP-22 is important in peripheral myelination and Schwann cell proliferation, and mutations in its gene cause diseases of peripheral nerves. Myelin plays a critical role in nervous system function and alterations in myelin-specific proteins cause a variety of neurologic disorders.

In specific embodiments, polypeptides of the invention comprise the following amino acid sequence: GVLPLPPLWGHQPPRVLHPT (SEQ ID NO:261); LCHRLPGRLQLLGVPVHAGPLWVYSGLPGTHDHRHPPGLPRPLAXHXGPAL HQHWGPGALQESQAGGXRRGPPHSGRYLRDGGXLLVRFNITRDFFDPLYPGT KYELGPXLYLGWSASLXSILGGLCLCSACCCGSDEDQPPAPGGPTXLPCP (SEQ ID NO:260); LCHRLPGRLQLLGVPVHAGPLWVYSGLPGTHDHR (SEQ ID NO:262); HPPGLPRPLAXHXGPALHQHWGPGALQESQAGGXRRG (SEQ ID NO:263); PPHSGRYLRDGGXLLVRFNITRDFFDPLYPGTKYE (SEQ ID NO:264); LGPXLYLGWSASLXSILGGLCLCSACCCGSDEDQPP (SEQ ID NO:265); and/or SACCCGSDEDQPPAPGGPTXLPC (SEQ ID NO:266). Polynucleotid&s encoding these polypeptides are also encompassed by the invention.

This gene is expressed primarily in endothelial and T cells.

Therefore, polynucleotides and polypeptides of the invention are useful as reagents for differential identification of the tissue(s) or cell type(s) present in a biological sample and for the diagnosis of diseases and conditions, which include, but are not limited to, neurological disorders related to myelin abnormalities, in addition to immune or endothelial disorders, particularly vascular conditions. Similarly, polypeptides and antibodies directed to those polypeptides are useful to provide immunological probes for differential identification of the tissue(s) or cell type(s). For a number of disorders of the above tissues or cells, particularly of the nervous system, expression of this gene at significantly higher or lower levels may be detected in certain tissues or cell types (e.g., neural, immune, vascular, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in immune cells, combined with the homology to an oligodendrocyte-specific protein related to PMP-22, members of the Claudin multigene family, and the transmembrane protein deleted in VCFS and DGS, suggests that the protein product of this clone is useful for the diagnosis and/or treatment of diseases of the nervous system, particularly those involving aberrant myelinization of the nerves, such as ALS and multiple sclerosis, or autoimmune disorders affecting neural tissues. Similarly, the protein product of this clone is useful for the detection/treatment of neurodegenerative disease states, behavioural disorders, or inflamatory conditions such as Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:23 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1197 of SEQ ID NO:23, b is an integer of 15 to 1211, where both a and b correspond to the positions of nucleotide residues shown in SEQ ID NO:23, and where b is greater than or equal to a +14.

Features of Protein Encoded by Gene No:14

The translation product of this gene shares high sequence homology at the nucleotide level with the human G protein-coupled receptor (EBI 1) gene, exon 1. The EBI1 gene is a lymphoid-specific member of the G-protein-coupled receptor family. This receptor, also reported as the Epstein-Barr-induced cDNA EBI1, is expressed in normal lymphoid tissues and in several B- and T-lymphocyte cell lines. While the function and the ligand for EBI1 remain unknown, its sequence and gene structure suggest that it is related to the receptors that recognize chemoattractants, such as interleukin-8, RANTES, C5a, and fMet-Leu-Phe. Like the chemoattractant receptors, EBI1 contains intervening sequences near its 5′ end; however, EBI1 is unique in that both of its introns interrupt the coding region of the first extracellular domain. The gene is encoded on human chromosome 17q12-q21.2. None of the other G-protein-coupled receptors has been mapped to this region, but the C—C chemokine family has been mapped to 17q11-q21. The mouse EBI1 cDNA has also been isolated and encodes a protein with 86% identity to the human homolog.

This gene is expressed primarily in spinal cord.

Therefore, polynucleotides and polypeptides of the invention are useful as reagents for differential identification of the tissue(s) or cell type(s) present in a biological sample and for the diagnosis of diseases and conditions, which include, but are not limited to, neural or inflammatory disorders. Similarly, polypeptides and antibodies directed to those polypeptides are useful to provide immunological probes for differential identification of the tissue(s) or cell type(s). For a number of disorders of the above tissues or cells, particularly of the central nervous system and immune system, expression of this gene at significantly higher or lower levels may be detected in certain tissues or cell types (e.g., neural, immune, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in spinal cord and homology to the EBI-1 gene suggests that the protein product of this clone is useful for developing diagnostics and small molecule therapeutics for affecting the action of chemoattractants similar to interleukin-8, RANTES, C5a, and fMet-Leu-Phe. In turn, this could be useful in the treatment of inflammatory diseases such as sepsis, inflammatory bowel syndrome, psoriasis, and rheumatoid arthritis. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:24 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1046 of SEQ ID NO:24, b is an integer of 15 to 1060, where both a and b correspond to the positions of nucleotide residues shown in SEQ ID NO:24, and where b is greater than or equal to a +14.

Features of Protein Encoded by Gene No:15

This gene is expressed primarily in osteoclastoma, and to a lesser extent, in T cell and fetal liver.

Therefore, polynucleotides and polypeptides of the invention are useful as reagents for differential identification of the tissue(s) or cell type(s) present in a biological sample and for the diagnosis of diseases and conditions, which include, but are not limited to, osteoclastoma; hematopoietic disorders; immune dysfunction; susceptibility to infection; or osteoporosis. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., skeletal tissues, immune or hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution of this clone is useful for the diagnosis and/or treatment of disorders of the hematopoietic system. In particular, the elevated expression of this gene product in osteoclastoma suggests that it may play a role particularly in the development of the osteoclast lineage, and thus may be particularly useful in conditions such as osteoporosis and osteopetrosis. Additionally, the gene product may play more generalized roles in hematopoiesis, as evidenced by expression in T cells and fetal liver. It may also be used to affect the proliferation, survival, activation, and/or differentiation of a variety of hematopoietic lineages. Thus, it may play roles in a variety of disease conditions, including lymphoma/leukemias; defects in immune modulation or immune surveilance; susceptibility to infection; and other hematopoietic disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:25 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1043 of SEQ ID NO:25, b is an integer of 15 to 1057, where both a and b correspond to the positions of nucleotide residues shown in SEQ ID NO:25, and where b is greater than or equal to a +14.

Features of Protein Encoded by Gene No:16

The translation product of this gene shares sequence homology with the mouse bup gene which is localized 5′ to the bmi gene locus. Retroviral insertions into this region are frequently correlated with accelerated lymphomagenesis (See Genbank Accession No. bbs|125119).

The gene encoding the disclosed cDNA is believed to reside on chromosome 10. Accordingly, polynucleotides related to this invention are useful as a marker in linkage analysis for chromosome 10.

This gene is expressed primarily in WI 38 lung fibroblasts, fetal lung, placenta, and to a lesser extent, in T cell lymphoma, fetal liver, and stromal cells.

Therefore, polynucleotides and polypeptides of the invention are useful as reagents for differential identification of the tissue(s) or cell type(s) present in a biological sample and for the diagnosis of diseases and conditions, which include, but are not limited to, T cell lymphoma, fibrosis, mesenchymal disorders; respiratory disorders; ARDS. Similarly, polypeptides and antibodies directed to those polypeptides are useful to provide immunological probes for differential identification of the tissue(s) or cell type(s). For a number of disorders of the above tissues or cells, particularly of the skeletal, reproductive, respiratory, and immune systems, expression of this gene at significantly higher or lower levels may be detected in certain tissues or cell types (e.g., reproductive, skeletal, pulmonary, immune, hematopoietic, and cancerous and wounded tissues) or bodily fluids (e.g., lymph, pulmonary surfactant and sputum, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 130 as residues: Gly-74 to Leu-83, Cys-90 to Arg-96, Glu-103 to Asn-109, Glu-133 to Gln-140, Gln-156 to Pro-164, Lys-183 to Arg-191.

The tissue distribution suggests that the protein product of this clone is useful for the diagnosis and/or treatment of disorders of the lung and, more generally, of mesenchymal cells. Expression of this gene product is elevated in fetal lung, as well as in a cell line derived from lung, suggesting a role in lung function. This suggests that the protein product of this clone is useful for the detection and treatment of disorders associated with developing lungs, particularly in premature infants where the lungs are the last tissues to develop. This also suggests that the protein product of this clone is useful for the diagnosis and intervention of lung tumors, since the gene may be involved in the regulation of cell division, particularly since it is expressed in fetal tissue. Expression of this gene is also elevated in mesenchymally-derived cells and tissues such as fibroblasts and endothelium.

The expression of this gene in T cell lymphoma and it's homology to the bup-1 suggest that the gene or protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. The gene product is also expressed at hematopoietic sites, such as fetal liver. Thus, it may also play a role in hematopoiesis, either in the survival, proliferation, and/or differentiation of various blood cell lineages. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:26 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 966 of SEQ ID NO:26, b is an integer of 15 to 980, where both a and b correspond to the positions of nucleotide residues shown in SEQ ID NO:26, and where b is greater than or equal to a +14.

Features of Protein Encoded by Gene No:17

This gene is expressed primarily in a breast cancer cell line and in Wilm's tumor samples, and to a lesser extent, in apoptotic and helper T cells, as well as activated macrophages.

Therefore, polynucleotides and polypeptides of the invention are useful as reagents for differential identification of the tissue(s) or cell type(s) present in a biological sample and for the diagnosis of diseases and conditions, which include, but are not limited to, breast cancer; wilm's tumor; nephroblastoma; hematopoietic disorders; immune dysfunction; acute renal failure. Similarly, polypeptides and antibodies directed to those polypeptides are useful to provide immunological probes for differential identification of the tissue(s) or cell type(s). For a number of disorders of the above tissues or cells, particularly of the breast, kidney, and immune system, expression of this gene at significantly higher or lower levels may be detected in certain tissues or cell types (e.g., breast, reproductive, immune, hematopoietic, and cancerous and wounded tissues) or bodily fluids (e.g., lymph, breast milk, serum, plasma, urine, synovial fluid or spinal fluid) taken from an individual having such a disorder, relative to the standard gene expression level, i.e., the expression level in healthy tissue from an individual not having the disorder.

The tissue distribution suggests that the protein product of this clone is useful for the diagnosis and/or treatment of cancer. This gene product is expressed at elevated levels in both breast cancer cells as well as Wilm's tumor. The tissue distribution-in tumors of kidney and breast origins suggests that the protein product of this clone is useful for the diagnosis and intervention of these tumors, in addition to other tumors where expression has been indicated. This observation also suggests that this gene product may play a role in the control of cell proliferation and/or survival, particularly since it is also observed in apoptotic T cells. Alternately, it may control other aspects of cell behavior or activation, as it is also observed in helper T cells and activated macrophages. Thus, it may play general roles in the immune system as well, either in the control of blood cell survival, proliferation, differentiation, or activation. Thus, this gene product may be useful in controlling immune modulation and immune surveillance as well. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:27 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 741 of SEQ ID NO:27, b is an integer of 15 to 755, where both a and b correspond to the positions of nucleotide residues shown in SEQ ID NO:27, and where b is greater than or equal to a +14.

Features of Protein Encoded by Gene No:18

This gene is expressed primarily in the synovium.

Therefore, polynucleotides and polypeptides of the invention are useful as reagents for differential identification of the tissue(s) or cell type(s) present in a biological sample and for the diagnosis of diseases and conditions, which include, but are not limited to, skeletal disorders, particularly joint disorders such as rheumatoid arthritis. Similarly, polypeptides and antibodies directed to those polypeptides are useful to provide immunological probes for differential identification of the tissue(s) or cell type(s). For a number of disorders of the above tissues or cells, particularly of the skeletal system, expression of this gene at significantly higher or lower levels may be detected in certain tissues or cell types (e.g., skeletal, synovium, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution suggests that the gene and protein product of this clone is useful for diagnosis of disorders of the joints as disregulation of genes encoding proteins secreted from synovial tissues is thought to affect normal function of the joints and may lead to autoimmune disorders such as rheumatoid arthritis, lupus, scleroderma, and dermatomyositis as well as dwarfism, spinal deformation, and specific joint abnormalities as well as chondrodysplasias (ie. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:28 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 932 of SEQ ID NO:28, b is an integer of 15 to 946, where both a and b correspond to the positions of nucleotide residues shown in SEQ ID NO:28, and where b is greater than or equal to a +14.

Features of Protein Encoded by Gene No:19

This gene is expressed primarily in amniotic cells, and to a lesser extent, in chronic lymphocytic leukemia cells of the spleen.

Therefore, polynucleotides and polypeptides of the invention are useful as reagents for differential identification of the tissue(s) or cell type(s) present in a biological sample and for the diagnosis of diseases and conditions, which include, but are not limited to, reproductive, developmental or immune disorders, particularly leukemia. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., developmental, immune, hematopoictic, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

The tissue distribution in leukemia cells suggests that the protein product of this clone is useful for the treatment or diagnosis of leukemia and other immune diseases. Similarly, this gene product may be useful in the regulation of the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:29 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 957 of SEQ ID NO:29, b is an integer of 15 to 971, where both a and b correspond to the positions of nucleotide residues shown in SEQ ID NO:29, and where b is greater than or equal to a +14.

Features of Protein Encoded by Gene No: 20

The translation product of this clone was found to have homology to the human protein, defender against cell death 1 gene, which is a known antagonist of apoptosis (See Genseq Accession No:P46966).

The gene encoding the disclosed cDNA is believed to reside on chromosome 14. Accordingly, polynucleotides related to this invention are useful as a marker in linkage analysis for chromosome 14.

This gene is expressed primarily in breast, lung, testes, B cells and T cells.

Therefore, polynucleotides and polypeptides of the invention are useful as reagents for differential identification of the tissue(s) or cell type(s) present in a biological sample and for the diagnosis of diseases and conditions, which include, but are not limited to, immune or pulmonary disorders, particularly cancer of the breast, lung, testes and B cells. Similarly, polypeptides and antibodies directed to those polypeptides are useful to provide immunological probes for differential identification of the tissue(s) or cell type(s). For a number of disorders of the above tissues or cells, particularly of the reproductive and immune system, expression of this gene at significantly higher or lower levels may be detected in certain tissues or cell types (e.g., immune, reproductive, pulmonary, and cancerous and wounded tissues) or bodily fluids (e.g., seminal fluid, lymph, breast milk, pulmonary surfactant or sputum, serum, plasma, urine, synovial fluid or spinal fluid) taken from an individual having such a disorder, relative to the standard gene expression level, i.e., the expression level in healthy tissue from an individual not having the disorder.

The tissue distribution suggests that the protein product of this clone is useful for the diagnosis and treatment of breast cancer, lung cancer, and B cell lymphoma. Similarly, expression within cellular sources marked by proliferating cells, combined with its homology to a conserved regulatory protein of apoptosis suggests that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:30 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 994 of SEQ ID NO:30, b is an integer of 15 to 1008, where both a and b correspond to the positions of nucleotide residues shown in SEQ ID NO:30, and where b is greater than or equal to a +14.

Features of Protein Encoded by Gene No: 21

The translation product of this gene shares sequence homology with human and murine surface glycoprotein which is thought to be important in cell-cell interactions and transducing cellular signals (See Genseq Accession No.gi|2997741) and to the multigene Tetraspanin family (e.g. see Genbank Acc. No. gi|3152701, gi|2997741, and gi|2997745). Tetraspanins (or TM4SF) are expressed in a wide variety of species and regulate cell adhesion, migration, proliferation and differentiation.

This gene is expressed primarily in testis.

Therefore, polynucleotides and polypeptides of the invention are useful as reagents for differential identification of the tissue(s) or cell type(s) present in a biological sample and for the diagnosis of diseases and conditions, which include, but are not limited to, male reproductive diseases or disorders. Similarly, polypeptides and antibodies directed to those polypeptides are useful to provide immunological probes for differential identification of the tissue(s) or cell type(s). For a number of disorders of the above tissues or cells, particularly of the male reproductive system, expression of this gene at significantly higher or lower levels may be detected in certain tissues or cell types (e.g., testicular, reproductive, immune, and cancerous and wounded tissues) or bodily fluids (e.g., lymph, seminal fluid, serum, plasma, urine, synovial fluid or spinal fluid) taken from an individual having such a disorder, relative to the standard gene expression level, i.e., the expression level in healthy tissue from an individual not having the disorder. Preferred epitopes include those comprising a sequence shown in SEQ ID NO. 135 as residues: Thr-6 to Leu-11.

The tissue distribution in testes combined with the homology to a conserved cell surface glycoprotein, and Tetraspanin protein family members suggests that the protein product of this clone is useful for treatment and diagnosis of diseases associated with the male reproductive system. The protein product of this clone is useful for the treatment and diagnosis of conditions concerning proper testicular function (e.g. endocrine function, sperm maturation), as well as cancer. Therefore, this gene product is useful in the treatment of male infertility and/or impotence. This gene product is also useful in assays designed to identify binding agents, as such agents (antagonists) are useful as male contraceptive agents. Similarly, the protein is believed to be useful in the treatment and/or diagnosis of testicular cancer.

The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications. In addition, expression of this gene product in the testis may implicate this gene product in normal testicular function. In addition, this gene product may be useful in the treatment of male infertility, and/or could be used as a male contraceptive. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:31 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 976 of SEQ ID NO:31, b is an integer of 15 to 990, where both a and b correspond to the positions of nucleotide residues shown in SEQ ID NO:31, and where b is greater than or equal to a +14.

Features of Protein Encoded by Gene No: 22

The translation product of this clone was found to have homology to the human myosin regulatory light chain which is thought to be important in muscle function (See Genbank Accession No.gi|189013).

In specific embodiments, polypeptides of the invention comprise the following amino acid sequence: FAPGARKEPFRPRPQVDQMFQFASIDVAGNLDYKALSY VITHGEEKEE (SEQ ID NO:269); VDQMFQFASIDVAGNLDYKALSYVITffGEE KEE (SEQ ID NO:267); and/or IRHEAYVILAVCLGG (SEQ ID NO:268).

Polynucleotides encoding these polypeptides are also encompassed by the invention.

The gene encoding the disclosed cDNA is believed to reside on chromosome 4. Accordingly, polynucleotides related to this invention are useful as a marker in linkage analysis for chromosome 4.

This gene is expressed primarily in lung, testis, and macrophage.

Therefore, polynucleotides and polypeptides of the invention are useful as reagents for differential identification of the tissue(s) or cell type(s) present in a biological sample and for the diagnosis of diseases and conditions, which include, but are not limited to, cancers and immune disorders, particularly afflicting the pulmonary or reproductive system. Similarly, polypeptides and antibodies directed to those polypeptides are useful to provide immunological probes for differential identification of the tissue(s) or cell type(s). For a number of disorders of the above tissues or cells, particularly of the respiratory system, immue system and male reproductive system, expression of this gene at significantly higher or lower levels may be detected in certain tissues or cell types (e.g., immune, pulmonary, reproductive, and cancerous and wounded tissues) or bodily fluids (e.g., lymph, pulmonary surfactant or sputum, seminal fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 136 as residues: Tyr-47 to Phe-54, Arg-144 to Ser-149, Thr-152 to Asp-161, Glu-194 to Asn-203, Glu-242 to Pro-250, Thr-258 to Gly-263, Ala-269 to Gly-274.

The tissue distribution in the testis, and in macrophages, suggests that the protein product of this clone is useful for the treatment and diagnosis of diseases of the immune system and male reproductive system. Alternatively, the homology to the conserved myosin regulatory light chain suggests that the protein product of this clone may be useful in the detection, treatment, and/or prevention of a variety of skeletal or cardiac muscle disorders, such as muscular sclerosis. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID Nb:32 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1117 of SEQ ID NO:32, b is an integer of 15 to 1131, where both a and b correspond to the positions of nucleotide residues shown in SEQ ID NO:32, and where b is greater than or equal to a +14.

Features of Protein Encoded by Gene No: 23

The translation product of this gene shares sequence homology with a Ca2+ activated potassium channel regulatory subunit (See Genbank Ace. No. gi|1527201) which is thought to be important in potassium ion channel regulation or regulation of cell proliferation.

In specific embodiments, polypeptides of the invention comprise the following amino acid sequence: WIQRIRHETNPKCSYIPPCKRENQKNLESVMNWQQYWK DEIGSQPFTCYFNQHQRPDDVLLHRTHDEIVLLHCFLWPLVTFVVGVLIVVLTI CAKSLAVKAEAMXEAQVLLKGKEACRKQSTEAVLIGTRPPAEPVFPGAGDG QGHDRALRGSSLSGNRNRHNWKTWNLKACIPSAVAMAKGSRS (SEQ ID NO:270); WIQRIRHETNPKCSYIPPCKRENQKNLESVMNWQQY (SEQ ID NO:271); WKDEIGSQPFTCYFNQHQRPDDVLLHRTHDEIVLL (SEQ ID NO:272); HCFLWPLVTFVVGVLIVVLTICAKSLAVKAEAMXE (SEQ ID NO:273); AQVLLKGKEACRKQSTEAVLIGTRPPAEPVFPGAGD (SEQ ID NO:274); and/or GQGHDRALRGSSLSGNRNRHNWKTWNLKACIPSAVAMAKGSRS (SEQ ID NO:275). Polynucleotides encoding these polypeptides are also encompassed by the invention.

The gene encoding the disclosed cDNA is believed to reside on chromosome 12. Accordingly, polynucleotides related to this invention are useful as a marker in linkage analysis for chromosome 12.

This gene is expressed primarily in the brain.

Therefore, polynucleotides and polypeptides of the invention are useful as reagents for differential identification of the tissue(s) or cell type(s) present in a biological sample and for the diagnosis of diseases and conditions, which include, but are not limited to, neural disorders, particularly neurodegenerative disorders, such as Alzheimer's Disease, Parkinson's Disease, or Huntington's Disease. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., neural, cancerous and wounded tissues) or bodily fluids (e.g., lymph, serum, plasma, urine, synovial fluid, cerebrospinal fluid, or spinal fluid) taken from an individual having such a disorder, relative to the standard gene expression level, i.e., the expression level in healthy tissue from an individual not having the disorder.

The tissue distribution and homology to a Ca2+ activated potassium channel regulatory subunit suggests that the protein product of this clone is useful for the diagnosis and treatment of diseases related to potassium channel malfunction in the brain. Similarly, the protein product of this clone is useful for the detection/treatment of neurodegenerative disease states, behavioural disorders, or inflamatory conditions such as Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, migranes, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:33 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1279 of SEQ ID NO:33, b is an integer of 15 to 1293, where both a and b correspond to the positions of nucleotide residues shown in SEQ ID NO:33, and where b is greater than or equal to a +14.

Features of Protein Encoded by Gene No: 24

The translation product of this gene shares sequence homology with oxidoreductase which is thought to be important in inflammatory reactions; and to several members of the short-chain dehydrogenase/reductase enzyme superfamily (e.g. see Genbank Acc. No. gi|3450832, gi|3450828 and gb|AFO61743|AF061743). The translation product of this gene contains the two consensus sequences of the SDR superfamily, an N-terminal Gly-X-X-X-Gly-X-Gly cofactor-binding motif and a Tyr-X-X-X-Lys segment essential for catalytic activity of SDR proteins. Based on the sequence similarity, the translation product of this clone is expected to share biological activities with such proteins. Such activities are known in the art, some of which are described elsewhere herein.

In specific embodiments, polypeptides of the invention comprise the following amino acid sequence: KLFYKKKCTCICQKLLYFMMFLKKVITSASITSLTCQSTV LLPNPTQEKATXKNT (SEQ ID NO:276). Polynucleotides encoding these polypeptides are also encompassed by the invention.

This gene is expressed primarily in human pancreas tumor.

Therefore, polynucleotides and polypeptides of the invention are useful as reagents for differential identification of the tissue(s) or cell type(s) present in a biological sample and for the diagnosis of diseases and conditions, which include, but are not limited to, metabolic or immune disorders, particularly proliferative conditions such as pancreas tumor. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., metabolic tissues, immune, and cancerous and wounded tissues) or bodily fluids (e.g., lymph, bile, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 138 as residues: Ue-72 to Asn-77, Asp-98 to Val-105, Val-210 to Ile-216.

The tissue distribution and homology to oxidoreductase and short-chain dehydrogenase/reductase enzyme family members suggests that the protein product of this clone is useful for diagnosis of pancreas tumor, metabolic disorders, and inflammatory diseases. The protein product of this clone is useful for for the diagnosis, prevention, and/or treatment of various metabolic disorders such as Tay-Sachs disease, phenylkenonuria, galactosemia, hyperlipidemias, porphyrias, and Hurler's syndrome. The tissue distribution in pancreas tumor suggests that the protein product of this clone is useful for the detection, treatment, and/or prevention of various endocrine disorders and cancers, particularly Addison's disease, Cushing's Syndrome, and disorders and/or cancers of the pancrease (e.g. diabetes mellitus), adrenal cortex, ovaries, pituitary (e.g., hyper-, hypopituitarism), thyroid (e.g. hyper-, hypothyroidism), parathyroid (e.g. hyper-, hypoparathyroidism), hypothallamus, and testes. Similarly, expression within cellular sources marked by proliferating cells suggests that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:34 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1000 of SEQ ID NO:34, b is an integer of 15 to 1014, where both a and b correspond to the positions of nucleotide residues shown in SEQ ID NO:34, and where b is greater than or equal to a +14.

Features of Protein Encoded by Gene No: 25

The translation product of this clone was shown to have homology to the rat TIP120, which is thought to be important in the regulation of basal as well as activated trascriptional metabolism (See Genbank Accession No. gnl|PID|d1014122). TIP120 is thought to participate in transcription regulation through the interaction with the TATA-binding protein (TBP).

In specific embodiments, polypeptides of the invention comprise the following amino acid sequence: HYEKVRLQVPIRNSRVDPRVXKFTISDHPQPIDPLLKNCIG DFLKTLEDPDLNVRRVALVTFNSAAHNKPSLIRDLLDTVLPHLYNETKVRKE LIREVEMGPFKHTVDDGLDIRKAAFECMYTLLDSCLDRLDIFEFLNHVEDGLK DHYDIK (SEQ ID NO:277); HYEKVRLQVPIRNSRVDPRVXKFTISDHPQPIDPLLK (SEQ ID NO:278); NCIGDFLKTLEDPDLNVRRVALVTFNSAAHNKPS (SEQ ID NO:279); LIRDLLDTVLPHLYNETKVRKELIREVEMGPFKHTVD (SEQ ID NO:280); and/or DGLDIRKAAFECMYTLLDSCLDRLDIFEFLNHVEDGLKDHY DIK (SEQ ID NO:281). Polynucleotides encoding these polypeptides are also encompassed by the invention.

The gene encoding the disclosed cDNA is believed to reside on chromosome 12. Accordingly, polynucleotides related to this invention are useful as a marker in linkage analysis for chromosome 12.

This gene is expressed primarily in infant brain and various cancers.

Therefore, polynucleotides and polypeptides of the invention are useful as reagents for differential identification of the tissue(s) or cell type(s) present in a biological sample and for the diagnosis of diseases and conditions, which include, but are not limited to, neural or developmental disorders, particularly cancers. Similarly, polypeptides and antibodies directed to those polypeptides are useful to provide immunological probes for differential identification of the tissue(s) or cell type(s). For a number of disorders of the above tissues or cells, particularly of the nervous or immune system, expression of this gene at significantly higher or lower levels may be detected in certain tissues or cell types (e.g., developmental, neural, and cancerous and wounded tissues) or bodily fluids (e.g., lymph, amniotic fluid, serum, plasma, urine, synovial fluid, cerebrospinal fluid, or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 139 as residues: Ser-41 to Lys-53, Ser-80 to Pro-86, Ile-95 to Ser-110.

The tissue distribution in fetal brain and various cancers, and its homology to a protein involved in transcriptional regulation, suggests that the protein product of this gene may play a role in the regulation of cellular division, and may show utility in the diagnosis and treatment of cancer and other proliferative disorders. Similarly, embryonic development also involves decisions involving cell differentiation and/or apoptosis in pattern formation. Thus, this protein may also be involved in apoptosis or tissue differentiation and could again be useful in cancer therapy. Similarly, the protein product of this clone is useful for the detection/treatment of a variety of neural disorders, which include, but are not limited to neurodegenerative disease states, behavioural disorders, or inflamatory conditions such as Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:35 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1208 of SEQ ID NO:35, b is an integer of 15 to 1222, where both a and b correspond to the positions of nucleotide residues shown in SEQ ID NO:35, and where b is greater than or equal to a +14.

Features of Protein Encoded by Gene No: 26

In specific embodiments, polypeptides of the invention comprise the following amino acid sequence: IRHEHLRGVQERVNLSAPLLPKEDPIFTYLSKRLGRSID DIGHLIHEGLQKNTSSWVLYNMASFYWRIKN EPYQVVECA (SEQ ID NO:282); IRHEHLRGVQERVNLSAPLLPKEDPIFTYLSKRLGRSIDDIG (SEQ ID NO:283); and/or HLIHEGLQKNTSSWVLYNMASFYWRIKNEPYQVVECA (SEQ ID NO:284). Polynucleotides encoding these polypeptides are also encompassed by the invention.

This gene is expressed primarily in brain, testes and Hodgkin's lymphoma.

Therefore, polynucleotides and polypeptides of the invention are useful as reagents for differential identification of the tissue(s) or cell type(s) present in a biological sample and for the diagnosis of diseases and conditions, which include, but are not limited to, neural, reproductive, or immune disorders, particularly Hodgkin's lymphoma. Similarly, polypeptides and antibodies directed to those polypeptides are useful to provide immunological probes for differential identification of the tissue(s) or cell type(s). For a number of disorders of the above tissues or cells, particularly of the immune system expression of this gene at significantly higher or lower levels may be detected in certain tissues or cell types (e.g., neural, reproductive, immune, hematopoietic, and cancerous and wounded tissues) or bodily fluids (e.g., lymph, seminal fluid, serum, plasma, urine, synovial fluid, cerebrospinal fluid, or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 140 as residues: Ser-7 to Asp-13, Gln-93 to Leu-99, Ser-105 to His-122, Arg-125 to Thr-132.

The tissue distribution suggests that the protein product of this clone is useful for the diagnosis and treatment of a variety of immune system disorders. Expression of this gene product in Hodgkin's lymphoma suggests a role in the regulation of the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:36 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 887 of SEQ ID NO:36, b is an integer of 15 to 901, where both a and b correspond to the positions of nucleotide residues shown in SEQ ID NO:36, and where b is greater than or equal to a +14.

Features of Protein Encoded by Gene No: 27

In specific embodiments, polypeptides of the invention comprise the following amino acid sequence: EFGTSPHQTCGRRPGTAAGWLLAHSTV (SEQ ID NO:285). Polynucleotides encoding these polypeptides are also encompassed by the invention.

The gene encoding the disclosed cDNA is believed to reside on chromosome 19. Accordingly, polynucleotides related to this invention are useful as a marker in linkage analysis for chromosome 19.

This gene is expressed primarily in epididymus, small intestine, and kidney.

Therefore, polynucleotides and polypeptides of the invention are useful as reagents for differential identification of the tissue(s) or cell type(s) present in a biological sample and for the diagnosis of diseases and conditions, which include, but are not limited to, reproductive, renal, or gastrointestinal disorders, particularly degenerative kidney disease, congenital digestive disorders, and male infertility. Similarly, polypeptides and antibodies directed to those polypeptides are useful to provide immunological probes for differential identification of the tissue(s) or cell type(s). For a number of disorders of the above tissues or cells, particularly of the urinary, digestive, and male reproductive systems, expression of this gene at significantly higher or lower levels may be detected in certain tissues or cell types (e.g., reproductive, urogenital, intestinal, endothelial, and cancerous and wounded tissues) or bodily fluids (e.g., lymph, seminal fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 141 as residues: Ala-59 to Thr-68, Glu-72 to Ser-108, Glu-115 to Lys-126.

The tissue distribution in kidney suggests that this gene or gene product could be used in the treatment and/or detection of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilm's Tumor Disease, and congenital kidney abnormalities such as horseshoe kidney, polycystic kidney, and Falconi's syndrome. Alternatively, expression within the epididymus suggests that the protein product of this clone may be useful for the detection, treatment, and/or prevention of a variety of reproductive disorders, particularly male infertility. Similarly, the protein product of this clone is useful for the treatment and diagnosis of conditions concerning proper testicular function (e.g. endocrine function, sperm maturation), as well as cancer. Therefore, this gene product is useful in the treatment of male infertility and/or impotence. This gene product is also useful in assays designed to identify binding agents, as such agents (antagonists) are useful as male contraceptive agents. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:37 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 940 of SEQ ID NO:37, b is an integer of 15 to 954, where both a and b correspond to the positions of nucleotide residues shown in SEQ ID NO:37, and where b is greater than or equal to a +14.

Features of Protein Encoded by Gene No: 28

The translated product of this gene shows homology to a fragment of a gene expressed in the brain (see Genbank Acc. No. gnl|PID|d1026388).

In specific embodiments, polypeptides of the invention comprise the following amino acid sequence: NSARDSLNTAIQAWQQNKCPEVEELVFSHFVICNDTQETLRFGQVDTDENILL ASLHSHQYSWRSHKSPQLLHICIEGWGNWRWSEPFSVDHAGTFIRTIQYRGRT ASLIIKVQQLNGVQKQIIICGRQIICSYLSQSIELKVVQHYIGQDGQAVVREHFD CLTAKQKLPSYILENNELTELCVKAKGDEDWSRDVCLESKAPEYSIVIQVPSS NSSIIYVWCTVLTLEPNSQVQQRMIVFSPLFIMRSHLPDPIIIHLEKRSLGLSETQ IIPGKGQEKP LQNIEPDLVHHLTFQA (SEQ ID NO:286); NSARDSLNTAIQAWQQNKC PEVEELVF (SEQ ID NO:292); NKCPEVEELVFSHFVICNDTQETLRF (SEQ ID NO:287); QETLRFGQVDTDENILLASLHSHQYSWRSHKSPQ (SEQ ID NO:293); HICIEGWGNWRWSEPFSVDHAGTFI (SEQ ID NO:288); QYRGRTASLIIKVQ QLNGVQKQIIICGRQIICSYLSQSIE (SEQ ID NO:294); VVREHFDCLTAKQKL PSYILENNELTE (SEQ ID NO:289); EDWSRDVCLESKAPEYSIVIQVPSSNS (SEQ ID NO:290); NSSIIYVWCTVLTLEPNSQVQQRMIVFSPLFIMRSHLPDPI (SEQ ID NO:295); and/or IIHLEKRSLGLSETQIIPGKGQEKPLQ (SEQ ID NO:291). Polynucleotides encoding these polypeptides are also encompassed by the invention.

The gene encoding the disclosed cDNA is believed to reside on chromosome 8. Accordingly, polynucleotides related to this invention are useful as a marker in linkage analysis for chromosome 8.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, disorders of the immune system, particularly immunodefiencies, such as AIDS. Similarly, polypeptides and antibodies directed to those polypeptides are useful to provide immunological probes for differential identification of the tissue(s) or cell type(s). For a number of disorders of the above tissues or cells, particularly of for those of the immune system, and central nervous system expression of this gene at significantly higher or lower levels may be detected in certain tissues or cell types (e.g., immune, hematopoietic, neural and cancerous and wounded tissues) or bodily fluids (e.g., lymph, serum, plasma, urine, synovial fluid, cerebrospinal fluid, or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 142 as residues: Met-1 to Gly-8, Thr-33 to Cys-38, Arg-79 to Arg-89.

The tissue distribution in immune cells suggests that the protein product of this clone is useful for the diagnosis and treatment of a variety of immune system disorders. Expression of this gene product in neutrophils suggests a role in the regulation of the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. In addition, the homology of this clone to a fragment of a gene which is expressed in brain tissues suggests that the protein product of this clone is useful for the diagnosis and/or treatment of disorders of the brain and nervous system. The protein product of this clone may also be useful for the detection/treatment of neurodegenerative disease states and behavioural disorders such as Alzheimer's Disease, Parkinson's Disease, Huntington's Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, the gene or gene product may also play a role in the treatment and/or detection of developmental disorders associated with the developing embryo, or sexually-linked disorders. Protein, as well as, antibodies directed against the protein may show utility as a tumor marker and/or immunotherapy targets for the above listed tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:38 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 876 of SEQ ID NO:38, b is an integer of 15 to 890, where both a and b correspond to the positions of nucleotide residues shown in SEQ ID NO:38, and where b is greater than or equal to a +14.

Features of Protein Encoded by Gene No: 29

It has been discovered that the translation product of this gene shares homology to a conserved Caenorhabditis elegans protein (See Genbank Accession No gi|5775⁴6) and to the C3HC4 type-RING finger family of proteins which contain zinc-finger binding domains (e.g., See Genbank Acc. No. gnl|PID|e1344077, and gi|3790569). Zinc-finger binding domain family members allow, on binding of zinc, a specific tertiary protein domain structure to be formed. The exact function of the domain is unknown, but may be involved in cell-cell communication and proliferation events, leading to migration or differentiation, and possibly apoptosis and cell death.

In specific embodiments, polypeptides of the invention comprise the following amino acid sequence: LITQDQTRRCHGLWHLPSLLWPLLWSSGTGLCRNVCRL HGIYHXVLXRVGHAYQTSFRQXVCXXWAADLCGRHEEGIIENTYRLSCNHV FHEFCIRGWCIVGKKQTCPYCKEKVDLKRMFSNPWERPHVMYGQLLDWLRY LVAWQPVIIGVVQGINYILGLE (SEQ ID NO:296); LIIQDQTRRCHGLWHLPSL LWPLLW (SEQ ID NO:298); SSGTGLCRNVCRLHGIYHXVLXRVGH (SEQ ID NO:299); AYQTSFRQXVCXXWAADLCGRHEE (SEQ ID NO:300); GIIENTYRL SCNHVFHEFCIRGWCIVGKKQ (SEQ ID NO:301); TCPYCKEKVDLKRMFSNP WERPHVMYGQLLD (SEQ ID NO:302); WLRYLVAWQPVIIGVVQGINYILGLE (SEQ ID NO:303); TAFVTFRATRKPLVQTTPRLVYKWFLLIYKISYATGIVGY MAVMFTLFGLNLLFKIKPEDAMDFGISLLFYGLYYGVLERDFAEMCADYMA STIXFXSESGMPTKHLSDSXCAXCGQQIFVDVMKRGSLRTRIGCPAIMS STSSASVAGASWERSKRVPTAKRR (SEQ ID NO:297); TAFVTFRATRKPLVQ TTPRLVYKWFLLI (SEQ ID NO:304); YKISYATGIVGYMAVMFTLFGLNLL FKIK (SEQ ID NO:305); PEDAMDFGISLLFYGLYYGVLE (SEQ ID NO:306); RDFAEMCADYMASTIXFXSESGMPTKHL (SEQ ID NO:307); SDSXCAXCG QQIFVDVMKRGSLRTRIGCPAIM (SEQ ID NO:308); HEFCIRGWCIVGKK QTCPYC (SEQ ID NO:310) and/or SSTSSASVAGASWERSKRVPTAKRR (SEQ ID NO:309). Polynucleotides encoding these polypeptides are also encompassed by the invention.

This gene is expressed primarily in embryonic brain.

Therefore, polynucleotides and polypeptides of the invention are useful as reagents for differential identification of the tissue(s) or cell type(s) present in a biological sample and for the diagnosis of diseases and conditions, which include, but are not limited to, neural disorders, particularly mental retardation of various types, seizures, and mood disorders. Similarly, polypeptides and antibodies directed to those polypeptides are useful to provide immunological probes for differential identification of thetissue(s) or cell type(s). For a number of disorders of the above tissues or cells, particularly of the central nervous system, expression of this gene at significantly higher or lower levels may be detected in certain tissues or cell types (e.g., neural, developmental, and cancerous and wounded tissues) or bodily fluids (e.g., lymph, amniotic fluid, serum, plasma, urine, synovial fluid, cerebrospinal fluid, or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 143 as residues: Ser-22 to Met-28.

The tissue distribution in neural tissue suggests that the protein product of this clone is useful for the detection/treatment of neurodegenerative disease states, behavioural disorders, or inflamatory conditions such as Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Alternatively, expression within embryonic tissue and it's homology to the zinc-finger binding domain family of proteins suggests that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:39 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1056 of SEQ ID NO:39, b is an integer of 15 to 1070, where both a and b correspond to the positions of nucleotide residues shown in SEQ ID NO:39, and where b is greater than or equal to a +14.

Features of Protein Encoded by Gene No: 30

In specific embodiments, polypeptides of the invention comprise the following amino acid sequence: ATSMKRLSHPSICRTGLPLSQQKRASLL (SEQ ID NO:311). Polynucleotides encoding these polypeptides are also encompassed by the invention.

When tested against Jurket cell lines, supernatants removed from cells containing this gene activated NF-kB (Nuclear Factor kB). Thus, it is likely that this gene activates immune cells through various signal transduction pathways. NF-KB is a transcription factor activated by a wide variety of agents, leading to cell activation, differentiation, or apoptosis. Reporter constructs utilizing the NF-kB promoter element are used to screen supernatants for such activity.

This gene is expressed primarily in early stage human embryos.

Therefore, polynucleotides and polypeptides of the invention are useful as reagents for differential identification of the tissue(s) or cell type(s) present in a biological sample and for the diagnosis of diseases and conditions, which include, but are not limited to, developmental disorders, particularly various types of birth defects and congenital conditions. Similarly, polypeptides and antibodies directed to those polypeptides are useful to provide immunological probes for differential identification of the tissue(s) or cell type(s). For a number of disorders of the above tissues or cells, particularly for those of the developing embryo, expression of this gene at significantly higher or lower levels may be detected in certain developing and, ultimately, adult, tissues (e.g., developmental, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

The tissue distribution within embryonic tissue, combined with the detected NF-kB biological activity, suggests that this protein may play a role in the regulation of cellular division, and may show utility in the diagnosis and treatment of cancer and other proliferative disorders. Similarly, developmental tissues rely on decisions involving cell differentiation and/or apoptosis in pattern formation. Thus, this protein may also be involved in apoptosis or tissue differentiation and could again be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:40 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 758 of SEQ ID NO:40, b is an integer of 15 to 772, where both a and b correspond to the positions of nucleotide residues shown in SEQ ID NO:40, and where b is greater than or equal to a +14.

Features of Protein Encoded by Gene No: 31

This gene is expressed primarily in breast.

Therefore, polynucleotides and polypeptides of the invention are useful as reagents for differential identification of the tissue(s) or cell type(s) present in a biological sample and for the diagnosis of breast cancer and related disorders and disease. Similarly, polypeptides and antibodies directed to those polypeptides are useful to provide immunological probes for differential identification of the tissue(s) or cell type(s). For a number of disorders of the above tissues or cells, particularly of the breast lymphatic system, expression of this gene at significantly higher or lower levels may be detected in certain tissues or cell types (e.g., breast, reproductive, endocrine, and cancerous and wounded tissues) or bodily fluids (e.g., lymph, breast milk, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 145 as residues: Lys-27 to Arg-41.

The tissue distribution in breast tissue suggests that the protein product of this clone may be useful for the detection, treatment, and/or prevention of disorders of the breast or reproductive tissue, particularly, breast neoplasia and breast cancers, including but not limited to, fibroadenoma, papillary carcinoma, ductal carcinoma, Paget's disease, medullary carcinoma, mucinous carcinoma, tubular carcinoma, secretory carcinoma and apocrine carcinoma, as well as juvenile hypertrophy and gynecomastia, mastitis and abscess, duct ectasia, fat necrosis and fibrocystic diseases. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:41 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 773 of SEQ ID NO:41, b is an integer of 15 to 787, where both a and b correspond to the positions of nucleotide residues shown in SEQ ID NO:41, and where b is greater than or equal to a +14.

Features of Protein Encoded by Gene No: 32

This gene is expressed primarily in osteosarcoma.

Therefore, polynucleotides and polypeptides of the invention are useful as reagents for differential identification of the tissue(s) or cell type(s) present in a biological sample and for the diagnosis of various skeletal disorders, paricularly of osteosarcoma and related disorders. Similarly, polypeptides and antibodies directed to those polypeptides are useful to provide immunological probes for differential identification of the tissue(s) or cell type(s). For a number of disorders of the above tissues or cells, particularly of the skeletal and immune systems, expression of this gene at significantly higher or lower levels may be detected in certain tissues or cell types (e.g., immune, skeletal, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 146 as residues: Trp-25 to Pro-33, Gln-88 to Pro-93.

The tissue distribution in skeletal tissue suggests that the protein product of this clone is useful for the diagnosis and/or treatment of a variety of skeletal disorders, such as osteosarcoma. Similarly, the expression of this gene product in osteo tissue would suggest a role in the detection and treatment of disorders and conditions affecting the skeletal system, in particular osteoporosis, bone cancer, as well as, disorders afflicting connective tissues (e.g. arthritis, trauma, tendonitis, chrondomalacia and inflammation), such as in the diagnosis or treatment of various autoimmune disorders such as rheumatoid arthritis, lupus, scleroderma, and dermatomyositis as well as dwarfism, spinal deformation, and specific joint abnormalities as well as chondrodysplasias (ie. spondyloepiphyseal dysplasia congenita, familial osteoarthritis, Atelosteogenesis type II, metaphyseal chondrodysplasia type Schmid). Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:42 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 638 of SEQ ID NO:42, b is an integer of 15 to 652, where both a and b correspond to the positions of nucleotide residues shown in SEQ ID NO:42, and where b is greater than or equal to a +14.

Features of Protein Encoded by Gene No: 33

The gene encoding the disclosed cDNA is believed to reside on chromosome 10. Accordingly, polynucleotides related to this invention are useful as a marker in linkage analysis for chromosome 10.

This gene is expressed primarily in microvascular endothelial cells and in fetal liver cells.

Therefore, polynucleotides and polypeptides of the invention are useful as reagents for differential identification of the tissue(s) or cell type(s) present in a biological sample and for the diagnosis of diseases and conditions, which include, but are not limited to, cardiovascular, hematopoietic, immunological, or developmental disorders. Similarly, polypeptides and antibodies directed to those polypeptides are useful to provide immunological probes for differential identification of the tissue(s) or cell type(s). For a number of disorders of the above tissues or cells, particularly of the hematopoietic system, expression of this gene at significantly higher or lower levels may be detected in certain tissues or cell types (e.g., cardiovascular, hematopoietic, immune, developmental, and cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

The tissue distribution in fetal liver suggests that the protein product of this clone is useful for the treatment and diagnosis of hematopoietic related disorders such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Alternatively, expression within vascular tissue suggests that the protein product of this clone is useful for the treatment, diagnosis, and/or prevention of a variety of vascular disorders, particularly cardiovascular disease, atherosclerosis, microvascular disease, stroke, embolism, or aneurysm. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:43 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1506 of SEQ ID NO:43, b is an integer of 15 to 1520, where both a and b correspond to the positions of nucleotide residues shown in SEQ ID NO:43, and where b is greater than or equal to a +14.

Features of Protein Encoded by Gene No: 34

When tested against PC12 cell lines, supernatants removed from cells containing this gene activated the EGR1 (early growth response gene 1) promoter element. Thus, it is likely that this gene activates sensory neuron cells through the EGR1 signal transduction pathway. EGR1 is a separate signal transduction pathway from Jak-STAT, genes containing the EGR1 promoter are induced in various tissues and cell types upon activation, leading the cells to undergo differentiation and proliferation.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, immune system disorders, particularly inflammatory disorders such as arthritis and related conditions. Similarly, polypeptides and antibodies directed to those polypeptides are useful to provide immunological probes for differential identification of the tissue(s) or cell type(s). For a number of disorders of the above tissues or cells, particularly of the central nervous system and immune system, expression of this gene at significantly higher or lower levels may be detected in certain tissues or cell types (e.g., neural, immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 148 as residues: Pro-18 to Glu-25.

The tissue distribution in immune cells combined with the detected EGR1 biological activity suggests that the protein product of this clone is useful for the diagnosis and treatment of a variety of immune system disorders. Expression of this gene product in neutrophils suggests a role in the regulation of the proliferation; survival; differentiation; and/or activation of hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:44 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 782 of SEQ ID NO:44, b is an integer of 15 to 796, where both a and b correspond to the positions of nucleotide residues shown in SEQ ID NO:44, and where b is greater than or equal to a +14.

Features of Protein Encoded by Gene No: 35

This gene is expressed primarily in brain.

Therefore, polynucleotides and polypeptides of the invention are useful as reagents for differential identification of the tissue(s) or cell type(s) present in a biological sample and for the diagnosis of diseases and conditions, which include, but are not limited to, neural disorders, particularly mental retardation, mood disorders, epilepsy, learning disorders, and dementia. Similarly, polypeptides and antibodies directed to those polypeptides are useful to provide immunological probes for differential identification of the tissue(s) or cell type(s). For a number of disorders of the above tissues or cells; particularly of the central nervous system, expression of this gene at significantly higher or lower levels may be detected in certain tissues or cell types (e.g., neural, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in neural tissue suggests that the protein product of this clone is useful for the detection/treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:45 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1364 of SEQ ID NO:45, b is an integer of 15 to 1378, where both a and b correspond to the positions of nucleotide residues shown in SEQ ID NO:45, and where b is greater than or equal to a +14.

Features of Protein Encoded by Gene No: 36

In specific embodiments, polypeptides of the invention comprise the following amino acid sequence: WIPRAAGIRHEPGRHLGSS (SEQ ID NO:312). Polynucleotides encoding these polypeptides are also encompassed by the invention.

This gene is expressed in stage B2 prostate cancer.

Therefore, polynucleotides and polypeptides of the invention are useful as reagents for differential identification of the tissue(s) or cell type(s) present in a biological sample and for the diagnosis of diseases and conditions, which include, but are not limited to, reproductive disorders, particularly proliferative disorders of the prostate including benign prostatic hypertrophy. Similarly, polypeptides and antibodies directed to those polypeptides are useful to provide immunological probes for differential identification of the tissue(s) or cell type(s). For a number of disorders of the above tissues or cells, particularly of the glandular or reproductive systems, expression of this gene at significantly higher or lower levels may be detected in certain tissues or cell types (e.g., reproductive, prostate, and cancerous and wounded tissues) or bodily fluids (e.g., lymph, seminal fluid, serum, plasma, urine, synovial fluid or spinal fluid) taken from an individual having such a disorder, relative to the standard gene expression level, i.e., the expression level in healthy tissue from an individual not having the disorder.

The tissue distribution in stage B2 prostate cancer tissue suggests that the protein product of this clone is useful for the diagnosis and/or treatment of prostate diseases including prostate cancer, or other reproductive conditions such as male infertility. Similarly, expression within cellular sources marked by proliferating cells suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases.

Many polynucleotide sequences, such as EST sequences, are publicly available and accessible through sequence databases. Some of these sequences are related to SEQ ID NO:46 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 583 of SEQ ID NO:46, b is an integer of 15 to 597, where both a and b correspond to the positions of nucleotide residues shown in SEQ ID NO:46, and where b is greater than or equal to a +14.

Features of Protein Encoded by Gene No: 37

When tested against U937 cell lines, supernatants removed from cells containing this gene activated the GAS (gamma activating sequence) promoter element. Thus, it is likely that this gene activates myeloid cells through the Jak-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

In specific embodiments, polypeptides of the invention comprise the following amino acid sequence: MIILSCCSLWIYDYLIHPVPSVGHRVCLCCLPESATGRISP LGEGPRKWHGLRRSPEHISLGGLLLSSRLMAFCNLSRAVLPGNRTMETETYQ LWASQYQRKWVSRSLSQVQCLRL (SEQ ID NO:313); CCSLWIYDYLIHPVPSV GHRV (SEQ ID NO:314); ISPLGEGPRKWHGLRRSPEHISLGGL (SEQ ID NO:315); and/or RAVLPGNRTMETETYQLWASQYQRKWVSR (SEQ ID NO:316). Polynucleotides encoding these polypeptides are also encompassed by the invention.

This gene is expressed primarily in colorectal tumors.

Therefore, polynucleotides and polypeptides of the invention are useful as reagents for differential identification of the tissue(s) or cell type(s) present in a biological sample and for the diagnosis of diseases and conditions, which include, but are not limited to, cancers of the colon, rectum or gastrointestinal tract. Similarly, polypeptides and antibodies directed to those polypeptides are useful to provide immunological probes for differential identification of the tissue(s) or cell type(s). For a number of disorders of the above tissues or cells, particularly of the digestive system, expression of this gene at significantly higher or lower levels may be detected in certain tissues or cell types (e.g., gastrointesinal, and cancerous and wounded tissues) or bodily fluids (e.g., lymph, bile, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 151 as residues: Phe-48 to Cys-54.

The tissue distribution in colorectal tumors suggests that the protein product of this clone is useful for the treatment or diagnosis of tumors of the gastrointestinal tract, particularly of the colon or rectum. Moreover, the expression within cellular sources marked by proliferating cells suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases.

Many polynucleotide sequences, such as EST sequences, are publicly available and accessible through sequence databases. Some of these sequences are related to SEQ ID NO:47 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 586 of SEQ ID NO:47, b is an integer of 15 to 600, where both a and b correspond to the positions of nucleotide residues shown in SEQ ID NO:47, and where b is greater than or equal to a +14.

Features of Protein Encoded by Gene No: 38

It is likely that the sequence of this polynucleotide continues upstream of the preferred signal peptide.

In specific embodiments, polypeptides of the invention comprise the following amino acid sequence: WIPRAAGIRHEHLSTLDRSVIWS KSILNARCKICRKKGDA ENMVLCDGCDRGHHTYCVRPKLKTVPEGDWFCPECRPKQRSRRLSSRQRPS LESDEDVEDSMGGEDDEVDGDEEEGQSEEEEYEVEQXEDDSXEEXEVRXVL XCN KMSQ (SEQ ID NO:317); MRVARYVERKA (SEQ ID NO:318); HLSTLDRSVIWSK SILNARCK (SEQ ID NO:319); and/or TVPEGDWFCPECRPKQRSRRLSSRQRPSL ESD (SEQ ID NO:320). Polynucleotides encoding these polypeptides are also encompassed by the invention.

This gene is expressed primarily in serum treated smooth muscle.

Therefore, polynucleotides and polypeptides of the invention are useful as reagents for differential identification of the tissue(s) or cell type(s) present in a biological sample and for the diagnosis of diseases and conditions, which include, but are not limited to, neuromuscular or vascular diseases, such as restenosis stroke, aneurysm, or atherosclerosis. Similarly, polypeptides and antibodies directed to those polypeptides are useful to provide immunological probes for differential identification of the tissue(s) or cell type(s). For a number of disorders of the above tissues or cells, particularly of the muscular and vascular sytems, expression of this gene at significantly higher or lower levels may be detected in certain tissues or cell types (e.g., vascular tissue, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 152 as residues: Ser-46 to Trp-54, Lys-76 to Arg-86.

The tissue distribution in smooth muscle tissue suggests that the protein product of this clone is useful for treating restenosis or muscular responses due to degenerative conditions or injury. Moreover, the protein is useful in the detection, treatment, and/or prevention of vascular conditions, which include, but are not limited to, microvascular disease, vascular leak syndrome, aneurysm, stroke, atherosclerosis, arteriosclerosis, or embolism. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:48 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 897 of SEQ ID NO:48, b is an integer of 15 to 911, where both a and b correspond to the positions of nucleotide residues shown in SEQ ID NO:48, and where b is greater than or equal to a +14.

Features of Protein Encoded by Gene No: 39

When tested against dermal fibroblast cell lines, supernatants removed from cells containing this gene activated the EGR1 (early growth response gene 1) promoter element. Thus, it is likely that this gene activates fibroblast cells, and to a lesser extent, other integmentary cells and tissues, through the EGR1 signal transduction pathway. EGR1 is a separate signal transduction pathway from Jak-STAT, genes containing the EGR1 promoter are induced in various tissues and cell types upon activation, leading the cells to undergo differentiation and proliferation.

In specific embodiments, polypeptides of the invention comprise the following amino acid sequence: IRHEDD (SEQ ID NO:321). Polynucleotides encoding these polypeptides are also encompassed by the invention.

The gene encoding the disclosed cDNA is believed to reside on chromosome 3. Accordingly, polynucleotides related to this invention are useful as a marker in linkage analysis for chromosome 3.

This gene is expressed in primary dendritic cells, and to a lesser extent, in human amygdala.

Therefore, polynucleotides and polypeptides of the invention are useful as reagents for diagnosis of the following diseases and conditions: immune or neural disorders. Similarly, polypeptides and antibodies directed to those polypeptides are useful to detect a number of disorders of the above tissues or cells, particularly of the vascular system. Expression of this gene at significantly higher or lower levels may be detected in certain tissues or cell types (e.g., immune, hematopoietic, neural, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 153 as residues: Glu-30 to Gln-42.

The tissue distribution in primary dendritic cells suggests that the protein product of this clone is useful for the treatment and diagnosis of hematopoietic related disorders such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Alternatively, expression within the human amygdala suggests the the protein product of this clone may be useful for the treatment and/or diagnosis of a variety of neural disorders, particularly those involving processesing of sensory information, including endocrine disorders as they relate to neural dysfunction. The protein is useful in modulating the immune response to aberrant neural proteins and peptides, as may be present in cancerous and proliferating tissues and cells. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:49 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1849 of SEQ ID NO:49, b is an integer of 15 to 1863, where both a and b correspond to the positions of nucleotide residues shown in SEQ ID NO:49, and where b is greater than or equal to a +14.

Features of Protein Encoded by Gene No: 40

The translation product of this gene shares sequence homology with the human rtvp-1 and glioma pathogenesis protein which are both glioma-specific proteins thought to be important in regulating the activity of extracellular proteases (See Genbank Accession No.gi|1030053 and gi|847722, respectively).

In specific embodiments, polypeptides of the invention comprise the following amino acid sequence: QRWLKHGANQCKFEHNDCLDKSYKCYAAXEXVGENI WLGGIKSFTPRHAITAWYNETQFYDFDSLSCSRVCGHYTQLVWANSFYVGX AXAMCPNLGGASTAIFVCNYGPAGNFANMPPYVRGESCSLCSKEEKCVKNL CK NPFLKPTGRAPQQTAFNPXQLRFSSSENLLMSFIYKRNS QMLK (SEQ ID NO:322); DPPHPS (SEQ ID NO:323); CLDKSYKCYAAXEXVGENIWLGGIKS FTP (SEQ ID NO:324); ETQFYDFDSLSCSRVCGHYTQLVWANSFYVGXAXA MCPNL (SEQ ID NO:325); STAIFVCNYGPAGNFANMPPYVRGESCS (SEQ ID NO:326); and/or PQQTAFNPXQLRFSSSENLLMSFIYK (SEQ ID NO:327). Polynucleotides encoding these polypeptides are also encompassed by the invention.

This gene is expressed primarily in testes.

Therefore, polynucleotides and polypeptides of the invention are useful as reagents for differential identification of the tissue(s) or cell type(s) present in a biological sample and for the diagnosis of diseases and conditions, which include, but are not limited to, reproductive disorders, particular those disorders where proteases are thought to regulate the levels of secreted proteins including growth factors. Similarly, polypeptides and antibodies directed to those polypeptides are useful to provide immunological probes for differential identification of the tissue(s) or cell type(s). For a number of disorders of the above tissues or cells, particularly of the reproductive system, expression of this gene at significantly higher or lower levels may be detected in certain tissues or cell types (e.g., reproductive, testes, and cancerous and wounded tissues) or bodily fluids (e.g., lymph, seminal fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 154 as residues: Glu-43 to Asn-49.

The tissue distribution in testes combined with the homology to two conserved glioma-specific proteins suggests that the protein product of this clone is useful for treating diseases of the reproductive system or diseases associated with increased degradation of secreted proteins or growth factors. The tissue distribution in testicular tissue indicates that polynucleotides and polypeptides corresponding to this gene are useful for the treatment and diagnosis of conditions concerning proper testicular function (e.g. endocrine function, sperm maturation), as well as cancer. Therefore, this gene product is useful in the treatment of male infertility and/or impotence. This gene product is also useful in assays designed to identify binding agents, as such agents (antagonists) are useful as male contraceptive agents. Similarly, the protein is believed to be useful in the treatment and/or diagnosis of testicular cancer. The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications.

The secreted protein can also be used to determine biological activity, to raise antibodies, as tissue markers, to isolate cognate ligands or receptors, to identify agents that modulate their interactions and as nutritional supplements. It may also have a very wide range of biological acitivities. Typical of these are cytolcine, cell proliferation/differentiation modulating activity or induction of other cytokines; immunostimulating/immunosuppressant activities (e.g. for treating human immunodeficiency virus infection, cancer, autoimmune diseases and allergy); regulation of hematopoiesis (e.g. for treating anaemia or as adjunct to chemotherapy); stimulation or growth of bone, cartilage, tendons, ligaments and/or nerves (e.g. for treating wounds, stimulation of follicle stimulating hormone (for control of fertility); chemotactic and chemokinetic activities (e.g. for treating infections, tumors); hemostatic or thrombolytic activity (e.g. for treating haemophilia, cardiac infarction etc.); anti-inflammatory activity (e.g. for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:50 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 796 of SEQ ID NO:50, b is an integer of 15 to 810, where both a and b correspond to the positions of nucleotide residues shown in SEQ ID NO:50, and where b is greater than or equal to a +14.

Features of Protein Encoded by Gene No: 41

It is likely that the sequence of this polynucleotide continues upstream of the preferred signal peptide.

In specific embodiments, polypeptides of the invention comprise the following amino acid sequence: TEGGCALVPNDMESLKQKLVRVLEENLILSEKIQQLEE GAAISIVSGQQSHTYDDLLHKNQQLTMQVACLNQELAQLKKLEKTVAILHES QRSLVVTNEYLLQQLNKEPKGYSGKALLPPEKGHHLGRSSPFGKSTLSSSSPV AHETGQYLIQSVLDAAPEPGL (SEQ ID NO:328); MESLKQKLVRVLEENLIL SEK IQQLEEGAAISIVSGQQ (SEQ ID NO:330); SMVSK (SEQ ID NO:329); DLLHKiNQQLTMQVACLNQELAQLKKLEKTVA (SEQ ID NO:331); and/or SSPFGKSTLSSSSPVAHETGQYLIQSV (SEQ ID NO:332). Polynucleotides encoding these polypeptides are also encompassed by the invention.

The gene encoding the disclosed cDNA is believed to reside on chromosome 16. Accordingly, polynucleotides related to this invention are useful as a marker in linkage analysis for chromosome 16.

This gene is expressed primarily in lung and testes.

Therefore, polynucleotides and polypeptides of the invention are useful as reagents for differential identification of the tissue(s) or cell type(s) present in a biological sample and for the diagnosis of diseases and conditions, which include, but are not limited to, pulmonary or reproductive diseases such as adult respiratory distress syndrome (ARDS), pulmonary fibrositis or cystic fibrosis, or male infertility. Similarly, polypeptides and antibodies directed to those polypeptides are useful to provide immunological probes for differential identification of the tissue(s) or cell type(s). For a number of disorders of the above tissues or cells, particularly of the respiratory system, expression of this gene at significantly higher or lower levels may be detected in certain tissues or cell types (e.g., reproductive, pulmonary, and cancerous and wounded tissues) or bodily fluids (e.g., lymph, pulmonary surfactant or sputum, seminal fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 155 as residues: Ser-36 to Trp-41, Pro-53 to Arg-58.

The tissue distribution in lung suggests that the protein product of this clone is useful for treating disorders of the lung such as pulmonary fibrosis, cystic fibrosis or acute respiratory distress syndrome. Alternatively, the protein product of this clone may also be useful for the treatment and/or diagnosis of a variety of reproductive disorders, particularly male infertility or impotence, including disorders associated with testosterone regulation and secretion. The tissue distribution in testicular tissue indicates that polynucleotides and polypeptides corresponding to this gene are useful for the treatment and diagnosis of conditions concerning proper testicular function (e.g. endocrine function, sperm maturation), as well as cancer. This gene product is also useful in assays designed to identify binding agents, as such agents (antagonists) are useful as male contraceptive agents.

Similarly, the protein is believed to be useful in the treatment and/or diagnosis of testicular cancer. The testes are also a site of active gene expression of transcripts that may be expressed, particularly at low levels, in other tissues of the body. Therefore, this gene product may be expressed in other specific tissues or organs where it may play related functional roles in other processes, such as hematopoiesis, inflammation, bone formation, and kidney function, to name a few possible target indications. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:51 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 942 of SEQ ID NO:51, b is an integer of 15 to 956, where both a and b correspond to the positions of nucleotide residues shown in SEQ ID NO:51, and where b is greater than or equal to a +14.

Features of Protein Encoded by Gene No: 42

The translation product of this gene shares sequence homology with metallothioneins which are thought to be important in binding zinc and protecting cells from degeneration.

This gene is expressed primarily in the thyroid.

Therefore, polynucleotides and polypeptides of the invention are useful as reagents for differential identification of the tissue(s) or cell type(s) present in a biological sample and for the diagnosis of diseases and conditions, which include, but are not limited to, endocrine disorders, particularly hypo- and hyper-thyroidism. Similarly, polypeptides and antibodies directed to those polypeptides are useful to provide immunological probes for differential identification of the tissue(s) or cell type(s). For a number of disorders of the above tissues or cells, particularly of the endocrine system, expression of this gene at significantly higher or lower levels may be detected in certain tissues or cell types (e.g., endocrine, metabolic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in endocrine tissue combined with the homology to metallothioneins suggests that the protein product of this clone is useful for treating disorders of the thyroid gland, particularly metabolic conditions. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:52 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 286 of SEQ ID NO:52, b is an integer of 15 to 300, where both a and b correspond to the positions of nucleotide residues shown in SEQ ID NO:52, and where b is greater than or equal to a +14.

Features of Protein Encoded by Gene No: 43

In specific embodiments, polypeptides of the invention comprise the following amino acid sequence: NTDWDQTVLIVLRISSTLPVALLRDEVPGWFLKXPEPQL ISKELIMLTEV (SEQ ID NO:333); and/or VLIVLRISSTLPVALLRDEVPGWFLK XPEPQ (SEQ ID NO:334). Polynucleotides encoding these polypeptides are also encompassed by the invention.

This gene is expressed primarily in retinoic acid treated HL60 cells.

Therefore, polynucleotides and polypeptides of the invention are useful as reagents for differential identification of the tissue(s) or cell type(s) present in a biological sample and for the diagnosis of diseases and conditions, which include, but are not limited to, modulation of the immune response to infectious agents, including acute or chronic inflammatory responses. Similarly, polypeptides and antibodies directed to those polypeptides are useful to provide immunological probes for differential identification of the tissue(s) or cell type(s). For example, in a number of disorders of the above tissues or cells, particularly of the immune system, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 157 as residues: Pro-42 to Ser-50, Leu-52 to Phe-58, Pro-61 to Gly-73, Pro-76 to Gln-84.

The tissue distribution in HL60 cells suggests that the protein product of this clone is useful for modulating the immune response to an acute or chronic inflammation or to an infection. The secreted protein can also be used to determine biological activity, to raise antibodies, as tissue markers, to isolate cognate ligands or receptors, to identify agents that modulate their interactions and as nutritional supplements. It may also have a very wide range of biological acitivities. Typical of these are cytokine, cell proliferation/differentiation modulating activity or induction of other cytokines; immunostimulating/immunosuppressant activities (e.g. for treating human immunodeficiency virus infection, cancer, autoimmune diseases and allergy); regulation of hematopoiesis (e.g. for treating anaemia or as adjunct to chemotherapy); stimulation or growth of bone, cartilage, tendons, ligaments and/or nerves (e.g. for treating wounds, stimulation of follicle stimulating hormone (for control of fertility); chemotactic and chemokinetic activities (e.g. for treating infections, tumors); hemostatic or thrombolytic activity (e.g. for treating haemophilia, cardiac infarction etc.); anti-inflammatory activity (e.g. for treating septic shock, Crohn's disease); as antimicrobials; for treating psoriasis or other hyperproliferative diseases; for regulation of metabolism, and behaviour. Also contemplated is the use of the corresponding nucleic acid in gene therapy procedures. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:53 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 827 of SEQ ID NO:53, b is an integer of 15 to 841, where both a and b correspond to the positions of nucleotide residues shown in SEQ ID NO:53, and where b is greater than or equal to a +14.

Features of Protein Encoded by Gene No: 44

This gene is expressed primarily in B-cell lymphoma.

Therefore, polynucleotides and polypeptides of the invention are useful as reagents for differential identification of the tissue(s) or cell type(s) present in a biological sample and for the diagnosis of diseases and conditions, which include, but are not limited to, immune and/or hematopoietic diseases and/or disorders, such as proliferative conditions of the blood. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 158 as residues: Pro-38 to Asp-47, Ser-64 to Asn-71.

The tissue distribution in immune tissue suggests that the protein product of this clone is useful for diagnosing and/or treating tumors of the blood including B-cell lymphomas. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:54 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 620 of SEQ ID NO:54, b is an integer of 15 to 634, where both a and b correspond to the positions of nucleotide residues shown in SEQ ID NO:54, and where b is greater than or equal to a +14.

Features of Protein Encoded by Gene No: 45

In specific embodiments, polypeptides of the invention comprise the following amino acid sequence: GXSSISAVVPAASLWVWPGLRVFR (SEQ ID NO:335). Polynucleotides encoding these polypeptides are also encompassed by the invention.

This gene is expressed primarily in cerebellum.

Therefore, polynucleotides and polypeptides of the invention are useful as reagents for differential identification of the tissue(s) or cell type(s) present in a biological sample and for the diagnosis of neuronal diseases and/or disorders, particularly neurodegenerative conditions. Similarly, polypeptides and antibodies directed to those polypeptides are useful to provide immunological probes for differential identification of the tissue(s) or cell type(s). For a number of disorders of the above tissues or cells, particularly of the cerebellum, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 159 as residues: Cys-56 to Ser-63, Met-67 to Leu-73.

The tissue distribution in cerebellum suggests that the protein product of this clone is useful for the diagnosis and/or treatment of neuronal disorders. The tissue distribution suggests that the protein product of this clone is useful for the detection/treatment of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:55 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 849 of SEQ ID NO:55, b is an integer of 15 to 863, where both a and b correspond to the positions of nucleotide residues shown in SEQ ID NO:55, and where b is greater than or equal to a +14.

Features of Protein Encoded by Gene No: 46

The gene encoding the disclosed cDNA is thought to reside on chromosome 14. Accordingly, polynucleotides related to this invention are useful as a marker in linkage analysis for chromosome 14.

This gene is expressed primarily in colon and neutrophils.

Therefore, polynucleotides and polypeptides of the invention are useful as reagents for differential identification of the tissue(s) or cell type(s) present in a biological sample and for the diagnosis of colon diseases, such as colon cancer. Similarly, polypeptides and antibodies directed to those polypeptides are useful to provide immunological probes for differential identification of the tissue(s) or cell type(s). For a number of disorders of the above tissues or cells, particularly of the colon, expression of this gene at significantly higher or lower levels may be detected in certain tissues or cell types (e.g., gastrointestinal, and cancerous and wounded tissues) or bodily fluids (e.g., lymph, serum, plasma, bile, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 160 as residues: Pro-26 to Asn-32.

The tissue distribution in colon tissue suggests that the protein product of this clone is useful for the diagnosis and/or treatment of colon-related diseases. This gene product may be involved in the regulation of cytokine production, antigen presentation, or other processes suggesting a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immunodeficiency diseases such as AIDS, leukemia, rheumatoid arthritis, granulomatous disease, inflammatory bowel disease, sepsis, acne, neutropenia, neutrophilia, psoriasis, hypersensitivities, such as T-cell mediated cytotoxicity; immune reactions to transplanted organs and tissues, such as host-versus-graft and graft-versus-host diseases, or autoimmunity disorders, such as autoimmune infertility, lense tissue injury, demyelination, systemic lupus erythematosis, drug induced hemolytic anemia, rheumatoid arthritis, Sjogren's disease, scleroderma and tissues. Moreover, the protein may represent a secreted factor that influences the differentiation or behavior of other blood cells, or that recruits hematopoietic cells to sites of injury. In addition, this gene product may have commercial utility in the expansion of stem cells and committed progenitors of various blood lineages, and in the differentiation and/or proliferation of various cell types. Protein is useful in modulating the immune response to aberrant gastrointestinal and metabolic-related polypeptides, as are present in cancerous and proliferative cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:56 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 698 of SEQ ID NO:56, b is an integer of 15 to 712, where both a and b correspond to the positions of nucleotide residues shown in SEQ ID NO:56, and where b is greater than or equal to a +14.

Features of Protein Encoded by Gene No: 47

In specific embodiments, polypeptides of the invention comprise the following amino acid sequence: VCQYCTAXMADFGISAGQFVAVVWDKSSPVEALKGL VDKLQALTGNEGRVSVENI (SEQ ID NO:336); and/or MADFGISAGQFVAVV WDKSSPVEALKGLVDKLQAL (SEQ ID NO:337). Polynucleotides encoding these polypeptides are also encompassed by the invention.

This gene is expressed primarily in a number of tumor tissues such as chondrosarcoma and synovial sarcoma, and to a lesser extent, in activated monocytes and T cells.

Therefore, polynucleotides and polypeptides of the invention are useful as reagents for differential identification of the tissue(s) or cell type(s) present in a biological sample and for the diagnosis of tumorigenesis and immune or hemapoietic diseases and/or disorders. Similarly, polypeptides and antibodies directed to those polypeptides are useful to provide immunological probes for differential identification of the tissue(s) or cell type(s). For a number of disorders of the above tissues or cells, particularly of the tumor and other fast growing tissues, expression of this gene at significantly higher or lower levels may be detected in certain tissues or cell types (e.g., hematopoietic, immune, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in chondrosarcoma and synovial sarcoma tissues suggests that the protein product of this clone is useful for the diagnosis and/or treatment of cell growth related disorders such as tumorigenesis and hemapoietic diseases. Moreover, the protein is useful in the treatment, detection, and/or prevention of conditions afflicting the skeletal system, in particular osteoporosis, bone cancer, as well as, disorders afflicting connective tissues (e.g. arthritis, trauma, tendonitis, chrondomalacia and inflammation), such as in the diagnosis or treatment of various autoimmune disorders such as rheumatoid arthritis, lupus, scleroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). The expression within cellular sources marked by proliferating cells suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:57 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 911 of SEQ ID NO:57, b is an integer of 15 to 925, where both a and b correspond to the positions of nucleotide residues shown in SEQ ID NO:57, and where b is greater than or equal to a +14.

Features of Protein Encoded by Gene No: 48

When tested against U937 cell lines, supernatants removed from cells containing this gene activated the GAS (gamma activating sequence) promoter element. Thus, it is likely that this gene activates myeloid cells, and to a lesser extent, immune and hematopoietic cells and tissues, through the JAK-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

In specific embodiments, polypeptides of the invention comprise the following amino acid sequence: SKCCITTTWKPL (SEQ ID NO:338). Polynucleotides encoding these polypeptides are also encompassed by the invention.

This gene is expressed primarily in breast tissue.

Therefore, polynucleotides and polypeptides of the invention are useful as reagents for differential identification of the tissue(s) or cell type(s) present in a biological sample and for the diagnosis of breast diseases such as breast cancer. Sirnilarly, polypeptides and antibodies directed to those polypeptides are useful to provide immunological probes for differential identification of the tissue(s) or cell type(s). For a number of disorders of the above tissues or cells, particularly of the breast, expression of this gene at significantly higher or lower levels may be detected in certain tissues or cell types (e.g., breast, reproductive, and cancerous and wounded tissues) or bodily fluids (e.g., lymph, serum, plasma, breast milk, urine, synovial fluid or spinal fluid) taken from an individual having such a disorder, relative to the standard gene expression level, i.e., the expression level in healthy tissue from an individual not having the disorder.

The tissue distribution in breast tissue, combined with the detected GAS biological activity, suggests that the protein product of this clone is useful for the diagnosis and/or treatment of breast disorders such as breast cancer, and other reproductive conditions. This protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues. Protein, as well as, antibodies directed against the protein may show utility as a tumor marker and/or immunotherapy targets for the above listed tissues. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:58 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 587 of SEQ ID NO:58, b is an integer of 15 to 601, where both a and b correspond to the positions of nucleotide residues shown in SEQ ID NO:58, and where b is greater than or equal to a +14.

Features of Protein Encoded by Gene No: 49

The translation product of this gene was shown to have homology to capacitative calcium entry channel I of Bos taurus, which is thought to play an integral role in signal calcium-dependent signal transduction pathways (See Genbank Accession: gnl|PID|e276474; See for example EMBO J,(1996), 15(22):6166-71, and PCT application WO9808979, which are hereby incorporated herein by reference). Based on the sequence similarity, the translation product of this clone is expected to share biological activities with other signal transduction-related proteins. Such activities are known in the art, some of which are described elsewhere herein. When tested against Jurkat T-cell lines, supernatants removed from cells containing this gene activated the NF-kB assay. Thus, it is likely that this gene initiates cellular activation, differentiation, or apoptosis, as demonstrated by the NF-kB assay results. NF-KB (Nuclear factor kB) is a transcription factor activated by a wide variety of agents, leading to cell activation, differentiation, or apoptosis. Reporter constructs utilizing the NF-kB promoter element are used to screen supernatants for such activity.

This gene is expressed primarily in chondrosarcoma tissue.

Therefore, polynucleotides and polypeptides of the invention are useful as reagents for differential identification of the tissue(s) or cell type(s) present in a biological sample and for the treatment and diagnosis of skeleltal diseases and/or disorders, particularly chondrosarcoma. Similarly, polypeptides and antibodies directed to those polypeptides are useful to provide immunological probes for differential identification of the tissue(s) or cell type(s). For a number of disorders of the above tissues or cells, particularly chondrosarcoma, expression of this gene at significantly higher or lower levels may be detected in certain tissues or cell types (e.g., skeletal, connective, autoimmune, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in chondrosarcoma tissue suggests that the protein product of this clone is useful for the diagnosis and/or treatment of chondrosarcoma. Moreover, this gene product is useful in the detection and/or treatment of disorders and conditions affecting the skeletal system, in particular osteoporosis, bone cancer, as well as, disorders afflicting connective tissues (e.g. arthritis, trauma, tendonitis, chrondomalacia and inflammation), such as in the diagnosis or treatment of various autoimmune disorders such as rheumatoid arthritis, lupus, scieroderma, and dermatomyositis as well as dwarfism, spinal deformation, and specific joint abnormalities as well as chondrodysplasias (i.e. spondyloepiphyseal dysplasia congenita, familial osteoarthritis, Atelosteogenesis type II, metaphyseal chondrodysplasia type Schmid). The expression within cellular sources marked by proliferating cells suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues, particularly considering the detected NF-Kb biological activity. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:59 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 716 of SEQ ID NO:59, b is an integer of 15 to 730, where both a and b correspond to the positions of nucleotide residues shown in SEQ ID NO:59, and where b is greater than or equal to a +14.

Features of Protein Encoded by Gene No: 50

This gene is expressed primarily in human embryo.

Therefore, polynucleotides and polypeptides of the invention are useful as reagents for differential identification of the tissue(s) or cell type(s) present in a biological sample and for the diagnosis of diseases and conditions, which include, but are not limited to, embryonic development disorders. Similarly, polypeptides and antibodies directed to those polypeptides are useful to provide immunological probes for differential identification of the tissue(s) or cell type(s). For a number of disorders of the above tissues or cells, particularly of the embryo, expression of this gene at significantly higher or lower levels may be detected in certain tissues or cell types (e.g., embryonic, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in embryonic tissue suggests that the protein product of this clone is useful for the diagnosis and/or treatment of embryonic development disorders. Embryonic development also involves decisions involving cell differentiation and/or apoptosis in pattern formation. Thus this protein may also be involved in apoptosis or tissue differentiation and could be useful in cancer therapy. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:60 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 831 of SEQ ID NO:60, b is an integer of 15 to 845, where both a and b correspond to the positions of nucleotide residues shown in SEQ ID NO:60, and where b is greater than or equal to a +14.

Features of Protein Encoded by Gene No: 51 In specific embodiments, polypeptides of the invention comprise the following amino acid sequence: MSSPLLTASSLGQAGTLRKIKPSLTTHHIQCPCSSLREE GRTSQ (SEQ ID NO:339). Polynucleotides encoding these polypeptides are also encompassed by the invention.

The gene encoding the disclosed cDNA is thought to reside on chromosome 9. Accordingly, polynucleotides related to this invention are useful as a marker in linkage analysis for chromosome 9.

This gene is expressed primarily in neuronal tissues, fetal tissues, and a number of cancer tissues.

Therefore, polynucleotides and polypeptides of the invention are useful as reagents for differential identification of the tissue(s) or cell type(s) present in a biological sample and for the diagnosis of diseases and conditions, which include, but are not limited to, neuronal disorders, early developmental disorders, and tumorigenesis. Similarly, polypeptides and antibodies directed to those polypeptides are useful to provide immunological probes for differential identification of the tissue(s) or cell type(s). For a number of disorders of the above tissues or cells, particularly of neuronal tissues, fetal tissues, and some cancer tissues, expression of this gene at significantly higher or lower levels may be detected in certain tissues or cell types (e.g., fetal tissues, brain, cancerous and wounded tissues) or bodily fluids (e.g., lymph, serum, plasma, urine, amniotic fluid, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 165 as residues: Met-1 to Ser-6, Gln-59 to Gly-67.

The tissue distribution in neuronal and fetal tissues suggests that the protein product of this clone is useful for the diagnosis and/or treatment of neuronal disorders, early developmental disorders, and tumorigenesis. Expression within fetal tissue and other cellular sources marked by proliferating cells suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues.

Morover, the protein product of this clone is useful for the detection, treatment, and/or prevention of neurodegenerative disease states, behavioral disorders, or inflammatory conditions which include, but are not limited to Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain suggests it plays a role in normal neural function.

Potentially, this gene product is involved in synapse formation, neurotransmission, learning, cognition, homeostasis, or neuronal differentiation or survival. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:61 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 944 of SEQ ID NO:61, b is an integer of 15 to 958, where both a and b correspond to the positions of nucleotide residues shown in SEQ ID NO:61, and where b is greater than or equal to a +14.

Features of Protein Encoded by Gene No: 52

In specific embodiments, polypeptides of the invention comprise the following amino acid sequence: GLWTGINHRNMI (SEQ ID NO:340). Polynucleotides encoding these polypeptides are also encompassed by the invention.

This gene is expressed primarily in fetal brain.

Therefore, polynucleotides and polypeptides of the invention are useful as reagents for differential identification of the tissue(s) or cell type(s) present in a biological sample and for the diagnosis of neuronal and development diseases and/or disorders. Similarly, polypeptides and antibodies directed to those polypeptides are useful to provide immunological probes for differential identification of the tissue(s) or cell type(s). For a number of disorders of the above tissues or cells, particularly of the fetal brain, expression of this gene at significantly higher or lower levels may be detected in certain tissues or cell types (e.g., brain, developmental, and cancerous and wounded tissues) or bodily fluids (e.g., lymph, serum, plasma, urine, amniotic fluid, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 166 as residues: Ser-25 to Tyr-35.

The tissue distribution in fetal brain tissue suggests that the protein product of this clone is useful for the diagnosis and treatment of neuronal development disorders, fetal deficiencies, and pre-natal disorders. Moreover, the expression within fetal tissue suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some tO cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant developmental and/or neural polypeptides, as may exist in proliferating and cancerous cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:62 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 568 of SEQ ID NO:62, b is an integer of 15 to 582, where both a and b correspond to the positions of nucleotide residues shown in SEQ ID NO:62, and where b is greater than or equal to a +14.

Features of Protein Encoded by Gene No: 53

When tested against both U937 myeloid and Jurkat T-cell cell lines, supernatants removed from cells containing this gene activated the GAS assay. Thus, it is likely that this gene activates myeloid cells and T-cells, and to a lesser extent, other immune or hemaopoietic cells and tissues, through the Jak-STAT signal transduction pathway. GAS (gamma activating sequence) is a a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

In specific embodiments, polypeptides of the invention comprise the following armino acid sequence: FQREVFAPPS (SEQ ID NO:341); IGQGRHSDSREKSLLL HLWKNFSHClYYYMFLTGVSLLLDREQVYLLLSPQPLDLGRLIVDIWEMLGK ERRGGERKDSMAMSKCPAMS (SEQ ID NO:342); KNFSHClYYYMFLTGVSL LLDREQVYLL (SEQ ID NO:343); and/or VDIWEMLGKERRGGERKDSMAM SKC (SEQ ID NO:344). Polynucleotides encoding these polypeptides are also encompassed by the invention.

This gene is expressed primarily in brain frontal cortex.

Therefore, polynucleotides and polypeptides of the invention are useful as reagents for differential identification of the tissue(s) or cell type(s) present in a biological sample and for the diagnosis of neurological diseases and/or disorders. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 167 as residues: Gly-36 to Arg-43, Glu-50 to Glu-58.

The tissue distribution in brain frontal cortex suggests that the protein product of this clone is useful for the detection/treatment of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. The protein is useful in modulating the immune response to aberrant neural polypeptides, as may exist in proliferating neural and brain cancer cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:63 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 738 of SEQ ID NO:63, b is an integer of 15 to 752, where both a and b correspond to the positions of nucleotide residues shown in SEQ ID NO:63, and where b is greater than or equal to a +14.

Features of Protein Encoded by Gene No: 54

In specific embodiments, polypeptides of the invention comprise the following amino acid sequence: KEIPTVWHQDLCDLQGACFPQQSLFYTTCSPHHPGPFH LLKNTELLFTVGPLNAYFSKFHSSTRLQEFSLREESKQVWPQLLEMAEERVFS LNGGGGSCVLGNPISPFIS (SEQ ID NO:345); CDLQGACFPQQSLFYTTCSPH HPGPFHLLKNT (SEQ ID NO:346); FTVGPLN AYFSKFHSSTRLQEFSLRE (SEQ ID NO:347); and/or VWPQLLEMAEERVFSLNGGGGSCVLGN (SEQ ID NO:348). Polynucleotides encoding these polypeptides are also encompassed by the invention.

This gene is expressed primarily in the endometrium.

Therefore, polynucleotides and polypeptides of the invention are useful as reagents for differential identification of the tissue(s) or cell type(s) present in a biological sample and for the diagnosis of reproductive disorders and endometrial diseases such as endometrial tumors. Similarly, polypeptides and antibodies directed to those polypeptides are useful to provide immunological probes for differential identification of the tissue(s) or cell type(s). For a number of disorders of the above tissues or cells, particularly of the endometrium, expression of this gene at significantly higher or lower levels may be detected in certain tissues or cell types (e.g., reproductive, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 168 as residues: Arg-7 to Ser-14, Pro-32 to Leu-39.

The tissue distribution in endometrial tissue suggests that the protein product of this clone is useful for the diagnosis and/or treatment of the endometrium diseases such as endometrium tumor. Given the tissue distribution, the protein product of this gene may also be useful in the treatment of reproductive disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:64 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 692 of SEQ ID NO:64, b is an integer of 15 to 706, where both a and b correspond to the positions of nucleotide residues shown in SEQ ID NO:64, and where b is greater than or equal to a +14.

Features of Protein Encoded by Gene No: 55

In specific embodiments, polypeptides of the invention comprise the following amino acid sequence: STHASALHGE (SEQ ID NO:349). Polynucleotides encoding these polypeptides are also encompassed by the invention.

This gene is expressed primarily in activated T cells.

Therefore, polynucleotides and polypeptides of the invention are useful as reagents for differential identification of the tissue(s) or cell type(s) present in a biological sample and for the diagnosis of immune and hematopoietic diseases and/or disorders. Similarly, polypeptides and antibodies directed to those polypeptides are useful to provide immunological probes for differential identification of the tissue(s) or cell type(s). For a number of disorders of the above tissues or cells, particularly of activated T-cells, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 169 as residues: Arg-35 to Gly-44.

The tissue distribution in T-cells suggests that the protein product of this clone is useful for the diagnosis and/or treatment of immune disorders. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. The protein is useful in modulating the immune response to aberrant polypeptides, as may exist in proliferating and cancerous cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:65 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 386 of SEQ ID NO:65, b is an integer of 15 to 400, where both a and b correspond to the positions of nucleotide residues shown in SEQ ID NO:65, and where b is greater than or equal to a +14.

Features of Protein Encoded by Gene No: 56

In specific embodiments, polypeptides of the invention comprise the following amino acid sequence: MGISACXLPPASLPFPAEAAPEPLPSR (SEQ ID NO:350). Polynucleotides encoding these polypeptides are also encompassed by the invention.

This gene is expressed primarily in skin tissue.

Therefore, polynucleotides and polypeptides of the invention are useful as reagents for differential identification of the tissue(s) or cell type(s) present in a biological sample and for the diagnosis of diseases and conditions relating to skin. Similarly, polypeptides and antibodies directed to those polypeptides are useful to provide immunological probes for differential identification of the tissue(s) or cell type(s). For a number of disorders of the above tissues or cells, particularly of the endocrine system, expression of this gene at significantly higher or lower levels may be detected in certain tissues or cell types (e.g., skin, integumentary, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in skin tissue suggests that the protein product of this clone is useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e.wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma.

Moreover, such disorders may predispose an individual to viral and bacterial infections of the skin (i.e. cold sores, warts, chickenpox, molluscum contagiosum, herpes zoster, boils, cellulitis, erysipelas, impetigo, tinea, althletes foot, and ringworm). The protein is useful in modulating the immune response to aberrant polypeptides, as may exist in proliferating and cancerous cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:66 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 759 of SEQ ID NO:66, b is an integer of 15 to 773, where both a and b correspond to the positions of nucleotide residues shown in SEQ ID NO:66, and where b is greater than or equal to a +14.

Features of Protein Encoded by Gene No: 57

This gene is expressed primarily in human fetal kidney.

Therefore, polynucleotides and polypeptides of the invention are useful as reagents for differential identification of the tissue(s) or cell type(s) present in a biological sample and for the diagnosis of renal diseases and/or disorders, in addition to developmental conditions. Similarly, polypeptides and antibodies directed to those polypeptides are useful to provide immunological probes for differential identification of the tissue(s) or cell type(s). For a number of disorders of the above tissues or cells, particularly of the urinary system, expression of this gene at significantly higher or lower levels may be detected in certain tissues or cell types (e.g., renal, developmental, and cancerous and wounded tissues) or bodily fluids (e.g., lymph, serum, plasma, urine, amniotic fluid, synovial fluid or spinal fluid) taken from an individual having such a disorder, relative to the standard gene expression level, i.e., the expression level in healthy tissue from an individual not having the disorder.

The tissue distribution in fetal kidney suggests that this gene or gene product could be used in the treatment and/or detection of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilms Tumor Disease, and congenital kidney abnormalities such as horseshoe kidney, polycystic kidney, and Falconi's syndrome. Moreover, the expression within fetal tissue suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant renal polypeptides, as may exist in proliferating and cancerous cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:67 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 633 of SEQ ID NO:67, b is an integer of 15 to 647, where both a and b correspond to the positions of nucleotide residues shown in SEQ ID NO:67, and where b is greater than or equal to a +14.

Features of Protein Encoded by Gene No: 58

In specific embodiments, polypeptides of the invention comprise the following amino acid sequence: GLLHSSGCKIYILLPEVDTFAWVLFKE (SEQ ID NO:351); DYSIPLDVKSTFSCLRWIRLLGFCLRRWGQQCVSGPVKCVLYPGFCLISVFSL AYQSHCRGYLVSESRTF PGCCGTD (SEQ ID NO:352); KSTFSCLRWIRLLGF CLRRWGQQCVS (SEQ ID NO:353); and/or LYPGFCLISVFSLAYQSHCRGYLV SESR (SEQ ID NO:354). Polynucleotides encoding these polypeptides are also encompassed by the invention.

This gene is expressed primarily in human fetal dura mater.

Therefore, polynucleotides and polypeptides of the invention are useful as reagents for differential identification of the tissue(s) or cell type(s) present in a biological sample and for the diagnosis of disorders related to the central nervous system, in addition to, developmental diseases and/or conditions. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., brain, developmental, and cancerous and wounded tissues) or bodily fluids (e.g., lymph, serum, plasma, urine, amniotic fluid, synovial fluid or spinal fluid) taken from an individual having such a disorder, relative to the standard gene expression level, i.e., the expression level in healthy tissue from an individual not having the disorder.

The tissue distribution in fetal dura mater suggests that the protein product of this clone is useful for the diagnosis and/or treatment of disorders of the brain and nervous system. Elevated expression of this gene product within the dura mater suggests that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's. Moreover, the expression within fetal tissue suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:68 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 661 of SEQ ID NO:68, b is an integer of 15 to 675, where both a and b correspond to the positions of nucleotide residues shown in SEQ ID NO:68, and where b is greater than or equal to a +14.

Features of Protein Encoded by Gene No: 59

The translation product of this gene shares sequence homology with human beta-galactosidase (GLB1) mRNA.

The gene encoding the disclosed cDNA is thought to reside on chromosome 3. Accordingly, polynucleotides related to this invention are useful as a marker in linkage analysis for chromosome 3.

This gene is expressed primarily in activated human neutrophil, and to a lesser extent in breast, kidney and gall-bladder tissue.

Therefore, polynucleotides and polypeptides of the invention are useful as reagents for differential identification of the tissue(s) or cell type(s) present in a biological sample and for the diagnosis of diseases and conditions, which include, but are not limited to, neutropenia and neutrophilia. Similarly, polypeptides and antibodies directed to those polypeptides are useful to provide immunological probes for differential identification of the tissue(s) or cell type(s). For a number of disorders of the above tissues or cells, particularly of the disorders relating to hemopoietic system, expression of this gene at significantly higher or lower levels may be detected in certain tissues or cell types (e.g., immune, reproductive, renal, metabolic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in neutrophils suggests that the protein product of this clone is useful for the diagnosis and/or treatment of immune disorders. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. The protein is useful in modulating the immune response to aberrant polypeptides, as may exist in proliferating and cancerous cells and tissues of the urogenital, reproductive, and hematopoietic system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:69 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 875 of SEQ ID NO:69, b is an integer of 15 to 889, where both a and b correspond to the positions of nucleotide residues shown in SEQ ID NO:69, and where b is greater than or equal to a +14.

Features of Protein Encoded by Gene No: 60

In specific embodiments, polypeptides of the invention comprise the following amino acid sequence: GTRTAVQS (SEQ ID NO:355). Polynucleotides encoding these polypeptides are also encompassed by the invention.

This gene is expressed primarily in human fetal kidney.

Therefore, polynucleotides and polypeptides of the invention are useful as reagents for differential identification of the tissue(s) or cell type(s) present in a biological sample and for the diagnosis of renal and developmental diseases and/or disorders. Similarly, polypeptides and antibodies directed to those polypeptides are useful to provide immunological probes for differential identification of the tissue(s) or cell type(s). For a number of disorders of the above tissues or cells, particularly of the urinary system, expression of this gene at significantly higher or lower levels may be detected in certain tissues or cell types (e.g., developmental, renal, cancerous and wounded tissues) or bodily fluids (e.g., lymph, amniotic fluid, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 174 as residues: Arg-27 to Asn-38, His-41 to Ser-54.

The tissue distribution in kidney suggests that this gene or gene product could be used in the treatment and/or detection of kidney diseases including renal failure, nephritus, renal tubular acidosis, proteinuria, pyuria, edema, pyelonephritis, hydronephritis, nephrotic syndrome, crush syndrome, glomerulonephritis, hematuria, renal colic and kidney stones, in addition to Wilms Tumor Disease, and congenital kidney abnormalities such as horseshoe kidney, polycystic kidney, and Falconi's syndrome. Moreover, the expression within fetal tissue suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:70 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 874 of SEQ ID NO:70, b is an integer of 15 to 888, where both a and b correspond to the positions of nucleotide residues shown in SEQ ID NO:70, and where b is greater than or equal to a +14.

Features of Protein Encoded by Gene No: 61

In specific embodiments, polypeptides of the invention comprise the following amino acid sequence: LTQEPCPISVS (SEQ ID NO:356). Polynucleotides encoding these polypeptides are also encompassed by the invention.

This gene is expressed primarily in human frontal cortex of an epileptic person.

Therefore, polynucleotides and polypeptides of the invention are useful as reagents for differential identification of the tissue(s) or cell type(s) present in a biological sample and for the diagnosis of neural diseases and/or disorders, particularly epilepsy. Similarly, polypeptides and antibodies directed to those polypeptides are useful to provide immunological probes for differential identification of the tissue(s) or cell type(s). For a number of disorders of the above tissues or cells, particularly of the ANS and central nervous system, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in frontal cortex tissue suggests that the protein product of this clone is useful for the diagnosis and/or treatment of epilepsy. Furthermore, the tissue distribution suggests that the protein product of this clone is useful for the diagnosis and/or treatment of disorders of the brain and nervous system. Elevated expression of this gene product within the frontal cortex of the brain suggests that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:71 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 782 of SEQ ID NO:71, b is an integer of 15 to 796, where both a and b correspond to the positions of nucleotide residues shown in SEQ ID NO:71, and where b is greater than or equal to a +14.

Features of Protein Encoded by Gene No: 62

In specific embodiments, polypeptides of the invention comprise the following amino acid sequence: LCIWTRFIFLFKVAIH (SEQ ID NO:357). Polynucleotides encoding these polypeptides are also encompassed by the invention.

This gene is expressed primarily in human frontal cortex in a person with Schizophrenia.

Therefore, polynucleotides and polypeptides of the invention are useful as reagents for differential identification of the tissue(s) or cell type(s) present in a biological sample and for the diagnosis of neural diseases and/or disorders, particularly schizophrenic disorders. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 176 as residues: Pro-49 to Gly-54.

The tissue distribution in frontal cortex suggests that the protein product of this clone is useful for the diagnosis and/or treatment of disorders of the brain and nervous system. Elevated expression of this gene product suggests that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's. In addition, elevated expression of this gene product in regions of the brain suggests it plays a role in normal neural function.

Potentially, this gene product is involved in synapse formation, neurotransmission, learning, cognition, homeostasis, or neuronal differentiation or survival. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:72 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 518 of SEQ ID NO:72, b is an integer of 15 to 532, where both a and b correspond to the positions of nucleotide residues shown in SEQ ID NO:72, and where b is greater than or equal to a +14.

Features of Protein Encoded by Gene No: 63

In specific embodiments, polypeptides of the invention comprise the following amino acid sequence: IFPKPHMTPVCFRLLEALEESIGVDEMESFKSCFGFCFCV WVFKESISCHVEENPGGGCPPTGRR (SEQ ID NO:358); and/or ESIGVDEMES FKSCFGFCFCVWVFKESI (SEQ ID NO:359). Polynucleotides encoding these polypeptides are also encompassed by the invention.

This gene is expressed primarily in hemangiopericytoma.

Therefore, polynucleotides and polypeptides of the invention are useful as reagents for differential identification of the tissue(s) or cell type(s) present in a biological sample and for the diagnosis of diseases and conditions, which include, but are not limited to, benign disorders related to pericytes and endothelium-lined vessels, including soft-tissue cancers. Similarly, polypeptides and antibodies directed to those polypeptides are useful to provide immunological probes for differential identification of the tissue(s) or cell type(s). For a number of disorders of the above tissues or cells, particularly of the nonmalignant character of neoplasm relating to pericytes and endothelial vessels, expression of this gene at significantly higher or lower levels may be detected in certain tissues or cell types (e.g., blood vessels, pericytes, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in hemangiopericytoma suggests that the protein product of this clone is useful for the diagnosis and/or treatment of proliferative diseases and/or disorders. Moreover, the expression within cellular sources marked by proliferating cells suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:73 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 532 of SEQ ID NO:73, b is an integer of 15 to 546, where both a and b correspond to the positions of nucleotide residues shown in SEQ ID NO:73, and where b is greater than or equal to a +14.

Features of Protein Encoded by Gene No: 64

In specific embodiments, polypeptides of the invention comprise the following amino acid sequence: DFLLFPHAGPNSKFPRAD (SEQ ID NO:360). Polynucleotides encoding these polypeptides are also encompassed by the invention.

This gene is expressed primarily in hemangiopericytoma, and to a lesser extent in human colon.

Therefore, polynucleotides and polypeptides of the invention are useful as reagents for differential identification of the tissue(s) or cell type(s) present in a biological sample and for the diagnosis of diseases and conditions, which include, but are not limited to, benign disorders related to pericytes and endothelium-lined vessels, in addition to, gastrointestinal diseases and/or disorders. Similarly, polypeptides and antibodies directed to those polypeptides are useful to provide immunological probes for differential identification of the tissue(s) or cell type(s). For a number of disorders of the above tissues or cells, particularly of the nonmalignant character of neoplasm relating to pericytes and endothelial vessels, expression of this gene at significantly higher or lower levels may be detected in certain tissues or cell types (e.g., gastrointestinal, brain, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 178 as residues: Lys-39 to Glu-45.

The tissue distribution in hemangiopericytoma suggests that the protein product of this clone is useful for the diagnosis and/or treatment of proliferative diseases and/or conditions. Moreover, the expression within cellular sources marked by proliferating cells (i.e., hemangiopericytoma, etc.) suggests this protein may play a role in the regulation of cellular division, and may show utility in the diagnosis, treatment, and/or prevention of developmental diseases and disorders, cancer, and other proliferative conditions.

Similarly, developmental tissues rely on decisions involving cell differentiation and/or apoptosis in pattern formation. Dysregulation of apoptosis can result in inappropriate suppression of cell death, as occurs in the development of some cancers, or in failure to control the extent of cell death, as is believed to occur in acquired immunodeficiency and certain neurodegenerative disorders, such as spinal muscular atrophy (SMA). Therefore, the polynucleotides and polypeptides of the present invention are useful in treating, detecting, and/or preventing said disorders and conditions, in addition to other types of degenerative conditions. Thus this protein may modulate apoptosis or tissue differentiation and would be useful in the detection, treatment, and/or prevention of degenerative or proliferative conditions and diseases. The protein is useful in modulating the immune response to aberrant polypeptides, as may exist in proliferating and cancerous cells and tissues. The protein can also be used to gain new insight into the regulation of cellular growth and proliferation. Protein, as well as, antibodies directed against the protein may show utility as a tumor marker and/or immunotherapy targets for the above listed tissues.

Many polynucleotide sequences, such as EST sequences, are publicly avlable and accessible through sequence databases. Some of these sequences are related to SEQ ID NO:74 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 701 of SEQ ID NO:74, b is an integer of 15 to 715, where both a and b correspond to the positions of nucleotide residues shown in SEQ ID NO:74, and where b is greater than or equal to a +14.

Features of Protein Encoded by Gene No: 65

This gene is expressed primarily in glioblastoma, and to a lesser extent in B-cell lymphoma and anergic T-cells.

Therefore, polynucleotides and polypeptides of the invention are useful as reagents for differential identification of the tissue(s) or cell type(s) present in a biological sample and for the diagnosis of diseases and conditions, which include, but are not limited to, disorders related to neuroglial and ependymal cells, as well as the immune system, including tumors. Similarly, polypeptides and antibodies directed to those polypeptides are useful to provide immunological probes for differential identification of the tissue(s) or cell type(s). For a number of disorders of the above tissues or cells, particularly of the central nervous system or immune system, expression of this gene at significantly higher or lower levels may be detected in certain tissues or cell types (e.g., brain, immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in glioblastoma cells and tissues suggests that the protein product of this clone is useful for the diagnosis and/or treatment of neural cell disorders. Furthermore, the tissue distribution suggests that the translation product of this clone is useful for the treatment and/or detection of tumors of the brain and immune system, such as glioblastomas and B-cell lymphomas. The protein is useful in modulating the immune response to aberrant polypeptides, as may exist in proliferating and cancerous cells and tissues (i.e. neural cancers). The protein can also be used to gain new insight into the regulation of cellular growth and proliferation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:75 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 392 of SEQ ID NO:75, b is an integer of 15 to 406, where both a and b correspond to the positions of nucleotide residues shown in SEQ ID NO:75, and where b is greater than or equal to a +14.

Features of Protein Encoded by Gene No: 66

This gene is expressed primarily in skin.

Therefore, polynucleotides and polypeptides of the invention are useful as reagents for differential identification of the tissue(s) or cell type(s) present in a biological sample and for the diagnosis of diseases and conditions relating to skin.

Similarly, polypeptides and antibodies directed to those polypeptides are useful to provide immunological probes for differential identification of the tissue(s) or cell type(s). For a number of disorders of the above tissues or cells, particularly of the endocrine system, expression of this gene at significantly higher or lower levels may be detected in certain tissues or cell types (e.g., skin, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 180 as residues: Pro-27 to Pro-40.

The tissue distribution in skin suggests that the protein product of this clone is useful for the treatment, diagnosis, and/or prevention of various skin disorders including congenital disorders (i.e. nevi, moles, freckles, Mongolian spots, hemangiomas, port-wine syndrome), integumentary tumors (i.e. keratoses, Bowen's disease, basal cell carcinoma, squamous cell carcinoma, malignant melanoma, Paget's disease, mycosis fungoides, and Kaposi's sarcoma), injuries and inflammation of the skin (i.e.wounds, rashes, prickly heat disorder, psoriasis, dermatitis), atherosclerosis, uticaria, eczema, photosensitivity, autoimmune disorders (i.e. lupus erythematosus, vitiligo, dermatomyositis, morphea, scleroderma, pemphigoid, and pemphigus), keloids, striae, erythema, petechiae, purpura, and xanthelasma. Moreover, such disorders may predispose an individual to viral and bacterial infections of the skin (i.e. cold sores, warts, chickenpox, molluscum contagiosum, herpes zoster, boils, cellulitis, erysipelas, impetigo, tinea, althletes foot, and ringworm). The protein is useful in modulating the immune response to aberrant polypeptides, as may exist in proliferating and cancerous cells and tissues (i.e. skin tumors, melatoma, etc.). The protein can also be used to gain new insight into the regulation of cellular growth and proliferation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:76 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 528 of SEQ ID NO:76, b is an integer of 15 to 542, where both a and b correspond to the positions of nucleotide residues shown in SEQ ID NO:76, and where b is greater than or equal to a +14.

Features of Protein Encoded by Gene No: 67

In specific embodiments, polypeptides of the invention comprise the following amino acid sequence: LHRELPLLWAKDKKECRLVSRMIKLHSAYSSRVRPVL VGFRAAFRPAGLRLP LMRM (SEQ ID NO:361). Polynucleotides encoding these polypeptides are also encompassed by the invention.

This gene is expressed primarily in brain frontal cortex.

Therefore, polynucleotides and polypeptides of the invention are useful as reagents for differential identification of the tissue(s) or cell type(s) present in a biological sample and for the diagnosis of diseases and conditions, which include, but are not limited to, neurological diseases and/or disorders. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 181 as residues: Gly-27 to Pro-34, Tyr-59 to Arg-65.

The tissue distribution in frontal cortex tissue suggests that the protein product of this clone is useful for the diagnosis and/or treatment of disorders of the brain and nervous system. Elevated expression of this gene product suggests that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:77 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 406 of SEQ ID NO:77, b is an integer of 15 to 420, where both a and b correspond to the positions of nucleotide residues shown in SEQ ID NO:77, and where b is greater than or equal to a +14.

Features of Protein Encoded by Gene No: 68

This gene is expressed primarily in human frontal cortex of a person exhibiting Schizophrenia.

Therefore, polynucleotides and polypeptides of the invention are useful as reagents for differential identification of the tissue(s) or cell type(s) present in a biological sample and for the diagnosis of neural disorders, particularly neurodegenerative conditions, such as Schizophrenia. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution human frontal cortex suggests that the protein product of this clone is useful for the diagnosis and/or treatment of disorders of the brain and nervous system. Moreover, the expression of this gene product suggests that it may be involved in neuronal survival; synapse formation; conductance; neural differentiation, etc. Such involvement may impact many processes, such as learning and cognition. It may also be useful in the treatment of such neurodegenerative disorders as schizophrenia; ALS; or Alzheimer's. The protein is useful in modulating the immune response to aberrant neural polypeptides, as may exist in proliferating and cancerous cells and tissues of the brain and spinal cord. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:78 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 451 of SEQ ID NO:78, b is an integer of 15 to 465, where both a and b correspond to the positions of nucleotide residues shown in SEQ ID NO:78, and where b is greater than or equal to a +14.

Features of Protein Encoded by Gene No: 69

This gene is expressed primarily in glioblastoma tissue.

Therefore, polynucleotides and polypeptides of the invention are useful as reagents for differential identification of the tissue(s) or cell type(s) present in a biological sample and for the diagnosis of diseases and conditions, which include, but are not limited to, disorders related to neuroglial and ependymal cells, including cancers. Similarly, polypeptides and antibodies directed to those polypeptides are useful to provide immunological probes for differential identification of the tissue(s) or cell type(s). For a number of disorders of the above tissues or cells, particularly of the central nervous system, expression of this gene at significantly higher or lower levels may be detected in certain tissues or cell types (e.g., brain, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in glioblastoma tissue suggests that the protein product of this clone is useful for the diagnosis and/or treatment of neural cell disorders. Furthermore, given the tissue distribution, the translation product of this gene may be useful for the intervention or detection of tumors of the brain, such as glioblastomas, as well as cancers of other tissues where expression of this gene has been observed. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:79 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 875 of SEQ ID NO:79, b is an integer of 15 to 889, where both a and b correspond to the positions of nucleotide residues shown in SEQ ID NO:79, and where b is greater than or equal to a +14.

Features of Protein Encoded by Gene No: 70

This gene is expressed primarily in human fetal brain tissue.

Therefore, polynucleotides and polypeptides of the invention are useful as reagents for differential identification of the tissue(s) or cell type(s) present in a biological sample and for the diagnosis of diseases and conditions, which include, but are not limited to, immune, growth, and neurologic disorders. Similarly, polypeptides and antibodies directed to those polypeptides are useful to provide immunological probes for differential identification of the tissue(s) or cell type(s). For a number of disorders of the above tissues or cells, particularly of the central nervous system and immune system, expression of this gene at significantly higher or lower levels may be detected in certain tissues or cell types (e.g., brain, immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 184 as residues: Lys-13 to Asn-19, Asn-27 to Asn-35.

The tissue distribution in fetal brain tissue suggests that the protein product of this clone is useful for the detection and/or treatment of disorders of the central nervous system and immune system. The tissue distribution suggests that the protein product of this clone is useful for the detection/treatment of neurodegenerative disease states and behavioural disorders such as Alzheimers Disease, Parkinsons Disease, Huntingtons Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, the gene or gene product may also play a role in the treatment and/or detection of developmental disorders associated with the developing embryo or sexually-linked disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:80 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 456 of SEQ ID NO:80, b is an integer of 15 to 470, where both a and b correspond to the positions of nucleotide residues shown in SEQ ID NO:80, and where b is greater than or equal to a +14.

Features of Protein Encoded by Gene No: 71

In specific embodiments, polypeptides of the invention comprise the following amino acid sequences: AFAKSYLGDTIEGTXAGTGPEFPGRPTRPPAWRPRRGA ATRRFASSLRIICGRVP (SEQ ID NO:362); and/or RRXKAFVTQDIPFYHXLVM KHLPGADPELVLLGRRYEELERIPLSEMTREEINALVQELGFYRKAAPDAQVP PEYVWAPAKPPEETSDHADL (SEQ ID NO:363). Polynucleotides encoding these polypeptides are also encompassed by the invention.

This gene is expressed primarily in human epithelioid sarcoma tissue, and to a lesser extent in breast cancer, endometrial stromal cells, and adrenal gland tumors.

Therefore, polynucleotides and polypeptides of the invention are useful as reagents for differential identification of the tissue(s) or cell type(s) present in a biological sample and for the diagnosis of diseases and conditions, which include, but are not limited to, disorders related to epithelium, and cancer. Similarly, polypeptides and antibodies directed to those polypeptides are useful to provide immunological probes for differential identification of the tissue(s) or cell type(s). For a number of disorders of the above tissues or cells, particularly of the endocrine system, expression of this gene at significantly higher or lower levels may be detected in certain tissues or cell types (e.g., epithelium, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in epithelioid sarcoma tissue suggests that the protein product of this clone is useful for the diagnosis and/or treatment of epithelial disorders. Furthermore, the tissue distribution in adrenal gland tumor tissue suggests that the protein product of this clone is useful for the detection, treatment, and/or prevention of various endocrine disorders and cancers, particularly Addison's disease, Cushing's Syndrome, and disorders and/or cancers of the pancrease (e.g. diabetes mellitus), adrenal cortex, pituitary (e.g., hyper-, hypopituitarism), thyroid (e.g. hyper-, hypothyroidism), parathyroid (e.g. hyper-,hypoparathyroidism), and hypothallamus. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:81 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1076 of SEQ ID NO: 81, b is an integer of 15 to 1090, where both a and b correspond to the positions of nucleotide residues shown in SEQ ID NO:81, and where b is greater than or equal to a +14.

Features of Protein Encoded by Gene No: 72

When tested against U937 cell lines, supernatants removed from cells containing this gene activated the GAS (gamma activating sequence) promoter element. Thus, it is likely that this gene activates myeloid cells, and to a lesser extent other cells, through the Jak-STAT signal transduction pathway. GAS is a promoter element found upstream of many genes which are involved in the Jak-STAT pathway. The Jak-STAT pathway is a large, signal transduction pathway involved in the differentiation and proliferation of cells. Therefore, activation of the Jak-STAT pathway, reflected by the binding of the GAS element, can be used to indicate proteins involved in the proliferation and differentiation of cells.

This gene is expressed primarily in brain-medulloblastoma tissue.

Therefore, polynucleotides and polypeptides of the invention are useful as reagents for differential identification of the tissue(s) or cell type(s) present in a biological sample and for the diagnosis of diseases and conditions, which include, but are not limited to, neural disorders, particularly proliferative conditions such as brain-medulloblastoma. Similarly, polypeptides and antibodies directed to those polypeptides are useful to provide immunological probes for differential identification of the tissue(s) or cell type(s). For a number of disorders of the above tissues or cells, particularly of the nervous system, expression of this gene at significantly higher or lower levels may be detected in certain tissues or cell types (e.g., neural, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 186 as residues: Asp-18 to His-25, Phe-55 to Tyr-69.

The tissue distribution in brain-medulloblastoma tissue suggests that the protein product of this clone is useful for the diagnosis and/or intervention of brain-medulloblastoma, as well as tumors of other tissues where expression has been observed. Additionally, the peptide may act in nerve tissue development and functions. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:82 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 684 of SEQ ID NO:82, b is an integer of 15 to 698, where both a and b correspond to the positions of nucleotide residues shown in SEQ ID NO:82, and where b is greater than or equal to a +14.

Features of Protein Encoded by Gene No: 73

In specific embodiments, polypeptides of the invention comprise the following amino acid sequence: VAESTEEPAGSNRGQYPEDSSSDGLRQREVLRNL SSPGWENISR (SEQ ID NO:364). Polynucleotides encoding these polypeptides are also encompassed by the invention.

This gene is expressed primarily in chronic lymphocytic leukemia.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic or immune disorders, particularly leukemic diseases. Similarly, polypeptides and antibodies directed to those polypeptides are useful to provide immunological probes for differential identification of the tissue(s) or cell type(s). For a number of disorders of the above tissues or cells, particularly of the hematopoietic system, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in chronic lymphocytic leukemia suggests that the protein product of this clone is useful for the diagnosis and/or intervention of leukemic diseases and hematopoietic disorders. Similarly, expression within hematopoietic cells suggests that the protein product of this clone is useful for the treatment and/or diagnosis of hematopoietic related disorders such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:83 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 854 of SEQ ID NO:83, b is an integer of 15 to 868, where both a and b correspond to the positions of nucleotide residues shown in SEQ ID NO:83, and where b is greater than or equal to a +14.

Features of Protein Encoded by Gene No: 74

In specific embodiments, polypeptides of the invention comprise the following amino acid sequence: AREPLGLTQDPLVFGMTSFLQTSSPIPNSC (SEQ ID NO:365). Polynucleotides encoding these polypeptides are also encompassed by the invention.

The gene encoding the disclosed cDNA is believed to reside on chromosome 11. Accordingly, polynucleotides related to this invention are useful as a marker in linkage analysis for chromosome 11.

This gene is expressed primarily in endothelial cells and in brain tissue.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and neurological disorders. Similarly, polypeptides and antibodies directed to those polypeptides are useful to provide immunological probes for differential identification of the tissue(s) or cell type(s). For a number of disorders of the above tissues or cells, particularly of the vascular and nervous systems, expression of this gene at significantly higher or lower levels may be detected in certain tissues or cell types (e.g., immune, hematopoietic, neural, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 188 as residues: Ser-34 to Ser-39.

The tissue distribution in neural tissue suggests that the protein product of this clone is useful for the detection and/or treatment of neurodegenerative disease states, behavioural disorders, or inflamatory conditions such as Alzheimers Disease, Parkinsons Disease, Huntingtons Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, panic disorder, learning disabilities, ALS, psychoses, autism, and altered behaviors, including disorders in feeding, sleep patterns, balance, and perception. In addition, elevated expression of this gene product in regions of the brain suggests that it plays a role in normal neural function.

Potentially, this gene product is involved in synapse formation, neurotransmission, learning, cognition, homeostasis, or neuronal differentiation or survival. Moreover, the gene or gene product may also play a role in the treatment and/or detection of developmental disorders associated with the developing embryo, sexually-linked disorders, or disorders of the cardiovascular system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:84 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 615 of SEQ ID NO:84, b is an integer of 15 to 629, where both a and b correspond to the positions of nucleotide residues shown in SEQ ID NO:84, and where b is greater than or equal to a +14.

Features of Protein Encoded by Gene No: 75

In specific embodiments, polypeptides of the invention comprise the following amino acid sequences: VLLCHQAGVQWHARLTATSTSRVAAILLPQPPE (SEQ ID NO:367), and/or FQAPASARTACSTLL (SEQ ID NO:366). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, immune and hematopoietic disorders. Similarly, polypeptides and antibodies directed to those polypeptides are useful to provide immunological probes for differential identification of the tissue(s) or cell type(s). For a number of disorders of the above tissues or cells, particularly of the immune and hematopoietic systems, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 189 as residues: Val-24 to Ser-29, Ser-53 to Ala-59, Glu-69 to Met-74.

The tissue distribution of this gene predominantly in neutrophils suggests that the gene could be important for the treatment and/or detection of immune or hematopoietic disorders including arthritis, asthma, immunodeficiency diseases, leukemia, transplant rejection, and microbial infections. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:85 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 823 of SEQ ID NO:85, b is an integer of 15 to 837, where both a and b correspond to the positions of nucleotide residues shown in SEQ ID NO:85, and where b is greater than or equal to a +14.

Features of Protein Encoded by Gene No: 76

In specific embodiments, polypeptides of the invention comprise the following amino acid sequence: AQPSPCPSCLAHSWPPFRLLSLPPPAGASLGDGRVCS (SEQ ID NO:368); and/or HSLPPALPAWLTPGHPSDSSLCLLQLAPHLVMAVSV PWPLPEXLGFSCCHCVSLTGPHAGFSYHFLHPAEPRAWQHQSSVVGMSRKQ ASFSMAQKGVCHLGKSXKRGSKKASCPXYPSFSK (SEQ ID NO:369). Polynucleotides encoding these polypeptides are also encompassed by the invention.

This gene is expressed primarily in endothelial cells.

Therefore, polynucleotides and polypeptides of the invention are useful as reagents for differential identification of the tissue(s) or cell type(s) present in a biological sample and for the diagnosis of diseases and conditions, which include, but are not limited to, vascular disorders. Similarly, polypeptides and antibodies directed to those polypeptides are useful to provide immunological probes for differential identification of the tissue(s).or cell type(s). For a number of disorders of the above tissues or cells, particularly of the hematopoietic and vascular systems, expression of this gene at significantly higher or lower levels may be detected in certain tissues or cell types (e.g., cardiovascular, immune, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution of this gene in endothelial cells suggests that the translation product of this gene is useful in the treatment and/or detection of hematopoietic, immune and/or vascular disorders, particularly atherosclerosis, embolism, stroke, or aneurysm. Furthermore, the tissue distribution in endothelial cells indicates that the protein product of this gene is useful for the diagnosis and treatment of conditions and pathologies of the cardiovascular system, such as heart disease, restenosis, angina, thrombosis, and wound healing. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:86 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 889 of SEQ ID NO:86, b is an integer of 15 to 903, where both a and b correspond to the positions of nucleotide residues shown in SEQ ID NO:86, and where b is greater than or equal to a +14.

Features of Protein Encoded by Gene No: 77

In specific embodiments, polypeptides of the invention comprise the following amino acid sequences: DANPGSRVPEQCSNYYPLLPLIHPMTFFCLTYTG (SEQ ID NO:370); and/or PSFVLPTLGCVWDMHFACCYLILAECIVLAICVYSQFR FCQASTMKEERGKGIEGAYKGVVREMDVKSKLGKLRSKDMI (SEQ ID NO:371). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 191 as residues: Gly-33 to Asn-44.

The tissue distribution in neutrophils suggests that the protein product of this clone is useful for the diagnosis and/or treatment of hematopoietic and immune disorders including: anemias, auto-immunities, immunodeficiencies (e.g. AIDS), immuno-supressive conditions (transplantation) and leukemias. In addition this gene product may be applicable in conditions of general microbial infection, arthritis, inflammation or cancer. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:87 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 711 of SEQ ID NO:87, b is an integer of 15 to 725, where both a and b correspond to the positions of nucleotide residues shown in SEQ ID NO:87, and where b is greater than or equal to a +14.

Features of Protein Encoded by Gene No: 78

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in neutrophils suggests that the protein product of this clone is useful for the diagnosis and/or treatment of hematopoietic and immune disorders including: anemias, auto-immunities, immunodeficiencies (e.g. AIDS), immuno-supressive conditions (transplantation) and leukemias. In addition this gene product may be applicable in conditions of general microbial infection, arthritis, inflammation or cancer. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:88 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 592 of SEQ ID NO:88, b is an integer of 15 to 606, where both a and b correspond to the positions of nucleotide residues shown in SEQ ID NO:88, and where b is greater than or equal to a +14.

Features of Protein Encoded by Gene No: 79

This gene is expressed primarily in hematopoietic cells including neutrophils, T-cells and activated monocytes.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, hematopoietic, and cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution of this gene predominantly in hematopoietic cell types such as neutrophils, T-cells, and activated monocytes suggests that the gene could be important for the treatment and/or detection of immune or hematopoietic disorders including arthritis, asthma, immunodeficiency diseases and leukemia. Morever, this clone would also be useful for the treatment and/or diagnosis of other hematopoietic related disorders such as anemia, pancytopenia, leukopenia, or thrombocytopenia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:89 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 1128 of SEQ ID NO:89, b is an integer of 15 to 1142, where both a and b correspond to the positions of nucleotide residues shown in SEQ ID NO:89, and where b is greater than or equal to a +14.

Features of Protein Encoded by Gene No: 80

In specific embodiments, polypeptides of the invention comprise the following amino acid sequence: QCSGISGSSLICKMRGSEQVISMFLPFLILLSVAYSLYG EFNKLY (SEQ ID NO:373); and/or IGIRVWYYRNQKINSKQMWIKCLGS (SEQ ID NO:372). Polynucleotides encoding these polypeptides are also encompassed by the invention.

The gene encoding the disclosed cDNA is believed to reside on chromosome 1. Accordingly, polynucleotides related to this invention are useful as a marker in linkage analysis for chromosome 1.

This gene is expressed primarily in endothelial cells.

Therefore, polynucleotides and polypeptides of the invention are useful as reagents for differential identification of the tissue(s) or cell type(s) present in a biological sample and for the diagnosis of diseases and conditions, which include, but are not limited to, vascular disorders. Similarly, polypeptides and antibodies directed to those polypeptides are useful to provide immunological probes for differential identification of the tissue(s) or cell type(s). For a number of disorders of the above tissues or cells, particularly of the vascular and hematopoietic systems, expression of this gene at significantly higher or lower levels may be detected in certain tissues or cell types (e.g., vascular, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution within vascular tissue suggests that the protein product of this clone may be useful in the treatment, and/or prevention of a variety of vascular conditions such as atherosclerosis, aneurysm, stroke, or embolism, as well as heart disease, restenosis, angina, thrombosis, and wound healing. As the gene is expressed in endothelial cells, it may also be of importance in the treatment and detection of hematopoietic, and/or immune disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:90 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 582 of SEQ ID NO:90, b is an integer of 15 to 596, where both a and b correspond to the positions of nucleotide residues shown in SEQ ID NO:90, and where b is greater than or equal to a +14.

Features of Protein Encoded by Gene No: 81

The translation product of this gene shares sequence homology with the bile acid CoA:amino acid N-acyltransferase (BAT), which is thought to be important as a liver enzyme that catalyzes the conjugation of bile acids with glycine or taurine (See Genbank Accession No.gnl|PID|e307059).

This gene is expressed primarily in hepatocellular tumor tissue.

Therefore, polynucleotides and polypeptides of the invention are useful as reagents for differential identification of the tissue(s) or cell type(s) present in a biological sample and for the diagnosis of diseases and conditions, which include, but are not limited to, liver diseases and hepatocellular carcinoma. Similarly, polypeptides and antibodies directed to those polypeptides are useful to provide immunological probes for differential identification of the tissue(s) or cell type(s). For a number of disorders of the above tissues or cells, particularly of the hepatocellular carcinoma, expression of this gene at significantly higher or lower levels may be detected in certain tissues or cell types (e.g., hepatic, and cancerous and wounded tissues) or bodily fluids (e.g., lymph, bile, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 195 as residues: Thr-55 to Gln-66, Asp-85 to Glu-92, Pro-125 to Ser-130, Gly-146 to Ala-154, Leu-170 to Lys-177.

The tissue distribution in hepatocellular tumor, and the homology to bile acid CoA:amino acid N-acyltransferase (BAT), suggests that the protein product of this clone is useful for the diagnosis and/or intervention of hepatocellular tumors, particularly as a new molecular prognostic marker in hepatocellular carcinoma patients, following hepatic resection. Furthermore, the translation product of this gene is useful for the detection and/or treatment of cancers of other tissues where expression has been observed. Moreover, the protein product of this clone is also useful for the detection and/or treatment of other liver disorders and cancers (e.g. hepatoblastoma, jaundice, hepatitis, liver metabolic diseases and conditions that are attributable to the differentiation of hepatocyte progenitor cells). The protein may also be useful in developmental abnormalities, fetal deficiencies, pre-natal disorders and various would-healing models and/or tissue trauma. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:91 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 619 of SEQ ID NO:91, b is an integer of 15 to 633, where both a and b correspond to the positions of nucleotide residues shown in SEQ ID NO:91, and where b is greater than or equal to a +14.

Features of Protein Encoded by Gene No: 82

In specific embodiments, polypeptides of the invention comprise the following amino acid sequences: YFMMIKPQFIYSPVDRQLGCFQFFAVTNTPVMGIILSPF YIDTKVSLRYIPRNGISEFLGYGHSQLY (SEQ ID NO:374). Polynucleotides encoding these polypeptides are also encompassed by the invention.

This gene is expressed primarily in bone marrow.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, bone, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution of this gene in bone marrow suggests that the gene could be important for the treatment and/or detection of immune or hematopoietic disorders including arthritis, asthma, immunodeficiency diseases, leukemia, and also in treatement of cancer patients with a depleted immune system. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:92 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 711 of SEQ ID NO:92, b is an integer of 15 to 725, where both a and b correspond to the positions of nucleotide residues shown in SEQ ID NO:92, and where b is greater than or equal to a +14.

Features of Protein Encoded by Gene No: 83

When tested against K562 leukemia cell lines, supernatants removed from cells containing this gene activated the ISRE assay. Thus, it is likely that this gene activates leukemia cells through the Jak-STAT signal transduction pathway. The ISRE (interferon-sensitive responsive element) is a promoter element found upstream in many genes involved in the Jak-STAT pathway. The Jak-STAT pathway is a large, signal transduction pathway involved in the differentiation and proliferation of cells. Therefore, activation of the Jak-STAT pathway, reflected by the binding of the ISRE element, can be used to indicate proteins involved in the proliferation and differentiation of cells.

In specific embodiments, polypeptides of the invention comprise the following amino acid sequence: KGCLTQLLREPVPQIQC (SEQ ID NO:375); and/or FCNLCFTIIREGGRRAGGETIYYFSGILTAWKKRETEKQSREGASHSEFNLSVK (SEQ ID NO:376). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, immunologically mediated disorders. Similarly, polypeptides and antibodies directed to those polypeptides are useful to provide immunological probes for differential identification of the tissue(s) or cell type(s). For a number of disorders of the above tissues or cells, particularly of the immune and hematopoietic systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution suggests that the protein product of this clone is useful for the diagnosis and treatment of hematopoietic and immune disorders including: anemias, auto-immunities, immunodeficiencies (e.g. AIDS), immuno-supressive conditions (transplantation) and leukernias. In addition this gene product may be applicable in conditions of general microbial infection, inflammation or cancer. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:93 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 587 of SEQ ID NO:93, b is an integer of 15 to 601, where both a and b correspond to the positions of nucleotide residues shown in SEQ ID NO:93, and where b is greater than or equal to a +14.

Features of Protein Encoded by Gene No: 84

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, immune and hematopoietic disorders. Similarly, polypeptides and antibodies directed to those polypeptides are useful to provide immunological probes for differential identification of the tissue(s) or cell type(s). For a number of disorders of the above tissues or cells, particularly of the immune and hematopoietic systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 198 as residues: Trp-22 to Trp-35, Ser-42 to Gly-50.

The tissue distribution of this gene predominantly in neutrophils suggests that the gene could be important for the treatment and/or detection of immune or hematopoietic disorders including arthritis, asthma, immunodeficiency diseases, leukemia, transplant rejection, and microbial infections. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:94 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 678 of SEQ ID NO:94, b is an integer of 15 to 692, where both a and b correspond to the positions of nucleotide residues shown in SEQ ID NO:94, and where b is greater than or equal to a +14.

Features of Protein Encoded by Gene No: 85

In specific embodiments, polypeptides of the invention comprise the following amino acid sequences: ARARAVGFPSVCSVGSEHSL (SEQ ID NO:377). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, immunologically mediated disorders. Similarly, polypeptides and antibodies directed to those polypeptides are useful to provide immunological probes for differential identification of the tissue(s) or cell type(s). For a number of disorders of the above tissues or cells, particularly of the immune and hematopoietic systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 199 as residues: Asn-51 to Asn-69.

The tissue distribution in neutrophils suggests that the protein product of this clone is useful for the diagnosis and/or treatment of hematopoietic and immune disorders including: anemias, auto-immunities, immunodeficiencies (e.g. AIDS), immuno-supressive conditions (transplantation) and leukemias. In addition this gene product may be applicable in conditions of general microbial infection, inflammation or cancer. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:95 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 991 of SEQ ID NO:95, b is an integer of 15 to 1005, where both a and b correspond to the positions of nucleotide residues shown in SEQ ID NO:95, and where b is greater than or equal to a +14.

Features of Protein Encoded by Gene No: 86

This gene is expressed primarily in brain medulloblastoma tissue.

Therefore, polynucleotides and polypeptides of the invention are useful as reagents for differential identification of the tissue(s) or cell type(s) present in a biological sample and for the diagnosis of diseases and conditions, which include, but are not limited to, cancer, neurodegenerative diseases and behavioural disorders. Similarly, polypeptides and antibodies directed to those polypeptides are useful to provide immunological probes for differential identification of the tissue(s) or cell type(s). For a number of disorders of the above tissues or cells, particularly of the nervous system, expression of this gene at significantly higher or lower levels may be detected in certain tissues or cell types (e.g., brain, neural, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in brain medulloblastoma tissue suggests that the protein product of this clone is useful for the diagnosis and/or treatment of cancers of the brain, such as medulloblastomas, as well as cancers of other tissues where expression has been observed. Furthermore, the tissue distribution also suggests that the translation product of this clone is useful for the detection and/or treatment of neurodegenerative disease states and behavioural disorders such as Alzheimer's Disease, Parkinson's Disease, Huntington's Disease, schizophrenia, mania, dementia, paranoia, obsessive compulsive disorder and panic disorder. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:96 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 598 of SEQ ID NO:96, b is an integer of 15 to 612, where both a and b correspond to the positions of nucleotide residues shown in SEQ ID NO:96, and where b is greater than or equal to a +14.

Features of Protein Encoded by Gene No: 87

In specific embodiments, polypeptides of the invention comprise the following amino acid sequences: KTKSPYPLHPCFWLMYG (SEQ ID NO:378). Polynucleotides encoding these polypeptides are also encompassed by the invention.

This gene is expressed primarily in brain, bone marrow, and lung tissues, and to a lesser extent in a wide variety of other tissues.

Therefore, polynucleotides and polypeptides of the invention are useful as reagents for differential identification of the tissue(s) or cell type(s) present in a biological sample and for the diagnosis of diseases and conditions, which include, but are not limited to, disorders of the brain and lungs. Similarly, polypeptides and antibodies directed to those polypeptides are useful to provide immunological probes for differential identification of the tissue(s) or cell type(s). For a number of disorders of the above tissues or cells, particularly of the immune, central nervous system, and pulmonary systems expression of this gene at significantly higher or lower levels may be detected in certain tissues or cell types (e.g., brain, lung, immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in bone marrow suggests that the protein product of this clone is useful for the treatment and diagnosis of hematopoietic related disorders such as anemia, pancytopenia, leukopenia, thrombocytopenia or leukemia since stromal cells are important in the production of cells of hematopoietic lineages.

The uses include bone marrow cell ex vivo culture, bone marrow transplantation, bone marrow reconstitution, radiotherapy or chemotherapy of neoplasia. The gene product may also be involved in lymphopoiesis, therefore, it can be used in immune disorders such as infection, inflammation, allergy, immunodeficiency etc. In addition, this gene product may have commercial utility in the expansion of stem cells and committed progenitors of various blood lineages, and in the differentiation and/or proliferation of various cell types. The tissue distribution in brain tissue suggests that the protein product of this clone is useful for the detection/treatment of neurodegenerative disease states and behavioural disorders such as Alzheimer's Disease, Parkinson's Disease, Huntington's Disease, Tourette Syndrome, schizophrenia, mania, dementia, paranoia, obsessive compulsive disorder, panic disorder, learning disabilities, ALS, psychoses, autism, and altered behaviors, including disorders in feeding, sleep patterns, balance, and perception. Alternatively, the tissue distribution in lung tissue suggests that the protein product of this clone is useful for the detection and/or treatment of disorders associated with disorders of the lungs. The tissue distribution suggests that the protein product of this clone is useful for the diagnosis and intervention of lung tumors as well. Protein, as well as, antibodies directed against the protein may show utility as a tumor marker and immunotherapy targets for the above listed tumors and tissues.

Many polynucleotide sequences, such as EST sequences, are publicly available and accessible through sequence databases. Some of these sequences are related to SEQ ID NO:97 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 656 of SEQ ID NO:97, b is an integer of 15 to 670, where both a and b correspond to the positions of nucleotide residues shown in SEQ ID NO:97, and where b is greater than or equal to a +14.

Features of Protein Encoded by Gene No: 88

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, hematopoietic, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in neutrophils suggests that the protein product of this clone is useful for the diagnosis and/or treatment of a variety of immune system disorders. Expression of this gene product in immune cells suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:98 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 605 of SEQ ID NO:98, b is an integer of 15 to 619, where both a and b correspond to the positions of nucleotide residues shown in SEQ ID NO:98, and where b is greater than or equal to a +14.

Features of Protein Encoded by Gene No: 89

In specific embodiments, polypeptides of the invention comprise the following amino acid sequences: PTVYQALGKGHSVREGMVPAGLSSPWACEENARLDL DYCKCQXWPSVGFRGRSELSWNLSFLPQFA (SEQ ID NO:379). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, hematopoietic, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in neutrophils suggests that the protein product of this clone is useful for the diagnosis and/or treatment of a variety of immune system disorders. Expression of this gene product in immune cells suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through-sequence databases. Some of these sequences are related to SEQ ID NO:99 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 689 of SEQ ID NO:99, b is an integer of 15 to 703, where both a and b correspond to the positions of nucleotide residues shown in SEQ ID NO:99, and where b is greater than or equal to a +14.

Features of Protein Encoded by Gene No: 90

In specific embodiments, polypeptides of the invention comprise the following amino acid sequences: LMPCLGSAPARNEGYRLWPITEQILNKHPLGVTLNGA CFSKLLPFLGSEQLSRELVSSAAPEHCAFXDFEKSFLKXPLGSLDQPKSKGFKR ANLIGTAHSPV (SEQ ID NO:380); LMPCLGSAPARNEGYRLWPITEQILNKHP LGVTLNGACFSKLLPFLGSEQLSRELVSSAAPEHCAFX (SEQ ID NO: 381); and/or DFEKSFLKXPLGSLDQPKSKGFKRANLIGTAHSPV (SEQ ID NO:382). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, hematopoietic, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in neutrophils suggests that the protein product of this clone is useful for the diagnosis and/or treatment of a variety of immune system disorders. Expression of this gene product in immune cells suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:100 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 748 of SEQ ID NO:100, b is an integer of 15 to 762, where both a and b correspond to the positions of nucleotide residues shown in SEQ ID NO:100, and where b is greater than or equal to a+14.

Features of Protein Encoded by Gene No: 91

Contact of cells with supernatant containing the expressed product of this gene increases the permeability of the plasma membrane of astrocytes to calcium. Thus, it is likely that the product of this gene is involved in a signal transduction pathway that is initiated when the product binds a receptor on the surface of the astrocytes. Thus, polynucleotides and polypeptides have uses which include, but are not limited to, activating astrocytes.

In specific embodiments, polypeptides of the invention comprise the following amino acid sequences: HEVSCPPQCGSVEGQKQGMGEGRWEGVTAARMRKAA RPAGSPES (SEQ ID NO:383); and/or VTGSRVLPNPPQKSVVKGPGHWGVES ARPDLLGVVSVGAIYPVLXTTPGQLRFVERPSHLLPALXPHRSLVGREN (SEQ ID NO:384). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 205 as residues: Met-1 to Glu-6.

The tissue distribution in neutrophils suggests that the protein product of this clone is useful for the diagnosis and/or treatment of a variety of immune system disorders. Expression of this gene product in immune cells suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:101 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 636 of SEQ ID NO:101, b is an integer of 15 to 650, where both a and b correspond to the positions of nucleotide residues shown in SEQ ID NO:101, and where b is greater than or equal to a+14.

Features of Protein Encoded by Gene No: 92

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 206 as residues: Ile-4 to Cys-9, Ser-36 to Asp-49, Ile-107 to Ile-115.

The tissue distribution in neutrophils suggests that the protein product of this clone is useful for the diagnosis and/or treatment of hematopoietic and immune disorders including: anemias, auto-immunities, immunodeficiencies (e.g. AIDS), immuno-supressive conditions (transplantation) and leukemias. In addition this gene product may be applicable in conditions of general microbial infection, arthritis, inflammation or cancer. Furthermore, expression of this gene product in neutrophils also strongly suggests a role for this protein in immune function and immune surveillance. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:102 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 346 of SEQ ID NO:102, b is an integer of 15 to 360, where both a and b correspond to the positions of nucleotide residues shown in SEQ ID NO:102, and where b is greater than or equal to a+14.

Features of Protein Encoded by Gene No: 93

In specific embodiments, polypeptides of the invention comprise the following amino acid sequences: HELRLRPERKAWGPPDSGPPGPPQVFGQRCPAHGSW GSNGCGFFLSVAWTCHWPRLYFLICDSGDHSSQFTVFGRGD (SEQ ID NO:385). Polynucleotides encoding these polypeptides are also encompassed by the invention.

This gene is expressed primarily in hemangiopericytoma tissue.

Therefore, polynucleotides and polypeptides of the invention are useful as reagents for differential identification of the tissue(s) or cell type(s) present in a biological sample and for the diagnosis of diseases and conditions, which include, but are not limited to, hemangiopericytoma. Similarly, polypeptides and antibodies directed to those polypeptides are useful to provide immunological probes for differential identification of the tissue(s) or cell type(s). For a number of disorders of the above tissues or cells, particularly of the capillaries and arterioles, expression of this gene at significantly higher or lower levels may be detected in certain tissues or cell types (e.g., circulatory, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 207 as residues: Thr-46 to Asp-52.

The tissue distribution in hemangiopericytoma tissue suggests that the protein product of this clone is useful for the diagnosis and/or intervention of hemangiopericytoma or other pericyte related diseases. Protein, as well as, antibodies directed against the protein may show utility as a tissue-specific marker and/or immunotherapy target for the above listed tissues.

Many polynucleotide sequences, such as EST sequences, are publicly available and accessible through sequence databases. Some of these sequences are related to SEQ ID NO:103 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 803 of SEQ ID NO:103, b is an integer of 15 to 817, where both a and b correspond to the positions of nucleotide residues shown in SEQ ID NO:103, and where b is greater than or equal to a +14.

Features of Protein Encoded by Gene No: 94

This gene is expressed primarily in bone marrow.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, bone, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution of this gene in bone marrow suggests that the gene could be important for the treatment and/or detection of immune or hematopoietic disorders including arthritis, asthma, immunodeficiency diseases, leukemia, and also in the treatement of cancer patients with a depleted immune system. The polypeptides or polynucleotides are also useful to enhance or protect proliferation, differentiation, and functional activation of hematopoietic progenitor cells (e.g., bone marrow cells), useful in treating cancer patients undergoing chemotherapy or patients undergoing bone marrow transplantation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:104 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 867 of SEQ ID NO:104, b is an integer of 15 to 881, where both a and b correspond to the positions of nucleotide residues shown in SEQ ID NO:104, and where b is greater than or equal to a+14.

Features of Protein Encoded by Gene No: 95

The gene encoding the disclosed cDNA is thought to reside on chromosome 4. Accordingly, polynucleotides related to this invention are useful as a marker in linkage analysis for chromosome 4.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, immune and hematopoietic disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution of this gene predominantly in neutrophils suggests that the gene could be important for the treatment and/or detection of immune or hematopoietic disorders including arthritis, asthma, immunodeficiency diseases, leukemia, transplant rejection, and microbial infections. Expression of this gene product in immune cells suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene or Protein, as well as, antibodies directed against the protein may show utility as a tumor marker and/or immunotherapy targets for the above listed tissues. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:105 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 641 of SEQ ID NO:105, b is an integer of 15 to 655, where both a and b correspond to the positions of nucleotide residues shown in SEQ ID NO:105, and where b is greater than or equal to a+14.

Features of Protein Encoded by Gene No: 96

In specific embodiments, polypeptides of the invention comprise the following amino acid sequences: KPLFLHSPQISFFSYNLVSLMCSTEVLFFCNNK (SEQ ID NO:386 and SEQ ID NO:387). Polynucleotides encoding these polypeptides are also encompassed by the invention.

This gene is expressed primarily in osteosarcoma tissue.

Therefore, polynucleotides and polypeptides of the invention are useful as reagents for differential identification of the tissue(s) or cell type(s) present in a biological sample and for the diagnosis of diseases and conditions, which include, but are not limited to, osteosarcoma and other cancers. Similarly, polypeptides and antibodies directed to those polypeptides are useful to provide immunological probes for differential identification of the tissue(s) or cell type(s). For a number of disorders of the above tissues or cells, particularly of bone, expression of this gene at significantly higher or lower levels may be detected in certain tissues or cell types (e.g., bone, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in osteosarcoma tissue suggests that the protein product of this clone is useful for the detection and/or treatment of: fractures and traumas, osteoporosis, osteosarcoma, osteoclastoma, chondrosarcoma, regulation of ossification and osteonecrosis, arthritis, tendonitis, chrondomalacia and inflammation. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:106 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 592 of SEQ ID NO:106, b is an integer of 15 to 606, where both a and b correspond to the positions of nucleotide residues shown in SEQ ID NO:106, and where b is greater than or equal to a+14.

Features of Protein Encoded by Gene No: 97

In specific embodiments, polypeptides of the invention comprise the following amino acid sequences: LHFSHTFLSTKNHESLNYSSSHRIESKYQRSHPFKTQFF HCSIRYVLYVR (SEQ ID NO:388). Polynucleotides encoding these polypeptides are also encompassed by the invention.

This gene is expressed primarily in salivary gland and osteosarcoma tissue.

Therefore, polynucleotides and polypeptides of the invention are useful as reagents for differential identification of the tissue(s) or cell type(s) present in a biological sample and for the diagnosis of diseases and conditions, which include, but are not limited to, osteosarcoma and other cancers, as well as digestive disorders. Similarly, polypeptides and antibodies directed to those polypeptides are useful to provide immunological probes for differential identification of the tissue(s) or cell type(s). For a number of disorders of the above tissues or cells, particularly of bone and the digestive system, expression of this gene at significantly higher or lower levels may be detected in certain tissues or cell types (e.g., bone, digestive, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in osteosarcoma tissue suggests that the protein product of this clone is useful for the detection and/or treatment of bone-related disorders and conditions, such as fractures and traumas, osteoperosis, osteosarcoma, osteoclastoma, chondrosarcoma, regulation of ossification and osteonecrosis, arthritis, tendonitis, chrondomalacia and inflammation. In addition, the expression in salivary glands suggest a possible role for this gene product in the detection and/or treatment of digestive system disorders. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:107 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 643 of SEQ ID NO:107, b is an integer of 15 to 657, where both a and b correspond to the positions of nucleotide residues shown in SEQ ID NO:107, and where b is greater than or equal to a +14.

Features of Protein Encoded by Gene No: 98

In specific embodiments, polypeptides of the invention comprise the following amino acid sequences: ERILCRKSKFFWTLPAY (SEQ ID NO:389). Polynucleotides encoding these polypeptides are also encompassed by the invention.

This gene is expressed primarily in neutrophils.

Therefore, polynucleotides and polypeptides of the invention are useful as reagents for differential identification of the tissue(s) or cell type(s) present in a biological sample and for the diagnosis of diseases and conditions, which include, but are not limited to, hematopoietic and immune disorders. Similarly, polypeptides and antibodies directed to those polypeptides are useful to provide immunological probes for differential identification of the tissue(s) or cell type(s). For a number of disorders of the above tissues or cells, particularly of the hematopoietic and immune systems, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

Preferred epitopes include those comprising a sequence shown in SEQ ID NO. 212 as residues: Trp-32 to Pro-40.

The tissue distribution in neutrophils suggests that the protein product of this clone is useful for the diagnosis and/or treatment of hematopoietic and immune disorders including: anemias, auto-immunities, immunodeficiencies (e.g. AIDS), immuno-supressive conditions (transplantation) and leukemias. In addition this gene product may be applicable in conditions of general microbial infection, arthritis, inflammation or cancer. Furthermore, expression of this gene product in neutrophils also strongly suggests a role for this protein in immune function and immune surveillance. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:108 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 591 of SEQ ID NO:108, b is an integer of 15 to 605, where both a and b correspond to the positions of nucleotide residues shown in SEQ ID NO:108, and where b is greater than or equal to a+14.

Features of Protein Encoded by Gene No: 99

This gene is expressed primarily in breast lymph node tissue.

Therefore, polynucleotides and polypeptides of the invention are useful as reagents for differential identification of the tissue(s) or cell type(s) present in a biological sample and for the diagnosis of diseases and conditions, which include, but are not limited to, breast cancer and other immune diseases. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in breast lymph node tissue suggests that the protein product of this clone is useful for the diagnosis and/or intervention of breast cancers and other immune diseases, as well as cancers of other tissues where expression of this gene has been observed. Expression of this gene product in lymph nodes suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:109 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 490 of SEQ ID NO:109, b is an integer of 15 to 504, where both a and b correspond to the positions of nucleotide residues shown in SEQ ID NO:109, and where b is greater than or equal to a +14.

Features of Protein Encoded by Gene No:100

This gene is expressed primarily in T-cell lymphoma and Hodgkin's lymphoma tissues, and to a lesser extent in human thymus tissue.

Therefore, polynucleotides and polypeptides of the invention are useful as reagents for differential identification of the tissue(s) or cell type(s) present in a biological sample and for the diagnosis of diseases and conditions, which include, but are not limited to, T-cell lymphomas and immune diseases and disorders. Similarly, polypeptides and antibodies directed to those polypeptides are useful to provide immunological probes for differential identification of the tissue(s) or cell type(s). For a number of disorders of the above tissues or cells, particularly of the immune system, expression of this gene at significantly higher or lower levels may be detected in certain tissues or cell types (e.g., immune, thymus, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in T-cell lymphoma suggests that the protein product of this clone is useful for the diagnosis and/or intervention of T-cell lymphomas and other immune diseases. Expression of this gene product in the thymus, as well as in T-cell lymphomas, suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:110 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 756 of SEQ ID NO:110, b is an integer of 15 to 770, where both a and b correspond to the positions of nucleotide residues shown in SEQ ID NO:110, and where b is greater than or equal to a+14.

Features of Protein Encoded by Gene No: 101

In specific embodiments, polypeptides of the invention comprise the following amino acid sequences: GFQTILKRLDVTCNVIEQFDDPGYYGSMKSPWFLELA CFYSGKNFLAPQLTA (SEQ ID NO:390). Polynucleotides encoding these polypeptides are also encompassed by the invention.

This gene is expressed primarily in spleen chronic lymphocytic leukemia tissue.

Therefore, polynucleotides and polypeptides of the invention are useful as reagents for differential identification of the tissue(s) or cell type(s) present in a biological sample and for the diagnosis of diseases and conditions, which include, but are not limited to, chronic lymphocytic leukemia. Similarly, polypeptides and antibodies directed to those polypeptides are useful to provide immunological probes for differential identification of the tissue(s) or cell type(s). For a number of disorders of the above tissues or cells, particularly of the hematopoietic system expression of this gene at significantly higher or lower levels may be detected in certain tissues or cell types (e.g., immune, cancerous and wounded tissues) or bodily fluids (e.g., lymph, serum, plasma, urine, synovial fluid or spinal fluid) taken from an individual having such a disorder, relative to the standard gene expression level, i.e., the expression level in healthy tissue from an individual not having the disorder.

The tissue distribution in spleen chronic lymphocytic leukemia tissue suggests that the protein product of this clone is useful for the diagnosis and/or intervention of leukemia diseases or hematopoietic disoders. Expression of this gene product in spleen suggests a role in the regulation of the proliferation; survival; differentiation; and/or activation of potentially all hematopoietic cell lineages, including blood stem cells. This gene product may be involved in the regulation of cytokine production, antigen presentation, or other processes that may also suggest a usefulness in the treatment of cancer (e.g. by boosting immune responses).

Since the gene is expressed in cells of lymphoid origin, the natural gene product may be involved in immune functions. Therefore it may be also used as an agent for immunological disorders including arthritis, asthma, immune deficiency diseases such as AIDS, leukemia, rheumatoid arthritis, inflammatory bowel disease, sepsis, acne, and psoriasis. In addition, this gene product may have commercial utility in the expansion of stem cells and committed progenitors of various blood lineages, and in the differentiation and/or proliferation of various cell types. Protein, as well as, antibodies directed against the protein may show utility as a tumor marker and/or immunotherapy targets for the above listed tissues.

Many polynucleotide sequences, such as EST sequences, are publicly available and accessible through sequence databases. Some of these sequences are related to SEQ ID NO:11 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a-b, where a is any integer between 1 to 737 of SEQ ID NO:111, b is an integer of 15 to 751, where both a and b correspond to the positions of nucleotide residues shown in SEQ ID NO:111, and where b is greater than or equal to a +14.

| | | | | | | | | | 5′ NT | | | | | |
|---|---|---|---|---|---|---|---|---|---|---|---|---|---|---|
| | | | | | | | | | of | | First | Last | | |
| | | ATCC | | NT | | 5′ NT | 3′ NT | 5′ NT | First | AA | AA | AA | First | Last |
| | | Deposit | | SEQ | Total | of | of | of | AA of | SEQ | of | of | AA of | AA |
| Gene | cDNA | Nr and | | ID | NT | Clone | Clone | Start | Signal | ID | Sig | Sig | Secreted | of |
| No. | Clone ID | Date | Vector | NO: X | Seq. | Seq. | Seq. | Codon | Pep | NO: Y | Pep | Pep | Portion | ORF |
| 1 | HCWCH14 | 209225 | ZAP Express | 11 | 552 | 65 | 552 | 183 | 183 | 115 | 1 | 29 | 30 | 86 |
| | | Aug. 28, 1997 | | | | | | | | | | | | |
| 1 | HCWCH14 | 209225 | ZAP Express | 112 | 543 | 1 | 543 | | 177 | 216 | 1 | 28 | 29 | 85 |
| | | Aug. 28, 1997 | | | | | | | | | | | | |
| 2 | HE2EB74 | 209225 | Uni-ZAP XR | 12 | 1434 | 311 | 1418 | 507 | 507 | 116 | 1 | 16 | 17 | 19 |
| | | Aug. 28, 1997 | | | | | | | | | | | | |
| 3 | HFGAD82 | 209225 | Uni-ZAP XR | 13 | 1881 | 772 | 1861 | 1019 | 1019 | 117 | 1 | 18 | 19 | 38 |
| | | Aug. 28, 1997 | | | | | | | | | | | | |
| 4 | HE9MI43 | 209225 | Uni-ZAP XR | 14 | 1060 | 1 | 1060 | 171 | 171 | 118 | 1 | 19 | 20 | 87 |
| | | Aug. 28, 1997 | | | | | | | | | | | | |
| 5 | HE9NH44 | 209225 | Uni-ZAP XR | 15 | 1255 | 37 | 1255 | 113 | 113 | 119 | 1 | 18 | 19 | 38 |
| | | Aug. 28, 1997 | | | | | | | | | | | | |
| 6 | HFKCK85 | 209225 | Uni-ZAP XR | 16 | 1036 | 1 | 1036 | 159 | 159 | 120 | 1 | 24 | 25 | 26 |
| | | Aug. 28, 1997 | | | | | | | | | | | | |
| 7 | HHFCY66 | 209225 | Uni-ZAP XR | 17 | 1014 | 1 | 1014 | 49 | 49 | 121 | 1 | 19 | 20 | 21 |
| | | Aug. 28, 1997 | | | | | | | | | | | | |
| 8 | HE2PI29 | 209225 | Uni-ZAP XR | 18 | 1287 | 1 | 1287 | 174 | 174 | 122 | 1 | 37 | 38 | 95 |
| | | Aug. 28, 1997 | | | | | | | | | | | | |
| 9 | HE9AN21 | 209225 | Uni-ZAP XR | 19 | 1105 | 1 | 1105 | 327 | 327 | 123 | 1 | 22 | 23 | 35 |
| | | Aug. 28, 1997 | | | | | | | | | | | | |
| 10 | HEPCE37 | 209225 | Uni-ZAP XR | 20 | 1089 | 1 | 1089 | 297 | 297 | 124 | 1 | 19 | 20 | 37 |
| | | Aug. 28, 1997 | | | | | | | | | | | | |
| 11 | HLHDP83 | 209226 | Uni-ZAP XR | 21 | 2831 | 395 | 1598 | 426 | 426 | 125 | 1 | 36 | 37 | 341 |
| | | Aug. 28, 1997 | | | | | | | | | | | | |
| 12 | HSIAS17 | 209226 | Uni-ZAP XR | 22 | 1448 | 1 | 1224 | 108 | 108 | 126 | 1 | 23 | 24 | 218 |
| | | Aug. 28, 1997 | | | | | | | | | | | | |
| 13 | HSIEF95 | 209226 | Uni-ZAP XR | 23 | 1211 | 136 | 1211 | 177 | 177 | 127 | 1 | 25 | 26 | 265 |
| | | Aug. 28, 1997 | | | | | | | | | | | | |
| 14 | HSDDC95 | 209226 | Uni-ZAP XR | 24 | 1060 | 1 | 1060 | 67 | 67 | 128 | 1 | 37 | 38 | 38 |
| | | Aug. 28, 1997 | | | | | | | | | | | | |
| 15 | HOSDG32 | 209226 | Uni-ZAP XR | 25 | 1057 | 1 | 1057 | 175 | 175 | 129 | 1 | 21 | 22 | 92 |
| | | Aug. 28, 1997 | | | | | | | | | | | | |
| 16 | HMUBU59 | 209226 | pCMVSport | 26 | 980 | 1 | 980 | 30 | 30 | 130 | 1 | 41 | 42 | 195 |
| | | Aug. 28, 1997 | 3.0 | | | | | | | | | | | |
| 17 | HWTCE21 | 209226 | Uni-ZAP XR | 27 | 755 | 1 | 744 | 339 | 339 | 131 | 1 | 16 | 17 | 49 |
| | | Aug. 28, 1997 | | | | | | | | | | | | |
| 18 | HFIUM15 | 209226 | pSport1 | 28 | 946 | 1 | 946 | 168 | 168 | 132 | 1 | 32 | 33 | 54 |
| | | Aug. 28, 1997 | | | | | | | | | | | | |
| 19 | HLYAN43 | 209226 | pSport1 | 29 | 971 | 26 | 946 | 135 | 135 | 133 | 1 | 23 | 24 | 32 |
| | | Aug. 28, 1997 | | | | | | | | | | | | |
| 20 | HBJFA56 | 209235 | Uni-ZAP XR | 30 | 1008 | 1 | 993 | 155 | 155 | 134 | 1 | 16 | 17 | 23 |
| | | Sep. 04, 1997 | | | | | | | | | | | | |
| 21 | HTLAF13 | 209235 | Uni-ZAP XR | 31 | 990 | 80 | 990 | 164 | 164 | 135 | 1 | 26 | 27 | 219 |
| | | Sep. 04, 1997 | | | | | | | | | | | | |
| 22 | HTLFI93 | 209235 | Uni-ZAP XR | 32 | 1131 | 1 | 1107 | 48 | 48 | 136 | 1 | 43 | 44 | 302 |
| | | Sep. 04, 1997 | | | | | | | | | | | | |
| 23 | HBXGI20 | 209235 | ZAP Express | 33 | 1293 | 1 | 1002 | 199 | 199 | 137 | 1 | 37 | 38 | 40 |
| | | Sep. 04, 1997 | | | | | | | | | | | | |
| 24 | HTPBH21 | 209235 | Uni-ZAP XR | 34 | 1014 | 1 | 1014 | 21 | 21 | 138 | 1 | 25 | 26 | 277 |
| | | Sep. 04, 1997 | | | | | | | | | | | | |
| 25 | HSQAB87 | 209235 | Uni-ZAP XR | 35 | 1222 | 375 | 1222 | 473 | 473 | 139 | 1 | 19 | 20 | 110 |
| | | Sep. 04, 1997 | | | | | | | | | | | | |
| 26 | HTEDJ94 | 209235 | Uni-ZAP XR | 36 | 901 | 1 | 901 | 240 | 240 | 140 | 1 | 46 | 47 | 132 |
| | | Sep. 04, 1997 | | | | | | | | | | | | |
| 27 | HKMLM11 | 209236 | pBluescript | 37 | 954 | 1 | 954 | 82 | 82 | 141 | 1 | 20 | 21 | 130 |
| | | Sep. 04, 1997 | | | | | | | | | | | | |
| 28 | HNEAC05 | 209236 | Uni-ZAP XR | 38 | 890 | 1 | 890 | 101 | 101 | 142 | 1 | 24 | 25 | 105 |
| | | Sep. 04, 1997 | | | | | | | | | | | | |
| 29 | HETEW02 | 209236 | Uni-ZAP XR | 39 | 1070 | 1 | 905 | 98 | 98 | 143 | 1 | 19 | 20 | 61 |
| | | Sep. 04, 1997 | | | | | | | | | | | | |
| 30 | HE8MG70 | 209236 | Uni-ZAP XR | 40 | 772 | 1 | 772 | 85 | 85 | 144 | 1 | 27 | 28 | 37 |
| | | Sep. 04, 1997 | | | | | | | | | | | | |
| 31 | HLMCA59 | 209236 | Uni-ZAP XR | 41 | 787 | 1 | 787 | 101 | 101 | 145 | 1 | 31 | 32 | 63 |
| | | Sep. 04, 1997 | | | | | | | | | | | | |
| 32 | HOAAC90 | 209236 | Uni-ZAP XR | 42 | 652 | 1 | 652 | 38 | 38 | 146 | 1 | 15 | 16 | 104 |
| | | Sep. 04, 1997 | | | | | | | | | | | | |
| 33 | HMEJQ68 | 209236 | Lambda ZAP II | 43 | 1520 | 1 | 1520 | 89 | 89 | 147 | 1 | 37 | 38 | 60 |
| | | Sep. 04, 1997 | | | | | | | | | | | | |
| 34 | HNGIJ31 | 209236 | Uni-ZAP XR | 44 | 796 | 1 | 796 | 135 | 135 | 148 | 1 | 16 | 17 | 36 |
| | | Sep. 04, 1997 | | | | | | | | | | | | |
| 35 | HFXJZ18 | 209236 | Lambda ZAP II | 45 | 1378 | 436 | 1378 | 692 | 692 | 149 | 1 | 27 | 28 | 31 |
| | | Sep. 04, 1997 | | | | | | | | | | | | |
| 36 | HPEBE79 | 209241 | Uni-ZAP XR | 46 | 597 | 1 | 597 | 79 | 79 | 150 | 1 | 13 | 14 | 15 |
| | | Sep. 12, 1997 | | | | | | | | | | | | |
| 37 | HRTAE58 | 209241 | pBluescript | 47 | 600 | 1 | 600 | 244 | 244 | 151 | 1 | 18 | 19 | 58 |
| | | Sep. 12, 1997 | SK- | | | | | | | | | | | |
| 38 | HSKNB54 | 209241 | pBluescript | 48 | 911 | 1 | 911 | 180 | 180 | 152 | 1 | 21 | 22 | 86 |
| | | Sep. 12, 1997 | | | | | | | | | | | | |
| 39 | HSKNT34 | 209241 | pBluescript | 49 | 1863 | 1 | 1094 | 21 | 21 | 153 | 1 | 22 | 23 | 52 |
| | | Sep. 12, 1997 | | | | | | | | | | | | |
| 40 | HTEDY42 | 209241 | Uni-ZAP XR | 50 | 810 | 1 | 810 | 19 | 19 | 154 | 1 | 23 | 24 | 77 |
| | | Sep. 12, 1997 | | | | | | | | | | | | |
| 41 | HTLAA40 | 209241 | Uni-ZAP XR | 51 | 956 | 1 | 956 | 33 | 33 | 155 | 1 | 28 | 29 | 71 |
| | | Sep. 12, 1997 | | | | | | | | | | | | |
| 42 | HTNBO91 | 209241 | pBluescript | 52 | 300 | 1 | 300 | 7 | 7 | 156 | 1 | 26 | 27 | 40 |
| | | Sep. 12, 1997 | SK- | | | | | | | | | | | |
| 43 | H6BSD90 | 209241 | Uni-ZAP XR | 53 | 841 | 1 | 841 | 188 | 188 | 157 | 1 | 23 | 24 | 84 |
| | | Sep. 12, 1997 | | | | | | | | | | | | |
| 44 | HBJBQ35 | 209241 | Uni-ZAP XR | 54 | 634 | 1 | 634 | 84 | 84 | 158 | 1 | 20 | 21 | 95 |
| | | Sep. 12, 1997 | | | | | | | | | | | | |
| 45 | HCE1Q89 | 209242 | Uni-ZAP XR | 55 | 863 | 1 | 863 | 74 | 74 | 159 | 1 | 17 | 18 | 88 |
| | | Sep. 12, 1997 | | | | | | | | | | | | |
| 46 | HCNSB61 | 209242 | pBluescript | 56 | 712 | 1 | 712 | 218 | 218 | 160 | 1 | 21 | 22 | 43 |
| | | Sep. 12, 1997 | | | | | | | | | | | | |
| 47 | HCDBO20 | 209242 | Uni-ZAP XR | 57 | 925 | 1 | 925 | 8 | 8 | 161 | 1 | 42 | 43 | 45 |
| | | Sep. 12, 1997 | | | | | | | | | | | | |
| 48 | HBNAW17 | 209242 | Uni-ZAP XR | 58 | 601 | 1 | 601 | 77 | 77 | 162 | 1 | 37 | 38 | 61 |
| | | Sep. 12, 1997 | | | | | | | | | | | | |
| 49 | HCDBW86 | 209242 | Uni-ZAP XR | 59 | 730 | 1 | 730 | 139 | 139 | 163 | 1 | 20 | 21 | 30 |
| | | Sep. 12, 1997 | | | | | | | | | | | | |
| 50 | HE6CL49 | 209242 | Uni-ZAP XR | 60 | 845 | 1 | 845 | 120 | 120 | 164 | 1 | 53 | 54 | 94 |
| | | Sep. 12, 1997 | | | | | | | | | | | | |
| 50 | HE6CL49 | 209242 | Uni-ZAP XR | 113 | 846 | 1 | 846 | 187 | 187 | 217 | 1 | | | 24 |
| | | Sep. 12, 1997 | | | | | | | | | | | | |
| 51 | HEAAH81 | 209242 | Uni-ZAP XR | 61 | 958 | 1 | 958 | 224 | 224 | 165 | 1 | 23 | 24 | 70 |
| | | Sep. 12, 1997 | | | | | | | | | | | | |
| 52 | HEBAE88 | 209242 | Uni-ZAP XR | 62 | 582 | 1 | 582 | 160 | 160 | 166 | 1 | 26 | 27 | 42 |
| | | Sep. 12, 1997 | | | | | | | | | | | | |
| 53 | HFXGV31 | 209242 | Lambda ZAP | 63 | 752 | 1 | 752 | 100 | 100 | 167 | 1 | 24 | 25 | 64 |
| | | Sep. 12, 1997 | II | | | | | | | | | | | |
| 54 | HEAAJ57 | 209242 | Uni-ZAP XR | 64 | 706 | 1 | 706 | 162 | 162 | 168 | 1 | 20 | 21 | 67 |
| | | Sep. 12, 1997 | | | | | | | | | | | | |
| 55 | HCFMV71 | 209242 | pSport1 | 65 | 400 | 1 | 400 | 31 | 31 | 169 | 1 | 24 | 25 | 58 |
| | | Sep. 12, 1997 | | | | | | | | | | | | |
| 56 | HERAM05 | 209242 | Uni-ZAP XR | 66 | 773 | 1 | 773 | 240 | 240 | 170 | 1 | 14 | 15 | 53 |
| | | Sep. 12, 1997 | | | | | | | | | | | | |
| 57 | HFKFY69 | 209242 | Uni-ZAP XR | 67 | 647 | 1 | 647 | 157 | 157 | 171 | 1 | | | 19 |
| | | Sep. 12, 1997 | | | | | | | | | | | | |
| 58 | HFTCR15 | 209242 | Uni-ZAP XR | 68 | 675 | 1 | 675 | 82 | 82 | 172 | 1 | 28 | 29 | 38 |
| | | Sep. 12, 1997 | | | | | | | | | | | | |
| 59 | HGBDL30 | 209242 | Uni-ZAP XR | 69 | 889 | 1 | 889 | 68 | 68 | 173 | 1 | 41 | 42 | 53 |
| | | Sep. 12, 1997 | | | | | | | | | | | | |
| 60 | HFKEN81 | 209242 | Uni-ZAP XR | 70 | 888 | 1 | 888 | 25 | 25 | 174 | 1 | 23 | 24 | 54 |
| | | Sep. 12, 1997 | | | | | | | | | | | | |
| 61 | HFPCX36 | 209242 | Uni-ZAP XR | 71 | 796 | 1 | 796 | 103 | 103 | 175 | 1 | 27 | 28 | 46 |
| | | Sep. 12, 1997 | | | | | | | | | | | | |
| 62 | HFRAN90 | 209242 | Uni-ZAP XR | 72 | 532 | 1 | 532 | 178 | 178 | 176 | 1 | 39 | 40 | 54 |
| | | Sep. 12, 1997 | | | | | | | | | | | | |
| 63 | HHGBO65 | 209242 | Lambda ZAP | 73 | 546 | 1 | 546 | 279 | 279 | 177 | 1 | 25 | 26 | 26 |
| | | Sep. 12, 1997 | II | | | | | | | | | | | |
| 64 | HHGBO91 | 209242 | Lambda ZAP | 74 | 715 | 1 | 715 | 140 | 140 | 178 | 1 | 28 | 29 | 49 |
| | | Sep. 12, 1997 | II | | | | | | | | | | | |
| 65 | HGLAL82 | 209242 | Uni-ZAP XR | 75 | 406 | 1 | 406 | 144 | 144 | 179 | 1 | 19 | 20 | 26 |
| | | Sep. 12, 1997 | | | | | | | | | | | | |
| 66 | HERAN54 | 209242 | Uni-ZAP XR | 76 | 542 | 1 | 542 | 99 | 99 | 180 | 1 | 28 | 29 | 40 |
| | | Sep. 12, 1997 | | | | | | | | | | | | |
| 67 | HFXDE67 | 209242 | Lambda ZAP | 77 | 420 | 1 | 420 | 224 | 224 | 181 | 1 | 27 | 28 | 65 |
| | | Sep. 12, 1997 | II | | | | | | | | | | | |
| 68 | HFRAC19 | 209242 | Uni-ZAP XR | 78 | 465 | 1 | 465 | 146 | 146 | 182 | 1 | 17 | 18 | 19 |
| | | Sep. 12, 1997 | | | | | | | | | | | | |
| 69 | HGLAJ51 | 209242 | Uni-ZAP XR | 79 | 889 | 1 | 889 | 218 | 218 | 183 | 1 | 33 | 34 | 42 |
| | | Sep. 12, 1997 | | | | | | | | | | | | |
| 69 | HGLAJ51 | 209242 | Uni-ZAP XR | 114 | 890 | 1 | 890 | 212 | 212 | 218 | 1 | | | 14 |
| | | Sep. 12, 1997 | | | | | | | | | | | | |
| 70 | HFFAD59 | 209242 | Lambda ZAP | 80 | 470 | 1 | 470 | 44 | 44 | 184 | 1 | 17 | 18 | 45 |
| | | Sep. 12, 1997 | II | | | | | | | | | | | |
| 71 | HESAJ10 | 209242 | Uni-ZAP XR | 81 | 1090 | 400 | 1090 | 405 | 405 | 185 | 1 | 23 | 24 | 71 |
| | | Sep. 12, 1997 | | | | | | | | | | | | |
| 72 | HMDAE65 | 209243 | Uni-ZAP XR | 82 | 698 | 1 | 698 | 179 | 179 | 186 | 1 | 17 | 18 | 77 |
| | | Sep. 12, 1997 | | | | | | | | | | | | |
| 73 | HLYBV47 | 209243 | pSport1 | 83 | 868 | 1 | 868 | 324 | 324 | 187 | 1 | 21 | 22 | 25 |
| | | Sep. 12, 1997 | | | | | | | | | | | | |
| 74 | HMEGF92 | 209243 | Lambda ZAP | 84 | 629 | 1 | 611 | 92 | 92 | 188 | 1 | 27 | 28 | 62 |
| | | Sep. 12, 1997 | II | | | | | | | | | | | |
| 75 | HNGIK36 | 209243 | Uni-ZAP XR | 85 | 837 | 1 | 837 | 48 | 48 | 189 | 1 | 41 | 42 | 91 |
| | | Sep. 12, 1997 | | | | | | | | | | | | |
| 76 | HMEJJ27 | 209243 | Lambda ZAP | 86 | 903 | 1 | 903 | 113 | 113 | 190 | 1 | 34 | 35 | 47 |
| | | Sep. 12, 1997 | II | | | | | | | | | | | |
| 77 | HNHCY64 | 209243 | Uni-ZAP XR | 87 | 725 | 1 | 725 | 258 | 258 | 191 | 1 | 34 | 35 | 44 |
| | | Sep. 12, 1997 | | | | | | | | | | | | |
| 78 | HNHCY94 | 209243 | Uni-ZAP XR | 88 | 606 | 1 | 606 | 78 | 78 | 192 | 1 | 25 | 26 | 48 |
| | | Sep. 12, 1997 | | | | | | | | | | | | |
| 79 | HNEBN76 | 209243 | Uni-ZAP XR | 89 | 1142 | 150 | 1142 | 346 | 346 | 193 | 1 | 24 | 25 | 81 |
| | | Sep. 12, 1997 | | | | | | | | | | | | |
| 80 | HMEFT54 | 209243 | Lambda ZAP | 90 | 596 | 1 | 596 | 332 | 332 | 194 | 1 | 24 | 25 | 39 |
| | | Sep. 12, 1997 | II | | | | | | | | | | | |
| 81 | HLQBE09 | 209243 | Lambda ZAP | 91 | 633 | 1 | 633 | 17 | 17 | 195 | 1 | 19 | 20 | 181 |
| | | Sep. 12, 1997 | II | | | | | | | | | | | |
| 82 | HMWBC11 | 209243 | Uni-Zap XR | 92 | 725 | 1 | 725 | 139 | 139 | 196 | 1 | 28 | 29 | 39 |
| | | Sep. 12, 1997 | | | | | | | | | | | | |
| 83 | HNGJR78 | 209243 | Uni-ZAP XR | 93 | 601 | 1 | 601 | 159 | 159 | 197 | 1 | 24 | 25 | 72 |
| | | Sep. 12, 1997 | | | | | | | | | | | | |
| 84 | HNGDP26 | 209243 | Uni-ZAP XR | 94 | 692 | 1 | 692 | 77 | 77 | 198 | 1 | 21 | 22 | 55 |
| | | Sep. 12, 1997 | | | | | | | | | | | | |
| 85 | HNGJH63 | 209243 | Uni-ZAP XR | 95 | 1005 | 1 | 1005 | 62 | 62 | 199 | 1 | 31 | 32 | 69 |
| | | Sep. 12, 1997 | | | | | | | | | | | | |
| 86 | HMDAL04 | 209243 | Uni-ZAP XR | 96 | 612 | 1 | 612 | 48 | 48 | 200 | 1 | 21 | 22 | 46 |
| | | Sep. 12, 1997 | | | | | | | | | | | | |
| 87 | HMWHX28 | 209243 | Uni-Zap XR | 97 | 670 | 1 | 670 | 128 | 128 | 201 | 1 | 21 | 22 | 54 |
| | | Sep. 12, 1997 | | | | | | | | | | | | |
| 88 | HNHAD65 | 209243 | Uni-ZAP XR | 98 | 619 | 1 | 619 | 27 | 27 | 202 | 1 | 22 | 23 | 37 |
| | | Sep. 12, 1997 | | | | | | | | | | | | |
| 89 | HNGAP93 | 209243 | Uni-ZAP XR | 99 | 703 | 1 | 703 | 50 | 50 | 203 | 1 | 20 | 21 | 33 |
| | | Sep. 12, 1997 | | | | | | | | | | | | |
| 90 | HNHCX60 | 209243 | Uni-ZAP XR | 100 | 762 | 1 | 762 | 158 | 158 | 204 | 1 | 21 | 22 | 21 |
| | | Sep. 12, 1997 | | | | | | | | | | | | |
| 91 | HNHGB09 | 209243 | Uni-ZAP XR | 101 | 650 | 1 | 650 | 135 | 135 | 205 | 1 | 26 | 27 | 55 |
| | | Sep. 12, 1997 | | | | | | | | | | | | |
| 92 | HNHHA15 | 209243 | Uni-ZAP XR | 102 | 360 | 1 | 360 | 11 | 11 | 206 | 1 | 32 | 33 | 116 |
| | | Sep. 12, 1997 | | | | | | | | | | | | |
| 93 | HHGDC01 | 209243 | Lambda ZAP | 103 | 817 | 1 | 817 | 234 | 234 | 207 | 1 | 45 | 46 | 83 |
| | | Sep. 12, 1997 | II | | | | | | | | | | | |
| 94 | HMWGU74 | 209243 | Uni-Zap XR | 104 | 881 | 1 | 881 | 147 | 147 | 208 | 1 | 25 | 26 | 45 |
| | | Sep. 12, 1997 | | | | | | | | | | | | |
| 95 | HNGCF72 | 209243 | Uni-ZAP XR | 105 | 655 | 1 | 655 | 154 | 154 | 209 | 1 | 20 | 21 | 41 |
| | | Sep. 12, 1997 | | | | | | | | | | | | |
| 96 | HOACB38 | 209243 | Uni-ZAP XR | 106 | 606 | 1 | 606 | 63 | 63 | 210 | 1 | 21 | 22 | 40 |
| | | Sep. 12, 1997 | | | | | | | | | | | | |
| 97 | HOACG37 | 209243 | Uni-ZAP XR | 107 | 657 | 1 | 622 | 219 | 219 | 211 | 1 | 24 | 25 | 30 |
| | | Sep. 12, 1997 | | | | | | | | | | | | |
| 98 | HNHBL26 | 209243 | Uni-ZAP XR | 108 | 605 | 1 | 605 | 465 | 465 | 212 | 1 | 24 | 25 | 40 |
| | | Sep. 12, 1997 | | | | | | | | | | | | |
| 99 | HLMFD11 | 209243 | Lambda ZAP | 109 | 504 | 1 | 504 | 40 | 40 | 213 | 1 | 31 | 32 | 99 |
| | | Sep. 12, 1997 | II | | | | | | | | | | | |
| 100 | HLTDV50 | 209243 | Uni-ZAP XR | 110 | 770 | 1 | 770 | 74 | 74 | 214 | 1 | 17 | 18 | 28 |
| | | Sep. 12, 1997 | | | | | | | | | | | | |
| 101 | HLYBA22 | 209243 | pSport1 | 111 | 751 | 1 | 751 | 153 | 153 | 215 | 1 | 31 | 32 | 46 |
| | | Sep. 12, 1997 | | | | | | | | | | | | |

Table 1 summarizes the information corresponding to each “Gene No.” described above. The nucleotide sequence identified as “NT SEQ ID NO:X” was assembled from partially homologous (“overlapping”) sequences obtained from the “cDNA clone ID” identified in Table 1 and, in some cases, from additional related DNA clones. The overlapping sequences were assembled into a single contiguous sequence of high redundancy (usually three to five overlapping sequences at each nucleotide position), resulting in a final sequence identified as SEQ ID NO:X.

The cDNA Clone ID was deposited on the date and given the corresponding deposit number listed in “ATCC Deposit No:Z and Date.” Some of the deposits contain multiple different clones corresponding to the same gene. “Vector” refers to the type of vector contained in the cDNA Clone ID.

“Total NT Seq.” refers to the total number of nucleotides in the contig identified by “Gene No.” The deposited clone may contain all or most of these sequences, reflected by the nucleotide position indicated as “5′ NT of Clone Seq.” and the “3′ NT of Clone Seq.” of SEQ ID NO:X. The nucleotide position of SEQ ID NO:X of the putative start codon (methionine) is identified as “5′ NT of Start Codon.” Similarly, the nucleotide position of SEQ ID NO:X of the predicted signal sequence is identified as “5′ NT of First AA of Signal Pep.”

The translated amino acid sequence, beginning with the methionine, is identified as “AA SEQ ID NO:Y,” although other reading frames can also be easily translated using known molecular biology techniques. The polypeptides produced by these alternative open reading frames are specifically contemplated by the present invention.

The first and last amino acid position of SEQ ID NO:Y of the predicted signal peptide is identified as “First AA of Sig Pep” and “Last AA of Sig Pep.” The predicted first amino acid position of SEQ ID NO:Y of the secreted portion is identified as “Predicted First AA of Secreted Portion.” Finally, the amino acid position of SEQ ID NO:Y of the last amino acid in the open reading frame is identified as “Last AA of ORF.”

SEQ ID NO:X and the translated SEQ ID NO:Y are sufficiently accurate and otherwise suitable for a variety of uses well known in the art and described further below. For instance, SEQ ID NO:X is useful for designing nucleic acid hybridization probes that will detect nucleic acid sequences contained in SEQ ID NO:X or the cDNA contained in the deposited clone. These probes will also hybridize to nucleic acid molecules in biological samples, thereby enabling a variety of forensic and diagnostic methods of the invention. Similarly, polypeptides identified from SEQ ID NO:Y may be used to generate antibodies which bind specifically to the secreted proteins encoded by the cDNA clones identified in Table 1.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:X and the predicted translated amino acid sequence identified as SEQ ID NO:Y, but also a sample of plasmid DNA containing a human cDNA of the invention deposited with the ATCC, as set forth in Table 1. The nucleotide sequence of each deposited clone can readily be determined by sequencing the deposited clone in accordance with known methods. The predicted amino acid sequence can then be verified from such deposits. Moreover, the amino acid sequence of the protein encoded by a particular clone can also be directly determined by peptide sequencing or by expressing the protein in a suitable host cell containing the deposited human cDNA, collecting the protein, and determining its sequence.

The present invention also relates to the genes corresponding to SEQ ID NO:X, SEQ ID NO:Y, or the deposited clone. The corresponding gene can be isolated in accordance with known methods using the sequence information disclosed herein. Such methods include preparing probes or primers from the disclosed sequence and identifying or amplifying the corresponding gene from appropriate sources of genomic material.

Also provided in the present invention are species homologs. Species homologs may be isolated and identified by making suitable probes or primers from the sequences provided herein and screening a suitable nucleic acid source for the desired homologue.

The polypeptides of the invention can be prepared in any suitable manner. Such polypeptides include isolated naturally occurring polypeptides, recombinantly produced polypeptides, synthetically produced polypeptides, or polypeptides produced by a combination of these methods. Means for preparing such polypeptides are well understood in the art.

The polypeptides may be in the form of the secreted protein, including the mature form, or may be a part of a larger protein, such as a fusion protein (see below). It is often advantageous to include an additional amino acid sequence which contains secretory or leader sequences, pro-sequences, sequences which aid in purification, such as multiple histidine residues, or an additional sequence for stability during recombinant production.

The polypeptides of the present invention are preferably provided in an isolated form, and preferably are substantially purified. A recombinantly produced version of a polypeptide, including the secreted polypeptide, can be substantially purified by the one-step method described in Smith and Johnson, Gene 67:31-40 (1988). Polypeptides of the invention also can be purified from natural or recombinant sources using antibodies of the invention raised against the secreted protein in methods which are well known in the art.

Signal Sequences

Methods for predicting whether a protein has a signal sequence, as well as the cleavage point for that sequence, are available. For instance, the method of McGeoch, Virus Res. 3:271-286 (1985), uses the information from a short N-terminal charged region and a subsequent uncharged region of the complete (uncleaved) protein. The method of von Heinje, Nucleic Acids Res. 14:4683-4690 (1986) uses the information from the residues surrounding the cleavage site, typically residues −13 to +2, where +1 indicates the amino terminus of the secreted protein. The accuracy of predicting the cleavage points of known mammalian secretory proteins for each of these methods is in the range of 75-80%. (von Heinje, supra.) However, the two methods do not always produce the same predicted cleavage point(s) for a given protein.

In the present case, the deduced amino acid sequence of the secreted polypeptide was analyzed by a computer program called SignalP (Henrik Nielsen et al., Protein Engineering 10:1-6 (1997)), which predicts the cellular location of a protein based on the amino acid sequence. As part of this computational prediction of localization, the methods of McGeoch and von Heinje are incorporated. The analysis of the amino acid sequences of the secreted proteins described herein by this program provided the results shown in Table 1.

As one of ordinary skill would appreciate, however, cleavage sites sometimes vary from organism to organism and cannot be predicted with absolute certainty. Accordingly, the present invention provides secreted polypeptides having a sequence shown in SEQ ID NO:Y which have an N-terminus beginning within 5 residues (i.e., + or −5 residues) of the predicted cleavage point. Similarly, it is also recognized that in some cases, cleavage of the signal sequence from a secreted protein is not entirely uniform, resulting in more than one secreted species. These polypeptides, and the polynucleotides encoding such polypeptides, are contemplated by the present invention.

Moreover, the signal sequence identified by the above analysis may not necessarily predict the naturally occurring signal sequence. For example, the naturally occurring signal sequence may be further upstream from the predicted signal sequence. However, it is likely that the predicted signal sequence will be capable of directing the secreted protein to the ER. These polypeptides, and the polynucleotides encoding such polypeptides, are contemplated by the present invention.

Polynucleotide and Polypeptide Variants

“Variant” refers to a polynucleotide or polypeptide differing from the polynucleotide or polypeptide of the present invention, but retaining essential properties thereof. Generally, variants are overall closely similar, and, in many regions, identical to the polynucleotide or polypeptide of the present invention.

By a polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence encoding the polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence shown in Table 1, the ORF (open reading frame), or any fragement specified as described herein.

As a practical matter, whether any particular nucleic acid molecule or polypeptide is at least 90%, 95%, 96%, 97%, 98% or 99% identical to a nucleotide sequence of the presence invention can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identiy are: Matrix=Unitary, k-tuple=4, Mismatch Penalty=1, Joining Penalty=30, Randomization Group Length=0, Cutoff Score=1, Gap Penalty=5, Gap Size Penalty 0.05, Window Size=500 or the lenght of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the FASTDB alignment does not show a matched/alignement of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequnce are manually corrected for. No other manual corrections are to made for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, (indels) or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequences shown in Table 1 or to the amino acid sequence encoded by deposited DNA clone can be determined conventionally using known computer programs. A preferred method for determing the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is becuase the FASTDB program does not account for N- and C-terminal truncations of the subject sequence when calculating global percent identity. For subject sequences truncated at the N- and C-termini, relative to the the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N-terminus. The 10 unpaired residues represent 10% of the sequence (number of residues at the N— and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence which are not matched/aligned with the query. In this case the percent identity calculated by FASTDB is not manually corrected. Once again, only residue positions outside the N- and C-terminal ends of the subject sequence, as displayed in the FASTDB alignment, which are not matched/aligned with the query sequnce are manually corrected for. No other manual corrections are to made for the purposes of the present invention.

The variants may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, variants in which 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host such as E. coli).

Naturally occurring variants are called “allelic variants,” and refer to one of several alternate forms of a gene occupying a given locus on a chromosome of an organism. (Genes II, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids can be deleted from the N-terminus or C-terminus of the secreted protein without substantial loss of biological function. The authors of Ron et al., J. Biol. Chem. 268: 2984-2988 (1993), reported variant KGF proteins having heparin binding activity even after deleting 3, 8, or 27 amino-terminal amino acid residues. Similarly, Interferon gamma exhibited up to ten times higher activity after deleting 8-10 amino acid residues from the carboxy terminus of this protein. (Dobeli et al., J. Biotechnology 7:199-216 (1988).)

Moreover, ample evidence demonstrates that variants often retain a biological activity similar to that of the naturally occurring protein. For example, Gayle and coworkers (J. Biol. Chem 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type.

Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which show substantial biological activity. Such variants include deletions, insertions, inversions, repeats, and substitutions selected according to general rules known in the art so as have little effect on activity. For example, guidance concerning how to make phenotypically silent amino acid substitutions is provided in Bowie, J. U. et al., Science 247:1306-1310 (1990), wherein the authors indicate that there are two main strategies for studying the tolerance of an amino acid sequence to change.

The first strategy exploits the tolerance of amino acid substitutions by natural selection during the process of evolution. By comparing amino acid sequences in different species, conserved amino acids can be identified. These conserved amino acids are likely important for protein function. In contrast, the amino acid positions where substitutions have been tolerated by natural selection indicates that these positions are not critical for protein function. Thus, positions tolerating amino acid substitution could be modified while still maintaining biological activity of the protein.

The second strategy uses genetic engineering to introduce amino acid changes at specific positions of a cloned gene to identify regions critical for protein function. For example, site directed mutagenesis or alanine-scanning mutagenesis (introduction of single alanine mutations at every residue in the molecule) can be used. (Cunningham and Wells, Science 244:1081-1085 (1989).) The resulting mutant molecules can then be tested for biological activity.

As the authors state, these two strategies have revealed that proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) substitutions with one or more of the non-conserved amino acid residues, where the substituted amino acid residues may or may not be one encoded by the genetic code, or (ii) substitution with one or more of amino acid residues having a substituent group, or (iii) fusion of the mature polypeptide with another compound, such as a compound to increase the stability and/or solubility of the polypeptide (for example, polyethylene glycol), or (iv) fusion of the polypeptide with additional amino acids, such as an IgG Fc fusion region peptide, or leader or secretory sequence, or a sequence facilitating purification. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. (Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).)

A further embodiment of the invention relates to a polypeptide which comprises the amino acid sequence of the present invention having an amino acid sequence which contains at least one amino acid substitution, but not more than 50 amino acid substitutions, even more preferably, not more than 40 amino acid substitutions, still more preferably, not more than 30 amino acid substitutions, and still even more preferably, not more than 20 amino acid substitutions. Of course, in order of ever-increasing preference, it is highly preferable for a polypeptide to have an amino acid sequence which comprises the amino acid sequence of the present invention, which contains at least one, but not more than 10, 9, 8, 7, 6, 5, 4, 3, 2 or 1 amino acid substitutions. In specific embodiments, the number of additions, substitutions, and/or deletions in the amino acid sequence of the present invention or fragments thereof (e.g., the mature form and/or other fragments described herein), is 1-5,5-10, 5-25, 5-50, 10-50 or 50-150, conservative amino acid substitutions are preferable.

Polynucleotide and Polypeptide Fragments

In the present invention, a “polynucleotide fragment” refers to a short polynucleotide having a nucleic acid sequence contained in the deposited clone or shown in SEQ ID NO:X. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clone or the nucleotide sequence shown in SEQ ID NO:X. These nucleotide fragments are useful as diagnostic probes and primers as discussed herein. Of course, larger fragments (e.g., 50, 150, 500, 600, 2000 nucleotides) are preferred.

Moreover, representative examples of polynucleotide fragments of the invention, include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950,951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO:X or the cDNA contained in the deposited clone. In this context “about” includes the particularly recited ranges, larger or smaller by several (5, 4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:Y or encoded by the cDNA contained in the deposited clone. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, or 161 to the end of the coding region. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted protein as well as the mature form. Further preferred polypeptide fragments include the secreted protein or the mature form having a continuous series of deleted residues from the amino or the carboxy terminus, or both. For example, any number of amino acids, ranging from 1-60, can be deleted from the amino terminus of either the secreted polypeptide or the mature form. Similarly, any number of amino acids, ranging from 1-30, can be deleted from the carboxy terminus of the secreted protein or mature form. Furthermore, any combination of the above amino and carboxy terminus deletions are preferred. Similarly, polynucleotide fragments encoding these polypeptide fragments are also preferred.

Also preferred are polypeptide and polynucleotide fragments characterized by structural or functional domains, such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Polypeptide fragments of SEQ ID NO:Y falling within conserved domains are specifically contemplated by the present invention. Moreover, polynucleotide fragments encoding these domains are also contemplated.

Other preferred fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

Epitopes & Antibodies

In the present invention, “epitopes” refer to polypeptide fragments having antigenic or immunogenic activity in an animal, especially in a human. A preferred embodiment of the present invention relates to a polypeptide fragment comprising an epitope, as well as the polynucleotide encoding this fragment. A region of a protein molecule to which an antibody can bind is defined as an “antigenic epitope.” In contrast, an “immunogenic epitope” is defined as a part of a protein that elicits an antibody response. (See, for instance, Geysen et al., Proc. Natl. Acad. Sci. USA 81:3998-4002 (1983).)

Fragments which function as epitopes may be produced by any conventional means. (See, e.g., Houghten, R. A., Proc. Natl. Acad. Sci. USA 82:5131-5135 (1985) further described in U.S. Pat. No. 4,631,211.) In the present invention, antigenic epitopes preferably contain a sequence of at least seven, more preferably at least nine, and most preferably between about 15 to about 30 amino acids. Antigenic epitopes are useful to raise antibodies, including monoclonal antibodies, that specifically bind the epitope. (See, for instance, Wilson et al., Cell 37:767-778 (1984); Sutcliffe, J. G. et al., Science 219:660-666 (1983).) Similarly, immunogenic epitopes can be used to induce antibodies according to methods well known in the art. (See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985).) A preferred immunogenic epitope includes the secreted protein. The immunogenic epitopes may be presented together with a carrier protein, such as an albumin, to an animal system (such as rabbit or mouse) or, if it is long enough (at least about 25 amino acids), without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting.)

As used herein, the term “antibody” (Ab) or “monoclonal antibody” (Mab) is meant to include intact molecules as well as antibody fragments (such as, for example, Fab and F(ab′)₂ fragments) which are capable of specifically binding to protein. Fab and F(ab′)₂ fragments lack the Fc fragment of intact antibody, clear more rapidly from the circulation, and may have less non-specific tissue binding than an intact antibody. (Wahl et al., J. Nucl. Med. 24:316-325 (1983).) Thus, these fragments are preferred, as well as the products of a FAB or other immunoglobulin expression library. Moreover, antibodies of the present invention include chimeric, single chain, and humanized antibodies.

Fusion Proteins

Any polypeptide of the present invention can be used to generate fusion proteins. For example, the polypeptide of the present invention, when fused to a second protein, can be used as an antigenic tag. Antibodies raised against the polypeptide of the present invention can be used to indirectly detect the second protein by binding to the polypeptide. Moreover, because secreted proteins target cellular locations based on trafficking signals, the polypeptides of the present invention can be used as targeting molecules once fused to other proteins.

Examples of domains that can be fused to polypeptides of the present invention include not only heterologous signal sequences, but also other heterologous functional regions. The fusion does not necessarily need to be direct, but may occur through linker sequences.

Moreover, fusion proteins may also be engineered to improve characteristics of the polypeptide of the present invention. For instance, a region of additional amino acids, particularly charged amino acids, may be added to the N-terminus of the polypeptide to improve stability and persistence during purification from the host cell or subsequent handling and storage. Also, peptide moieties may be added to the polypeptide to facilitate purification. Such regions may be removed prior to final preparation of the polypeptide. The addition of peptide moieties to facilitate handling of polypeptides are familiar and routine techniques in the art.

Moreover, polypeptides of the present invention, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the polypeptides of the present invention can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the polynucleotides or the polypeptides of the present invention.

Vectors, Host Cells, and Protein Production

The present invention also relates to vectors containing the polynucleotide of the present invention, host cells, and the production of polypeptides by recombinant techniques. The vector may be, for example, a phage, plasmid, viral, or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter, such as the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

A polypeptide of this invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

Polypeptides of the present invention, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., coding sequence), and/or to include genetic material (e.g., heterologous polynucleotide sequences) that is operably associated with the polynucleotides of the invention, and which activates, alters, and/or amplifies endogenous polynucleotides. For example, techniques known in the art may be used to operably associate heterologous control regions (e.g., promoter and/or enhancer) and endogenous polynucleotide sequences via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989), the disclosures of each of which are incorporated by reference in their entireties).

Uses of the Polynucleotides

Each of the polynucleotides identified herein can be used in numerous ways as reagents. The following description should be considered exemplary and utilizes known techniques.

The polynucleotides of the present invention are useful for chromosome identification. There exists an ongoing need to identify new chromosome markers, since few chromosome marking reagents, based on actual sequence data (repeat polymorphisms), are presently available. Each polynucleotide of the present invention can be used as a chromosome marker.

Briefly, sequences can be mapped to chromosomes by preparing PCR primers (preferably 15-25 bp) from the sequences shown in SEQ ID NO:X. Primers can be selected using computer analysis so that primers do not span more than one predicted exon in the genomic DNA. These primers are then used for PCR screening of somatic cell hybrids containing individual human chromosomes. Only those hybrids containing the human gene corresponding to the SEQ ID NO:X will yield an amplified fragment.

Similarly, somatic hybrids provide a rapid method of PCR mapping the polynucleotides to particular chromosomes. Three or more clones can be assigned per day using a single thermal cycler. Moreover, sublocalization of the polynucleotides can be achieved with panels of specific chromosome fragments. Other gene mapping strategies that can be used include in situ hybridization, prescreening with labeled flow-sorted chromosomes, and preselection by hybridization to construct chromosome specific-cDNA libraries.

Precise chromosomal location of the polynucleotides can also be achieved using fluorescence in situ hybridization (FISH) of a metaphase chromosomal spread. This technique uses polynucleotides as short as 500 or 600 bases; however, polynucleotides 2,000-4,000 bp are preferred. For a review of this technique, see Verma et al., “Human Chromosomes: a Manual of Basic Techniques,” Pergamon Press, New York (1988).

For chromosome mapping, the polynucleotides can be used individually (to mark a single chromosome or a single site on that chromosome) or in panels (for marking multiple sites and/or multiple chromosomes). Preferred polynucleotides correspond to the noncoding regions of the cDNAs because the coding sequences are more likely conserved within gene families, thus increasing the chance of cross hybridization during chromosomal mapping.

Once a polynucleotide has been mapped to a precise chromosomal location, the physical position of the polynucleotide can be used in linkage analysis. Linkage analysis establishes coinheritance between a chromosomal location and presentation of a particular disease. (Disease mapping data are found, for example, in V. McKusick, Mendelian Inheritance in Man (available on line through Johns Hopkins University Welch Medical Library).) Assuming 1 megabase mapping resolution and one gene per 20 kb, a cDNA precisely localized to a chromosomal region associated with the disease could be one of 50-500 potential causative genes.

Thus, once coinheritance is established, differences in the polynucleotide and the corresponding gene between affected and unaffected individuals can be examined. First, visible structural alterations in the chromosomes, such as deletions or translocations, are examined in chromosome spreads or by PCR. If no structural alterations exist, the presence of point mutations are ascertained. Mutations observed in some or all affected individuals, but not in normal individuals, indicates that the mutation may cause the disease. However, complete sequencing of the polypeptide and the corresponding gene from several normal individuals is required to distinguish the mutation from a polymorphism. If a new polymorphism is identified, this polymorphic polypeptide can be used for further linkage analysis.

Furthermore, increased or decreased expression of the gene in affected individuals as compared to unaffected individuals can be assessed using polynucleotides of the present invention. Any of these alterations (altered expression, chromosomal rearrangement, or mutation) can be used as a diagnostic or prognostic marker.

In addition to the foregoing, a polynucleotide can be used to control gene expression through triple helix formation or antisense DNA or RNA. Both methods rely on binding of the polynucleotide to DNA or RNA. For these techniques, preferred polynucleotides are usually 20 to 40 bases in length and complementary to either the region of the gene involved in transcription (triple helix—see Lee et al., Nucl. Acids Res. 6:3073 (1979); Cooney et al., Science 241:456 (1988); and Dervan et al., Science 251:1360 (1991)) or to the mRNA itself (antisense—Okano, J. Neurochem. 56:560 (1991); Oligodeoxy-nucleotides as Antisense Inhibitors of Gene Expression, CRC Press, Boca Raton, Fla. (1988).) Triple helix formation optimally results in a shut-off of RNA transcription from DNA, while antisense RNA hybridization blocks translation of an mRNA molecule into polypeptide. Both techniques are effective in model systems, and the information disclosed herein can be used to design antisense or triple helix polynucleotides in an effort to treat disease.

Polynucleotides of the present invention are also useful in gene therapy. One goal of gene therapy is to insert a normal gene into an organism having a defective gene, in an effort to correct the genetic defect. The polynucleotides disclosed in the present invention offer a means of targeting such genetic defects in a highly accurate manner. Another goal is to insert a new gene that was not present in the host genome, thereby producing a new trait in the host cell.

The polynucleotides are also useful for identifying individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identifying personnel. This method does not suffer from the current limitations of “Dog Tags” which can be lost, switched, or stolen, making positive identification difficult. The polynucleotides of the present invention can be used as additional DNA markers for RFLP.

The polynucleotides of the present invention can also be used as an alternative to RFLP, by determining the actual base-by-base DNA sequence of selected portions of an individual's genome. These sequences can be used to prepare PCR primers for amplifying and isolating such selected DNA, which can then be sequenced. Using this technique, individuals can be identified because each individual will have a unique set of DNA sequences. Once an unique ID database is established for an individual, positive identification of that individual, living or dead, can be made from extremely small tissue samples.

Forensic biology also benefits from using DNA-based identification techniques as disclosed herein. DNA sequences taken from very small biological samples such as tissues, e.g., hair or skin, or body fluids, e.g., blood, saliva, semen, etc., can be amplified using PCR. In one prior art technique, gene sequences amplified from polymorphic loci, such as DQa class II HLA gene, are used in forensic biology to identify individuals. (Erlich, H., PCR Technology, Freeman and Co. (1992).) Once these specific polymorphic loci are amplified, they are digested with one or more restriction enzymes, yielding an identifying set of bands on a Southern blot probed with DNA corresponding to the DQa class II HLA gene. Similarly, polynucleotides of the present invention can be used as polymorphic markers for forensic purposes.

There is also a need for reagents capable of identifying the source of a particular tissue. Such need arises, for example, in forensics when presented with tissue of unknown origin. Appropriate reagents can comprise, for example, DNA probes or primers specific to particular tissue prepared from the sequences of the present invention. Panels of such reagents can identify tissue by species and/or by organ type. In a similar fashion, these reagents can be used to screen tissue cultures for contamination.

In the very least, the polynucleotides of the present invention can be used as molecular weight markers on Southern gels, as diagnostic probes for the presence of a specific mRNA in a particular cell type, as a probe to “subtract-out” known sequences in the process of discovering novel polynucleotides, for selecting and making oligomers for attachment to a “gene chip” or other support, to raise anti-DNA antibodies using DNA immunization techniques, and as an antigen to elicit an immune response.

Uses of the Polypeptides

Each of the polypeptides identified herein can be used in numerous ways. The following description should be considered exemplary and utilizes known techniques.

A polypeptide of the present invention can be used to assay protein levels in a biological sample using antibody-based techniques. For example, protein expression in tissues can be studied with classical immunohistological methods. (Jalkanen, M., et al., J. Cell. Biol. 101:976-985 (1985); Jalkanen, M., et al., J. Cell. Biol. 105:3087-3096 (1987).) Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (125I, 121I), carbon (14C), sulfur (35S), tritium (3H), indium (112In), and technetium (99mTc), and fluorescent labels, such as fluorescein and rhodamine, and biotin.

In addition to assaying secreted protein levels in a biological sample, proteins can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A protein-specific antibody or antibody fragment which has been labeled with an appropriate detectable imaging moiety, such as a radioisotope (for example, 131I, 112In, 99mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99mTc. The labeled antibody or antibody fragment will then preferentially accumulate at the location of cells which contain the specific protein. In vivo tumor imaging is described in S.W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).)

Thus, the invention provides a diagnostic method of a disorder, which involves (a) assaying the expression of a polypeptide of the present invention in cells or body fluid of an individual; (b) comparing the level of gene expression with a standard gene expression level, whereby an increase or decrease in the assayed polypeptide gene expression level compared to the standard expression level is indicative of a disorder.

Moreover, polypeptides of the present invention can be used to treat disease. For example, patients can be administered a polypeptide of the present invention in an effort to replace absent or decreased levels of the polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibodies directed to a polypeptide of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane (receptor).

At the very least, the polypeptides of the present invention can be used as molecular weight markers on SDS-PAGE gels or on molecular sieve gel filtration columns using methods well known to those of skill in the art. Polypeptides can also be used to raise antibodies, which in turn are used to measure protein expression from a recombinant cell, as a way of assessing transformation of the host cell. Moreover, the polypeptides of the present invention can be used to test the following biological activities.

Biological Activities

The polynucleotides and polypeptides of the present invention can be used in assays to test for one or more biological activities. If these polynucleotides and polypeptides do exhibit activity in a particular assay, it is likely that these molecules may be involved in the diseases associated with the biological activity. Thus, the polynucleotides and polypeptides could be used to treat the associated disease.

Immune Activity

A polypeptide or polynucleotide of the present invention may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, a polynucleotide or polypeptide of the present invention can be used as a marker or detector of a particular immune system disease or disorder.

A polynucleotide or polypeptide of the present invention may be useful in treating or detecting deficiencies or disorders of hematopoietic cells. A polypeptide or polynucleotide of the present invention could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, a polypeptide or polynucleotide of the present invention could also be used to modulate hemostatic (the stopping of bleeding) or thrombolytic activity (clot formation). For example, by increasing hemostatic or thrombolytic activity, a polynucleotide or polypeptide of the present invention could be used to treat blood coagulation disorders (e.g., afibrinogenemia, factor deficiencies), blood platelet disorders (e.g. thrombocytopenia), or wounds resulting from trauma, surgery, or other causes. Alternatively, a polynucleotide or polypeptide of the present invention that can decrease hemostatic or thrombolytic activity could be used to inhibit or dissolve clotting. These molecules could be important in the treatment of heart attacks (infarction), strokes, or scarring.

A polynucleotide or polypeptide of the present invention may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of a polypeptide or polynucleotide of the present invention that inhibits an immune response, particularly the proliferation, differentiation, or chemotaxis of T-cells, may be an effective therapy in preventing autoimmune disorders.

Examples of autoimmune disorders that can be treated or detected by the present invention include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by a polypeptide or polynucleotide of the present invention. Moreover, these molecules can be used to treat anaphylaxis, hypersensitivity to an antigenic molecule, or blood group incompatibility.

A polynucleotide or polypeptide of the present invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of a polypeptide or polynucleotide of the present invention that inhibits an immune response, particularly the proliferation, differentiation, or chemotaxis of T-cells, may be an effective therapy in preventing organ rejection or GVHD.

Similarly, a polypeptide or polynucleotide of the present invention may also be used to modulate inflammation. For example, the polypeptide or polynucleotide may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

A polypeptide or polynucleotide can be used to treat or detect hyperproliferative disorders, including neoplasms. A polypeptide or polynucleotide of the present invention may inhibit the proliferation of the disorder through direct or indirect interactions. Alternatively, a polypeptide or polynucleotide of the present invention may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by a polynucleotide or polypeptide of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by a polynucleotide or polypeptide of the present invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Infectious Disease

A polypeptide or polynucleotide of the present invention can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, the polypeptide or polynucleotide of the present invention may also directly inhibit the infectious agent, without necessarily eliciting an immune response.

Viruses are one example of an infectious agent that can cause disease or symptoms that can be treated or detected by a polynucleotide or polypeptide of the present invention. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. A polypeptide or polynucleotide of the present invention can be used to treat or detect any of these symptoms or diseases.

Similarly, bacterial or fungal agents that can cause disease or symptoms and that can be treated or detected by a polynucleotide or polypeptide of the present invention include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (KIebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacterermia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

Regeneration

A polynucleotide or polypeptide of the present invention can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, burns, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, a polynucleotide or polypeptide of the present invention may increase regeneration of tissues difficult to heal. For example, increased tendon/ligament regeneration would quicken recovery time after damage. A polynucleotide or polypeptide of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using a polynucleotide or polypeptide of the present invention to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the polynucleotide or polypeptide of the present invention.

Chemotaxis

A polynucleotide or polypeptide of the present invention may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as inflammation, infection, or site of hyperproliferation. The mobilized cells can then fight off and/or heal the particular trauma or abnormality.

A polynucleotide or polypeptide of the present invention may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. Chemotactic molecules of the present invention can also attract fibroblasts, which can be used to treat wounds.

It is also contemplated that a polynucleotide or polypeptide of the present invention may inhibit chemotactic activity. These molecules could also be used to treat disorders. Thus, a polynucleotide or polypeptide of the present invention could be used as an inhibitor of chemotaxis.

Binding Activity

A polypeptide of the present invention may be used to screen for molecules that bind to the polypeptide or for molecules to which the polypeptide binds. The binding of the polypeptide and the molecule may activate (agonist), increase, inhibit (antagonist), or decrease activity of the polypeptide or the molecule bound. Examples of such molecules include antibodies, oligonucleotides, proteins (e.g., receptors), or small molecules.

Preferably, the molecule is closely related to the natural ligand of the polypeptide, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which the polypeptide binds, or at least, a fragment of the receptor capable of being bound by the polypeptide (e.g., active site). In either case, the molecule can be rationally designed using known techniques.

Preferably, the screening for these molecules involves producing appropriate cells which express the polypeptide, either as a secreted protein or on the cell membrane. Preferred cells include cells from mammals, yeast, Drosophila, or E. coli. Cells expressing the polypeptide (or cell membrane containing the expressed polypeptide) are then preferably contacted with a test compound potentially containing the molecule to observe binding, stimulation, or inhibition of activity of either the polypeptide or the molecule.

The assay may simply test binding of a candidate compound to the polypeptide, wherein binding is detected by a label, or in an assay involving competition with a labeled competitor. Further, the assay may test whether the candidate compound results in a signal generated by binding to the polypeptide.

Alternatively, the assay can be carried out using cell-free preparations, polypeptide/molecule affixed to a solid support, chemical libraries, or natural product mixtures. The assay may also simply comprise the steps of mixing a candidate compound with a solution containing a polypeptide, measuring polypeptide/molecule activity or binding, and comparing the polypeptide/molecule activity or binding to a standard.

Preferably, an ELISA assay can measure polypeptide level or activity in a sample (e.g., biological sample) using a monoclonal or polyclonal antibody. The antibody can measure polypeptide level or activity by either binding, directly or indirectly, to the polypeptide or by competing with the polypeptide for a substrate.

All of these above assays can be used as diagnostic or prognostic markers. The molecules discovered using these assays can be used to treat disease or to bring about a particular result in a patient (e.g., blood vessel growth) by activating or inhibiting the polypeptide/molecule. Moreover, the assays can discover agents which may inhibit or enhance the production of the polypeptide from suitably manipulated cells or tissues.

Therefore, the invention includes a method of identifying compounds which bind to a polypeptide of the invention comprising the steps of: (a) incubating a candidate binding compound with a polypeptide of the invention; and (b) determining if binding has occurred. Moreover, the invention includes a method of identifying agonists/antagonists comprising the steps of: (a) incubating a candidate compound with a polypeptide of the invention, (b) assaying a biological activity, and (b) determining if a biological activity of the polypeptide has been altered.

Other Activities

A polypeptide or polynucleotide of the present invention may also increase or decrease the differentiation or proliferation of embryonic stem cells, besides, as discussed above, hematopoietic lineage.

A polypeptide or polynucleotide of the present invention may also be used to modulate mammalian characteristics, such as body height, weight, hair color, eye color, skin, percentage of adipose tissue, pigmentation, size, and shape (e.g., cosmetic surgery). Similarly, a polypeptide or polynucleotide of the present invention may be used to modulate mammalian metabolism affecting catabolism, anabolism, processing, utilization, and storage of energy.

A polypeptide or polynucleotide of the present invention may be used to change a mammal's mental state or physical state by influencing biorhythms, caricadic rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

A polypeptide or polynucleotide of the present invention may also be used as a food additive or preservative, such as to increase or decrease storage capabilities, fat content, lipid, protein, carbohydrate, vitamins, minerals, cofactors or other nutritional components.

Other Preferred Embodiments

Other preferred embodiments of the claimed invention include an isolated nucleic acid molecule comprising a nucleotide sequence which is at least 95% identical to a sequence of at least about 50 contiguous nucleotides in the nucleotide sequence of SEQ ID NO:X wherein X is any integer as defined in Table 1.

Also preferred is a nucleic acid molecule wherein said sequence of contiguous nucleotides is included in the nucleotide sequence of SEQ ID NO:X in the range of positions beginning with the nucleotide at about the position of the 5′ Nucleotide of the Clone Sequence and ending with the nucleotide at about the position of the 3′ Nucleotide of the Clone Sequence as defined for SEQ ID NO:X in Table 1.

Also preferred is a nucleic acid molecule wherein said sequence of contiguous nucleotides is included in the nucleotide sequence of SEQ ID NO:X in the range of positions beginning with the nucleotide at about the position of the 5′ Nucleotide of the Start Codon and ending with the nucleotide at about the position of the 3′ Nucleotide of the Clone Sequence as defined for SEQ ID NO:X in Table 1.

Similarly preferred is a nucleic acid molecule wherein said sequence of contiguous nucleotides is included in the nucleotide sequence of SEQ ID NO:X in the range of positions beginning with the nucleotide at about the position of the 5′ Nucleotide of the First Amino Acid of the Signal Peptide and ending with the nucleotide at about the position of the 3′ Nucleotide of the Clone Sequence as defined for SEQ ID NO:X in Table 1.

Also preferred is an isolated nucleic acid molecule comprising a nucleotide sequence which is at least 95% identical to a sequence of at least about 150 contiguous nucleotides in the nucleotide sequence of SEQ ID NO:X.

Further preferred is an isolated nucleic acid molecule comprising a nucleotide sequence which is at least 95% identical to a sequence of at least about 500 contiguous nucleotides in the nucleotide sequence of SEQ ID NO:X.

A further preferred embodiment is a nucleic acid molecule comprising a nucleotide sequence which is at least 95% identical to the nucleotide sequence of SEQ ID NO:X beginning with the nucleotide at about the position of the 5′ Nucleotide of the First Amino Acid of the Signal Peptide and ending with the nucleotide at about the position of the 3′ Nucleotide of the Clone Sequence as defined for SEQ ID NO:X in Table 1.

A further preferred embodiment is an isolated nucleic acid molecule comprising a nucleotide sequence which is at least 95% identical to the complete nucleotide sequence of SEQ ID NO:X.

Also preferred is an isolated nucleic acid molecule which hybridizes under stringent hybridization conditions to a nucleic acid molecule, wherein said nucleic acid molecule which hybridizes does not hybridize under stringent hybridization conditions to a nucleic acid molecule having a nucleotide sequence consisting of only A residues or of only T residues.

Also preferred is a composition of matter comprising a DNA molecule which comprises a human cDNA clone identified by a cDNA Clone Identifier in Table 1, which DNA molecule is contained in the material deposited with the American Type Culture Collection and given the ATCC Deposit Number shown in Table 1 for said cDNA Clone Identifier.

Also preferred is an isolated nucleic acid molecule comprising a nucleotide sequence which is at least 95% identical to a sequence of at least 50 contiguous nucleotides in the nucleotide sequence of a human cDNA clone identified by a cDNA Clone Identifier in Table 1, which DNA molecule is contained in the deposit given the ATCC Deposit Number shown in Table 1.

Also preferred is an isolated nucleic acid molecule, wherein said sequence of at least 50 contiguous nucleotides is included in the nucleotide sequence of the complete open reading frame sequence encoded by said human cDNA clone.

Also preferred is an isolated nucleic acid molecule comprising a nucleotide sequence which is at least 95% identical to sequence of at least 150 contiguous nucleotides in the nucleotide sequence encoded by said human cDNA clone.

A further preferred embodiment is an isolated nucleic acid molecule comprising a nucleotide sequence which is at least 95% identical to sequence of at least 500 contiguous nucleotides in the nucleotide sequence encoded by said human cDNA clone.

A further preferred embodiment is an isolated nucleic acid molecule comprising a nucleotide sequence which is at least 95% identical to the complete nucleotide sequence encoded by said human cDNA clone.

A further preferred embodiment is a method for detecting in a biological sample a nucleic acid molecule comprising a nucleotide sequence which is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1; which method comprises a step of comparing a nucleotide sequence of at least one nucleic acid molecule in said sample with a sequence selected from said group and determining whether the sequence of said nucleic acid molecule in said sample is at least 95% identical to said selected sequence.

Also preferred is the above method wherein said step of comparing sequences comprises determining the extent of nucleic acid hybridization between nucleic acid molecules in said sample and a nucleic acid molecule comprising said sequence selected from said group. Similarly, also preferred is the above method wherein said step of comparing sequences is performed by comparing the nucleotide sequence determined from a nucleic acid molecule in said sample with said sequence selected from said group. The nucleic acid molecules can comprise DNA molecules or RNA molecules.

A further preferred embodiment is a method for identifying the species, tissue or cell type of a biological sample which method comprises a step of detecting nucleic acid molecules in said sample, if any, comprising a nucleotide sequence that is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1.

The method for identifying the species, tissue or cell type of a biological sample can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

Also preferred is a method for diagnosing in a subject a pathological condition associated with abnormal structure or expression of a gene encoding a secreted protein identified in Table 1, which method comprises a step of detecting in a biological sample obtained from said subject nucleic acid molecules, if any, comprising a nucleotide sequence that is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1.

The method for diagnosing a pathological condition can comprise a step of detecting nucleic acid molecules comprising a nucleotide sequence in a panel of at least two nucleotide sequences, wherein at least one sequence in said panel is at least 95% identical to a sequence of at least 50 contiguous nucleotides in a sequence selected from said group.

Also preferred is a composition of matter comprising isolated nucleic acid molecules wherein the nucleotide sequences of said nucleic acid molecules comprise a panel of at least two nucleotide sequences, wherein at least one sequence in said panel is at least 95% identical to a sequence of at least 50 contiguous nucleotides in a sequence selected from the group consisting of: a nucleotide sequence of SEQ ID NO:X wherein X is any integer as defined in Table 1; and a nucleotide sequence encoded by a human cDNA clone identified by a cDNA Clone Identifier in Table 1 and contained in the deposit with the ATCC Deposit Number shown for said cDNA clone in Table 1. The nucleic acid molecules can comprise DNA molecules or RNA molecules.

Also preferred is an isolated polypeptide comprising an amino acid sequence at least 90% identical to a sequence of at least about 10 contiguous amino acids in the amino acid sequence of SEQ ID NO:Y wherein Y is any integer as defined in Table 1.

Also preferred is a polypeptide, wherein said sequence of contiguous amino acids is included in the amino acid sequence of SEQ ID NO:Y in the range of positions beginning with the residue at about the position of the First Amino Acid of the Secreted Portion and ending with the residue at about the Last Amino Acid of the Open Reading Frame as set forth for SEQ ID NO:Y in Table 1.

Also preferred is an isolated polypeptide comprising an amino acid sequence at least 95% identical to a sequence of at least about 30 contiguous amino acids in the amino acid sequence of SEQ ID NO:Y.

Further preferred is an isolated polypeptide comprising an amino acid sequence at least 95% identical to a sequence of at least about 100 contiguous amino acids in the amino acid sequence of SEQ ID NO:Y.

Further preferred is an isolated polypeptide comprising an amino acid sequence at least 95% identical to the complete amino acid sequence of SEQ ID NO:Y.

Further preferred is an isolated polypeptide comprising an amino acid sequence at least 90% identical to a sequence of at least about 10 contiguous amino acids in the complete amino acid sequence of a secreted protein encoded by a human cDNA clone identified by a cDNA Clone Identifier in Table 1 and contained in the deposit with the ATCC Deposit Number shown for said cDNA clone in Table 1.

Also preferred is a polypeptide wherein said sequence of contiguous amino acids is included in the amino acid sequence of a secreted portion of the secreted protein encoded by a human cDNA clone identified by a cDNA Clone Identifier in Table 1 and contained in the deposit with the ATCC Deposit Number shown for said cDNA clone in Table 1.

Also preferred is an isolated polypeptide comprising an amino acid sequence at least 95% identical to a sequence of at least about 30 contiguous amino acids in the amino acid sequence of the secreted portion of the protein encoded by a human cDNA clone identified by a cDNA Clone Identifier in Table 1 and contained in the deposit with the ATCC Deposit Number shown for said cDNA clone in Table 1.

Also preferred is an isolated polypeptide comprising an amino acid sequence at least 95% identical to a sequence of at least about 100 contiguous amino acids in the amino acid sequence of the secreted portion of the protein encoded by a human cDNA clone identified by a cDNA Clone Identifier in Table 1 and contained in the deposit with the ATCC Deposit Number shown for said cDNA clone in Table 1.

Also preferred is an isolated polypeptide comprising an amino acid sequence at least 95% identical to the amino acid sequence of the secreted portion of the protein encoded by a human cDNA clone identified by a cDNA Clone Identifier in Table 1 and contained in the deposit with the ATCC Deposit Number shown for said cDNA clone in Table 1.

Further preferred is an isolated antibody which binds specifically to a polypeptide comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC Deposit Number shown for said cDNA clone in Table 1.

Further preferred is a method for detecting in a biological sample a polypeptide comprising an amino acid sequence which is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC Deposit Number shown for said cDNA clone in Table 1; which method comprises a step of comparing an amino acid sequence of at least one polypeptide molecule in said sample with a sequence selected from said group and determining whether the sequence of said polypeptide molecule in said sample is at least 90% identical to said sequence of at least 10 contiguous amino acids. Also preferred is the above method wherein said step of comparing an amino acid sequence of at least one polypeptide molecule in said sample with a sequence selected from said group comprises determining the extent of specific binding of polypeptides in said sample to an antibody which binds specifically to a polypeptide comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a protein encoded by a human cDNA clone identified by a cDNA Clone Identifier in Table 1 and contained in the deposit with the ATCC Deposit Number shown for said cDNA clone in Table 1.

Also preferred is the above method wherein said step of comparing sequences is performed by comparing the amino acid sequence determined from a polypeptide molecule in said sample with said sequence selected from said group.

Also preferred is a method for identifying the species, tissue or cell type of a biological sample which method comprises a step of detecting polypeptide molecules in said sample, if any, comprising an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

Also preferred is the above method for identifying the species, tissue or cell type of a biological sample, which method comprises a step of detecting polypeptide molecules comprising an amino acid sequence in a panel of at least two amino acid sequences, wherein at least one sequence in said panel is at least 90% identical to a sequence of at least 10 contiguous amino acids in a sequence selected from the above group.

Also preferred is a method for diagnosing in a subject a pathological condition associated with abnormal structure or expression of a gene encoding a secreted protein identified in Table 1, which method comprises a step of detecting in a biological sample obtained from said subject polypeptide molecules comprising an amino acid sequence in a panel of at least two amino acid sequences, wherein at least one sequence in said panel is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

In any of these methods, the step of detecting said polypeptide molecules includes using an antibody.

Also preferred is an isolated nucleic acid molecule comprising a nucleotide sequence which is at least 95% identical to a nucleotide sequence encoding a polypeptide wherein said polypeptide comprises an amino acid sequence that is at least 90% identical to a sequence of at least 10 contiguous amino acids in a sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

Also preferred is an isolated nucleic acid molecule, wherein said nucleotide sequence encoding a polypeptide has been optimized for expression of said polypeptide in a prokaryotic host.

Also preferred is an isolated nucleic acid molecule, wherein said polypeptide comprises an amino acid sequence selected from the group consisting of: an amino acid sequence of SEQ ID NO:Y wherein Y is any integer as defined in Table 1; and a complete amino acid sequence of a secreted protein encoded by a human cDNA clone identified by a cDNA Clone Identifier in Table 1 and contained in the deposit with the ATCC Deposit Number shown for said cDNA clone in Table 1.

Further preferred is a method of making a recombinant vector comprising inserting any of the above isolated nucleic acid molecule into a vector. Also preferred is the recombinant vector produced by this method. Also preferred is a method of making a recombinant host cell comprising introducing the vector into a host cell, as well as the recombinant host cell produced by this method.

Also preferred is a method of making an isolated polypeptide comprising culturing this recombinant host cell under conditions such that said polypeptide is expressed and recovering said polypeptide. Also preferred is this method of making an isolated polypeptide, wherein said recombinant host cell is a eukaryotic cell and said polypeptide is a secreted portion of a human secreted protein comprising an amino acid sequence selected from the group consisting of: an amino acid sequence of SEQ ID NO:Y beginning with the residue at the position of the First Amino Acid of the Secreted Portion of SEQ ID NO:Y wherein Y is an integer set forth in Table 1 and said position of the First Amino Acid of the Secreted Portion of SEQ ID NO:Y is defined in Table 1; and an amino acid sequence of a secreted portion of a protein encoded by a human cDNA clone identified by a cDNA Clone Identifier in Table 1 and contained in the deposit with the ATCC Deposit Number shown for said cDNA clone in Table 1. The isolated polypeptide produced by this method is also preferred.

Also preferred is a method of treatment of an individual in need of an increased level of a secreted protein activity, which method comprises administering to such an individual a pharmaceutical composition comprising an amount of an isolated polypeptide, polynucleotide, or antibody of the claimed invention effective to increase the level of said protein activity in said individual.

Having generally described the invention, the same will be more readily understood by reference to the following examples, which are provided by way of illustration and are not intended as limiting.

### EXAMPLES

### Example 1

### Isolation of a Selected cDNA Clone from the Deposited Sample

Each cDNA clone in a cited ATCC deposit is contained in a plasmid vector. Table I identifies the vectors used to construct the cDNA library from which each clone was isolated. In many cases, the vector used to construct the library is a phage vector from which a plasmid has been excised. The table immediately below correlates the related plasmid for each phage vector used in constructing the cDNA library. For example, where a particular clone is identified in Table 1 as being isolated in the vector “Lambda Zap,” the corresponding deposited clone is in “pBluescript.”

| Vector Used to Construct Library | |
|---|---|
| Plasmid | Corresponding Deposited |
| Lambda Zap | pBluescript (pBS) |
| Uni-Zap XR | pBluescript (pBS) |
| Zap Express | pBK |
| lafmid BA | plafmid BA |
| pSport1 | pSport1 |
| pCMVSport 2.0 | pCMVSport 2.0 |
| pCMVSport 3.0 | pCMVSport 3.0 |
| pCR ®2.1 | pCR ®2.1 |

Vectors Lambda Zap (U.S. Pat. Nos. 5,128,256 and 5,286,636), Uni-Zap XR (U.S. Pat. Nos. 5,128, 256 and 5,286,636), Zap Express (U.S. Pat. Nos. 5,128,256 and 5,286,636), pBluescript (pBS) (Short, J. M. et al., Nucleic Acids Res. 16:7583-7600 (1988); Alting-Mees, M. A. and Short, J. M., Nucleic Acids Res. 17:9494 (1989)) and pBK (Alting-Mees, M. A. et al., Strategies 5:58-61 (1992)) are commercially available from Stratagene Cloning Systems, Inc., 11011 N. Torrey Pines Road, La Jolla, Calif., 92037. pBS contains an ampicillin resistance gene and pBK contains a neomycin resistance gene. Both can be transformed into E. coli strain XL-1 Blue, also available from Stratagene. pBS comes in 4 forms SK+, SK−, KS+ and KS. The S and K refers to the orientation of the polylinker to the T7 and T3 primer sequences which flank the polylinker region (“S” is for SacI and “K” is for KpnI which are the first sites on each respective end of the linker). “+” or “−” refer to the orientation of the f1 origin of replication (“ori”), such that in one orientation, single stranded rescue initiated from the f1 ori generates sense strand DNA and in the other, antisense. Vectors pSport1, pCMVSport 2.0 and pCMVSport 3.0, were obtained from Life Technologies, Inc., P. O. Box 6009, Gaithersburg, Md. 20897. All Sport vectors contain an ampicillin resistance gene and may be transformed into E. coli strain DH10B, also available from Life Technologies. (See, for instance, Gruber, C. E., et al., Focus 15:59 (1993).) Vector lafmid BA (Bento Soares, Columbia University, NY) contains an ampicillin resistance gene and can be transformed into E. coli strain XL-1 Blue. Vector pCR®2.1, which is available from Invitrogen, 1600 Faraday Avenue, Carlsbad, Calif. 92008, contains an ampicillin resistance gene and may be transformed into E. coli strain DH10B, available from Life Technologies. (See, for instance, Clark, J. M., Nuc. Acids Res. 16:9677-9686 (1988) and Mead, D. et al., Bio/Technology 9: (1991).) Preferably, a polynucleotide of the present invention does not comprise the phage vector sequences identified for the particular clone in Table 1, as well as the corresponding plasmid vector sequences designated above.

The deposited material in the sample assigned the ATCC Deposit Number cited in Table 1 for any given cDNA clone also may contain one or more additional plasmids, each comprisirrg a cDNA clone different from that given clone. Thus, deposits sharing the same ATCC Deposit Number contain at least a plasmid for each cDNA clone identified in Table 1. Typically, each ATCC deposit sample cited in Table 1 comprises a mixture of approximately equal amounts (by weight) of about 50 plasmid DNAs, each containing a different cDNA clone; but such a deposit sample may include plasmids for more or less than 50 cDNA clones, up to about 500 cDNA clones.

Two approaches can be used to isolate a particular clone from the deposited sample of plasmid DNAs cited for that clone in Table 1. First, a plasmid is directly isolated by screening the clones using a polynucleotide probe corresponding to SEQ ID NO:X.

Particularly, a specific polynucleotide with 30-40 nucleotides is synthesized using an Applied Biosystems DNA synthesizer according to the sequence reported. The oligonucleotide is labeled, for instance, with ³²P-γ-ATP using T4 polynucleotide kinase and purified according to routine methods. (E.g., Maniatis et al., Molecular Cloning: A Laboratory Manual, Cold Spring Harbor Press, Cold Spring, N.Y. (1982).) The plasmid mixture is transformed into a suitable host, as indicated above (such as XL-1 Blue (Stratagene)) using techniques known to those of skill in the art, such as those provided by the vector supplier or in related publications or patents cited above. The transformants are plated on 1.5% agar plates (containing the appropriate selection agent, e.g., ampicillin) to a density of about 150 transformants (colonies) per plate. These plates are screened using Nylon membranes according to routine methods for bacterial colony screening (e.g., Sambrook et al., Molecular Cloning: A Laboratory Manual, 2nd Edit., (1989), Cold Spring Harbor Laboratory Press, pages 1.93 to 1.104), or other techniques known to those of skill in the art.

Alternatively, two primers of 17-20 nucleotides derived from both ends of the SEQ ID NO:X (i.e., within the region of SEQ ID NO:X bounded by the 5′ NT and the 3′ NT of the clone defined in Table 1) are synthesized and used to amplify the desired cDNA using the deposited cDNA plasmid as a template. The polymerase chain reaction is carried out under routine conditions, for instance, in 25 μl of reaction mixture with 0.5 ug of the above cDNA template. A convenient reaction mixture is 1.5-5 mM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 nin; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of a gene which may not be present in the deposited clone. These methods include but are not limited to, filter probing, clone enrichment using specific probes, and protocols similar or identical to 5′ and 3′ “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).) Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene.

### Example 2

### Isolation of Genomic Clones Corresponding to a Polynucleotide

A human genomic P1 library (Genomic Systems, Inc.) is screened by PCR using primers selected for the cDNA sequence corresponding to SEQ ID NO:X., according to the method described in Example 1. (See also, Sambrook.)

### Example 3

### Tissue Distribution of Polypeptide

Tissue distribution of mRNA expression of polynucleotides of the present invention is determined using protocols for Northern blot analysis, described by, among others, Sambrook et al. For example, a cDNA probe produced by the method described in Example 1 is labeled with p³² using the rediprime™ DNA labeling system (Amersham Life Science), according to manufacturer's instructions. After labeling, the probe is purified using CHROMA SPIN-100Tm column (Clontech Laboratories, Inc.), according to manufacturer's protocol number PT1200-1. The purified labeled probe is then used to examine various human tissues for mRNA expression.

Multiple Tissue Northern (MTN) blots containing various human tissues (H) or human immune system tissues (IM) (Clontech) are examined with the labeled probe using ExpressHyb™ hybridization solution (Clontech) according to manufacturer's protocol number PT1190-1. Following hybridization and washing, the blots are mounted and exposed to film at −70° C. ovemight, and the films developed according to standard procedures.

### Example 4

### Chromosomal Mapping of the Polynucleotides

An oligonucleotide primer set is designed according to the sequence at the 5′ end of SEQ ID NO:X. This primer preferably spans about 100 nucleotides. This primer set is then used in a polymerase chain reaction under the following set of conditions: 30 seconds, 95° C.; 1 minute, 56° C.; 1 minute, 70° C. This cycle is repeated 32 times followed by one 5 minute cycle at 70° C. Human, mouse, and hamster DNA is used as template in addition to a somatic cell hybrid panel containing individual chromosomes or chromosome fragments (Bios, Inc). The reactions is analyzed on either 8% polyacrylamide gels or 3.5% agarose gels. Chromosome mapping is determined by the presence of an approximately 100 bp PCR fragment in the particular somatic cell hybrid.

### Example 5

### Bacterial Expression of a Polypeptide

A polynucleotide encoding a polypeptide of the present invention is amplified using PCR oligonucleotide primers corresponding to the 5′ and 3′ ends of the DNA sequence, as outlined in Example 1, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000× g). The cell pellet is solubilized in the chaotropic agent 6 Molar Guanidine HCl by stirring for 3-4 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QlAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 niM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified protein is stored at 4° C. or frozen at −80° C.

In addition to the above expression vector, the present invention further includes an expression vector comprising phage operator and promoter elements operatively linked to a polynucleotide of the present invention, called pHE4a. (ATCC Accession Number 209645, deposited on Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically.

DNA can be inserted into the pHEa by restricting the vector with NdeI and XbaI, BamHI, XhoI, or Asp718, running the restricted product on a gel, and isolating the larger fragment (the stuffer fragment should be about 310 base pairs). The DNA insert is generated according to the PCR protocol described in Example 1, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 6

### Purification of a Polypeptide from an Inclusion Body

The following alternative method can be used to purify a polypeptide expressed in E coli when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then rruxed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000× g centrifugation for 15 min., the pellet is discarded and the polypeptide containing supernatant is incubated at 4° C. overnight to allow further GuHCl extraction.

Following high speed centrifugation (30,000× g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 μm membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Commassie blue stained 16% SDS-PAGE gel when 5 μg of purified protein is loaded. The purified protein can also be tested for endotoxin/LPS contamination, and typically the LPS content is less than 0.1 ng/ml according to LAL assays.

### Example 7

### Cloning and Expression of a Polypeptide in a Baculovirus Expression System

In this example, the plasmid shuttle vector pA2 is used to insert a polynucleotide into a baculovirus to express a polypeptide. This expression vector contains the strong polyhedrin promoter of the Autographa californica nuclear polyhedrosis virus (AcMNPV) followed by convenient restriction sites such as BamHI, Xba I and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Mahy other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the cDNA sequence contained in the deposited clone, including the AUG initiation codon and the naturally associated leader sequence identified in Table 1, is amplified using the PCR protocol described in Example 1. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five μg of a plasmid containing the polynucleotide is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One μg of BaculoGold TMvirus DNA and 5 μg of the plasmid are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced protein.

### Example 8

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human, actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-dhfr (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of the gene of interest. The vectors also contain the 3′ intron, the polyadenylation and termination signal of the rat preproinsulin gene, and the mouse DHFR gene under control of the SV40 early promoter.

Specifically, the plasmid pC6, for example, is digested with appropriate restriction enzymes and then dephosphorylated using calf intestinal phosphates by procedures known in the art. The vector is then isolated from a 1% agarose gel.

A polynucleotide of the present invention is amplified according to the protocol outlined in Example 1. If the naturally occurring signal sequence is used to produce the secreted protein, the vector does not need a second signal peptide. Alternatively, if the naturally occurring signal sequence is not used, the vector can be modified to include a heterologous signal sequence. (See, e.g., WO 96/34891.)

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transfection. Five μg of the expression plasmid pC6 is cotransfected with 0.5 μg of the plasmid pSVneo using lipofectin (Felgner et al., slipra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/mil G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 9

### Protein Fusions

The polypeptides of the present invention are preferably fused to other proteins. These fusion proteins can be used for a variety of applications. For example, fusion of the present polypeptides to His-tag, HA-tag, protein A, IgG domains, and maltose binding protein facilitates purification. (See Example 5; see also EP A 394,827; Traunecker, et al., Nature 331:84-86 (1988).) Similarly, fusion to IgG-1, IgG-3, and albumin increases the halflife time in vivo. Nuclear localization signals fused to the polypeptides of the present invention can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 5.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and a polynucleotide of the present invention, isolated by the PCR protocol described in Example 1, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fe region:

(SEQ ID NO:1)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAACC
ACAGGTGTACACCCTOCCCCCATCCCGGGATGAGCTGACCAAGAACCAGG
TCAGCCTGACCTGCCTGGTCAAAGGCTTCTATCCAAGCGACATCGCCGTG
GAGTGGGAGAGCAATGGGCAGCCGGAGAACAACTACAAGACCACGCCTCC
CGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCAT
GAGGCTCTGCACAACCACTACACGCAGAAGAGCCTCTCCCTGTCTCCGGG
TAAATGAGTGCGACGGCCGCGACTCTAGAGGAT

### Example 10

### Production of an Antibody from a Polypeptide

The antibodies of the present invention can be prepared by a variety of methods. (See, Current Protocols, Chapter 2.) For example, cells expressing a polypeptide of the present invention is administered to an animal to induce the production of sera containing polyclonal antibodies. In a preferred method, a preparation of the secreted protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a secreted polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, secreted protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 11

### Production of Secreted Protein for High-Throughput Screening Assays

The following protocol produces a supernatant containing a polypeptide to be tested. This supernatant can then be used in the Screening Assays described in Examples 13-20.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 ug/ml. Add 200 ul of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM(Dulbecco's Modified Eagle Medium)(with 4.5 G/L glucose and L-glutamine (12-604F Biowhittaker))/10% heat inactivated FBS(14-503F Biowhittaker)/1× Penstrep(17-602E Biowhittaker). Let the cells grow overnight.

The next day, mix together in a sterile solution basin: 300 ul Lipofectamine (18324-012 Gibco/BRL) and 5 ml Optimem I (31985070 Gibco/BRL)/96-well plate. With a small volume multi-channel pipetter, aliquot approximately 2 ug of an expression vector containing a polynucleotide insert, produced by the methods described in Examples 8 or 9, into an appropriately labeled 96-well round bottom plate. With a multi-channel pipetter, add 50 ul of the Lipofectamine/Optimem I mixture to each well. Pipette up and down gently to mix. Incubate at RT 15-45 minutes. After about 20 minutes, use a multi-channel pipetter to add 150 ul Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 mL PBS. Person A then aspirates off PBS rinse, and person B, using al2-channel pipetter with tips on every other channel, adds the 200 ul of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with 1× penstrep, or CHO-5 media (116.6 mg/L of CaCl2 (anhyd); 0.00130 mg/L CuSO₄.5H₂O; 0.050 mg/L of Fe(NO₃)₃-9H₂O; 0.417 mg/L of FeSO₄.7H₂O; 311.80 mg/L of Kcl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO4; 0.4320 mg/L of ZnSO₄0.7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L—Alanine; 147.50 mg/ml of L—Arginine-HCL; 7.50 mg/ml of L-Asparagine-H₂O; 6.65 mg/mil of L-Aspartic Acid; 29.56 mg/ml of L-Cystine-2HCL-H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL-H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/ml of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; and 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; and 10 mg/L of Methyl-B-Cyclodextrin complexed with Retinal) with 2 mm glutamine and 1× penstrep. (BSA (81-068-3 Bayer) 100gm dissolved in IL DMEM for a 10% BSA stock solution). Filter the media and collect 50 ul for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 ul multichannel pipetter, aliquot 600 ul in one 1 ml deep well plate and the remaining supernatant into a 2 ml deep well. The supernatants from each well can then be used in the assays described in Examples 13-20.

It is specifically understood that when activity is obtained in any of the assays described below using a supernatant, the activity originates from either the polypeptide directly (e.g.; as a secreted protein) or by the polypeptide inducing expression of other proteins, which are then secreted into the supernatant. Thus, the invention further provides a method of identifying the protein in the supernatant characterized by an activity in a particular assay.

### Example 12

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, 1L-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proximal region encoding Trp-Ser-Xxx-Trp-Ser (SEQ ID NO:2)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | JAKs | | | | | |
|---|---|---|---|---|---|---|
| Ligand | tyk2 | Jak1 | Jak2 | Jak3 | STATS | GAS (elements) or ISRE |
| IFN family | | | | | | |
| IFN-a/B | + | + | − | − | 1,2,3 | ISRE |
| IFN-g | | + | + | − | 1 | GAS (IRF1 > Lys6 > IFP) |
| Il-10 | + | ? | ? | − | 1,3 | |
| gp130 family | | | | | | |
| IL-6 (Pleiotropic) | + | + | + | ? | 1,3 | GAS (IRF1 > Lys6 > IFP) |
| Il-11 (Pleiotropic) | ? | + | ? | ? | 1,3 | |
| OnM (Pleiotropic) | ? | + | + | ? | 1,3 | |
| LIF (Pleiotropic) | ? | + | + | ? | 1,3 | |
| CNTF (Pleiotropic) | −/+ | + | + | ? | 1,3 | |
| G-CSF (Pleiotropic) | ? | + | ? | ? | 1,3 | |
| IL-12 (Pleiotropic) | + | − | + | + | 1,3 | |
| g-C family | | | | | | |
| IL-2 (lymphocytes) | − | + | − | + | 1,3,5 | GAS |
| IL-4 (lymph/myeloid) | − | + | − | + | 6 | GAS (IRF1 = IFP >> Ly6)(IgH) |
| IL-7 (lymphocytes) | − | + | − | + | 5 | GAS |
| IL-9 (lymphocytes) | − | + | − | + | 5 | GAS |
| IL-13 (lymphocyte) | − | + | ? | ? | 6 | GAS |
| IL-15 | ? | + | ? | + | 5 | GAS |
| gp140 family | | | | | | |
| IL-3 (myeloid) | − | − | + | − | 5 | GAS (IRF1 > IFP >> Ly6) |
| IL-5 (myeloid) | − | − | + | − | 5 | GAS |
| GM-CSF (myeloid) | − | − | + | − | 5 | GAS |
| Growth hormone family | | | | | | |
| GH | ? | − | + | − | 5 | |
| PRL | ? | +/− | + | − | 1,3,5 | |
| EPO | ? | − | + | − | 5 | GAS (B-CAS > IRF1 = IFP >> Ly6) |
| Receptor Tyrosine Kinases | | | | | | |
| EGF | ? | + | + | − | 1,3 | GAS (IRF1) |
| PDGF | ? | + | + | − | 1,3 | |
| CSF-1 | ? | + | + | − | 1,3 | GAS (not IRF1) |

To construct a synthetic GAS containing promoter element, which is used in the Biological Assays described in Examples 13-14, a PCR based strategy is employed to generate a GAS-SV40 promoter sequence. The 5′ primer contains four tandem copies of the GAS binding site found in the IRFI promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

(SEQ ID NO:3)

5′:GCGCCTCGAGATTTCCCCGAAATCTAGATTTCCCCGAAATGATTTCC
CCGAAATGATTTCCCCGAAATATCTGCCATCTCAATTAG:3′

The downstream primer is complementary to the SV40 promoter and is flanked with a Hind III site: 5′:GCGGCAAGCTTTTTGCAAAGCCTAGGC:3′ (SEQ ID NO:4)

PCR amplification is performed using the SV40 promoter template present in the B-gal:promoter plasmid obtained from Clontech. The resulting PCR fragment is digested with XhoI/Hind III and subcloned into BLSK2-. (Stratagene.) Sequencing with forward and reverse primers confirms that the insert contains the following sequence:

(SEQ ID NO:5)

5′:CTCGAGATTTCCCCGAAATCTAGATTTCCCCGAAATGATTTCCCCGA
AATGATTTCCCCGAAATATCTGCCATCTCAATTAGTCAGCAACCATAGTC
CCGTGATCCCCTAACTCCGCCCATCCCGCCCCTAACTCCGCCCAGTTCCG
CCCATTCTTGATCCGCCCCATGGCTGACTAATTTTTTTTATTTATGCAGA
GGCCGAGGCCGCCTGATTCGGCCTCTGAGCTATTCCAGAAGTAGTGAGGA
GGCTTTTTTGGAGGCCTTGATAGGCTTTTGCAAAAAGCTT:3′

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can be instead of SEAP, in this or in any of the other Examples. Well known reporter molecules that can be used instead of SEAP include chloramphenicol acetyltransferase (CAT), luciferase, alkaline phosphatase, B-galactosidase, green fluorescent protein (GFP), or any protein detectable by an antibody.

The above sequence confirmed synthetic GAS-SV40 promoter element is subcloned into the pSEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 13-14.

Other constructs can be made using the above description and replacing GAS with a different promoter sequence. For example, construction of reporter molecules containing NFK-B and EGR promoter sequences are described in Examples 15 and 16. However, many other promoters can be substituted using the protocols described in these Examples. For instance, SRE, IL-2, NFAT, or Osteocalcin promoters can be substituted, alone or in combination (e.g., GAS/NF-KB/EGR, GAS/NF-KB, II-2/NFAT, or NF-KB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 13

### High-Throughput Screening Assay for T-cell Activity

The following protocol is used to assess T-cell activity by identifying factors, such as growth factors and cytokines, that may proliferate or differentiate T-cells. T-cell activity is assessed using the GAS/SEAP/Neo construct produced in Example 12. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4+ Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 ul of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 ug of plasmid DNA in a T25 flask. Add 2.5 ml OPTI-MEM containing 50 ul of DMRIE-C and incubate at room temperature for 15-45 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing a polypeptide as produced by the protocol described in Example 11.

On the day of treatment with the supernatant, the cells should be washed and resuspended in fresh, RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 ul of cells into each well (therefore adding 100, 000 cells per well).

After all the plates have been seeded, 50 ul of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 ul samples from each well are then transferred to an opaque 96 well plate using a 12 channel pipette. The opaque plates should be covered (using sellophene covers) and stored at −20° C. until SEAP assays are performed according to Example 17. The plates containing the remaining treated cells are placed at 4° C. and serve as a source of material for repeating the assay on a specific well if desired.

As a positive control, 100 Unit/rl interferon gamma can be used which is known to activate Jurkat T cells. Over 30 fold induction is typically observed in the positive control wells.

The above protocol may be used in the generation of both transient, as well as, stable transfected cells, which would be apparent to those of skill in the art.

### Example 14

### High-Throughput Screening Assay Identifying Myeloid Activity

The following protocol is used to assess myeloid activity by identifying factors, such as growth factors and cytokines, that may proliferate or differentiate myeloid cells. Myeloid cell activity is assessed using the GAS/SEAP/Neo construct produced in Example 12. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 12, a DEAE-Dextran method (Kharbanda et. al., 1994, Cell Growth & Differentiation, 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄.7H₂O, 1 mM MgCl₂, and 675 uM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAPIU937 stable cells are obtained by growing the cells in 400 ug/ml G418. The G418-free medium is used for routine growth but every one to two months, the cells should be re-grown in 400 ug/mil G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 ul cells per well in the 96-well plate (or 1×10⁵ cells/well).

Add 50 ul of the supernatant prepared by the protocol described in Example 11. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 17.

### Example 15

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC 12 cells can be assessed.

The EGR/SEAP reporter construct can be assembled by the following protocol. The EGR-1 promoter sequence (-633 to +1)(Sakamoto K et al., Oncogene 6:867-871 (1991)) can be PCR amplified from human genomic DNA using the following primers:

| | |
|---|---|
| 5′ GCGCTCGAGGGATGACAGCGATAGAACCCCGG-3′ | (SEQ ID NO:6) |
| 5′ GCGAAGCTTCGCGACTCCCCGGATCCGCCTC-3′ | (SEQ ID NO:7) |

Using the GAS:SEAP/Neo vector produced in Example 12, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC 12 using the Lipofectamine protocol described in Example 11. EGR-SEAP/PC12 stable cells are obtained by growing the cells in 300 ugmil G418. The G418-free medium is used for routine growth but every one to two months, the cells should be re-grown in 300 ug/ml G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 ul of the cell suspension to each well of 96-well plate (equivalent to 1×10⁵ cells/well). Add 50 ul supernatant produced by Example 11, 37° C. for 48 to 72 hr. As a positive control, a growth factor known to activate PCl₂ cells through EGR can be used, such as 50 ng/ul of Neuronal Growth Factor (NGF). Over fifty-fold induction of SEAP is typically seen in the positive control wells. SEAP assay the supernatant according to Example 17.

### Example 16

### High-Throughput Screening Assay for T-cell Activity

NF-κB (Nuclear Factor κB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-κB regulates the expression of genes involved in immune cell activation, control of apoptosis (NF-κB appears to shield cells from apoptosis), B and T-cell development, anti-viral and antimicrobial responses, and multiple stress responses.

In non-stimulated conditions, NF-κB is retained in the cytoplasm with I-κB (Inhibitor κB). However, upon stimulation, I-κB is phosphorylated and degraded, causing NF-κB to shuttle to the nucleus, thereby activating transcription of target genes. Target genes activated by NF-κB include IL-2, IL-6, GM-CSF, ICAM-1 and class 1 MHC.

Due to its central role and ability to respond to a range of stimuli, reporter constructs utilizing the NF-κB promoter element are used to screen the supernatants produced in Example 11. Activators or inhibitors of NF-kB would be useful in treating diseases. For example, inhibitors of NF-κB could be used to treat those diseases related to the acute or chronic activation of NF-KB, such as rheumatoid arthritis.

To construct a vector containing the NF-κB promoter element, a PCR based strategy is employed. The upstream primer contains four tandem copies of the NF-κB binding site (GGGGACTTTCCC) (SEQ ID NO:8), 18 bp of sequence complementary to the 5′ end of the SV40 early promoter sequence, and is flanked with an XhoI site:

(SEQ ID NO:9)

5′:GCGGCCTCGAGGGGACTTTCCCGGGGACTTTCCGGGGACTTTCCGGG
ACTTTCCATCCTGCCATCTCAATTAG:3′

The downstream primer is complementary to the 3′ end of the SV40 promoter and is flanked with a Hind III site:

5′:GCGGCAAGCTTTTTGCAAAGCCTAGGC:3′ (SEQ ID NO:4)

PCR amplification is performed using the SV40 promoter template present in the pB-gal:promoter plasmid obtained from Clontech. The resulting PCR fragment is digested with XhoI and Hind III and subcloned into BLSK2-. (Stratagene) Sequencing with the T7 and T3 primers confirms the insert contains the following sequence:

(SEQ ID NO:10)

5′:CTCGAGGGGACTTTCCCGGGGACTTTCCGGGGACTTTCCGGGACTTT
CCATCTGCCATCTCAATTAGTCAGCAACCATAGTCCCGCCCCTAACTCCG
CCCATCCCGCCCCTAACTCCGCCCAGTTCCGCCCATTCTCCGCCCCATGG
CTGACTAATTTTTTTTATTTATGCAGAGGCCGAGGCCGCCTCGGCCTCTG
AGCTATTCCAGAAGTAGTGAGGAGGCTTTTTTGGAGGCCTAGGCTTTTGC
AAAAAGCTT:3′

Next, replace the SV40 minimal promoter element present in the pSEAP2-promoter plasmid (Clontech) with this NF-κB/SV40 fragment using XhoI and HindIII. However, this vector does not contain a neomycin resistance gene, and therefore, is not preferred for mammalian expression systems.

In order to generate stable mammalian cell lines, the NF-κB/SV40/SEAP cassette is removed from the above NF-KB/SEAP vector using restriction enzymes SalI and NotI, and inserted into a vector containing neomycin resistance. Particularly, the NF-κB/SV40/SEAP cassette was inserted into pGFP-1 (Clontech), replacing the GFP gene, after restricting pGFP-1 with SalI and NotI.

Once NF-κB/SV40/SEAP/Neo vector is created, stable Jurkat T-cells are created and maintained according to the protocol described in Example 13. Similarly, the method for assaying supernatants with these stable Jurkat T-cells is also described in Example 13. As a positive control, exogenous TNF alpha (0.1, 1, 10 ng) is added to wells H9, H10, and H11, with a 5-10 fold activation typically observed.

### Example 17

### Assay for SEAP Activity

As a reporter molecule for the assays described in Examples 13-16, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 μl of 2.5× dilution buffer into Optiplates containing 35 μl of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 μl Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

### Example 18

### High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-4 (Molecular Probes, Inc. catalog no. F-14202), used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 ul of HBSS (Hank's Balanced Salt Solution) leaving 100 ul of buffer after the final wash.

A stock solution of 1 mg/ml fluo-4 is made in 10% pluronic acid DMSO. To load the cells with fluo-4, 50 ul of 12 ug/ml fluo-4 is added to each well. The plate is incubated at 37° C. in a CO₂ incubator for 60 min. The plate is washed four times in the Biotek washer with HBSS leaving 100 ul of buffer.

For non-adherent cells, the cells are spun down from culture media. Cells are re-suspended to 2-5×10⁶ cells/ml with HBSS in a 50-ml conical tube. 4 ul of 1 mg/ml fluo-4 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 ul/well. The plate is centrifuged at 1000 rpm for 5 min. The plate is then washed once in Denley CellWash with 200 ul, followed by an aspiration step to 100 ul final volume.

For a non-cell based assay, each well contains a fluorescent molecule, such as fluo-4. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 ul. Increased emission at 530 nm indicates an extracellular signaling event which has resulted in an increase in the intracellular Ca⁺⁺ concentration.

### Example 19

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, Ick, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, the identification of novel human secreted proteins capable of activating tyrosine kinase signal transduction pathways are of interest. Therefore, the following protocol is designed to identify those novel human secreted proteins capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 40C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 ninutes treatment with EGF (60 ng/ml) or 50 ul of the supernatant produced in Example 11, the medium was removed and 100 ml of extraction buffer ((20 mM HEPES pH 7.5, 0.15 M NaCl, 1% Triton X-100, 0.1% SDS, 2 mM Na3VO4, 2 mM Na4P2O7 and a cocktail of protease inhibitors (#1836170) obtained from Boeheringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000× g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSKI (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5uM Biotinylated Peptide, then 10 ul ATP/Mg₂₊ (5 mM ATP/50 mM MgCl₂), then 10 ul of 5× Assay Buffer (40 mM imidazole hydrochloride, pH 7.3, 40 mM beta-glycerophosphate, 1 mM EGTA, 100 mM MgCl₂, 5 mM MnCl₂, 0.5 mg/ml BSA), then 5 ul of Sodium Vanadate(1 mM), and then 5 ul of water. Mix the components gently and preincubate the reaction mix at 30° C. for 2 min. Initial the reaction by adding 10 ul of the control enzyme or the filtered supernatant.

The tyrosine kinase assay reaction is then terminated by adding 10 ul of 120mm EDTA and place the reactions on ice.

Tyrosine kinase activity is determined by transferring 50 ul aliquot of reaction mixture to a microtiter plate (MTP) module and incubating at 37° C. for 20 min. This allows the streptavadin coated 96 well plate to associate with the biotinylated peptide. Wash the MTP module with 300 ul/well of PBS four times. Next add 75 ul of anti-phospotyrosine antibody conjugated to horse radish peroxidase(anti-P-Tyr-POD(0.5 u/ml)) to each well and incubate at 37° C. for one hour. Wash the well as above.

Next add 100 ul of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 20

### High-Throughput Screening Assay Identifying Phosphorylation Activity

As a potential alternative and/or compliment to the assay of protein tyrosine kinase activity described in Example 19, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), TRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.ml of protein G (1 ug/ml) for 2 hr at room temp, (RT). The plates are then rinsed with PBS and blocked with 3% BSA/PBS for 1 hr at RT. The protein G plates are then treated with 2 commercial monoclonal antibodies (1000 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 40° C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 ul of the supernatants obtained in Example 11 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (lug/mil) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation.

### Example 21

### Method of Determining Alterations in a Gene Corresponding to a Polynucleotide

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:X. Suggested PCR conditions consist of 35 cycles at 95° C. for 30 seconds; 60-120 seconds at 52-58° C.; and 60-120 seconds at 70° C., using buffer solutions described in Sidransky, D., et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons is also determined and genomic PCR products analyzed to confirm the results. PCR products harboring suspected mutations is then cloned and sequenced to validate the results of the direct sequencing.

PCR products is cloned into T-tailed vectors as described in Holton, T. A. and Graham, M. W., Nucleic Acids Research, 19:1156 (1991) and sequenced with T7 polymerase (United States Biochemical). Affected individuals are identified by mutations not present in unaffected individuals.

Genomic rearrangements are also observed as a method of determining alterations in a gene corresponding to a polynucleotide. Genomic clones isolated according to Example 2 are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the corresponding genomic locus.

Chromosomes are counterstained with 4,6-diamino-2-phenylidole and propidium iodide, producing a combination of C- and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl., 8:75 (1991).) Image collection, analysis and chromosomal fractional length measurements are performed using the ISee Graphical Program System. (Inovision Corporation, Durham, N.C.) Chromosome alterations of the genomic region hybridized by the probe are identified as insertions, deletions, and translocations. These alterations are used as a diagnostic marker for an associated disease.

### Example 22

### Method of Detecting Abnormal Levels of a Polypeptide in a Biological Sample

A polypeptide of the present invention can be detected in a biological sample, and if an increased or decreased level of the polypeptide is detected, this polypeptide is a marker for a particular phenotype. Methods of detection are numerous, and thus, it is understood that one skilled in the art can modify the following assay to fit their particular needs.

For example, antibody-sandwich ELISAs are used to detect polypeptides in a sample, preferably a biological sample. Wells of a microtiter plate are coated with specific antibodies, at a final concentration of 0.2 to 10 ug/ml. The antibodies are either monoclonal or polyclonal and are produced by the method described in Example 10. The wells are blocked so that non-specific binding of the polypeptide to the well is reduced.

The coated wells are then incubated for >2 hours at RT with a sample containing the polypeptide. Preferably, serial dilutions of the sample should be used to validate results. The plates are then washed three times with deionized or distilled water to remove unbounded polypeptide.

Next, 50 ul of specific antibody-alkaline phosphatase conjugate, at a concentration of 25-400 ng, is added and incubated for 2 hours at room temperature. The plates are again washed three times with deionized or distilled water to remove unbounded conjugate.

Add 75 ul of 4-methylumbelliferyl phosphate (MUP) or p-nitrophenyl phosphate (NPP) substrate solution to each well and incubate 1 hour at room temperature. Measure the reaction by a microtiter plate reader. Prepare a standard curve, using serial dilutions of a control sample, and plot polypeptide concentration on the X-axis (log scale) and fluorescence or absorbance of the Y-axis (linear scale). Interpolate the concentration of the polypeptide in the sample using the standard curve.

### Example 23

### Formulating a Polypeptide

The secreted polypeptide composition will be formulated and dosed in a fashion consistent with good medical practice, taking into account the clinical condition of the individual patient (especially the side effects of treatment with the secreted polypeptide alone), the site of delivery, the method of adninistration, the scheduling of administration, and other factors known to practitioners. The “effective amount” for purposes herein is thus determined by such considerations.

As a general proposition, the total pharmaceutically effective amount of secreted polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the secreted polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the secreted protein of the invention are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The secreted polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(−)-3-hydroxybutyric acid (EP 133,988). Sustained-release compositions also include liposomally entrapped polypeptides. Liposomes containing the secreted polypeptide are prepared by methods known per se: DE 3,218,121; Epstein et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, the secreted polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The secreted polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

Any polypeptide to be used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

Polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous polypeptide solution, and the resulting mixture is lyophilized. The infusion solution is prepared by reconstituting the lyophilized polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

### Example 24

### Method of Treating Decreased Levels of the Polypeptide

It will be appreciated that conditions caused by a decrease in the standard or normal expression level of a secreted protein in an individual can be treated by administering the polypeptide of the present invention, preferably in the secreted form. Thus, the invention also provides a method of treatment of an individual in need of an increased level of the polypeptide comprising administering to such an individual a pharmaceutical composition comprising an amount of the polypeptide to increase the activity level of the polypeptide in such an individual.

For example, a patient with decreased levels of a polypeptide receives a daily dose 0.1-100 ug/kg of the polypeptide for six consecutive days. Preferably, the polypeptide is in the secreted form. The exact details of the dosing scheme, based on administration and formulation, are provided in Example 23.

### Example 25

### Method of Treating Increased Levels of the Polypeptide

Antisense technology is used to inhibit production of a polypeptide of the present invention. This technology is one example of a method of decreasing levels of a polypeptide, preferably a secreted form, due to a variety of etiologies, such as cancer.

For example, a patient diagnosed with abnormally increased levels of a polypeptide is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense f polynucleotide is provided in Example 23.

### Example 26

### Method of Treatment Using Gene Therapy

One method of gene therapy transplants fibroblasts, which are capable of expressing a polypeptide, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al., DNA, 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention can be amplified using PCR primers which correspond to the 5′ and 3′ end sequences respectively as set forth in Example 1. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB101, which are then plated onto agar containing kanamycin for the purpose of confirming that the vector has the gene of interest properly inserted.

The arnphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether protein is produced.

The engineered fibroblasts are then transplanted onto the host, either alone or after having been grown to confluence on cytodex 3 microcarrier beads.

### Example 27

### Method of Treatment Using Gene Therapy—in vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. Nos. 5,693,622, 5,705,151, 5,580,859; Tabata H. et al. (1997) Cardiovasc. Res. 35(3):470-479, Chao J et al. (1997) Pharmacol. Res. 35(6):517-522, Wolff J.A. (1997) Neuromuscul. Disord. 7(5):314-318, Schwartz B. et al. (1996) Gene Ther. 3(5):405-411, Tsurumi Y. et al. (1996) Circulation 94(12):3281-3290 (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Felgner P. L. et al. (1995) Ann. NY Acad. Sci. 772:126-139 and Abdallah B. et al. (1995) Biol. Cell 85(1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 28

### Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tall tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

### Example 29

### Knock-Out Animals

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying conditions and/or disorders associated with aberrant expression, and in screening for compounds effective in ameliorating such conditions and/or disorders.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

What is claimed is:

1. An isolated nucleic acid molecule comprising a polynucleotide having a nucleotide sequence at least 95% identical to a sequence selected from the group consisting of:
(a) a polynucleotide fragment of SEQ ID NO:X or a polynucleotide fragment of the cDNA sequence included in ATCC Deposit No:Z, which is hybridizable to SEQ ID NO:X;
(b) a polynucleotide encoding a polypeptide fragment of SEQ ID NO:Y or a polypeptide fragment encoded by the cDNA sequence included in ATCC Deposit No:Z, which is hybridizable to SEQ ID NO:X;
(c) a polynucleotide encoding a polypeptide domain of SEQ ID NO:Y or a polypeptide domain encoded by the cDNA sequence included in ATCC Deposit No:Z, which is hybridizable to SEQ ID NO:X;
(d) a polynucleotide encoding a polypeptide epitope of SEQ ID NO:Y or a polypeptide epitope encoded by the cDNA sequence included in ATCC Deposit No:Z, which is hybridizable to SEQ ID NO:X;
(e) a polynucleotide encoding a polypeptide of SEQ ID NO:Y or the cDNA sequence included in ATCC Deposit No:Z, which is hybridizable to SEQ ID NO:X, having biological activity;
(f) a polynucleotide which is a variant of SEQ ID NO:X;
(g) a polynucleotide which is an allelic variant of SEQ ID NO:X;
(h) a polynucleotide which encodes a species homologue of the SEQ ID NO:Y;
(i) a polynucleotide capable of hybridizing under stringent conditions to any one of the polynucleotides specified in (a)-(h), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment comprises a nucleotide sequence encoding a secreted protein.

3. The isolated nucleic acid molecule of claim 1, wherein the polynucleotide fragment comprises a nucleotide sequence encoding the sequence identified as SEQ ID NO:Y or the polypeptide encoded by the cDNA sequence included in ATCC Deposit No:Z, which is hybridizable to SEQ ID NO:X.

4. The isolated nucleic acid molecule of claim 1, wherein the polynucleotide fragment comprises the entire nucleotide sequence of SEQ ID NO:X or the cDNA sequence included in ATCC Deposit No:Z, which is hybridizable to SEQ ID NO:X.

5. The isolated nucleic acid molecule of claim 2, wherein the nucleotide sequence comprises sequential nucleotide deletions from either the C-terminus or the N-terminus.

6. The isolated nucleic acid molecule of claim 3, wherein the nucteotide sequence comprises sequential nucleotide deletions from either the C-terminus or the N-terminus.

7. A recombinant vector comprising the isolated nucleic acid molecule of claim 1.

8. A method of making a recombinant host cell comprising the isolated nucleic acid molecule of claim 1.

9. A recombinant host cell produced by the method of claim 8.

10. The recombinant host cell of claim 9 comprising vector sequences.

11. An isolated polypeptide comprising an amino acid sequence at least 95% identical to a sequence selected from the group consisting of:
(a) a polypeptide fragment of SEQ ID NO:Y or the encoded sequence included in ATCC Deposit No:Z;
(b) a polypeptide fragment of SEQ ID NO:Y or the encoded sequence included in ATCC Deposit No:Z, having biological activity;
(c) a polypeptide domain of SEQ ID NO:Y or the encoded sequence included in ATCC Deposit No:Z;
(d) a polypeptide epitope of SEQ ID NO:Y or the encoded sequence included in ATCC Deposit No:Z;
(e) a secreted form of SEQ ID NO:Y or the encoded sequence included in ATCC Deposit No:Z;
(f) a full length protein of SEQ ID NO:Y or the encoded sequence included in ATCC Deposit No:Z;
(g) a variant of SEQ ID NO:Y;
(h) an allelic variant of SEQ ID NO:Y; or
(i) a species homologue of the SEQ ID NO:Y.

12. The isolated polypeptide of claim 11, wherein the secreted form or the full length protein comprises sequential amino acid deletions from either the C-terminus or the N-terminus.

13. An isolated antibody that binds specifically to the isolated polypeptide of claim 11.

14. A recombinant host cell that expresses the isolated polypeptide of claim 11.

15. A method of making an isolated polypeptide comprising:
(a) culturing the recombinant host cell of claim 14 under conditions such that said polypeptide is expressed; and
(b) recovering said polypeptide.

16. The polypeptide produced by claim 15.

17. A method for preventing, treating, or ameliorating a medical condition, comprising administering to a mammalian subject a therapeutically effective amount of the polypeptide of claim 11 or the polynucleotide of claim 1.

18. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

19. A method of diagnosing a pathological condition or a susceptibility to a pathological condition in a subject comprising:
(a) determining the presence or amount of expression of the polypeptide of claim 11 in a biological sample; and
(b) diagnosing a pathological condition or a susceptibility to a pathological condition based on the presence or amount of expression of the polypeptide.

20. A method for identifying a binding partner to the polypeptide of claim 11 comprising:
(a) contacting the polypeptide of claim 11 with a binding partner; and
(b) determining whether the binding partner effects an activity of the polypeptide.

21. The gene corresponding to the cDNA sequence of SEQ ID NO:Y.

22. A method of identifying an activity in a biological assay, wherein the method comprises:
(a) expressing SEQ ID NO:X in a cell;
(b) isolating the supernatant;
(c) detecting an activity in a biological assay; and
(d) identifying the protein in the supernatant having the activity.

23. The product produced by the method of claim 20.
